(19) United States
(12) Patent Application Publication
(10) Pub. No.: US 2026/0347451 A1
(43) Pub. Date: Nov. 5, 2026

(54) ANTI-AMYLOID BETA PEPTIDE ANTIBODIES AND METHODS OF USE THEREOF

(71) Applicant: Alector LLC, South San Francisco, CA (US)

(72) Inventors: Arnon ROSENTHAL, Woodside, CA (US); Thunga BIENLY, Daly City, CA (US); Eric BROWN, South San Francisco, CA (US); Belvin GONG, Pleasanton, CA (US); Dominik HADDAD, Belmont, CA (US); Hua LONG, San Carlos, CA (US); Tarangsri NIVITCHANYONG, Castro Valley, CA (US); Lu SHAN, Belmont, CA (US); Maya SOKOLOVSKY, Alamo, CA (US); Jiahong SUN, San Mateo, CA (US); Margaret L. TANG, Mountain View, CA (US); Raymond Ka-Hang TONG, South San Francisco, CA (US); Angie Grace YEE, San Francisco, CA (US)

(21) Appl. No.: 19/542,026

(22) Filed: Feb. 17, 2026

Related U.S. Application Data
(60) Provisional application No. 63/759,812, filed on Feb. 18, 2025.
(60) Provisional application No. 63/801,714, filed on May 7, 2025.
(60) Provisional application No. 63/819,343, filed on Jun. 6, 2025.
(60) Provisional application No. 63/884,900, filed on Sep. 19, 2025.
(60) Provisional application No. 63/911,900, filed on Nov. 5, 2025.
(60) Provisional application No. 63/979,679, filed on Feb. 10, 2026.

(51) Int. Cl. C07K 16/18; A61K 9/00; A61K 39/00; A61P 25/28; C07K 16/28

(52) U.S. Cl. CPC C07K 16/18; A61K 9/0019; A61P 25/28; C07K 16/2881; C07K 16/2896; A61K 2039/505; A61K 2039/54; C07K 2317/31; C07K 2317/52; C07K 2317/565; C07K 2317/622; C07K 2317/70

(57) ABSTRACT

The present disclosure relates to anti-amyloid beta peptide antibodies that specifically bind N-terminal truncated pyroglutamate amyloid beta peptide (pyroGlu3 Abeta (Aβ)), multi-specific binding proteins comprising a pyroGlu3 Abeta (Aβ) antigen-binding domain and a blood brain barrier (BBB) antigen-binding domain that specifically binds to a BBB target selected from human transferrin receptor (TfR) or human CD98 heavy chain (CD98hc), and uses (e.g., therapeutic uses) thereof.

### CROSS-REFERENCE TO RELATED APPLICATIONS

This application claims the priority benefit of U.S. Provisional Appl. No. 63/759,812, filed Feb. 18, 2025; U.S. Provisional Appl. No. 63/801,714, filed May 7, 2025; U.S. Provisional Appl. No. 63/819,343, filed Jun. 6, 2025; U.S. Provisional Appl. No. 63/884,900, filed Sep. 19, 2025; U.S. Provisional Appl. No. 63/911,900, filed Nov. 5, 2025; and U.S. Provisional Appl. No. 63/979,679, filed Feb. 10, 2025; each of which is herein incorporated by reference in its entirety.

### REFERENCE TO SEQUENCE LISTING SUBMITTED ELECTRONICALLY

The content of the electronically submitted sequence listing (Name: 4503_0380006_SequenceListing_ST26.xml; Size: 348,066 bytes; and Date of Creation: Feb. 17, 2026) is herein incorporated by reference in its entirety.

### FIELD OF THE PRESENT DISCLOSURE

The present disclosure relates to anti-amyloid beta peptide antibodies that specifically bind N-terminal truncated pyroglutamate amyloid beta peptide (pyroGlu3 Aβ), multi-specific binding proteins comprising a pyroGlu3 Aβ antigen-binding domain and a blood brain barrier (BBB) antigen-binding domain that specifically binds to a BBB target selected from human transferrin receptor (TfR) or human CD98 heavy chain (CD98hc), and uses (e.g., therapeutic uses) thereof.

### BACKGROUND

Amyloid beta (Aβ, Abeta, or A-beta) is formed by the proteolytic cleavage of a larger glycoprotein called amyloid precursor protein (APP). APP is an integral membrane protein expressed in many tissues, but especially in neuron synapses. APP is cleaved by g-secretase to release the Aβ peptide, which encompasses peptides ranges from 37-49 amino acid residues. Aβ monomers aggregate into several types of higher order structures, such as oligomers, protofibrils, and amyloid fibrils. Amyloid oligomers are soluble and may spread throughout the brain, while amyloid fibrils are larger, insoluble, and can further aggregate to form amyloid deposits or plaques. Amyloid deposits found in brains of human patients include a heterogeneous mixture of Aβ peptides, some of which include N-terminal truncations and further may include N-terminal modifications, such as an N-terminal pyroglutamate residue (pGlu).

Amyloid plaques found in human patients include a heterogeneous mixture of Aβ peptides. PyroGlu3 Abeta (also referred to as pyroGlu3 Aβ, N3pE Aβ, N3pGlu Aβ, Aβ pE3-42) is a truncated form of Aβ peptide and is found only in amyloid plaques. N3pE Aβ lacks the first two amino acid residues at the N-terminus of human Aβ and has a pyroglutamate which is derived from glutamic acid at the third amino acid position of Aβ. Although pyroGlu3 Aβ is a minor component of the deposited Aβ in the brain, studies suggest that pyroGlu3 Aβ has aggressive aggregation properties and accumulates early in the deposition cascade. Passive immunization by long term chronic administration of antibodies against Aβ found in plaques, including pyroGlu3 Aβ, has been shown to disrupt the Aβ aggregates and promote the clearance of plaques in the brain.

Anti-Ap antibodies, including anti-pyroGlu3 Aβ antibodies, have been previously described. See, e.g., U.S. Pat. No. 7,122,374, WO2002/046237, WO2004/080419, WO2012/021469, WO2012/021475, WO2017/123517, WO2017/160555, WO2018/005282, WO2018/031361, WO2018/194951, WO2022/150735, WO2022/192636, WO2022/251048, WO2024/020470, WO2024/107683, WO2003/070760, WO2014/056816, WO2015/120280, WO2014/089500, WO2016/087944, WO2017/211827, WO2019/040612, WO2021/081101, WO2022/261026, WO2023/081194, WO2020/023530, WO2021/186245, WO2023/111618, WO2024/118665, WO2024/206161, WO2023/170295, WO2023/170291, WO2023/170290, WO2018/011353, and WO2024/133925.

However, such anti-pyroGlu3 Aβ antibody therapies intended for central nervous system (CNS) disorders may be limited in their effectiveness due to their limited ability to cross the blood brain barrier (BBB). Therefore, there is a need for novel anti-pyroGlu3 Aβ therapies having improved delivery across the BBB.

### SUMMARY OF THE PRESENT DISCLOSURE

Provided herein is an isolated antibody or antigen-binding fragment thereof that specifically binds to human N-terminal truncated pyroglutamate amyloid beta peptide (pyroGlu3 Abeta) and comprises the heavy chain variable region (VH) complementarity determining region (CDR) 1, VH CDR2, VH CDR3 and light chain variable region (VL) CDR1, CDR2, and CDR3 sequences of: SEQ ID NOs: 4, 5, 6, 21, 22, and 23, respectively; SEQ ID NOs: 7, 8, 9, 24, 25, and 26, respectively; SEQ ID NOs: 10, 11, 12, 27, 28, and 29, respectively; SEQ ID NOs: 13, 14, 15, 24, 30, and 31, respectively; SEQ ID NOs: 16, 17, 18, 32, 33, and 34, respectively; SEQ ID NOs: 4, 5, 6, 21, 22, and 23, respectively; SEQ ID NOs: 7, 8, 9, 24, 25, and 26, respectively; SEQ ID NOs: 10, 19, 12, 27, 35, and 29, respectively; SEQ ID NOs: 13, 14, 15, 24, 30, and 31, respectively; or SEQ ID NOs: 16, 20, 18, 36, 33, and 34, respectively.

Also provided herein is an antibody or antigen-binding fragment thereof that specifically binds to human N-terminal truncated pyroglutamate amyloid beta peptide (pyroGlu3 Abeta) selected from the group consisting of: (a) an antibody comprising a VH comprising the amino acid sequence of SEQ ID NO: 37 and a VL comprising the amino acid sequence of SEQ ID NO: 38; (b) an antibody comprising a VH comprising the amino acid sequence of SEQ ID NO: 39 and a VL comprising the amino acid sequence of SEQ ID NO: 40; (c) an antibody comprising a VH comprising the amino acid sequence of SEQ ID NO: 41 and a VL comprising the amino acid sequence of SEQ ID NO: 42; (d) an antibody comprising a VH comprising the amino acid sequence of SEQ ID NO: 43 and a VL comprising the amino acid sequence of SEQ ID NO: 44; (e) an antibody comprising a VH comprising the amino acid sequence of SEQ ID NO: 45 and a VL comprising the amino acid sequence of SEQ ID NO: 46; (f) an antibody comprising a VH comprising the amino acid sequence of SEQ ID NO: 47 and a VL comprising the amino acid sequence of SEQ ID NO: 48; (g) an antibody comprising a VH comprising the amino acid sequence of SEQ ID NO: 49 and a VL comprising the amino acid sequence of SEQ ID NO: 50; (h) an antibody comprising a VH comprising the amino acid sequence of SEQ ID NO: 51 and a VL comprising the amino acid sequence of SEQ ID NO: 52; (i) an antibody comprising a VH comprising the amino acid sequence of SEQ ID NO: 43 and a VL comprising the amino acid sequence of SEQ ID NO: 53; (j) an antibody comprising a VH comprising the amino acid sequence of SEQ ID NO: 54 and a VL comprising the amino acid sequence of SEQ ID NO: 55; and (k) an antibody that binds to the same human N-terminal truncated pyroglutamate amyloid beta peptide (pyroGlu3 Abeta) epitope as the antibody or antigen-binding fragment thereof of any one of (a)-(j).

Also provided herein is an antibody or antigen-binding fragment thereof that specifically binds to human N-terminal truncated pyroglutamate amyloid beta peptide (pyroGlu3 Abeta) selected from the group consisting of: (a) an antibody comprising a VH comprising the amino acid sequence of SEQ ID NO: 37 and a VL comprising the amino acid sequence of SEQ ID NO: 38; (b) an antibody comprising a VH comprising the amino acid sequence of SEQ ID NO: 39 and a VL comprising the amino acid sequence of SEQ ID NO: 40; (c) an antibody comprising a VH comprising the amino acid sequence of SEQ ID NO: 41 and a VL comprising the amino acid sequence of SEQ ID NO: 42; (d) an antibody comprising a VH comprising the amino acid sequence of SEQ ID NO: 43 and a VL comprising the amino acid sequence of SEQ ID NO: 44; (e) an antibody comprising a VH comprising the amino acid sequence of SEQ ID NO: 45 and a VL comprising the amino acid sequence of SEQ ID NO: 46; (f) an antibody comprising a VH comprising the amino acid sequence of SEQ ID NO: 47 and a VL comprising the amino acid sequence of SEQ ID NO: 48; (g) an antibody comprising a VH comprising the amino acid sequence of SEQ ID NO: 49 and a VL comprising the amino acid sequence of SEQ ID NO: 50; (h) an antibody comprising a VH comprising the amino acid sequence of SEQ ID NO: 51 and a VL comprising the amino acid sequence of SEQ ID NO: 52; (i) an antibody comprising a VH comprising the amino acid sequence of SEQ ID NO: 43 and a VL comprising the amino acid sequence of SEQ ID NO: 53; (j) an antibody comprising a VH comprising the amino acid sequence of SEQ ID NO: 54 and a VL comprising the amino acid sequence of SEQ ID NO: 55; and (k) an antibody that competitively inhibits the binding of any one of (a)-(j) to human N-terminal truncated pyroglutamate amyloid beta peptide (pyroGlu3 Abeta).

In some aspects, the antibody or antigen-binding fragment disclosed herein binds at least 2-fold more strongly to human pyro-Glu3-24 Abeta polypeptide than to human non-modified E3-24 Ab polypeptide. In some aspects, the antibody or antigen-binding fragment thereof binds about 2-fold to about 5-fold more strongly to human pyro-Glu3-24 Ab polypeptide than to human non-modified E3-24 Abeta polypeptide.

In some aspects, the antibody or antigen-binding fragment disclosed herein binds at least 40-fold more strongly to human pyro-Glu3-24 Abeta polypeptide than to human non-modified E3-40 Abeta polypeptide or wherein the antibody or antigen-binding fragment thereof binds about 40-fold to about 65-fold more strongly to human pyro-Glu3-24 Abeta polypeptide than to human non-modified E3-40 Abeta polypeptide.

In some aspects, the the antibody or antigen-binding fragment promotes phagocytosis of Abeta plaques, optionally wherein 0.1 μg/mL increases phagocytosis of Abeta plaques by about 50% as compared to a control.

In some aspects, the antibody or antigen-binding fragment promotes uptake of pyroGlu3 Abeta by microglia in the CNS.

In some aspects, the antibody or antigen-binding fragment comprises a heavy chain variable region (VH) that is at least 90%, at least 95%, at least 97%, at least 99%, or 100% identical to an amino acid sequence selected from any one of SEQ ID NOs: 37, 39, 41, 43, 45, 47, 49, 51, and 54.

In some aspects, the antibody or antigen-binding fragment comprises a light chain variable region (VL) that is at least 90%, at least 95%, at least 97%, at least 99%, or 100% identical to an amino acid sequence selected from any one of SEQ ID NOs: 38, 40, 42, 44, 46, 48, 50, 52, 53, and 55.

In some aspects, the antibody or antigen-binding fragment comprises a VH and a VL, wherein the VH and VL comprise amino acid sequences at least 80%, at least 85%, at least 90%, at least 95%, at least 99%, or 100% identical to the amino acid sequences of: (a) SEQ ID NOs: 37 and 38, respectively; (b) SEQ ID NOs: 39 and 40, respectively; (c) SEQ ID NOs: 41 and 42, respectively; (d) SEQ ID NOs: 43 and 44, respectively; (e) SEQ ID NOs: 45 and 46, respectively; (f) SEQ ID NOs: 47 and 48, respectively; (g) SEQ ID NOs: 49 and 50, respectively; (h) SEQ ID NOs: 51 and 52, respectively; (i) SEQ ID NOs: 43 and 53, respectively; or (j) SEQ ID NOs: 54 and 55, respectively.

In some aspects, the antibody or antigen-binding fragment comprises a heavy chain variable region and/or a light chain variable region comprising the amino acid sequences of: (a) SEQ ID NOs: 37 and 38, respectively; (b) SEQ ID NOs: 39 and 40, respectively; (c) SEQ ID NOs: 41 and 42, respectively; (d) SEQ ID NOs: 43 and 44, respectively; (e) SEQ ID NOs: 45 and 46, respectively; (f) SEQ ID NOs: 47 and 48, respectively; (g) SEQ ID NOs: 49 and 50, respectively; (h) SEQ ID NOs: 51 and 52, respectively; (i) SEQ ID NOs: 43 and 53, respectively; or (j) SEQ ID NOs: 54 and 55, respectively.

In some aspects, the antibody or antigen-binding fragment comprises a heavy chain constant region and a light chain constant region.

In some aspects, the heavy chain constant region is an isotype selected from the group consisting of human IgG1, IgG2, IgG3, and IgG4 isotypes.

In some aspects, the antibody or antigen-binding fragment comprises a heavy chain constant region and a light chain constant region, In some aspects, the heavy chain constant region is a human IgG₁ heavy chain constant region, and the light chain constant region is a human IgGκ light chain constant region.

In some aspects, the antibody or antigen-binding fragment is a monoclonal antibody.

In some aspects, the antibody or antigen-binding fragment thereof is a murine, chimeric, humanized, or human antibody or antigen-binding fragment thereof.

In some aspects, the antibody or antigen-binding fragment thereof is a full length antibody

In some aspects, the antibody or antigen-binding fragment thereof is an antigen binding fragment. In some aspects, the antigen-binding fragment is a Fab, Fab′, F(ab′)2, single chain Fv (scFv), disulfide linked Fv, V-NAR domain, IgNar, intrabody, IgGΔCH2, minibody, F(ab′)3, tetrabody, triabody, diabody, single-domain antibody, DVD-Ig, Fcab, mAb2, (scFv)2, or scFv-Fc.

In some aspects, the antibody or antigen-binding fragment thereof is monospecific.

In some aspects, the antibody or antigen-binding fragment thereof is bispecific.

In some aspects, the antibody or antigen-binding fragment thereof is multispecific.

In some aspects, the antibody or antigen-binding fragment thereof further comprises an antigen-binding domain that binds to blood brain barrier (BBB) target.

Provided herein is a complex comprising the antibody or antigen-binding fragment disclosed herein and an antigen-binding domain that binds to a BBB target.

In some aspects, the antibody or antigen-binding fragment or complex discloses herein, further comprises a detectable label.

In some aspects, the BBB target is human transferrin receptor (TfR) or human CD98 heavy chain (CD98hc).

In some aspects, (a) the BBB target is human TfR and wherein the antibody or antigen-binding fragment or complex comprises a human TfR antigen-binding domain comprising heavy chain variable region (VH) complementarity determining region (CDR) 1, VH CDR2, VH CDR3 and light chain variable region (VL) CDR1, CDR2, and CDR3 sequences comprising the amino acid sequences of:
SEQ ID NOs: 56, 57, 58, 66, 67, and 68, respectively;
SEQ ID NOs: 56, 57, 58, 69, 67, and 68, respectively;
SEQ ID NOs: 56, 57, 59, 69, 67, and 68, respectively;
SEQ ID NOs: 56, 57, 60, 69, 67, and 68, respectively;
SEQ ID NOs: 56, 57, 59, 70, 67, and 68, respectively;
SEQ ID NOs: 56, 57, 59, 71, 67, and 68, respectively;
SEQ ID NOs: 56, 57, 59, 72, 67, and 68, respectively;
SEQ ID NOs: 56, 57, 59, 73, 67, and 68, respectively;
SEQ ID NOs: 56, 57, 61, 69, 67, and 68, respectively;
SEQ ID NOs: 62, 63, 64, 74, 75, and 76, respectively;
SEQ ID NOs: 62, 65, 64, 77, 75, and 76, respectively;
SEQ ID NOs: 62, 63, 64, 78, 75, and 76, respectively;
SEQ ID NOs: 62, 63, 64, 79, 75, and 76, respectively;
SEQ ID NOs: 62, 65, 64, 80, 75, and 76, respectively;
SEQ ID NOs:56, 57, 59, 66, 67, and 68, respectively;
SEQ ID NOs:62, 275, 64, 276, 75, and 277, respectively; or
SEQ ID NOs:62, 275, 64, 77, 75, and 76, respectively; or
   (b) the BBB target is human CD98hc and wherein the antibody or antigen-binding fragment or complex comprises a human CD98hc antigen-binding domain comprising heavy chain variable region (VH) complementarity determining region (CDR) 1, VH CDR2, VH CDR3 and light chain variable region (VL) CDR1, CDR2, and CDR3 sequences comprising the amino acid sequences of:
SEQ ID NOs: 98, 99, 100, 122, 123, and 124, respectively;
SEQ ID NOs: 98, 101, 100, 122, 123, and 124, respectively;
SEQ ID NOs: 98, 102, 100, 122, 123, and 124, respectively;
SEQ ID NOs: 98, 103, 100, 122, 123, and 124, respectively;
SEQ ID NOs:98, 104, 100, 122, 123, and 124, respectively;
SEQ ID NOs: 98, 105, 100, 122, 123, and 124, respectively;
SEQ ID NOs: 98, 106, 100, 122, 123, and 124, respectively;
SEQ ID NOs: 98, 107, 100, 122, 123, and 124, respectively;
SEQ ID NOs: 98, 108, 100, 122, 123, and 124, respectively;
SEQ ID NOs: 98, 109, 100, 122, 123, and 124, respectively;
SEQ ID NOs: 98, 110, 100, 122, 123, and 124, respectively;
SEQ ID NOs: 98, 99, 100, 125, 123, and 124, respectively;
SEQ ID NOs: 98, 99, 100, 126, 123, and 124, respectively;
SEQ ID NOs: 98, 99, 100, 127, 123, and 124, respectively;
SEQ ID NOs: 98, 99, 100, 128, 123, and 124, respectively;
SEQ ID NOs: 98, 99, 100, 129, 123, and 124, respectively;
SEQ ID NOs: 98, 99, 100, 130, 123, and 124, respectively;
SEQ ID NOs: 98, 99, 100, 131, 123, and 124, respectively;
SEQ ID NOs: 98, 106, 100, 127, 123, and 124, respectively;
SEQ ID NOs: 98, 111, 100, 122, 123, and 124, respectively;
SEQ ID NOs: 98, 99, 112, 122, 123, and 124, respectively;
SEQ ID NOs: 98, 99, 113, 122, 123, and 124, respectively;
SEQ ID NOs: 98, 99, 114, 122, 123, and 124, respectively;
SEQ ID NOs: 98, 99, 115, 122, 123, and 124, respectively;
SEQ ID NOs: 98, 99, 116, 122, 123, and 124, respectively;
SEQ ID NOs: 98, 99, 117, 122, 123, and 124, respectively;
SEQ ID NOs: 98, 99, 118, 122, 123, and 124, respectively;
SEQ ID NOs: 98, 99, 119, 122, 123, and 124, respectively;
SEQ ID NOs: 98, 99, 120, 122, 123, and 124, respectively;
SEQ ID NOs: 98, 99, 100, 132, 123, and 124, respectively;
SEQ ID NOs: 98, 99, 100, 133, 123, and 124, respectively;
SEQ ID NOs: 98, 99, 100, 134, 123, and 124, respectively;
SEQ ID NOs: 98, 99, 100, 135, 123, and 124, respectively;
SEQ ID NOs: 98, 99, 100, 136, 123, and 124, respectively;
SEQ ID NOs: 98, 99, 100, 137, 123, and 124, respectively;
SEQ ID NOs: 98, 99, 100, 122, 123, and 138, respectively;
SEQ ID NOs: 98, 99, 100, 122, 123, and 139, respectively;
SEQ ID NOs: 98, 99, 100, 122, 123, and 140, respectively;
SEQ ID NOs: 98, 99, 100, 122, 123, and 141, respectively;
SEQ ID NOs: 98, 99, 100, 122, 123, and 142, respectively;
SEQ ID NOs: 98, 99, 100, 122, 123, and 143, respectively;
SEQ ID NOs: 98, 99, 100, 122, 123, and 144, respectively;
SEQ ID NOs: 98, 99, 100, 122, 123, and 145, respectively;
SEQ ID NOs: 98, 99, 100, 122, 123, and 146, respectively;
SEQ ID NOs: 98, 99, 100, 122, 123, and 147, respectively;
SEQ ID NOs: 121, 99, 100, 122, 123, and 124, respectively; or
SEQ ID NOs: 121, 99, 100, 122, 123, and 147, respectively.

In some aspects, (a) the BBB target is human TfR and wherein the human TfR antigen-binding domain comprises a VH and a VL, wherein the VH and VL comprise amino acid sequences at least 80%, at least 85%, at least 90%, at least 95%, at least 99%, or 100% identical to the amino acid sequences of:
SEQ ID NOs: 81 and 82, respectively;
SEQ ID NOs: 81 and 83, respectively;
SEQ ID NOs: 84 and 83, respectively;
SEQ ID NOs: 85 and 83, respectively;
SEQ ID NOs: 84 and 86, respectively;
SEQ ID NOs: 84 and 87, respectively;
SEQ ID NOs: 84 and 88, respectively;
SEQ ID NOs: 84 and 89, respectively;
SEQ ID NOs: 90 and 83, respectively;
SEQ ID NOs: 91 and 92, respectively;
SEQ ID NOs: 93 and 94, respectively;
SEQ ID NOs: 91 and 95, respectively;
SEQ ID NOs: 91 and 96, respectively;
SEQ ID NOs: 93 and 97, respectively;
SEQ ID NOs: 269 and 270, respectively;
SEQ ID NOs: 271 and 272, respectively; or
SEQ ID NOs: 273 and 274, respectively; or
   (b) the BBB target is human CD98hc and wherein the human CD98hc antigen-binding domain comprises a VH and a VL, wherein the VH and VL comprise amino acid sequences at least 80%, at least 85%, at least 90%, at least 95%, at least 99%, or 100% identical to the amino acid sequences of:
SEQ ID NOs: 148 and 149, respectively;
SEQ ID NOs: 150 and 149, respectively;
SEQ ID NOs: 151 and 149, respectively;
SEQ ID NOs: 152 and 149, respectively;
SEQ ID NOs: 153 and 149, respectively;
SEQ ID NOs: 154 and 149, respectively;
SEQ ID NOs: 155 and 149, respectively;
SEQ ID NOs: 156 and 149, respectively;
SEQ ID NOs: 157 and 149, respectively;
SEQ ID NOs: 158 and 149, respectively;
SEQ ID NOs: 159 and 149, respectively;
SEQ ID NOs: 148 and 160, respectively;
SEQ ID NOs: 148 and 161, respectively;
SEQ ID NOs: 148 and 162, respectively;
SEQ ID NOs: 148 and 163, respectively;
SEQ ID NOs: 148 and 164, respectively;
SEQ ID NOs: 148 and 165, respectively;
SEQ ID NOs: 148 and 166, respectively;
SEQ ID NOs: 155 and 162, respectively;
SEQ ID NOs: 167 and 149, respectively;
SEQ ID NOs: 168 and 149, respectively;
SEQ ID NOs: 169 and 149, respectively;
SEQ ID NOs: 170 and 149, respectively;
SEQ ID NOs: 171 and 149, respectively;
SEQ ID NOs: 172 and 149, respectively;
SEQ ID NOs: 173 and 149, respectively;
SEQ ID NOs: 174 and 149, respectively;
SEQ ID NOs: 175 and 149, respectively;
SEQ ID NOs: 176 and 149, respectively;
SEQ ID NOs: 148 and 177, respectively;
SEQ ID NOs: 148 and 178, respectively;
SEQ ID NOs: 148 and 179, respectively;
SEQ ID NOs: 148 and 180, respectively;
SEQ ID NOs: 148 and 181, respectively;
SEQ ID NOs: 148 and 182, respectively;
SEQ ID NOs: 148 and 183, respectively;
SEQ ID NOs: 148 and 184, respectively;
SEQ ID NOs: 148 and 185, respectively;
SEQ ID NOs: 148 and 186, respectively;
SEQ ID NOs: 148 and 187, respectively;
SEQ ID NOs: 148 and 188, respectively;
SEQ ID NOs: 148 and 189, respectively;
SEQ ID NOs: 148 and 190, respectively;
SEQ ID NOs: 148 and 191, respectively;
SEQ ID NOs: 148 and 192, respectively;
SEQ ID NOs: 193 and 194, respectively; or
SEQ ID NOs: 193 and 192, respectively

In some aspects, the human TfR antigen-binding domain comprises a disulfide staple.

In some aspects, the human TfR antigen-binding domain comprises the amino acid sequence of SEQ ID NO:202 or SEQ ID NO:203.

In some aspects, the antibody or antigen-binding fragment thereof that specifically binds to human N-terminal truncated pyroglutamate amyloid beta peptide (pyroGlu3 Abeta) comprises the VH CDR 1, VH CDR2, VH CDR3 and VL CDR1, CDR2, and CDR3 sequences of: SEQ ID NOs: 13, 14, 15, 24, 30, and 31, respectively; and the human TfR antigen-binding domain comprises VH CDR1, VH CDR2, VH CDR3 and VL CDR1, CDR2, and CDR3 sequences comprising the amino acid sequences of SEQ ID NOs: 56, 57, 59, 69, 67, and 68, respectively. In some aspects, the antibody or antigen-binding fragment thereof that specifically binds to human N-terminal truncated pyroglutamate amyloid beta peptide (pyroGlu3 Abeta) comprises the VH CDR 1, VH CDR2, VH CDR3 and VL CDR1, CDR2, and CDR3 sequences of: SEQ ID NOs: 13, 14, 15, 24, 30, and 31, respectively; and the human TfR antigen-binding domain comprises VH CDR1, VH CDR2, VH CDR3 and VL CDR1, CDR2, and CDR3 sequences comprising the amino acid sequences of SEQ ID NOs: 56, 57, 59, 72, 67, and 68, respectively. In some aspects, the antibody or antigen-binding fragment thereof that specifically binds to human N-terminal truncated pyroglutamate amyloid beta peptide (pyroGlu3 Abeta) comprises the VH CDR 1, VH CDR2, VH CDR3 and VL CDR1, CDR2, and CDR3 sequences of: SEQ ID NOs: 13, 14, 15, 24, 30, and 31, respectively; and the human TfR antigen-binding domain comprises VH CDR1, VH CDR2, VH CDR3 and VL CDR1, CDR2, and CDR3 sequences comprising the amino acid sequences of SEQ ID NOs: 56, 57, 59, 73, 67, and 68, respectively.

In some aspects, the antibody or antigen-binding fragment thereof that specifically binds to human N-terminal truncated pyroglutamate amyloid beta peptide (pyroGlu3 Abeta) comprises a VH and a VL comprising the amino acid sequences of SEQ ID NO:43 and 53 respectively; and the human TfR antigen-binding domain comprises a VH and a VL comprising the amino acid sequences of SEQ ID NOs: 84 and 83, respectively. In some aspects, the antibody or antigen-binding fragment thereof that specifically binds to human N-terminal truncated pyroglutamate amyloid beta peptide (pyroGlu3 Abeta) comprises a VH and a VL comprising the amino acid sequences of SEQ ID NO:43 and 53 respectively; and the human TfR antigen-binding domain comprises a VH and a VL comprising the amino acid sequences of SEQ ID NOs: 84 and 88, respectively. In some aspects, the antibody or antigen-binding fragment thereof that specifically binds to human N-terminal truncated pyroglutamate amyloid beta peptide (pyroGlu3 Abeta) comprises a VH and a VL comprising the amino acid sequences of SEQ ID NO:47 and 48 respectively; and the human TfR antigen-binding domain comprises a VH and a VL comprising the amino acid sequences of SEQ ID NOs: 84 and 89, respectively.

In some aspects, the (i) the antibody or antigen-binding fragment that binds to pyroGlu3 Abeta is a full-length antibody, a single-chain fragment variable (scFv), a VHH, or a Fab, and (ii) the anti-BBB antigen-binding domain is a single-chain fragment variable (scFv).

In some aspects, the (i) the antibody or antigen-binding fragment that binds to pyroGlu3 Abeta is a full-length antibody, a single-chain fragment variable (scFv), a VHH, or a Fab and (ii) the anti-BBB antigen-binding domain is a VHH.

In some aspects, the (i) the antibody or antigen-binding fragment that binds to pyroGlu3 Abeta is a full-length antibody, a single-chain fragment variable (scFv), a VHH, or a Fab and (ii) the anti-BBB antigen-binding domain is a Fab.

In some aspects, the antibody or antigen-binding fragment that binds to pyroGlu3 Abeta and the anti-BBB antigen-binding domain are connected by a linker. In some aspects, the linker connects the C-terminus of the heavy chain Fc of the antibody or antigen-binding fragment that binds to pyroGlu3 Abeta and the N-terminus of the anti-BBB antigen-binding domain. In some aspects, the linker comprises GGSGG (SEQ ID NO: 214). In some aspects, the antibody or antigen-binding fragment that binds to pyroGlu3 Abeta and the anti-BBB antigen-binding domain are directly connected. In some aspects, the antibody or antigen-binding fragment that binds to pyroGlu3 Abeta and the anti-BBB antigen-binding domain comprises the amino acid sequence of SEQ ID NO:280, SEQ ID NO:281, or SEQ ID NO:282.

In some aspects, the antibody or antigen-binding fragment or complex is bivalent.

In some aspects, the antibody or antigen-binding fragment or complex is trivalent, optionally the antibody or antigen-binding fragment or complex comprises (i) one antigen-binding domain that binds to the BBB target and two antigen-binding domains that bind to pyroGlu3 Abeta or (ii) two antigen-binding domains that bind to the BBB target and one antigen-binding domain that binds to pyroGlu3 Abeta.

In some aspects, the antibody or antigen-binding fragment or complex is tetravalent, optionally the antibody or antigen-binding fragment or complex comprises two antigen-binding domains that bind to the BBB target and two antigen-binding domains that bind to pyroGlu3 Abeta.

In some aspects, the antibody or antigen-binding fragment or complex described herein comprise the amino acid sequences of (a) SEQ ID NOs: 223, 224, and 225, (b) SEQ ID NOs: 226, 227, and 228; (c) SEQ ID NOs: 229, 230, and 231; (d) SEQ ID NOs: 241, 224, and 225; (e) SEQ ID NOs: 242, 224, and 225; (f) SEQ ID NOs: 243, 224, and 225; (g) SEQ ID NOs: 244, 224, and 225; (h) SEQ ID NOs 245, 224, and 225; (i) SEQ ID NOs: 246, 224, and 225; (j) SEQ ID NOs: 247, 224, and 225; (k) SEQ ID NOs: 248, 224, and 225; (1) SEQ ID NOs: 249, 224, and 225; (m) SEQ ID NOs: 250, 224, and 225; (n) SEQ ID NOs: 251, 230, and 231; (o) SEQ ID NOs: 252, 230, and 231; (p) SEQ ID NOs: 253, 230, and 231; (q) SEQ ID NOs: 254, 230, and 231; (r) SEQ ID NOs: 255, 230, and 231; (s) SEQ ID NOs: 256, 230, and 231; (t) SEQ ID NOs: 257, 230, and 231; (u) SEQ ID NOs: 258, 230, and 231; (v) SEQ ID NOs: 259, 230, and 231; (w) SEQ ID NOs: 260, 230, and 231; (x) SEQ ID NOs: 261, 230, and 231; (y) SEQ ID NOs: 262, 230, and 231; (z) SEQ ID NOs: 263, 230, and 231; (aa) SEQ ID NOs: 264, 230, and 231; (bb) SEQ ID NOs: 265, 230, and 231; SEQ ID NOs: 266, 230, and 231; (cc) SEQ ID NOs: 278, 224, and 225; or (dd) SEQ ID NOs: 279, 230, and 231.

In some aspects, the antibody or antigen-binding fragment or complex enhances brain uptake of the antibody or antigen-binding fragment thereof that specifically binds to human N-terminal truncated pyroGlu3 Abeta in one or more of the frontal cortex, hippocampus, and thalamus of an animal.

In some aspects, the antibody or antigen-binding fragment or complex increases the concentration of the antibody or antigen-binding fragment thereof that specifically binds to human N-terminal truncated pyroGlu3 Abeta in the CSF of an animal.

Provided herein is an isolated polynucleotide comprising a nucleic acid molecule encoding the heavy chain variable region or heavy chain of the antibody or antigen-binding fragment thereof described herein.

In some aspects, the nucleic acid molecule encodes the VH of any one of SEQ ID NOs: 37, 39, 41, 43, 45, 47, 49, 51, and 54.

Provided herein is an isolated polynucleotide comprising a nucleic acid molecule encoding the light chain variable region or light chain of the antibody or antigen-binding fragment thereof described herein.

In some aspects, the nucleic acid molecule encodes the VL of any one of SEQ ID NOs: 38, 40, 42, 44, 46, 48, 50, 52, 53, and 55.

Provided herein is an isolated polynucleotide comprising a nucleic acid molecule encoding the heavy chain variable region or heavy chain of the antibody or antigen-binding fragment thereof described herein and the light chain variable region or light chain of the antibody or antigen-binding fragment thereof described herein.

Provided herein is an isolated vector comprising the polynucleotide described herein.

Provided herein is a host cell comprising (a) the polynucleotide described herein (b) the vector described herein, or (c) a first vector comprising the polynucleotide described herein and a second vector comprising the polynucleotide described herein.

In some aspects, the host cell is selected from the group consisting of E. coli, Pseudomonas, Bacillus, Streptomyces, yeast, CHO, YB/20, NS0, PER-C6, HEK-293T, NIH-3T3, HeLa, BHK, Hep G2, SP2/0, R1.1, B-W, L-M, COS 1, COS 7, BSC1, BSC40, BMT10 cell, plant cell, insect cell, and human cell in tissue culture, optionally wherein the host cell is a HEK-293 cell.

Provided herein is a method of producing an antibody or antigen-binding fragment thereof that binds to human pyroGlu3 Abeta comprising culturing the host cell described herein so that the nucleic acid molecule is expressed and the antibody or antigen-binding fragment thereof is produced, optionally the method further comprises isolating the antibody or antigen-binding fragment thereof from the culture.

Provided herein is an isolated antibody or antigen-binding fragment thereof that specifically binds to human pyroGlu3 Abeta and is encoded by the polynucleotide described herein, encoded by the vector described herein, or produced by the method described herein.

Provided herein is a pharmaceutical composition comprising the antibody or antigen-binding fragment thereof or complex described herein and a pharmaceutically acceptable excipient.

Provided herein is a method of promoting phagocytosis of Abeta plaques comprising contacting a composition comprising Abeta plaques and macrophages and/or microglia with the antibody or antigen binding fragment thereof or complex described herein or the pharmaceutical composition described herein.

In some aspects, the contacting is in vitro. In some aspects, the contact is in a subject.

Provided herein is a method of slowing the progression of neurodegeneration in a subject in need thereof, the method comprising administering to the subject a therapeutically effective amount of the antibody or antigen binding fragment thereof or complex described herein or the pharmaceutical composition described herein. As used herein “slowing the progression” of neurodegeneration refers to decreasing the speed at which degeneration occurs as compared to a subject who did not receive the administration. In some aspects, the administration provided herein can prevent or delay conversion from mild cognitive impairment (MCI) to Alzheimer's disease.

Provided herein is a method of reducing neurodegeneration in a subject in need thereof, the method comprising administering to the subject a therapeutically effective amount of the antibody or antigen binding fragment thereof or complex described herein or the pharmaceutical composition described herein.

Provided herein is a method of decreasing cognitive decline in a subject in need thereof, the method comprising administering to the subject a therapeutically effective amount of the antibody or antigen binding fragment thereof or complex described herein or the pharmaceutical composition described herein.

In some aspects, the subject has Alzheimer's disease.

Provided herein is a method of treating a neurodegenerative disease in a subject, the method comprising administering to the subject a therapeutically effective amount of the antibody or antigen binding fragment thereof or complex described herein or the pharmaceutical composition described herein.

Provided herein is a method of preventing a neurodegenerative disease in a subject, the method comprising administering to the subject a therapeutically effective amount of the antibody or antigen binding fragment thereof or complex described herein or the pharmaceutical composition described herein.

In some aspects, the neurodegenerative disease is Alzheimer's disease, early Alzheimer's disease, or mild cognitive impairment (MCI).

Provided herein is a method of treating Alzheimer's disease in a subject, the method comprising administering to the subject a therapeutically effective amount of the antibody or antigen binding fragment thereof or complex described herein or the pharmaceutical composition described herein.

Provided herein is a method of preventing Alzheimer's disease in a subject, the method comprising administering to the subject a therapeutically effective amount of the antibody or antigen binding fragment thereof or complex described herein or the pharmaceutical composition described herein.

In some aspects, the Alzheimer's disease is familial Alzheimer's disease.

In some aspects, a method provided herein comprises administering to the subject a therapeutically effective amount of an antibody or antigen binding fragment thereof or complex described herein or the pharmaceutical described herein, wherein the BBB target is TfR.

In some aspects, the administration results in minimal hematological adverse events. In some aspects, the administration results in no adverse or long-term reduction in reticulocyte count in the subject. In some aspects, the administration results in no adverse or long-term reduction in red blood cell count in the subject. In some aspects, the administration results in no adverse or long-term reduction in hematocrit in the subject. In some aspects, the administration results in no adverse or long-term reduction in hemoglobin level in the subject. In some aspects, the administration results in rapid clearance or reduction of Abeta in the brain of the subject. In some aspects, the administration requires less frequent monitoring of Abeta levels or no monitoring of Abeta levels in the brain of the subject as compared to the monitoring required of other anti-Abeta antibody therapies. In some aspects, the subject has not been APOE genotyped prior to the administration. In some aspects, the administration is associated with less ARIA as compared to the ARIA associated with other anti-Abeta antibody therapies.

In some aspects, the administration is intravenous or subcutaneous.

In some aspects, a method provided herein further comprises administering an additional active agent. In some aspects, the additional active agent is one or more of a tau-targeting agent, an ApoER2-targeting agent, an ApoE4-targeting agent, an ApoE2-targeting agent, a NLRP3-targeting agent, a GSK3B targeting agent, and a GLP-1 targeting agent.

Provided herein is an antibody or antigen-binding fragment thereof or complex described herein or the pharmaceutical composition described herein for use in the method described herein or for use in the manufacture of a medicament for use in the method described herein.

Provided herein is the use of the antibody or antigen-binding fragment thereof or complex described herein or the pharmaceutical composition described herein in the method described herein.

### BRIEF DESCRIPTION OF THE DRAWINGS

FIG. 1A (FIG. 1A) shows an exemplary 2+1 multi-specific binding protein containing an scFv that binds to a BBB target (e.g., TfR or CD98hc) linked to a C-terminus of the heavy chain (with an Fc “hole” mutation) of a full-length antibody that binds to pyroGlu3 ABeta (Aβ).

FIG. 1B (FIG. 1B) shows an exemplary 2+1 multi-specific binding protein containing an scFv that binds to a BBB target (e.g., TfR or CD98hc) linked to a C-terminus of the heavy chain (with an Fc “knob” mutation) of a full-length antibody that binds to pyroGlu3 Abeta (Aβ).

FIG. 2 (FIG. 2) shows an exemplary 2+2 multi-specific binding protein with two copies of an scFv that binds to a BBB target (e.g., TfR or CD98hc), wherein one copy is linked to the C-terminus of each heavy chain of a full-length antibody that binds to pyroGlu3 Abeta (Aβ).

FIG. 3 (FIG. 3) shows exemplary multi-specific binding proteins with heterodimeric Fc regions (e.g., wherein one polypeptide of the Fc region contains a knob mutation and the other polypeptide of the Fc region contains a hole mutation). FIG. 3 includes (i) an exemplary 2+1 multi-specific binding protein with an scFv that binds to a BBB target (e.g., TfR or CD98hc) linked to the C-terminus of a heavy chain of an antibody that binds to pyroGlu3 Abeta (Aβ); (ii) an exemplary 1+1 multi-specific binding protein wherein a Fab of a full-length antibody that binds to pyroGlu3 Abeta (Aβ) is replaced with an scFv that binds to a BBB target (e.g., TfR or CD98hc); and (iii) an exemplary 1+1 multi-specific binding protein where one VHH that binds to a BBB target (e.g., TfR or CD98hc) is linked to the N-terminus of one polypeptide of an Fc region and one VHH that binds to pyroGlu3 Abeta (Aβ) is linked to the N-terminus of a second polypeptide of the Fc region.

FIG. 4 (FIG. 4) shows ex vivo plaque phagocytosis by human macrophages 24 h after addition of 0.1 g/mL anti-pyroGlu3 Abeta (Aβ) antibodies of the present disclosure. Anti-pyroGlu3 Abeta (Aβ) antibodies AB-6, AB-7, AB-8, AB-9, and AB-10 induced a similar extent of plaque phagocytosis as anti-Abeta (Aβ) antibodies donanemab and remternetug, compared to isotype control. One way ANOVA with Tukey's multiple comparisons test. ****: p<0.0001.

FIG. 5 (FIG. 5) shows uptake of pyroGlu3 Abeta peptide by iPSC-derived microglia following addition of 0.1 g/mL of anti-pyroGlu3 Abeta (Aβ) antibodies of the present disclosure. Anti-pyroGlu3 Abeta (Aβ) antibodies AB-6, AB-7, AB-8, AB-9, and AB-10 displayed similar uptake of pyroGlu3-24 Abeta (Aβ) peptide as observed with anti-Abeta (Aβ) antibodies donanemab and remternetug. Anti-pyroGlu3 Abeta (Aβ) antibodies AB-3 and AB-8 showed a smaller increase of pyroGlu3-24 Abeta (Aβ) peptide uptake compared to other antibodies tested in this assay. One way ANOVA with Tukey's multiple comparisons test. **: p<0.005; ***: p<0.001; ****: p<0.0001.

FIG. 6 (FIG. 6) shows representative images of target engagement with amyloid plaque of anti-pyroGlu3 Abeta (Aβ) antibodies of the present disclosure in 6-month-old 5XFAD mice 4 days post administration of 50 mg/kg i.v. dose. Anti-pyroGlu3-24 Abeta (Aβ) antibodies AB-6, AB-7, and AB-8 show binding to amyloid plaques in vivo.

FIG. 7 (FIG. 7) shows quantification of huIgG positive area (i.e., amyloid plaque) in brains after dosing aged 5XFAD mice with 50 mg/kg of anti-pyroGlu3 Abeta (Aβ) antibodies of the present disclosure for 4 days.

FIG. 8 (FIG. 8) shows serum pharmacokinetics of anti-pyroGlu3 Abeta (Aβ) antibodies (50 mg/kg) of the present disclosure administered to aged 5XFAD mice over the course of 4 days.

FIG. 9 (FIG. 9) sets forth isoaffinity plots showing equilibrium dissociation constants (K_{D}), as a function of the association and dissociation rate constants (k-on and k-off), of anti-pyroGlu3 Abeta (Aβ) huIgG antibodies of the present disclosure to pyroGlu3 Abeta (Aβ) peptide (pE3-24).

FIG. 10 (FIG. 10) shows the effect of anti-TfR antibodies of the present disclosure on levels of circulating reticulocytes and TfR expression in huTfR KI mice. Mice were administered 3 mg/kg, 10 mg/kg, or 30 mg/kg of Iso-TfR antibody variants (L7-2 hIgG1 WT, L10-16 hIgG1 WT, 39.38-hIgG1 WT), and levels of circulating reticulocytes and TfR expression levels were measured 1 day (D1) and 7 days (D7) post-dose. Levels of circulating reticulocytes are shown as percent reticulocytes.

FIG. 11 (FIG. 11) shows serum antibody levels in huTfR KI mice after administration of 3 mg/kg, 10 mg/kg, or 30 mg/kg of Iso-TfR antibody variants (L7-2 hIgG1 WT, L10-16 hIgG1 WT, 39.38-hIgG1 WT). Serum PK was measured 1 day (D1) and 7 days (D7) post-dose.

FIG. 12 (FIG. 12) shows serum antibody levels in huTfR KI mice administered 3 mg/kg of remternetug and TfR-remternetug antibody variants (L7-2 hIgG1 WT, L10-16 hIgG1 WT, 39.38-hIgG1 WT). Mice were dosed twice, 14 days apart. Serum PK was measured at day 1 (D1), day 7 (D7), and day 14 (D14) after the first dose and 24 h after the second dose (day 15; D15).

FIG. 13 (FIG. 13) shows brain uptake of antibodies in huTfR KI mice dosed with 3 mg/kg of remternetug and TfR-remternetug antibody variants (L7-2 hIgG1 WT, L10-16 hIgG1 WT, 39.38-hIgG1 WT). Antibody concentration (ng/mg tissue) in vessel-depleted brain after 24h was measured.

FIG. 14 (FIG. 14) shows ex vivo plaque phagocytosis by primary human macrophages with anti-pyroGlu3 Abeta (Aβ) antibodies of the present disclosure, showing a similar extent of plaque phagocytosis as donanemab and remternetug, compared to isotype control. One way ANOVA with Tukey's multiple comparisons test. ****: p<0.0001.

FIGS. 15A and 15B (FIG. 15A and FIG. 15B) show the uptake of pyroGlu3 Abeta (Aβ) polypeptide by iPSC-derived microglia following incubation with anti-pyroGlu3 Abeta (Aβ) antibodies of the present disclosure. FIG. 15A shows data from the 24 hr time point (one way ANOVA with Tukey's multiple comparisons test. **: p<0.005; ***: p<0.001; ****: p<0.0001). FIG. 15B shows the time course of the Abeta (Aβ) peptide uptake in this study.

FIG. 16 (FIG. 16) shows cell uptake by immunohistochemistry of anti-pyroGlu3 Abeta (Aβ) antibodies of the present disclosure in hCMEC/D3 cell line.

FIG. 17 (FIG. 17) shows in vivo brain uptake of anti-pyroGlu3 Abeta (Aβ) antibodies of the present disclosure in hu-TfR knock-in (KI) mice following a single dose.

FIG. 18 (FIG. 18) shows serum pharmacokinetics of anti-pyroGlu3 Abeta (Aβ) antibodies of the present disclosure following administration of three i.v. doses to hu-TfR knock-in (KI) mice.

FIG. 19 (FIG. 19) shows in vivo brain uptake of anti-pyroGlu3 Abeta (Aβ) antibodies of the present disclosure in hu-TfR knock-in (KI) mice in a multi-dose study.

FIG. 20 (FIG. 20) shows in vivo brain uptake of brain uptake of anti-pyroGlu3 Abeta (Aβ) antibodies of the present disclosure in aged (9-month) 5XFAD mice following a single dose.

FIG. 21 (FIG. 21) shows serum pharmacokinetics of anti-pyroGlu3 Abeta (Aβ) antibodies of the present disclosure following administration of three i.v. doses to aged 5XFAD mice in a multi-dose study.

FIG. 22 (FIG. 22) shows in vivo brain uptake of anti-pyroGlu3 Abeta (Aβ) antibodies of the present disclosure in aged 5XFAD mice in a multi-dose study.

FIG. 23 (FIG. 23) shows immunofluorescent staining of amyloid plaques in aged 5XFAD mouse brain sections with anti-pyroGlu3 Abeta (Aβ) antibodies of the present disclosure.

FIG. 24 (FIG. 24) shows anti-pyroGlu3 Abeta (Aβ)-msTfR antibodies of the present disclosure in brain parenchyma administered to aged 5XFAD mice.

FIG. 25 (FIG. 25) shows reduction in Abeta₁₋₄₂ (Aβ₁₋₄₂) levels in mouse brain lysates following administration of anti-pyroGlu3 Abeta (Aβ)-msTfR antibodies of the present disclosure.

FIGS. 26A and 26B (FIG. 26A and FIG. 26B) show enhanced uptake of Abeta (pyroGlu3 Aβ) by iMicroglia using a human CNS triple culture cell model at antibody concentrations of 1 μg/ml and 10 μg/ml, respectively.

FIGS. 27A and 27B (FIG. 27A and FIG. 27B) show uptake of Abeta (pyroGlu3 Aβ) by anti-Abeta (Aβ) antibody remternetug-TfR39.38 having an effectorless Fc mutation LALAPS or a wildtype Fc in microglia in triple cultures and human fetal astrocytes+neuron cell population, respectively.

FIG. 28 (FIG. 28) shows transcytosis of anti-pyroGlu3 Abeta (Aβ)-TfR antibodies across human brain endothelial cell line hCMEC/D3.

FIG. 29 (FIG. 29) shows transcytosis of anti-pyroGlu3 Abeta (Aβ)-huTfR antibodies across primary cynomolgus brain endothelial cells.

FIG. 30 (FIG. 30) shows the level of ADCC induced by anti-pyroGlu3 Abeta (Aβ)-huTfR antibodies of the present disclosure.

FIGS. 31A to 31D (FIG. 31A-FIG. 31D) show that binding kinetics for 2+1 format antibodies of the present disclosure to human and cynomolgus Fc gamma receptors have kinetic profiles comparable with that of wildtype IgG1 Fc binding.

FIGS. 32A and 32B (FIG. 32A and FIG. 32B) show binding kinetics of anti-pyroGlu3 Abeta (Aβ)-TfR antibodies of the present disclosure to human (FIG. 32A) and cynomolgus (FIG. 32B) Fc gamma receptors, the binding of which are not distinguishable from wildtype IgG1 Fc.

FIG. 33 (FIG. 33) shows serum pharmacokinetics of antibodies in huTfR KI mice that received two weekly doses of 3 mg/kg or 30 mg/kg of antibody AB-8 huIgG1 and various AB-8-TfR variants. Mice were dosed on D1 (day 1) and D8 (day 8). Serum PK was measured at D2 (day 2), D8 (day 8) before dosing, and D9 (day 9).

FIGS. 34A and 34B (FIG. 34A and FIG. 34B) show reticulocyte percentages in peripheral whole blood assessed at D1 (day 1) and D7 (day 7) (FIG. 34A) post first dose and on D8 (day 8) (FIG. 34B) at study termination in huTfR KI mice.

FIG. 35 (FIG. 35) shows brain uptake of antibodies in huTfR KI mice that received two weekly doses of 3 mg/kg or 30 mg/kg of antibody AB-8 huIgG1 and various AB-8-TfR variants. Antibody levels (ng/mg protein) in vessel-depleted brain at 24 hours after the second dose was measured.

FIG. 36 (FIG. 36) shows reticulocyte levels as percent change from baseline in whole blood at day 10 from non-human primates (NHPs) dosed twice over 8 days with antibody AB-8 huIgG1 and various AB-8-TfR variants dosed at 3 mg/kg and 30 mg/kg.

FIG. 37 (FIG. 37) shows red blood cells (RBCs) levels as a percent change from baseline in whole blood at day 10 from NHPs receiving 2 doses over 8 days of antibody AB-8 huIgG1 and various AB-8-TfR variants dosed at 3 mg/kg and 30 mg/kg.

FIG. 38 (FIG. 38) shows serum pharmacokinetic profile from an NHP study with antibody AB-8 huIgG1 and AB-8-TfR variants.

FIG. 39 (FIG. 39) shows brain uptake of dosed antibodies from an NHP study with antibody AB-8 huIgG1 and AB-8-TfR variants. Enhanced brain uptake was observed at both 3 mg/kg and 30 mg/kg of all TfR antibodies compared to that observed with AB-8 hu IgG naked control antibody.

FIG. 40 (FIG. 40) shows AB-8-TfR42Q and AB-8-TfRL10-8 colocalizes with Abeta (Aβ) plaques on human Alzheimer's disease (AD) brain sections.

FIG. 41 (FIG. 41) shows quantification of plaque and dosed antibody colocalized area in the hippocampus of 5XFAD mice.

FIG. 42 (FIG. 42) shows TfR enhanced CSF uptake of AB-8-TfR42Q in NHPs.

FIG. 43 (FIG. 43) shows a bar graph of the CSF concentrations after the 3 mg/kg dose of AB-8 huIgG (circle data points) and AB-8-TfR42Q (triangle data points) at 2, 4, and 10 days after dosing.

FIG. 44 (FIG. 44) shows TfR levels in various brain regions from NHP dosed with anti-pyroGlu3 Abeta (Aβ) antibodies.

FIG. 45 (FIG. 45) shows serum and brain antibody levels after various time points in hu TfR KI mice dosed either intravenously or subcutaneously with 3 mg/kg of anti-pyroGlu3 Abeta (Aβ) antibodies.

### DETAILED DESCRIPTION OF THE PRESENT DISCLOSURE

The present disclosure relates to anti-beta amyloid antibodies that specifically bind N-terminal truncated pyroglutamate amyloid beta peptide (pyroGlu3 Abeta (Aβ)), multi-specific binding proteins comprising an anti-Abeta (pyroGlu3 Aβ) antigen-binding domain and a blood brain barrier (BBB) antigen-binding domain that specifically binds to a BBB target selected from human transferrin receptor (TfR) or human CD98 heavy chain (CD98hc), and uses (e.g., therapeutic uses) thereof.

Amyloid beta (Abeta; Aβ) peptide in circulating form is composed of 38-43 amino acids (mostly 38, 40, or 42 amino acids) resulting from the cleavage of amyloid precursor protein (APP). Conversion of Abeta (Aβ) from soluble to insoluble forms, and the deposition of these insoluble forms as neuritic and cerebrovascular plaques in the brain has been associated with a number of conditions and diseases, including Alzheimer's disease (AD), Down's syndrome, cerebral amyloid angiopathy (CAA), and other amyloid-beta associated disorders.

The deposits found in plaques are comprised mainly of a heterogeneous mixture of Abeta (Aβ) peptides. PyroGlu3 Abeta (Aβ) (also referred to as N3pE amyloid beta, N3pE Abeta (Aβ), N3pGlu Abeta (Aβ), or Abeta_{p3_42} (Ap_{p3_42})) is a truncated form of Abeta (Aβ) peptide found only amyloid plaques. PyroGlu3 Abeta (Aβ) lacks the first two amino acid residues at the N-terminus of Abeta (Aβ) and has a pyroglutamate derived from the glutamic acid at the third amino acid position. Although PyroGlu3 Abeta (Aβ) peptide is a minor component of deposited Abeta (Aβ) in the brain, studies have demonstrated that PyroGlu3 Abeta (Aβ) peptide has aggressive aggregation properties and accumulates early in the deposition cascade.

Passive transfer of substances from blood to brain is restricted by the blood brain barrier (BBB). The BBB provides precise control of central nervous system (CNS) homeostasis allowing for proper neuronal function and also protecting neural tissue from toxins and pathogens. However, the BBB poses a problem with regard to delivering therapeutics to the CNS. While recombinant proteins and antibody therapeutics have shown much success outside the CNS, such biologics do not cross the BBB efficiently. As a result, delivery of certain therapeutics to the CNS has relied on injection of the therapeutic directly into the CNS; an invasive procedure with limited efficacy due to rapid export of cerebral spinal fluid (CSF) containing the therapeutic from the brain to the blood. Alternatively, a therapeutic intended for the CNS may be administered systemically at doses high enough to allow for sufficient penetration of the BBB by the therapeutic. However, this approach may result in unintended effects due to the high dose in the periphery or increased manufacturing and formulation burdens to achieve the high dose.

Antibodies that target Abeta (Aβ) have been previously described, as well as polyclonal and monoclonal antibodies that are directed specifically against pyroGlu3 Abeta (Aβ) (e.g., bapineuzumab, gantenerumab, aducanumab, GSK933776, Alia-1758, BAN 2802, solanezumab, crenezumab, ponezumab, lecanemab and trontinemab; see, e.g., U.S. Pat. No. 7,122,374, WO2002/046237, WO2004/080419, WO2012/021469, WO2012/021475, WO2017/123517, WO2017/160555, WO2018/005282, WO2018/031361, WO2018/194951, WO2022/150735, WO2022/192636, WO2022/251048, WO2024/020470, WO2024/107683, WO2003/070760, WO2014/056816, WO2015/120280, WO2014/089500, WO2016/087944, WO2017/211827, WO2019/040612, WO2021/081101, WO2022/261026, WO2023/081194, WO2020/023530, WO2021/186245, WO2023/111618, WO2024/118665, WO2024/206161, WO2023/170295, WO2023/170291, WO2023/170290, WO2018/011353, and WO2024/133925). Despite recent approval of two anti-Abeta (Aβ) antibodies for the treatment of Alzheimer's Disease (donanemab-azbt, lecanemab-irmb), there is still a need for therapeutically useful antibody antagonists to pyroGlu3 Abeta (Aβ) possessing a number of desirable properties, such as, for example, high affinity, plaque-clearance, and the ability to effectively and efficiently cross the blood brain barrier.

The present disclosure provides antibodies that specifically bind pyroGlu3 Abeta (Aβ).

The present disclosure further provides anti-pyroGlu3 Abeta (Aβ) multi-specific binding proteins. Anti-pyroGlu3 Abeta (Aβ) multi-specific binding proteins provided herein are capable of crossing the blood brain barrier and capable of transporting an anti-pyroGlu3 Abeta (Aβ) antigen binding domain, antibody, or antigen-binding fragment thereof across the blood brain barrier, thus enhancing or increasing exposure of the anti-pyroGlu3 Abeta (Aβ) antigen binding domain, antibody, or antigen-binding fragment thereof to the CNS.

The present disclosure provides various multi-specific binding protein formats comprising an anti-blood brain barrier antigen-binding domain linked to an anti-pyroGlu3 Abeta (Aβ) antigen-binding domain (e.g., an antibody antigen binding domain or antibody or antigen-binding fragment thereof that binds specifically to pyroGlu3 Abeta (Aβ)).

In some aspects, a multi-specific binding protein of the present disclosure with a “1+1 multi-specific binding protein format” comprises a TfR antigen-binding domain in a bivalent, bi-specific format comprising (i) one antigen-binding domain that binds to human TfR; and (ii) one antigen-binding domain that binds to pyroGlu3 Abeta (Aβ). Such a “1+1” format can also comprise an Fc domain or Fc region. In some aspects, a multi-specific binding protein of the present disclosure with a “1+1 multi-specific binding protein format” comprises a CD98hc antigen-binding domain in a bivalent, bi-specific format comprising (i) one antigen-binding domain that binds to human CD98hc; and (ii) one antigen-binding domain that binds to pyroGlu3 Abeta (Aβ). Such a “1+1” format can also comprise an Fc domain or Fc region.

In some aspects, a multi-specific binding protein of the present disclosure with a “2+1 multi-specific binding protein format” comprises a TfR antigen-binding domain in a trivalent, bi-specific format comprising (i) an antigen-binding domain that binds to human TfR and (ii) an antibody comprising two anti-pyroGlu3 Abeta (Aβ) antigen-binding domains, wherein the antibody comprises two heavy chains and two light chains; wherein the antigen-binding domain that binds to human TfR is linked to the C-terminus of one of the two antibody heavy chains. In some aspects, a multi-specific binding protein of the present disclosure with a “2+1 multi-specific binding protein format” comprises a CD98hc antigen-binding domain in a trivalent, bi-specific format comprising (i) an antigen-binding domain that binds to human CD98hc, and (ii) an antibody comprising two anti-pyroGlu3 Abeta (Aβ) antigen-binding domains, wherein the antibody comprises two heavy chains and two light chains; wherein the antigen-binding domain that binds to human CD98hc is linked to the C-terminus of one of the two antibody heavy chains.

In some embodiments, the multi-specific protein comprises a “1+2” multi-specific protein format, wherein the multi-specific protein is trivalent and bi-specific. In some embodiments, the multi-specific protein comprises: (i) two antigen-binding domains that specifically bind to human TfR and (ii) an antibody or antigen-binding fragment thereof that specifically binds to pyroGlu3 Abeta (Aβ), wherein the antibody or antigen-binding fragment thereof is a scFv, VHH, or Fab, optionally comprising an Fc domain. In some embodiments, the multi-specific protein comprises a “1+2” multi-specific protein format, wherein the multi-specific protein is trivalent and bi-specific. In some embodiments, the multi-specific protein comprises: (i) two antigen-binding domains that specifically binds to human CD98hc and (ii) an antibody or antigen-binding fragment thereof that specifically binds to pyroGlu3 Abeta (Aβ), wherein the antibody or antigen-binding fragment thereof is a scFv, VHH, or Fab, optionally comprising an Fc domain.

In some aspects, a multi-specific binding protein of the present disclosure with a “2+2 multi-specific binding protein format” comprises a TfR antigen-binding domain in a tetravalent, bi-specific format comprising (i) two antigen-binding domains that bind to human TfR and (ii) an antibody comprising two anti-pyroGlu3 Abeta (Aβ) antigen-binding domains, wherein the antibody comprises two heavy chains and two light chains; wherein one antigen-binding domain that binds to human TfR is linked to the C-terminus of one of the two antibody heavy chains, and the other antigen-binding domain that binds to human TfR is linked to the C-terminus of the other of the two antibody heavy chains. The two antigen-binding domains that bind to human TfR can comprise the same amino acid sequence. The two antigen-binding domains that bind to human TfR can comprise different amino acid sequences. In some aspects, a multi-specific binding protein of the present disclosure with a “2+2 multi-specific binding protein format” comprises a CD98hc antigen-binding domain in a tetravalent, bi-specific format comprising (i) two antigen-binding domains that bind to human CD98hc and (ii) an antibody comprising two anti-pyroGlu3 Abeta (Aβ) antigen-binding domains, wherein the antibody comprises two heavy chains and two light chains; wherein one antigen-binding domain that binds to human CD98hc is linked to the C-terminus of one of the two antibody heavy chains, and the other antigen-binding domain that binds to human CD98hc is linked to the C-terminus of the other of the two antibody heavy chains. In some aspects, a multi-specific binding protein of the present disclosure with a “2+2 multi-specific binding protein format” comprises a TfR antigen-binding domain in a tetravalent, bi-specific format comprising (i) two antigen-binding domains that bind to human pyroGlu3 Abeta (Aβ) and (ii) an antibody comprising two anti-TfR antigen-binding domains, wherein the antibody comprises two heavy chains and two light chains; wherein one antigen-binding domain that binds to human pyroGlu3 Abeta (Aβ) is linked to the C-terminus of one of the two antibody heavy chains, and the other antigen-binding domain that binds to human pyroGlu3 Abeta (Aβ) is linked to the C-terminus of the other of the two antibody heavy chains. The two antigen-binding domains that bind to human pyroGlu3 Abeta (Aβ) can comprise the same amino acid sequence. The two antigen-binding domains that bind to human pyroGlu3 Abeta (Aβ) can comprise different amino acid sequences. In some aspects, a multi-specific binding protein of the present disclosure with a “2+2 multi-specific binding protein format” comprises a CD98hc antigen-binding domain in a tetravalent, bi-specific format comprising (i) two antigen-binding domains that bind to human pyroGlu3 Abeta (Aβ) and (ii) an antibody comprising two anti-CD98hc antigen-binding domains, wherein the antibody comprises two heavy chains and two light chains; wherein one antigen-binding domain that binds to human pyroGlu3 Abeta (Aβ) is linked to the C-terminus of one of the two antibody heavy chains, and the other antigen-binding domain that binds to human pyroGlu3 Abeta (Aβ) is linked to the C-terminus of the other of the two antibody heavy chains.

The techniques and procedures described or referenced herein are generally well understood and commonly employed using conventional methodology by those skilled in the art, such as, for example, the widely utilized methodologies such as those described in Sambrook et al. Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003); Monoclonal Antibodies: A Practical Approach (P. Shepherd and C. Dean, eds., Oxford University Press, 2000).

### DEFINITIONS

The terms “amyloid beta,” “Abeta,” “Aβ,” and “A-beta,” used interchangeably herein, refer to the peptide formed by the proteolytic cleavage of a larger glycoprotein called amyloid precursor protein (APP).

The terms “pyroGlu3 Aβ,” “N3pE Aβ,” or “N3pGlu Aβ” peptide, polypeptide, or protein, used interchangeably herein, refer to any native Aβ peptide or protein bearing amino-terminal pyroglutamate at amino acid position 3 from any vertebrate source, including mammals such as primates (e.g., humans and cynomolgus monkeys (cynos)) and rodents (e.g., mice and rats), unless otherwise indicated. In some aspects, the term encompasses both wild-type sequences and naturally occurring variant sequences, e.g., splice variants or allelic variants. In some aspects, the pyroGlu3 Aβ is human pyroGlu3 Aβ.

The terms “anti-pyroGlu3 Aβ antibody,” and “antibody that binds to pyroGlu3 Aβ,” and “antibody that specifically binds pyroGlu3 Aβ” refer to an antibody that is capable of binding pyroGlu3 Aβ with sufficient affinity such that the antibody is useful as a diagnostic and/or therapeutic agent in targeting pyroGlu3 Aβ.

The terms “central nervous system” or “CNS” refer to the complex of nerve tissues that control bodily function and includes the brain and spinal cord.

The terms “blood brain barrier” or “BBB” refer to a network of brain capillary endothelial cells that are closely sealed by tight junctions.

A “central nervous system antigen” or “CNS antigen” is an antigen expressed in the CNS, including the brain.

A “brain antigen” is a CNS antigen expressed in the brain.

The terms “BBB target,” “BBB protein,” “BBB receptor,” and “BBB antigen” refer to a target/protein/receptor/antigen expressed on blood brain barrier cells (e.g., TfR or CD98hc). In some aspects, an antigen-binding domain (e.g., in an antibody, scFv, or Fab) that binds to the BBB target/protein/receptor/antigen allows transportation of a molecule or compound associated with said antigen-binding domain across the BBB.

A “neurological disorder” as used herein refers to a disease or disorder which affects the CNS and/or which has an etiology in the CNS. Exemplary CNS diseases or disorders include, but are not limited to, neuropathy, amyloidosis, Alzheimer's disease, and neurodegenerative diseases and disorders.

The terms “Transferrin receptor,” “TfR,” “TfR polypeptide,” and “TfR protein” are used interchangeably herein to refer to any native TfR from any vertebrate source, including mammals such as primates (e.g., humans and cynomolgus monkeys (cynos)) and rodents (e.g., mice and rats), unless otherwise indicated. TfR is also referred to as transferrin receptor protein 1, TR, tfR1, Trfr, T9, and p90. In some aspects, the term encompasses both wild-type sequences and naturally occurring variant sequences, e.g., splice variants or allelic variants. In some aspects, the term encompasses “full-length,” unprocessed TfR, as well as any form of TfR that results from processing in the cell. Full-length transferrin receptor protein includes a short N-terminal intracellular region, a transmembrane region, and a large extracellular domain. The extracellular domain is characterized by three domains: a protease-like domain, a helical domain, and an apical domain. In some aspects, the TfR is human TfR. As used herein, the term “human TfR” refers to a polypeptide with the following amino acid sequence:

(SEQ ID NO: 2)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLQWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

As used herein, the terms “CD98hc,” “CD98hc polypeptide,” and “CD98hc protein” are used interchangeably herein to refer to any native CD98hc from any vertebrate source, including mammals such as primates (e.g., humans and cynomolgus monkeys (cynos)) and rodents (e.g., mice and rats), unless otherwise indicated. CD98hc is also referred to as 4F2 cell-surface antigen heavy chain, 4F2 hc, 4F2 heavy chain antigen, lymphocyte activation antigen 4F2 large subunit, solute carrier family 3 member 2, and CD98. CD98hc protein is encoded by the SLC3A2 gene and is part of the large amino acid transporter (LAT) complex. In some aspects, the term encompasses both wild-type sequences and naturally occurring variant sequences, e.g., splice variants or allelic variants. In some aspects, the term encompasses “full-length,” unprocessed CD98hc, as well as any form of CD98hc that results from processing in the cell. In some aspects, the CD98hc is human CD98hc. As used herein, the term “human CD98hc” refers to a polypeptide with the following amino acid sequence:

(SEQ ID NO: 3)
MELQPPEASIAVVSIPRQLPGSHSEAGVQGLSAGDDSELGSHCVAQTGL
ELLASGDPLPSASQNAEMIETGSDCVTQAGLQLLASSDPPALASKNAEV
TGTMSQDTEVDMKEVELNELEPEKQPMNAASGAAMSLAGAEKNGLVKIK
VAEDEAEAAAAAKFTGLSKEELLKVAGSPGWVRTRWALLLLFWLGWLGM
LAGAVVIIVRAPRCRELPAQKWWHTGALYRIGDLQAFQGHGAGNLAGLK
GRLDYLSSLKVKGLVLGPIHKNQKDDVAQTDLLQIDPNFGSKEDFDSLL
QSAKKKSIRVILDLTPNYRGENSWFSTQVDTVATKVKDALEFWLQAGVD
GFQVRDIENLKDASSFLAEWQNITKGFSEDRLLIAGTNSSDLQQILSLL
ESNKDLLLTSSYLSDSGSTGEHTKSLVTQYLNATGNRWCSWSLSQARLL
TSFLPAQLLRLYQLMLFTLPGTPVFSYGDEIGLDAAALPGQPMEAPVML
WDESSFPDIPGAVSANMTVKGQSEDPGSLLSLFRRLSDQRSKERSLLHG
DFHAFSAGPGLFSYIRHWDQNERFLVVLNFGDVGLSAGLQASDLPASAS
LPAKADLLLSTQPGREEGSPLELERLKLEPHEGLLLRFPYAA.

As used herein, the terms “antibody” and “immunoglobulin” are used interchangeably and refer to an antibody molecule that recognizes and specifically binds to a target, such as a protein, polypeptide, peptide, carbohydrate, polynucleotide, lipid, or combinations of the foregoing (e.g., a glycoprotein), through at least one antigen recognition site within the variable region of the immunoglobulin molecule. The term “antibody” encompasses monoclonal antibodies, chimeric antibodies, humanized antibodies, human antibodies, multi-specific (e.g., bi-specific) antibodies, and any other immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgAQ1, and IgA2), based on the identity of their heavy-chain constant regions referred to as alpha, delta, epsilon, gamma, and mu, respectively. The different classes of antibodies have different and well-known subunit structures and three-dimensional configurations. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6.

The terms “full-length antibody,” “intact antibody,” or “whole antibody” are used interchangeably to refer to an antibody, such as an anti-pyroGlu3 Aβ antibody, in its substantially intact form, as opposed to an antibody fragment. Specifically, full-length antibodies include those with 2 light chains and 2 heavy chains including an Fc region. The constant domains may be native sequence constant domains (e.g., human native sequence constant domains) or amino acid sequence variants thereof. In some cases, the intact antibody may have one or more effector functions.

The terms “monovalent antibody” or “monoarm antibody” refers to an antibody having a single antigen-binding recognition domain that is specific to a target antigen (i.e., the antibody comprises no more than one antigen-binding domain). In some embodiments, a single antigen-binding domain comprises a single variable region heavy chain polypeptide and a single variable region light chain polypeptide. An antibody that is “monovalent” for a target comprises no more than one antigen-binding domain for that target.

The terms “anti-TfR antibody,” “antibody that binds to TfR,” and “antibody that specifically binds TfR” refer to an antibody that is capable of binding TfR with sufficient affinity such that the antibody is useful as a diagnostic and/or therapeutic agent in targeting TfR. In some aspects, the anti-TfR antibody is capable of transporting another diagnostic and/or therapeutic agent into the brain. In certain aspects, an anti-TfR antibody binds to an epitope of TfR that is conserved among TfR from different species.

The terms “anti-TfR antigen-binding domain,” “antigen-binding domain that binds to TfR,” “anti-TfR antigen-binding region,” “antigen-binding region that binds to TfR,” and “TfR binding domain” refer to an antigen-binding domain that binds to TfR with sufficient affinity such that the antigen-binding domain is useful as a diagnostic and/or therapeutic agent in targeting TfR. In one aspect, the extent of binding of an anti-TfR antigen-binding domain to an unrelated, non-TfR polypeptide is less than about 10% of the binding of the antigen-binding domain to TfR as measured, e.g., by a radioimmunoassay (RIA). In certain embodiments, an anti-TfR antigen-binding domain binds to an epitope of TfR that is conserved among TfR from different species.

The terms “anti-CD98hc antibody,” “antibody that binds to CD98hc,” and “antibody that specifically binds CD98hc” refer to an antibody that is capable of binding CD98hc with sufficient affinity such that the antibody is useful as a diagnostic and/or therapeutic agent in targeting CD98hc. In some aspects, the anti-CD98hc antibody is capable of transporting another diagnostic and/or therapeutic agent into the brain. In certain aspects, an anti-CD98hc antibody binds to an epitope of CD98hc that is conserved among CD98hc from different species.

The terms “anti-CD98hc antigen-binding domain,” “antigen-binding domain that binds to CD98hc,” “anti-CD98hc antigen-binding region,” “antigen-binding region that binds to CD98hc,” and “CD98hc binding domain” refer to an antigen-binding domain that binds to CD98hc with sufficient affinity such that the antigen-binding domain is useful for targeting CD98hc and/or useful as a diagnostic agent, a therapeutic agent, or for transporting a molecule or compound across the BBB. In one aspect, the extent of binding of an anti-CD98hc antigen-binding domain to an unrelated, non-CD98hc polypeptide is less than about 10% of the binding of the antigen-binding domain to CD98hc as measured, e.g., by a radioimmunoassay (RIA). In certain aspects, an anti-CD98hc antigen-binding domain binds to an epitope of CD98hc that is conserved among CD98hc from different species.

An “antigen-binding domain” or “antigen-binding region” refers to a monovalent portion of an antibody that binds to an antigen. An “antigen-binding domain” can comprise the antigenic determining regions of an antibody (e.g., the complementarity determining regions (CDRs)). An antibody or antigen-binding fragment thereof (including mono-specific and multi-specific (e.g., bi-specific) antibodies or antigen-binding fragments thereof can comprise an antigen-binding domain. In some aspects, an antigen-binding domain is not present in the context of an antibody. In some aspects, provided herein is an antibody, a VHH, a Fab, a Fab′, a Fab′-SH, a F(ab′)2, a Fv, or a scFv comprising an antigen-binding domain.

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant regions can be native sequence constant regions (e.g., human native sequence constant regions) or amino acid sequence variants thereof. In some cases, the intact antibody can have one or more effector functions. The C-terminal lysine (residue 447 according to the EU numbering system) of an intact antibody can be removed, for example, during production or purification of the antibody, or by recombinantly engineering the nucleic acid encoding a heavy chain of the antibody. Accordingly, a composition of “full-length antibodies,” “intact antibodies,” or “whole antibodies” can comprise antibody populations with all K447 residues removed, antibody populations with no K447 residues removed, and antibody populations having a mixture of antibodies with and without the K447 residue.

The term “native IgG antibodies” refers to heterotetrameric glycoproteins of about 150,000 Daltons, composed of two identical light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The terms “VH” and “VH domain” are used interchangeably to refer to the heavy chain variable region of an antibody.

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG₁, IgG₂, IgG₃, and IgG₄. Heavy chain amino acid sequences are well known in the art. In some aspects, the heavy chain is a human heavy chain.

The terms “VL” and “VL domain” are used interchangeably to refer to the light chain variable region of an antibody.

As used herein, the term “light chain” when used in reference to an antibody can refer to any distinct type, e.g., kappa (κ) or lambda (λ) based on the amino acid sequence of the constant regions. Light chain amino acid sequences are well known in the art. In some aspects, the light chain is a human light chain.

The terms “variable region” or “variable domain” refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. Generally, the variable region or variable domain is typically about the amino-terminal 110 to 120 amino acids or 110 to 125 amino acids in the mature heavy chain and about 90 to 115 amino acids in the mature light chain.

The term “Fv” or “variable fragment” refers to the minimum antibody fragment which comprises a complete antigen-binding site and consists of a dimer of one heavy-chain variable region (VH) and one light-chain variable region (VL). From the folding of these two domains emanate six hypervariable loops (3 loops each from the H and L chain) that contribute the amino acid residues for antigen binding and confer antigen binding specificity to the antibody.

As used herein, the term “constant region” is a region of an antibody that is not the variable region of the antibody, e.g., a carboxyl terminal portion of a light and/or heavy chain which is not directly involved in binding of an antibody to antigen, but which can exhibit various effector functions, such as interaction with the Fc receptor. The constant region of an immunoglobulin molecule generally has a more conserved amino acid sequence relative to an immunoglobulin variable domain. In certain aspects, an antibody or antigen-binding fragment comprises a constant region or portion thereof that is sufficient for antibody-dependent cell-mediated cytotoxicity (ADCC).

A “constant domain” means a domain within a constant region that is capable of forming an immunoglobulin fold. Constant domains include the CH1, CH2, CH3, and CL domains.

The term “antibody fragment” refers to a portion of an antibody. An “antigen-binding fragment” of an antibody refers to a portion of an antibody that binds to an antigen. An antigen-binding fragment of an antibody can comprise the antigenic determining regions of an antibody (e.g., the complementarity determining regions (CDRs)). Examples of antigen-binding fragments of antibodies include, but are not limited to Fab, Fab′, F(ab′)2, and Fv fragments, linear antibodies, and single chain antibodies. An antigen-binding fragment of an antibody can be monovalent or multi-valent (e.g., bi-valent). An antigen-binding fragment of an antibody can be monospecific or multi-specific (e.g., bi-specific.) An antigen-binding fragment of an antibody can be derived from any animal species, such as rodents (e.g., mouse, rat, or hamster) and humans or can be artificially produced.

The term “Fab” or “fragment antigen-binding region” refers to a region on an antibody that binds to antigens. It is composed of one constant domain, one variable domain of the heavy chain and one variable domain of the light chain. Each Fab fragment is monovalent with respect to antigen binding, i.e., it has a single antigen-binding site.

The term “F(ab′)2 fragment” refers to antibody fragments that are generated by pepsin digestion of whole IgG antibodies to remove most of the Fc region while leaving intact some of the hinge region. F(ab′)2 fragments have two antigen-binding F(ab) portions linked together by disulfide bonds, and therefore are divalent with a molecular weight of about 110 kDa. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group.

As used herein, a “Fc fragment,” “fragment crystallizable region,” or “Fc region” is composed of two or more polypeptides, each being an antibody heavy chain fragment and each containing at least one (e.g., two or three) heavy chain constant domains. In some aspects, an Fc region is composed of two heavy chain fragments, each containing a CH2 domain and a CH3 domain. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region can be removed, for example, during production or purification of the antibody, or by recombinantly engineering the nucleic acid encoding a heavy chain of the antibody. Accordingly, an Fc region may not contain any K447 residues, may contain at least one polypeptide containing a K447 residue and at least one polypeptide that does not contain a K447 residue, or may only contain polypeptides that include a K447 residue. Suitable native-sequence Fc regions for use in the present disclosure include human IgG1, IgG2, IgG3 and IgG4. In a native antibody, an Fc region refers to the region of an antibody that interacts with cell surface receptors called Fc receptors and some proteins of the complement system. However, as used herein, an Fc region can be modified to increase, decrease, or eliminate interaction with Fc receptors and/or proteins of the compliment system. In native IgG, IgA and IgD antibody isotypes, the Fc region is composed of two identical protein fragments, derived from the second and third constant domains of the antibody's two heavy chains. However, as used herein, the two or more polypeptides in an “Fc region” do not need to have identical sequences. In some aspects, an “Fc region” comprises a first polypeptide comprising an Fc domain (e.g., IgG1 Fc domain) with a knob mutation and a second polypeptide comprising an Fc domain (e.g., IgG1 Fc domain) with a hole mutation. In native IgM and IgE antibody isotypes, the Fc region contains three heavy chain constant domains (CH domains 2-4) in each polypeptide chain.

The term “Fc domain” refers to one or more constant region domains within an Fc region, such as a CH2 or CH3 domain, in a single polypeptide. In some aspects, the Fc domain includes at least one amino acid deletion, addition, or substitution as compared to the amino acid sequence of a native Fc domain, such as by including a set of “knob-into-hole” deletions, additions, or substitutions or including amino acid deletions, additions, or substitutions to effect electrostatic steering of the Fc domain to favor attractive interactions among different polypeptide chains. In some aspects, the Fc domain is in a “knob” format. In some aspects, the Fc domain is in a “hole” format.

The term “single-chain Fv”, also abbreviated as “sFv” or “scFv”, refers to antibody fragments that comprise the VH and VL antibody domains that form a single polypeptide chain. In some aspects, the scFv polypeptide comprises a polypeptide linker between the VH and VL domains, which enables the scFv to form the desired structure for antigen binding.

The term “diabodies” refers to small antibody fragments prepared by constructing scFv fragments with short linkers (about 5-10 residues) between the VH and VL domains, such that inter-chain but not intra-chain pairing of the variable domains is achieved, thereby resulting in a bivalent fragment (i.e., a fragment having two antigen-binding sites). Bispecific diabodies are heterodimers of two “crossover” scFv fragments in which the VH and VL domains of the two antibodies are present on different polypeptide chains.

The term “CDR” or “complementarity determining region” refers to hypervariable regions in the variable region of an immunoglobulin that determine antibody diversity and antigen specificity.

The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDRH1), amino acid positions 50 to 65 (CDRH2), and amino acid positions 95 to 102 (CDRH3). Using the Kabat numbering system, CDRs within an antibody light chain molecule are typically present at amino acid positions 24 to 34 (CDRL1), amino acid positions 50 to 56 (CDRL2), and amino acid positions 89 to 97 (CDRL3).

The term “Chothia” refers to the location of the structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). In some aspects, the Chothia residues are numbered as shown in the table below.

The AbM hypervariable regions represent a compromise between the Kabat CDRs and Chothia structural loops and are used by Oxford Molecular's AbM antibody modeling software.

In some aspects, the CDRs can be “contact” CDRs. The “contact” CDRs are based on an analysis of the available complex crystal structures.

The residues from each of these CDRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat |
| | | | | numbering) |
| H1 | H31-H35 | H26-H35 | H26-H32 | H30-H35 |
| | | | | (Chothia |
| | | | | numbering) |
| H2 | H50-H65 | H50-H58 | H52-H56 | H47-H58 |
| H3 | H95-H102 | H95-H102 | H96-H101 | H93-H101 |

CDRs can comprise “extended CDRs” as follows: 24-36 or 24-34 (L1), 46-56 or 50-56 (L2), and 89-97 or 89-96 (L3) in the VL, and 26-35 (H1), 50-65 or 49-65 (H2), and 93-102, 94-102, or 95-102 (H3) in the VH. The variable-domain residues are numbered according to Kabat et al., supra, for each of these extended-CDR definitions.

CDRs can also be identified according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

The term “monoclonal” when referring to an antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins or complexes comprising an antibody or antibody portion, and any other modified immunoglobulin molecule comprising an antigen recognition site. Furthermore, a “monoclonal” antibody or antigen-binding fragment thereof refers to such antibodies and antigen-binding fragments thereof made in any number of manners including but not limited to by hybridoma, phage selection, recombinant expression, and transgenic animals.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g., mouse, rat, rabbit, etc.) with the desired specificity, affinity, and capability, while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g., murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementarity determining regions (CDRs) are replaced by residues from the CDRs of a molecule originating from a non-human species (e.g., mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). The humanized antibody or antigen-binding fragment thereof can be further modified by the substitution of additional residues either in the Fv framework region and/or within the replaced non-human residues to refine and optimize the specificity, affinity, and/or capability of the antibody or antigen-binding fragment thereof. In general, the humanized antibody or antigen-binding fragment thereof will comprise VH and VL that comprise substantially all of at least one, and typically two or three, of the CDR regions that correspond to the non-human immunoglobulin, whereas all or substantially all of the FR regions are those of a human immunoglobulin consensus sequence. The humanized antibody or antigen-binding fragment thereof can also comprise at least a portion of an immunoglobulin constant region or Fc region, typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Framework” or “FR” residues are those variable-domain residues other than the CDR residues as herein defined.

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework can comprise the same amino acid sequence thereof, or it can comprise pre-existing amino acid sequence changes. In some aspects, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, in some aspects those changes occur at only three, two, or one of positions 71H, 73H and 78H; for instance, the amino acid residues at those positions can be 71A, 73T and/or 78A. In some aspects, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the V_{L}, the subgroup can be subgroup kappa I, kappa II, kappa III or kappa IV as in Kabat et al., supra. Additionally, for the V_{H}, the subgroup can be subgroup I, subgroup II, or subgroup III as in Kabat et al., supra.

An “amino-acid modification” at a specified position, e.g., of an antibody of the present disclosure, refers to the substitution or deletion of the specified residue, or the insertion of at least one amino acid residue adjacent the specified residue. Insertion “adjacent” to a specified residue means insertion within one to two residues thereof. The insertion can be N-terminal or C-terminal to the specified residue. In some aspects, an amino acid modification is a substitution.

Antibody “effector functions” refer to those biological activities attributable to the Fc region (a native sequence Fc region or amino acid sequence variant Fc region) of an antibody and vary with the antibody isotype.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well.

A “variant Fc region” comprises an amino acid sequence which differs from that of a native sequence Fc region by virtue of at least one amino acid modification, in some aspects two or more amino acid substitution(s). In some aspects, the variant Fc region has at least one amino acid substitution compared to a native sequence Fc region or to the Fc region of a parent polypeptide, e.g., from about one to about ten amino acid substitutions, and in some aspects from about one to about five amino acid substitutions compared to a native sequence Fc region or in the Fc region of the parent polypeptide. In some aspects, the variant Fc region possesses at least 80% homology with a native sequence Fc region and/or with an Fc region of a parent polypeptide, at least 90% homology therewith, or at least 95% homology therewith.

“Fc receptor” or “FcR” describes a receptor that binds to the Fc region of an antibody. In some aspects, an FcR is a native sequence human FcR. In some aspects, a FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the equilibrium dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{off} refers to the dissociation rate constant of, e.g., an antibody or antigen-binding fragment thereof from an antigen. The kₒₙ and k_{off} can be determined by techniques known to one of ordinary skill in the art, such as BIAcore® or KinExA. Dissociation constants may also be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. (See, e.g., Estep et al, (2013) MAbs 5(2):270-8.)

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds” or is “specific for” a particular polypeptide or an epitope on a particular polypeptide target as used herein can be exhibited, for example, by a molecule having a KD for the target of about any of 10⁻⁴ M or lower, 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In some aspects, the term “specific binding” refers to binding where a molecule binds to a particular polypeptide or epitope on a particular polypeptide without substantially binding to any other polypeptide or polypeptide epitope.

The term “linker” or “linked” refers to the covalent linkage between two polypeptides or two heterologous molecules. In some aspects, a linker is a chemical linker. In some aspects, the linker comprises a peptide bond, and the two polypeptides or two heterologous molecules are linked to each other either directly to or via one or more additional amino acids. A glycine linker is one that comprises one or more glycines, but no other amino acids, e.g., GGGG (SEQ ID NO: 204). A glycine-rich linker is one that comprises one or more glycines and can contain other amino acids as long as glycine is the predominant species in the linker e.g., GGGNGG (SEQ ID NO: 205), wherein N is any amino acid. A glycine-serine linker is one which contains both glycine and serine in any proportion, e.g., GGGS (SEQ ID NO: 206). Similarly, a proline linker is one that comprises one or more prolines but no other amino acids. A proline-rich linker is one that comprises one or more prolines and can contain other amino acids so long as proline is the predominant species in the linker.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as identical matches. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some aspects, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

The term “expression system” refers to one or more nucleic acid molecules comprising coding sequence and control sequence(s) in operable linkage, along with a host cell and/or other in vitro transcription and translation machinery, such that one or more proteins encoded by the nucleic acid molecule(s) are capable of being produced.

The term “vector,” as used herein, is intended to refer to a nucleic acid molecule capable of transporting another nucleic acid to which it has been linked. One type of vector is a “plasmid,” which refers to a circular double stranded DNA into which additional DNA segments can be ligated. Another type of vector is a phage vector. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” can be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure. In some aspects, the host cell is an isolated host cell.

“Carriers” as used herein include pharmaceutically acceptable carriers, excipients, or stabilizers that are nontoxic to the cell or mammal being exposed thereto at the dosages and concentrations employed.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

The terms “administer,” “administering,” “administration,” and the like, as used herein, refer to methods that can be used to deliver a drug, e.g., an anti-human antibody or antigen-binding fragment thereof, to the desired site of biological action.

An “effective amount” refers to at least an amount effective, at dosages and for periods of time necessary, to achieve the desired therapeutic result. An effective amount can be provided in one or more administrations. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” can be considered in the context of administering one or more therapeutic agents, and a single agent can be considered to be given in an effective amount if, in conjunction with one or more other agents, a desirable result can be or is achieved.

As used herein, the terms “subject” and “patient” are used interchangeably. The subject can be a mammal such as a non-human animal (e.g., cow, pig, horse, cat, dog, rat, mouse, monkey or other primate, etc.). In some aspects, the subject is a cynomolgus monkey. In some aspects, the subject is a human.

As used herein, administration “in conjunction” or “in combination” with another compound or composition includes simultaneous administration and/or administration at different times. Administration in conjunction also encompasses administration as a co-formulation or administration as separate compositions, including at different dosing frequencies or intervals, and using the same route of administration or different routes of administration. In some aspects, administration in conjunction is administration as a part of the same treatment regimen.

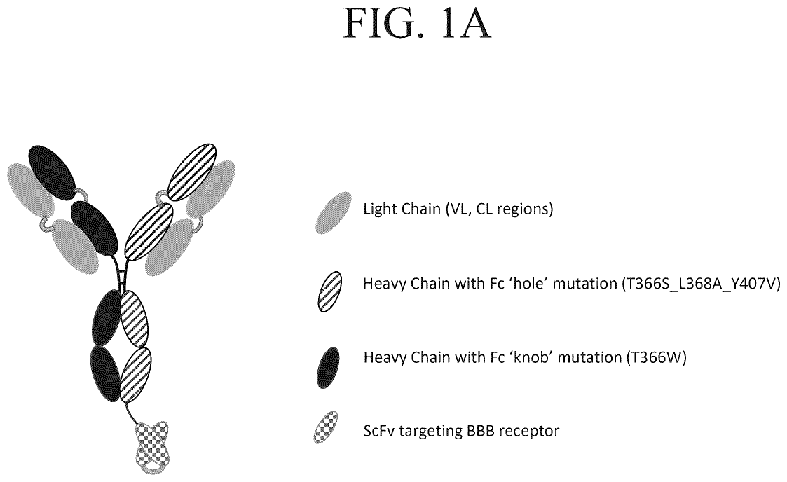

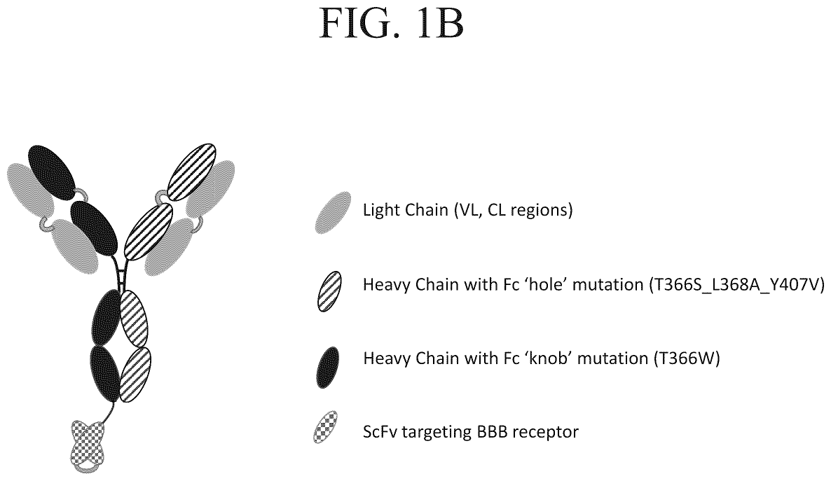

As used herein, a “complex” refers to one or more proteins comprising connected parts. The parts can be connected e.g., via a peptide bond (e.g., in a fusion protein or complex), a linker (e.g., a peptide linker), or via noncovalent protein-protein interactions such as disulfide bonds (e.g., in an antibody). Exemplary parts that can be included in a complex include an antigen-binding domain that specifically binds to N3pE Aβ and an antigen-binding domain that specifically binds to CD98hc or TfR, an Fc region, and/or an Fc domain. Accordingly, non-limiting examples of a “complex” comprising an anti-N3pE Aβ antigen-binding domain and an anti-CD98hc or anti-TfR antigen-binding domain include (a) a fusion protein or complex comprising the anti-pyroGlu3 Aβ antigen-binding domain and the anti-CD98hc or anti-TfR antigen-binding domain in a single polypeptide chain and (b) four proteins connected via noncovalent protein-protein interactions, wherein the first protein contains a heavy chain of anti-pyroGlu3 Aβ antibody comprising an anti-pyroGlu3 Aβ antigen-binding domain, the second protein contains a heavy chain of the anti-pyroGlu3 Aβ fused to an scFv containing an anti-CD98hc or anti-TfR scFv containing an anti-CD98hc or anti-TfR antigen-binding domain, and the third and fourth proteins contain light chains of the anti-pyroGlu3 Aβ antibody (e.g., as shown in FIG. 1). Formats of other exemplary complexes are provided in FIGS. 1-3.

As used herein, the term “composition” refers to pharmaceutical combination of components. The components can be connected to each other via covalent bonds or non-covalent bonds or can merely be contained in the same mixture or solution. Accordingly, a “composition” comprising an antigen-binding domain can refer, e.g., to an antibody, scFv, or Fab comprising the antigen-binding domain, can refer to a fusion protein or complex comprising the antigen-binding domain, or can refer to a mixture of solution comprising such an antigen-binding domain, antibody, scFv, Fab, fusion protein or complex. In some aspects, a composition is a “pharmaceutical composition.”

As used herein the term “pharmaceutical composition” refers to a combination comprising an active agent (e.g., an antigen-binding domain, an antibody, a scFv, or Fab comprising the antigen-binding domain disclosed herein) with at least one inert pharmaceutically acceptable agent (e.g., an excipient or a carrier).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range are also provided.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially of” are open-ended, allowing for the presence of more than that which is recited so long as basic or novel characteristics of that which is recited is not changed by the presence of more than that which is recited, but excludes prior art aspects.

All references cited herein, including patent applications and publications, are hereby incorporated by reference in their entirety.

The present disclosure will be more fully understood by reference to the following Examples. They should not, however, be construed as limiting the scope of the present disclosure. All citations throughout the disclosure are hereby expressly incorporated by reference.

### Antigen-Binding Domains and Compositions Comprising Antigen-Binding Domains

In some aspects, compositions provided herein may comprise an antigen-binding domain.

In some aspects, compositions provided herein may comprise a fusion protein or complex. In some aspects, the fusion protein or complex comprises an antigen-binding domain.

In some aspects, compositions provided herein may comprise a multi-specific protein.

In some aspects, the multi-specific protein comprises an antigen-binding domain.

In some aspects, compositions provided herein may comprise an antibody or an antibody fragment thereof.

In some aspects, compositions provided herein may comprise an antigen-binding fragment. Antigen-binding fragments of antibodies include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that can be bivalent and/or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In some aspects, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

As provided herein, a composition comprising an antigen-binding domain provided herein can be chimeric. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody.

As provided herein, a composition comprising an antigen-binding domain provided herein can be humanized. Typically, a non-human antibody is humanized to reduce immunogenicity to humans, while retaining the specificity and affinity of the parental non-human antibody. In some aspects, a humanized antibody is substantially non-immunogenic in humans. In some aspects, a humanized antibody has substantially the same affinity for a target as an antibody from another species from which the humanized antibody is derived. See, e.g., U.S. Pat. Nos. 5,530,101; 5,693,761; 5,693,762; and 5,585,089. In some aspects, amino acids of an antibody variable domain that can be modified without diminishing the native affinity of the antigen-binding domain while reducing its immunogenicity are identified. See, e.g., U.S. Pat. Nos. 5,766,886 and 5,869,619. Generally, a humanized antibody comprises one or more variable domains in which CDRs (or portions thereof) are derived from a non-human antibody, and framework regions (FRs) (or portions thereof) are derived from human antibody sequences. A humanized antibody can comprise at least a portion of a human constant region. In some aspects, some FR residues in a humanized antibody are substituted with corresponding residues from a non-human antibody (e.g., the antibody from which the CDR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337; 7,527,791; 6,982,321; and 7,087,409. Human framework regions that can be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

As provided herein, a composition comprising an antigen-binding domain provided herein can be human. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies can be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human mAbs with the desired specificity can be produced and selected. Certain exemplary methods are described in U.S. Pat. No. 5,545,807, EP546073, and EP546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals can be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al. Proc. Natd. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3):927-937 (2005) and Vollmers et al. Methods and Findings in Experimental and Clinical Pharmacology 27(3):185-91 (2005). Human antibodies can also be generated by isolating Fv clone variable domain sequences selected from human-derived phage display libraries. Such variable domain sequences can then be combined with a desired human constant domain. Techniques for selecting human antibodies from antibody libraries are described below.

In some aspects, an antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J. Mol. Biol. 338(2): 299-310, 2004; Lee et al. J. Mol. Biol. 340(5): 1073-1093, 2004; Fellouse Proc. Natl. Acad. Sci. USA 101(34):12467-12472 (2004); and Lee et al. J. Immunol. Methods 284(2):1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12: 725-734 (1993). Finally, naive libraries can also be made synthetically by cloning unrearranged V-gene segments from stem cells, and using PCR primers comprising random sequence to encode the highly variable CDR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and U.S. Patent Publication Nos. 2007/0292936 and 2009/0002360. Antibodies isolated from human antibody libraries are considered human antibodies or human antibody fragments herein.

### Fc Domains and Regions

A composition provided herein can comprise an Fc domain or region or fragment thereof. In some aspects, an Fc domain or region is of IgG class, the IgM class, or the IgA class. In some aspects, an Fc domain or region or fragment thereof is an IgG Fc domain or region or fragment thereof. In some aspects, an Fc domain or region or fragment thereof is a human IgG Fc domain or region or fragment thereof. In some aspects, an Fc domain or region or fragment thereof is a human IgG1 Fc domain or region or fragment thereof. In some aspects, an Fc domain or region or fragment thereof is a human IgG2 Fc domain or region or fragment thereof. In some aspects, an Fc domain or region or fragment thereof is a human IgG4 Fc domain or region or fragment thereof.

In some aspects provided herein, a composition provided herein comprises a modified Fc domain or region or fragment thereof. In some aspects, the modified Fc domain or region or fragment thereof is a modified IgG1 Fc comprising one or more modifications. For example, in some aspects, the IgG1 modified Fc comprises one or more amino acid substitutions (e.g., relative to a wild-type Fc domain of the same isotype).

In some aspects, the Fc domain or region or fragment thereof is a wildtype IgG2. In some aspects, the modified Fc domain or region or fragment thereof is a modified IgG2 Fc comprising one or more modifications. For example, in some aspects, the modified IgG2 Fc comprises one or more amino acid substitutions (e.g., relative to a wild-type IgG2 Fc).

In some aspects, the Fc domain or region or fragment thereof is a wildtype IgG4. In some aspects, the modified Fc domain or region or fragment thereof is a modified IgG4 Fc comprising one or more modifications. For example, in some aspects, the modified IgG4 Fc comprises one or more amino acid substitutions (e.g., relative to a wild-type IgG4 Fc). In some aspects, the one or more amino acid substitutions in the modified IgG4 Fc are IgG4-S228P or IgG4-S228P/L235E, where the amino acid position is according to the EU numbering convention.

In some aspects, the Fc domain or region or fragment thereof is a human IgG1 and the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), K322A (Hezareh et al. (2001) J Virol. 75(24) 12161-12168), S267E, L328F, A330L, M252Y, S254T, E430G, and/or T256E, where the amino acid position is according to the EU numbering convention. In some aspects, the IgG1 Fc comprises the amino acid substitutions L234A, L235A, and P329S (LALAPS) according to EU numbering. In some aspects, the IgG1 Fc comprises the amino acid substitutions L234A, L235A, and P331S (LALAPSv2) according to EU numbering. In some aspects, the IgG1 Fc comprises the amino acid substitutions L234A, L235A, and P329G (LALAPG) according to EU numbering. In some aspects, the IgG1 Fc comprises the amino acid substitutions L234A, L235A, and P331G (LALAPGv2) according to EU numbering. In some aspects, the IgG1 Fc comprises N325S and L328F mutations according to EU numbering. In some aspects, the IgG1 Fc comprises P329G or P329S according to EU numbering. In some aspects, the IgG1 Fc comprises K322A according to EU numbering.

TABLE 1
Exemplary Fc Domains

| Fc mutations | |
|---|---|
| (according to EU | |
| numbering) | Description |
| huIgG1 WT | Human IgG1 Fc |
| | ASTKGPSVFPLAPSSKSTSGGTAALGCLVKDYFPEPVTVSW |
| | NSGALTSGVHTFPAVLQSSGLYSLSSVVTVPSSSLGTQTYIC |
| | NVNHKPSNTKVDKKVEPKSCDKTHTCPPCPAPELLGGPSVF |
| | LFPPKPKDTLMISRTPEVTCVVVDVSHEDPEVKFNWYVDG |
| | VEVHNAKTKPREEQYNSTYRVVSVLTVLHQDWLNGKEYK |
| | CKVSNKALPAPIEKTISKAKGQPREPQVYTLPPSRDELTKNQ |
| | VSLTCLVKGFYPSDIAVEWESNGQPENNYKTTPPVLDSDGS |
| | FFLYSKLTVDKSRWQQGNVFSCSVMHEALHNHYTQKSLSL |
| | SPGK (SEQ ID NO: 211) |
| huIgG1 NSLF (N325S, | Both polypeptides of huIgG1 Fc contain N325S and L328F |
| L328F) | mutations, which decreases binding to FcGR3a and increase |
| | binding to FcGR2a |
| huIgG1 LALAPS (L234A, | Both polypeptides of huIgG1 Fc contain L234A, L235A, and |
| L235A, P329S) | P329S mutations, which minimize effector function (FcGR2a, |
| | FcgR3a and complement binding) |
| huIgG1 LALAPSv2 (L234A, | Both polypeptides of huIgG1 Fc contain L234A, L235A, and |
| L235A, P331S) | P331S mutations, which minimize effector function (FcGR2a, |
| | FcgR3a and complement binding) |
| huIgG1 LALA (L234A, | Both polypeptides of huIgG1 Fc contain L234A, L235A |
| L235A) | mutations, which minimize effector function (FcGR2a, FcgR3a |
| | binding) |
| huIgG1 K322A | Both polypeptides of huIgG1 Fc contain K332A mutations, which |
| | minimizes complement activated effector function. |
| huIgG1 YTE (M252Y, | Both polypeptides of huIgG1 Fc contain M252Y, S254T, T256E |
| S254T, T256E) | mutations, which enhance affinity to FcRn to extend antibody half- |
| | life |
| huIgG1 LS (M428L, | Both huIgG1 Fc contain M428L, N434S mutations. |
| N434S) | This enhances affinity to FcRn to extend antibody half-life |
| huIgG1 LALAPS (L234A, | One huIgG1 Fc polypeptide contains L234A, L235A, and P329S |
| L235A, P329S)/huIgG1 | mutations. The other huIgG1 Fc polypeptide is wild-type. |
| WT | This tunes effector function activity (FcgR2, FcgR3a and |
| | complement binding) |
| huIgG1 LALAPSv2 (L234A, | One huIgG1 Fc polypeptide contains L234A, L235A, and P331S |
| L235A, P331S)/huIgG1 | mutations. The other huIgG1 Fc polypeptide is wild-type. |
| WT | This tunes effector function activity (FcgR2, FcgR3a and |
| | complement binding) |
| huIgG1 LALA (L234A, | One huIgG1 Fc polypeptide contains L234A and L235A |
| L235A)/huIgG1 WT | mutations. |
| | The other huIgG1 Fc polypeptide is wild-type. |
| | This tunes effector function activity (FcgR2, and FcgR3a binding) |
| HuIgG1 LALA (L234A, | One huIgG1 Fc polypeptide contains L234A and L235A |
| L235A)/hu IgG1 PS | mutations. |
| (P329S) | The other huIgG1 Fc polypeptide contains a P329S mutation. |
| | This tunes effector function activity (FcgR2, FcgR3a and |
| | complement binding). |
| HuIgG1 LALA (L234A, | One huIgG1 Fc polypeptide contains L234A and L235A |
| L235A)/huIgG1 PSv2 | mutations. |
| (P331S) | The other huIgG1 Fc polypeptide contains a P329S mutation. |
| | This tunes effector function activity (FcgR2, FcgR3a and |
| | complement binding). |
| huIgG2 WT | Human IgG2 Fc |
| | ASTKGPSVFPLAPCSRSTSESTAALGCLVKDYFPEPVTVSW |
| | NSGALTSGVHTFPAVLQSSGLYSLSSVVTVPSSNFGTQTYT |
| | CNVDHKPSNTKVDKTVERKCCVECPPCPAPPVAGPSVFLFP |
| | PKPKDTLMISRTPEVTCVVVDVSHEDPEVQFNWYVDGVEV |
| | HNAKTKPREEQFNSTFRVVSVLTVVHQDWLNGKEYKCKV |
| | SNKGLPAPIEKTISKTKGQPREPQVYTLPPSREEMTKNQVSL |
| | TCLVKGFYPSDIAVEWESNGQPENNYKTTPPMLDSDGSFFL |
| | YSKLTVDKSRWQQGNVFSCSVMHEALHNHYTQKSLSLSPG |
| | K (SEQ ID NO: 212) |
| huIgG4 WT | Human IgG4 Fc |
| | ASTKGPSVFPLAPCSRSTSESTAALGCLVKDYFPEPVTVSW |
| | NSGALTSGVHTFPAVLQSSGLYSLSSVVTVPSSSLGTKTYTC |
| | NVDHKPSNTKVDKRVESKYGPPCPSCPAPEFLGGPSVFLFPP |
| | KPKDTLMISRTPEVTCVVVDVSQEDPEVQFNWYVDGVEVH |
| | NAKTKPREEQFNSTYRVVSVLTVLHQDWLNGKEYKCKVS |
| | NKGLPSSIEKTISKAKGQPREPQVYTLPPSQEEMTKNQVSLT |
| | CLVKGFYPSDIAVEWESNGQPENNYKTTPPVLDSDGSFFLY |
| | SRLTVDKSRWQEGNVFSCSVMHEALHNHYTQKSLSLSLGK |
| | (SEQ ID NO: 213) |
| huIgG4 S228P | Both human IgG4 Fc polypeptides contain S228P mutations. |
| | This minimizes Fab arm exchange present in wt IgG4 Fc |
| huIgG4 S228P/L235E | Both human IgG4 Fc polypeptides contain S228P and L235E |
| | mutations. |
| | This minimizes Fab arm exchange present in wt IgG4 Fc and |
| | minimizes effector function (FcGR2a, FcgR3a binding). |

In some aspects provided herein, a composition provided herein comprises one or more mutations to promote heterodimerization of Fc domains. In some aspects, a Fc region of a bispecific complex provided herein is formed by Fc domains that contain amino acid mutations, substitutions, additions, or deletions to promote heterodimerization in which different polypeptides comprising different Fc domains can dimerize to yield a heterodimer configuration. In some aspects, a bispecific of the present disclosure comprises a first Fc sequence comprising a first CH3 region, and a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions.

Methods to promote heterodimerization of Fe domains include amino acid deletions, additions, or substitutions of the amino acid sequence of the Fc domain, such as by including a set of “knob-into-hole” deletions, additions, or substitutions or including amino acid deletions, additions, or substitutions to effect electrostatic steering of the Fc to favor attractive interactions among different polypeptide chains. Methods for promoting heterodimerization of complementary Fc polypeptides have been previously described in, for example, Ridgway et al, 1996, Protein Eng, 9:617-621; Merchant et al, 1998, Nature Biotechnol, 16:677-681; Moore et al, 2011, MAbs, 3:546-557; Von Kreudenstein et al, 2013, 5:646-654; Gunasekaran et al, 2010, J Biol Chem, 285:19637-19464; Leaver-Fay et al, 2016, Structure, 24:641-651; Ha et al, 2016, Frontiers in Immunology, 7:1; Davis et al, 2010, Protein Eng Des Sel, 23:195-202; PCT Pub. Nos. WO1996/027011; WO1998/050431; WO2006/028936; WO2009/089004; WO2011/143545; WO2014/067011; WO2012/058768; WO2018/027025; US Pub. Nos. US2014/0363426; US2015/0307628; US2018/0016354; US2015/0239991; US2017/0058054; U.S. Pat. Nos. 5,731,168; 7,183,076; 9,701,759; 9,605,084; 9,650,446; 8,216,805; 8,765,412; and 8,258,268.

In some aspects, complementary Fc polypeptides of an Fc heterodimer include a mutation to alter charge polarity across the Fc dimer interface such that co-expression of electrostatically matched Fc domains support favorable attractive interactions, thereby promoting desired Fc heterodimer formation; whereas unfavorable repulsive charge interactions suppress unwanted Fc homodimer formation (Guneskaran et al, 2010, J Biol Chem, 285:19637-19646). When co-expressed in a cell, association between the polypeptide chains is possible but the chains do not substantially self-associate due to charge repulsion.

“Knob-hole” or “knob-into-hole” configurations are complementary Fc polypeptides of an Fc heterodimer that promote heterodimerization of two Fc polypeptides. “Knob-into-hole” technology is described in U.S. Pat. Nos. 5,731,168; 7,695,936; 8,216,805; 8,765,412; Ridgway et al., Prot Eng 9, 617-621 (1996); and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). The protuberance and cavity can be made by altering the nucleic acid encoding the polypeptides, e.g., by site-specific mutagenesis, or by peptide synthesis.

In some aspects, a composition provided herein comprises a Fc dimer comprising a “knob” mutation in one Fc domain and a “hole” mutation in the other Fc domain. In some aspects, the “knob” mutation comprises the amino acid substitution T366W according to EU numbering. In some aspects, the “hole” mutation comprises the amino acids substitutions T366S, L368A, and Y407V according to EU numbering. In some aspects, the “knob” mutation comprises the amino acid substitution T366W in one of the two subunits of the Fc dimer, and the “hole” mutation comprises the amino acid substitutions T366S, L368A and Y407V in the other subunit of the Fc dimer. In some aspects, the subunit of the Fc dimer comprising the “knob” mutation additionally comprises the amino acid substitution S354C, and the subunit of the Fc dimer comprising the “hole” mutation additionally comprises the amino acid substitution Y349C. Introduction of these two cysteine residues results in the formation of a disulfide bridge between the two subunits of the Fc dimer, thus further stabilizing the dimer (Carter, J Immunol Methods 248, 7-15 (2001)). Thus, in such configurations, a first Fc polypeptide comprises amino acid modifications to form the “knob” and a second Fc polypeptide comprises amino acid modifications to form the “hole” thus forming an Fc heterodimer comprising complementary Fc polypeptides.

Exemplary paired amino acid modifications of complementary Fc polypeptides of an Fc heterodimeric configuration are set forth below in Table 2 (EU numbering). In some aspects, the Fc polypeptide is a wildtype human IgG1 Fc having one Fc polypeptide comprising a knob configuration and one Fc polypeptide comprising a hole configuration, e.g., as set forth: ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PSVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR DELTKNQVSLWCLVKGFYPSDIAVEWESNGQPENNYKTTPPVLDSDGSFFLYSKLTVDK SRWQQGNVFSCSVMHEALHNHYTQKSLSLSPGK (knob: SEQ ID NO:267) and ASTKGPSVFPLAPSSKSTSGGTAALGCLVKDYFPEPVTVSWNSGALTSGVHTFPAVLQSS GLYSL SSVVTVPSSSLGTQTYICNVNHKPSNTKVDKKVEPKSCDKTHTCPPCPAPELLGG PSVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVYTLPPSR DELTKNQVSLSCAVKGFYPSDIAVEWESNGQPENNYKTTPPVLDSDGSFFLVSKLTVDK SRWQQGNVFSCSVMHEALHNHYTQKSLSLSPGK (hole; SEQ ID NO: 268), respectively.

TABLE 2
Exemplary paired Fc modifications for heterodimeric Fc domains

| First Fc polypeptide | Second Fc polypeptide |
|---|---|
| T366Y | Y407T |
| T366W | T366S/L368W/Y407V |
| T366W | T366S/L368A/Y407V |
| T366W/S354C | T366S/L368A/Y407V/Y349C |
| T350V/L351Y/F405A/Y407V | T350V/T366L/K392L/T394W |
| K360D/D399M/Y407A | E345R/Q347R/T366V/K409V |
| K409D/K392D | D399K/E356K |
| K360E/K409W | Q347R/D399V/F405T |
| L360E/K409W/Y349C | Q347R/D399V/F405T/S354C |
| K370E/K409W | E357N/D399V/F405T |

### Exemplary Anti-PyroGlu3 Abeta (Aβ) Antibodies

The terms “anti-pyroGlu3 Abeta (Aβ) antibody,” “anti-pyroGlu3 Abeta (Aβ),” “antibody that binds to pyroGlu3 Abeta (Aβ),” used interchangeably herein, refer to an antibody that is capable of binding pyroGlu3 Abeta (Aβ) with sufficient affinity such that the antibody is useful as a diagnostic and/or therapeutic agent in targeting Abeta (Aβ).

Provided herein are antibodies to pyroGlu3 Abeta (Aβ). Anti-pyroGlu3 Abeta (Aβ) antibodies of the present disclosure provided herein are useful, e.g., for the treatment of disorders associated with amyloid beta, such as, for example, Alzherimer's disease.

Table 3 below lists the heavy chain CDR amino acid sequences (according to Kabat numbering) of anti-pyroGlu3 Abeta (Aβ) antibodies of the present disclosure. Table 4 below lists the light chain CDR amino acid sequences (according to Kabat numbering) of anti-pyroGlu3 Abeta (Aβ) antibodies of the present disclosure. Table 5 below lists the heavy chain variable region and light chain variable region amino acid sequences of anti-pyroGlu3 Abeta (Aβ) antibodies of the present disclosure.

TABLE 3
Heavy Chain CDR Sequences of Anti-pyroGlu3 Abeta (Aβ) Antibodies

| Antibody | CDR-H1 | CDR-H2 | CDR-H3 |
|---|---|---|---|
| AB-1 | SGSYYWG | SFYYSGTIYYNPSLKS | QREYSSSSNYYYYYMDV |
| | (SEQ ID NO: 4) | (SEQ ID NO: 5) | (SEQ ID NO: 6) |
| AB-2 | SSSYYWG | SIYFSGSTYYNPSLKS | HREMATIRDYHYYYMDV |
| | (SEQ ID NO: 7) | (SEQ ID NO: 8) | (SEQ ID NO: 9) |
| AB-5 | DYAMH (SEQ | GISWNNNNIDYADSVK | DREYSGSYYDAFDI (SEQ ID |
| | ID NO: 10) | G (SEQ ID NO: 11) | NO: 12) |
| AB-3 | SNYMS (SEQ | VIYSGGTKHYADSVKG | SYYYGSGSSDAFDI (SEQ ID |
| | ID NO: 13) | (SEQ ID NO: 14) | NO: 15) |
| AB-4 | NFGMS (SEQ | GIYWNGVSTSYPDSVK | GGSPYGDYGAFDI (SEQ ID |
| | ID NO: 16) | G (SEQ ID NO: 17) | NO: 18) |
| AB-6 | SGSYYWG | SFYYSGTIYYNPSLKS | QREYSSSSNYYYYYMDV |
| | (SEQ ID NO: 4) | (SEQ ID NO: 5) | (SEQ ID NO: 6) |
| AB-7 | SSSYYWG | SIYFSGSTYYNPSLKS | HREMATIRDYHYYYMDV |
| | (SEQ ID NO: 7) | (SEQ ID NO: 8) | (SEQ ID NO: 9) |
| AB-10 | DYAMH (SEQ | GISWNQGNIDYADSVK | DREYSGSYYDAFDI (SEQ ID |
| | ID NO: 10) | G (SEQ ID NO: 19) | NO: 12) |
| AB-8 | SNYMS (SEQ | VIYSGGTKHYADSVKG | SYYYGSGSSDAFDI (SEQ ID |
| | ID NO: 13) | (SEQ ID NO: 14) | NO: 15) |
| AB-9 | NFGMS (SEQ | GIYWNAVSTSYPDSVK | GGSPYGDYGAFDI (SEQ ID |
| | ID NO: 16) | G (SEQ ID NO: 20) | NO: 18) |

TABLE 4
Light Chain CDR Sequences of Anti-pyroGlu3 Abeta (Aβ) Antibodies

| Antibody | CDR-L1 | CDR-L2 | CDR-L3 |
|---|---|---|---|
| AB-1 | RASQSIRFYLN (SEQ | GASSLQS (SEQ ID | QQTYSTPIT |
| | ID NO: 21) | NO: 22) | (SEQ ID NO: 23) |
| AB-2 | RASQSISNYLN (SEQ | AASSLQS (SEQ ID | QQSYSTPLT |
| | ID NO: 24) | NO: 25) | (SEQ ID NO: 26) |
| AB-5 | RASQSISSWLA (SEQ | KASSLEN (SEQ ID | QEYNSYNT |
| | ID NO: 27) | NO: 28) | (SEQ ID NO: 29) |
| AB-3 | RASQSISNYLN (SEQ | DAYSLQI (SEQ ID | QQSYSIPPT |
| | ID NO: 24) | NO: 30) | (SEQ ID NO: 31) |
| AB-4 | RSSQSLVHSDGNTYLS | KISNRFS (SEQ ID | MQTTQFPYT |
| | (SEQ ID NO: 32) | NO: 33) | (SEQ ID NO: 34) |
| AB-6 | RASQSIRFYLN (SEQ | GASSLQS (SEQ ID | QQTYSTPIT |
| | ID NO: 21) | NO: 22) | (SEQ ID NO: 23) |
| AB-7 | RASQSISNYLN (SEQ | AASSLQS (SEQ ID | QQSYSTPLT |
| | ID NO: 24) | NO: 25) | (SEQ ID NO: 26) |
| AB-10 | RASQSISSWLA (SEQ | KASSLES (SEQ ID | QEYNSYNT |
| | ID NO: 27) | NO: 35) | (SEQ ID NO: 29) |
| AB-8 | RASQSISNYLN (SEQ | DAYSLQI (SEQ ID | QQSYSIPPT |
| | ID NO: 24) | NO: 30) | (SEQ ID NO: 31) |
| AB-9 | RSSQSLVHSEGNTYLS | KISNRFS (SEQ ID | MQTTQFPYT |
| | (SEQ ID NO: 36) | NO: 33) | (SEQ ID NO: 34) |

TABLE 5
VH and VL Sequences of Anti-pyroGlu3 Abeta (Aβ) Antibodies

| Antibody | VH sequence | VL sequence |
|---|---|---|
| AB-1 | QLQLQESGPGLVKPSETLSLTCT | DIQMTQSPSSLSASVGDRVTIT |
| | VSGGSISSGSYYWGWIRQPPGK | CRASQSIRFYLNWYQQKPGKA |
| | GLEWIGSFYYSGTIYYNPSLKSR | PKVLIYGASSLQSGVPSRFSGS |
| | VTISVDTSKNQFSLKLRSVTAAD | GSGTDFTLTISSLQPEDFATYH |
| | TAVYYCARQREYSSSSNYYYYY | CQQTYSTPITFGQGTRLEIK |
| | MDVWGKGTTVTVSS (SEQ ID | (SEQ ID NO: 38) |
| | NO: 37) | |
| AB-2 | QLHLQESGPGLVKPSETLSLTCT | DIQMTQSPSSLSASVGDRVTIT |
| | VSGGSTSSSSYYWGWIRQPPGK | CRASQSISNYLNWYQQKPGKA |
| | GLEWIGSIYFSGSTYYNPSLKSR | PKLLIYAASSLQSGVPSRFSGS |
| | VTISVDTSKNQFSLKLKTVTAA | GSGTDFTLTISSLQPEDFATYY |
| | DTAVYYCVRHREMATIRDYHY | CQQSYSTPLTFGGGTRVEIK |
| | YYMDVWGKGTTVTVSS (SEQ | (SEQ ID NO: 40) |
| | ID NO: 39) | |
| AB-5 | EVOLVESGGGLVQPGRSLRLSC | DIQMTQSPSTLSASVGDRVTIT |
| | AASGFTFDDYAMHWVRQAPGK | CRASQSISSWLAWYQQKPGK |
| | GLEWVSGISWNNNNIDYADSVK | APKVLIYKASSLENGVPSRFSG |
| | GRFTISRDNAKNSLYLQMNSLR | SGSGTEFTLTISSLQPDDFATY |
| | AEDTALYYCVKDREYSGSYYD | YCQEYNSYNTFGQGTKLEIK |
| | AFDIWGQGTMVTVSS (SEQ ID | (SEQ ID NO: 42) |
| | NO: 41) | |
| AB-3 | EVOLVESGGGLIQPGGSLRLSCA | DIQMTQSPSSLSASVGDRVTIT |
| | ASGFTVSSNYMSWVRQAPGKG | CRASQSISNYLNWYQQKPGKA |
| | LEWVSVIYSGGTKHYADSVKGR | PNLLIYDAYSLQIGVPSRFSGS |
| | FTISRDNSKNTLYLQMNSLRAE | GSGTDFTLTISSLQPEDFATYS |
| | DTAVYYCARSYYYGSGSSDAFD | CQQSYSIPPTFGGGTKVEIK |
| | IWGQGTMVTVSS (SEQ ID NO: | (SEQ ID NO: 44) |
| | 43) | |
| AB-4 | EVOLVESGGGVVRPGGSLRLSC | DIVMTQTPLSSPVTLGQPASIS |
| | AASGFTFDNFGMSWVRQAPGK | CRSSQSLVHSDGNTYLSWLQQ |
| | GLEWVSGIYWNGVSTSYPDSVK | RPGQPPRLLIYKISNRFSGVPD |
| | GRFTISRDNAKNSLYLQMNSLR | RFSGSGAGTDFTLKISRVEAED |
| | GEDTALYYCARGGSPYGDYGA | VGVYYCMQTTQFPYTFGQGT |
| | FDIWGQGTVVTVSS (SEQ ID | KLEIK (SEQ ID NO: 46) |
| | NO: 45) | |
| AB-6 | QLQLQESGPGLVKPSETLSLTCT | DIQMTQSPSSLSASVGDRVTIT |
| | VSGGSISSGSYYWGWIRQPPGK | CRASQSIRFYLNWYQQKPGKA |
| | GLEWIGSFYYSGTIYYNPSLKSR | PKVVIYGASSLQSGVPSRFSGS |
| | VTISVDTSKNQFSLKLSSVTAAD | GSGTDFTLTISSLQPEDFATYY |
| | TAVYYCARQREYSSSSNYYYYY | CQQTYSTPITFGQGTRLEIK |
| | MDVWGKGTTVTVSS (SEQ ID | (SEQ ID NO: 48) |
| | NO: 47) | |
| AB-7 | QLQLQESGPGLVKPSETLSLTCT | DIQMTQSPSSLSASVGDRVTIT |
| | VSGGSTSSSSYYWGWIRQPPGK | CRASQSISNYLNWYQQKPGKA |
| | GLEWIGSIYFSGSTYYNPSLKSR | PKLLIYAASSLQSGVPSRFSGS |
| | VTISVDTSKNQFSLKLSSVTAAD | GSGTDFTLTISSLQPEDFATYY |
| | TAVYYCVRHREMATIRDYHYY | CQQSYSTPLTFGGGTKVEIK |
| | YMDVWGKGTTVTVSS (SEQ ID | (SEQ ID NO: 50) |
| | NO: 49) | |
| AB-10 | EVOLVESGGGLVQPGRSLRLSC | DIQMTQSPSTLSASVGDRVTIT |
| | AASGFTFDDYAMHWVRQAPGK | CRASQSISSWLAWYQQKPGK |
| | GLEWVSGISWNQGNIDYADSVK | APKVVIYKASSLESGVPSRFSG |
| | GRFTISRDNAKNSLYLQMNSLR | SGSGTEFTLTISSLQPDDFATY |
| | AEDTALYYCVKDREYSGSYYD | YCQEYNSYNTFGQGTKLEIK |
| | AFDIWGQGTMVTVSS (SEQ ID | (SEQ ID NO: 52) |
| | NO: 51) | |
| AB-8 | EVOLVESGGGLIQPGGSLRLSCA | DIQMTQSPSSLSASVGDRVTIT |
| | ASGFTVSSNYMSWVRQAPGKG | CRASQSISNYLNWYQQKPGKA |
| | LEWVSVIYSGGTKHYADSVKGR | PKLLIYDAYSLQIGVPSRFSGS |
| | FTISRDNSKNTLYLQMNSLRAE | GSGTDFTLTISSLQPEDFATYY |
| | DTAVYYCARSYYYGSGSSDAFD | CQQSYSIPPTFGGGTK VEIK |
| | IWGQGTMVTVSS (SEQ ID NO: | (SEQ ID NO: 53) |
| | 43) | |
| AB-9 | EVOLVESGGGVVRPGGSLRLSC | DIVMTQTPLSSPVTLGQPASIS |
| | AASGFTFDNFGMSWVRQAPGK | CRSSQSLVHSEGNTYLSWLQQ |
| | GLEWVSGIYWNAVSTSYPDSVK | RPGQPPRLLIYKISNRFSGVPD |
| | GRFTISRDNAKNSLYLQMNSLR | RFSGSGAGTDFTLKISRVEAED |
| | AEDTALYYCARGGSPYGDYGA | VGVYYCMQTTQFPYTFGQGT |
| | FDIWGQGTMVTVSS (SEQ ID | KLEIK (SEQ ID NO: 55) |
| | NO: 54) | |

In some aspects, provided herein are anti-pyroGlu3 Abeta (Aβ) antibodies comprising: (a) HVR-H1 comprising the amino acid sequence of SEQ ID NO:4; (b) HVR-H2 comprising the amino acid sequence of SEQ ID NO:5; (c) HVR-H3 comprising the amino acid sequence of SEQ ID NO:6; (d) HVR-L1 comprising the amino acid sequence of SEQ ID NO:21; (e) HVR-L2 comprising the amino acid sequence of SEQ ID NO:22; and (f) HVR-L3 comprising the amino acid sequence of SEQ ID NO:23; (a) HVR-H1 comprising the amino acid sequence of SEQ ID NO:7; (b) HVR-H2 comprising the amino acid sequence of SEQ ID NO:8; (c) HVR-H3 comprising the amino acid sequence of SEQ ID NO:9; (d) HVR-L1 comprising the amino acid sequence of SEQ ID NO:24; (e) HVR-L2 comprising the amino acid sequence of SEQ ID NO:25; and (f) HVR-L3 comprising the amino acid sequence of SEQ ID NO:26; (a) HVR-H1 comprising the amino acid sequence of SEQ ID NO:10; (b) HVR-H2 comprising the amino acid sequence of SEQ ID NO:11; (c) HVR-H3 comprising the amino acid sequence of SEQ ID NO:12; (d) HVR-L1 comprising the amino acid sequence of SEQ ID NO:27; (e) HVR-L2 comprising the amino acid sequence of SEQ ID NO:28; and (f) HVR-L3 comprising the amino acid sequence of SEQ ID NO:29; (a) HVR-H1 comprising the amino acid sequence of SEQ ID NO:13; (b) HVR-H2 comprising the amino acid sequence of SEQ ID NO:14; (c) HVR-H3 comprising the amino acid sequence of SEQ ID NO:15; (d) HVR-L1 comprising the amino acid sequence of SEQ ID NO:24; (e) HVR-L2 comprising the amino acid sequence of SEQ ID NO:30; and (f) HVR-L3 comprising the amino acid sequence of SEQ ID NO:31; (a) HVR-H1 comprising the amino acid sequence of SEQ ID NO:16; (b) HVR-H2 comprising the amino acid sequence of SEQ ID NO:17; (c) HVR-H3 comprising the amino acid sequence of SEQ ID NO:18; (d) HVR-L1 comprising the amino acid sequence of SEQ ID NO:32; (e) HVR-L2 comprising the amino acid sequence of SEQ ID NO:33; and (f) HVR-L3 comprising the amino acid sequence of SEQ ID NO:34; (a) HVR-H1 comprising the amino acid sequence of SEQ ID NO:4; (b) HVR-H2 comprising the amino acid sequence of SEQ ID NO:5; (c) HVR-H3 comprising the amino acid sequence of SEQ ID NO:6; (d) HVR-L1 comprising the amino acid sequence of SEQ ID NO:21; (e) HVR-L2 comprising the amino acid sequence of SEQ ID NO:22; and (f) HVR-L3 comprising the amino acid sequence of SEQ ID NO:23; (a) HVR-H1 comprising the amino acid sequence of SEQ ID NO:7; (b) HVR-H2 comprising the amino acid sequence of SEQ ID NO:8; (c) HVR-H3 comprising the amino acid sequence of SEQ ID NO:9; (d) HVR-L1 comprising the amino acid sequence of SEQ ID NO:24; (e) HVR-L2 comprising the amino acid sequence of SEQ ID NO:25; and (f) HVR-L3 comprising the amino acid sequence of SEQ ID NO:26; (a) HVR-H1 comprising the amino acid sequence of SEQ ID NO:10; (b) HVR-H2 comprising the amino acid sequence of SEQ ID NO:19; (c) HVR-H3 comprising the amino acid sequence of SEQ ID NO:12; (d) HVR-L1 comprising the amino acid sequence of SEQ ID NO:27; (e) HVR-L2 comprising the amino acid sequence of SEQ ID NO:35; and (f) HVR-L3 comprising the amino acid sequence of SEQ ID NO:29; (a) HVR-H1 comprising the amino acid sequence of SEQ ID NO:13; (b) HVR-H2 comprising the amino acid sequence of SEQ ID NO:14; (c) HVR-H3 comprising the amino acid sequence of SEQ ID NO:15; (d) HVR-L1 comprising the amino acid sequence of SEQ ID NO:24; (e) HVR-L2 comprising the amino acid sequence of SEQ ID NO:30; and (f) HVR-L3 comprising the amino acid sequence of SEQ ID NO:31; and (a) HVR-H1 comprising the amino acid sequence of SEQ ID NO:16; (b) HVR-H2 comprising the amino acid sequence of SEQ ID NO:20; (c) HVR-H3 comprising the amino acid sequence of SEQ ID NO:18; (d) HVR-L1 comprising the amino acid sequence of SEQ ID NO:36; (e) HVR-L2 comprising the amino acid sequence of SEQ ID NO:33; and (f) HVR-L3 comprising the amino acid sequence of SEQ ID NO:34.

In another aspect, an anti-pyroGlu3 Abeta (Aβ) antibody comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs:37, 39, 41, 43, 45, 47, 49, 51, and 54. In certain aspects, a VH sequence having at least 90%, 91%, 92%, 93%, 94%, 95%, 96%, 97%, 98%, or 99% identity to an amino acid sequence selected from the group consisting of SEQ ID NOs: 37, 39, 41, 43, 45, 47, 49, 51, and 54 contains substitutions (e.g., conservative substitutions), insertions, or deletions relative to the reference sequence, but an anti-pyroGlu3 A Abeta (β) antibody comprising that sequence retains the ability to bind to pyroGlu3 Abeta (Aβ). In certain aspects, a total of 1 to 10 amino acids have been substituted, inserted, and/or deleted in SEQ ID NO: 37, 39, 41, 43, 45, 47, 49, 51, or 54. In certain aspects, a total of 1 to 5 amino acids have been substituted, inserted and/or deleted in SEQ ID NO: 37, 39, 41, 43, 45, 47, 49, 51, or 54. In certain aspects, substitutions, insertions, or deletions occur in regions outside the HVRs (i.e., in the FRs). Optionally, the anti-pyroGlu3 Abeta (Aβ) antibody comprises the V_{H} sequence of SEQ ID NO: 37, 39, 41, 43, 45, 47, 49, 51, and 54, including post-translational modifications of that sequence. In a particular aspect, the VH comprises one, two or three HVRs selected from: (a) HVR-H1 comprising an amino acid sequence selected from the group consisting of SEQ ID NOs:4, 7, 10, 13, and 16; (b) HVR-H2 comprising an amino acid sequence selected from the group consisting of SEQ ID NOs:5, 8, 11, 14, 17, 19, and 20; and (c) HVR-H3 comprising an amino acid sequence selected from the group consisting of SEQ ID NOs:6, 9, 12, 15, and 18.

In another aspect, an anti-pyroGlu3 Abeta (Aβ) antibody is provided, wherein the antibody comprises a light chain variable domain (VL) having at least 90%, 91%, 92%, 93%, 94%, 95%, 96%, 97%, 98%, 99%, or 100% sequence identity to an amino acid sequence selected from the group consisting of SEQ ID NOs:38, 40, 42, 44, 46, 48, 50, 52, 53, and 55. In certain aspects, a VL sequence having at least 90%, 91%, 92%, 93%, 94%, 95%, 96%, 97%, 98%, or 99% identity to an amino acid sequence selected from the group consisting of SEQ ID NOs: 38, 40, 42, 44, 46, 48, 50, 52, 53, and 55, and contains substitutions (e.g., conservative substitutions), insertions, or deletions relative to the reference sequence, but an anti-pyroGlu3 Abeta (Aβ) antibody comprising that sequence retains the ability to bind to pyroGlu3 Abeta (Aβ). In some aspects, a total of 1 to 10 amino acids have been substituted, inserted and/or deleted in SEQ ID NO: 38, 40, 42, 44, 46, 48, 50, 52, 53, or 55. In certain aspects, a total of 1 to 5 amino acids have been substituted, inserted and/or deleted in SEQ ID NO: 38, 40, 42, 44, 46, 48, 50, 52, 53, or 55. In certain aspects, the substitutions, insertions, or deletions occur in regions outside the HVRs (i.e., in the FRs). Optionally, the anti-pyroGlu3 Abeta (Aβ) antibody comprises the VL sequence of SEQ ID NO: 38, 40, 42, 44, 46, 48, 50, 52, 53, and 55, including post-translational modifications of that sequence. In a particular aspect, the VL comprises one, two or three HVRs selected from (a) HVR-L1 comprising an amino acid sequence selected from the group consisting of SEQ ID NOs:21, 24, 27, 32, and 36; (b) HVR-L2 comprising an amino acid sequence selected from the group consisting of SEQ ID NOs:22, 25, 28, 30, 33, and 35; and (c) HVR-L3 comprising an amino acid sequence selected from the group consisting of SEQ ID NOs:23, 26, 29, 31, and 34.

In some aspects, an anti-pyroGlu3 Abeta (Aβ) antibody is provided, wherein the antibody comprises a V_{H} as in any of the aspects provided above, and a V_{L} as in any of the aspects provided above. In some aspects, provided herein are anti-pyroGlu3 Abeta (Aβ) antibodies, wherein the antibody comprises a V_{H} as in any of the aspects provided above, and a V_{L} as in any of the aspects provided above. In one aspect, the antibody comprises the V_{H} and V_{L} sequences in SEQ ID NOs:37, 39, 41, 43, 45, 47, 49, 51, and 54, and SEQ ID NOs: 38, 40, 42, 44, 46, 48, 50, 52, 53, and 55, respectively, including post-translational modifications of those sequences.

In some aspects, provided herein are anti-pyroGlu3 Abeta (Aβ) antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO:37 and V_{L} comprising the amino acid sequence of SEQ ID NO:38; V_{H} comprising the amino acid sequence of SEQ ID NO:39 and V_{L} comprising the amino acid sequence of SEQ ID NO:40; V_{H} comprising the amino acid sequence of SEQ ID NO:41 and V_{L} comprising the amino acid sequence of SEQ ID NO:42; V_{H} comprising the amino acid sequence of SEQ ID NO:43 and V_{L} comprising the amino acid sequence of SEQ ID NO:44; V_{H} comprising the amino acid sequence of SEQ ID NO:45 and V_{L} comprising the amino acid sequence of SEQ ID NO:46; V_{H} comprising the amino acid sequence of SEQ ID NO:47 and V_{L} comprising the amino acid sequence of SEQ ID NO:48; V_{H} comprising the amino acid sequence of SEQ ID NO:49 and V_{L} comprising the amino acid sequence of SEQ ID NO:50; V_{H} comprising the amino acid sequence of SEQ ID NO:51 and V_{L} comprising the amino acid sequence of SEQ ID NO:52; V_{H} comprising the amino acid sequence of SEQ ID NO:43 and V_{L} comprising the amino acid sequence of SEQ ID NO:53; and V_{H} comprising the amino acid sequence of SEQ ID NO:54 and V_{L} comprising the amino acid sequence of SEQ ID NO:55.

In some aspects, an anti-Abeta (pyroGlu3 Aβ) antibody of the present disclosure competitively inhibits binding of at least one reference antibody selected from anti-pyroGlu3 Abeta (Aβ) antibody AB-1, AB-2, AB-3, AB-4, AB-5, AB-6, AB-7, AB-8, AB-9, and AB-10, and any combination thereof, for binding to pyroGlu3 Abeta (Aβ).

In some aspects, an anti-pyroGlu3 Abeta (Aβ) antibody of the present disclosure binds to an epitope of human pyroGlu3 Abeta (Aβ) that is the same as or overlaps with the pyroGlu3 Abeta (Aβ) epitope bound by at least one reference antibody selected from anti-pyroGlu3 Abeta (Aβ) antibody AB-1, AB-2, AB-3, AB-4, AB-5, AB-6, AB-7, AB-8, AB-9, and AB-10. Detailed exemplary methods for mapping an epitope to which an antibody binds are provided in Morris (1996) “Epitope Mapping Protocols,” in Methods in Molecular Biology vol. 66 (Humana Press, Totowa, NJ).

In some aspects, an anti-pyroGlu3 Abeta (Aβ) antibody of the present disclosure competitively inhibits binding of at least one reference antibody, or binds to an epitope of human pyroGlu3 Abeta (Aβ) that is the same as or overlaps with the pyroGlu3 Abeta (Aβ) epitope bound by at least one reference antibody, wherein the reference antibody is an anti-pyroGlu3 Abeta (Aβ) antibody comprising a heavy chain variable domain (V_{H}) and a light chain variable domain (V_{L}), wherein the V_{H} and V_{L} are selected from the group consisting of: V_{H} comprising the amino acid sequence of SEQ ID NO:37 and V_{L} comprising the amino acid sequence of SEQ ID NO:38; V_{H} comprising the amino acid sequence of SEQ ID NO:39 and V_{L} comprising the amino acid sequence of SEQ ID NO:40; V_{H} comprising the amino acid sequence of SEQ ID NO:41 and V_{L} comprising the amino acid sequence of SEQ ID NO:42; V_{H} comprising the amino acid sequence of SEQ ID NO:43 and V_{L} comprising the amino acid sequence of SEQ ID NO:44; V_{H} comprising the amino acid sequence of SEQ ID NO:45 and V_{L} comprising the amino acid sequence of SEQ ID NO:46; V_{H} comprising the amino acid sequence of SEQ ID NO:47 and V_{L} comprising the amino acid sequence of SEQ ID NO:48; V_{H} comprising the amino acid sequence of SEQ ID NO:49 and V_{L} comprising the amino acid sequence of SEQ ID NO:50; V_{H} comprising the amino acid sequence of SEQ ID NO:51 and V_{L} comprising the amino acid sequence of SEQ ID NO:52; V_{H} comprising the amino acid sequence of SEQ ID NO:43 and V_{L} comprising the amino acid sequence of SEQ ID NO:53; and V_{H} comprising the amino acid sequence of SEQ ID NO:54 and V_{L} comprising the amino acid sequence of SEQ ID NO:55.

In some aspects, the anti-pyroGlu3 Abeta (Aβ) antibody according to any of the above aspects is a monoclonal antibody, including a humanized and/or human antibody. In some aspects, the anti-pyroGlu3 Abeta (Aβ) antibody is an antibody fragment, e.g., a Fv, Fab, Fab′, scFv, diabody, or F(ab′)2 fragment. In some aspects, the anti-pyroGlu3 Abeta (Aβ) antibody is a substantially full-length antibody, e.g., an IgG1 antibody, IgG2a antibody, IgG4 antibody or other antibody class or isotype as defined herein.

In some aspects, an anti-pyroGlu3 Abeta (Aβ) antibody according to any of the above aspects may incorporate any of the features, singly or in combination, as described below.

Multi-Specific Binding Proteins that Bind to Blood-Brain Barrier Receptors or Proteins

Provided herein are multi-specific binding proteins that specifically bind to pyroGlu3 Abeta (Aβ) and specifically bind to human receptors or proteins of the blood-brain barrier. Such multi-specific binding proteins are capable of crossing the blood brain barrier (BBB) and capable of transporting other agents (e.g., therapeutically active agents, such as anti-pyroGlu3 Abeta (Aβ) antibodies, antigen-binding domains, or fragments thereof) with the composition across the blood brain barrier. Exemplary human receptors or proteins of the blood-brain barrier are transferrin receptor (TfR) and CD98 heavy chain (CD98hc).

In some aspects, the compositions provided herein comprise a pyroGlu3 Abeta (Aβ) antigen-binding domain and a TfR antigen-binding domain. In some aspects, the compositions provided herein comprise a pyroGlu3 Abeta (Aβ) antigen-binding domain and a CD98hc antigen-binding domain.

### Antigen-Binding Domains That Bind to TfR

Provided herein are antigen-binding domains that specifically bind to human TfR. Such anti-TfR antigen-binding domains are capable of crossing the blood brain barrier (BBB) and capable of transporting other agents (e.g., therapeutically active agents) associated with the antigen-binding domain across the blood brain barrier. Accordingly, in some aspects, provided herein are antigen-binding domains that specifically bind to human TfR and are capable of being internalized in blood brain barrier epithelial cells.

Antibodies (e.g., antigen-binding domains) to human transferrin receptor were obtained, characterized, and recombinantly expressed as described previously in WO2024/026472 (filed Jul. 28, 2023) and WO2025/166045 (International Application No. PCT/US2025/013846; filed Jan. 30, 2025), each of which is incorporated herein by reference in its entirety.

Table 6 below lists the heavy chain CDR amino acid sequences (according to Kabat numbering) of anti-TfR antigen-binding domains of the present disclosure. Table 7 below lists the light chain CDR amino acid sequences (according to Kabat numbering) of anti-TfR antigen-binding domains of the present disclosure. Table 8 below lists the heavy chain variable region and light chain variable region amino acid sequences of anti-TfR antigen-binding domains of the present disclosure.

TABLE 6
Heavy Chain CDR Sequences of Anti-TfR Antigen Binding Domains

| Antibody | CDR-H1 | CDR-H2 | CDR-H3 |
|---|---|---|---|
| TfR.15.WH8.1.24A | SYYMH (SEQ | NIYPGSGSSNYAQKFQG (SEQ | SAYYGNFDTMDN (SEQ |
| | ID NO: 56) | ID NO: 57) | ID NO: 58) |
| TfR.15.WH8.1.24A.42Q | SYYMH (SEQ | NIYPGSGSSNYAQKFQG (SEQ | SAYYGNFDTMDN (SEQ |
| | ID NO: 56) | ID NO: 57) | ID NO: 58) |
| TfR.15.WH8.1.42Q | SYYMH (SEQ | NIYPGSGSSNYAQKFQG (SEQ | SSYYGNFDTMDN (SEQ |
| | ID NO: 56) | ID NO: 57) | ID NO: 59) |
| TfR.15.WH8.1.42Q.H6-4 | SYYMH (SEQ | NIYPGSGSSNYAQKFQG (SEQ | SSYYGRFDTMDN (SEQ |
| | ID NO: 56) | ID NO: 57) | ID NO: 60) |
| TfR.15.WH8.1.42Q.L7-2 | SYYMH (SEQ | NIYPGSGSSNYAQKFQG (SEQ | SSYYGNFDTMDN (SEQ |
| | ID NO: 56) | ID NO: 57) | ID NO: 59) |
| TfR. 15.WH8.1.42Q.L10-1 | SYYMH (SEQ | NIYPGSGSSNYAQKFQG (SEQ | SSYYGNFDTMDN (SEQ |
| | ID NO: 56) | ID NO: 57) | ID NO: 59) |
| TfR.15.WH8.1.42Q.L10-8 | SYYMH (SEQ | NIYPGSGSSNYAQKFQG (SEQ | SSYYGNFDTMDN (SEQ |
| | ID NO: 56) | ID NO: 57) | ID NO: 59) |
| TfR.15.WH8.1.42Q.L10-16 | SYYMH (SEQ | NIYPGSGSSNYAQKFQG (SEQ | SSYYGNFDTMDN (SEQ |
| | ID NO: 56) | ID NO: 57) | ID NO: 59) |
| TfR.15.WH8.1.42Q.H3-7 | SYYMH (SEQ | NIYPGSGSSNYAQKFQG (SEQ | SSNYGNFDTMDN (SEQ |
| | ID NO: 56) | ID NO: 57) | ID NO: 61) |
| TfR.9.1B.39.38 | EYAMH (SEQ | GIAPNTGGTSYNQKFKG (SEQ | QGWLLRGGDY (SEQ ID |
| | ID NO: 62) | ID NO: 63) | NO: 64) |
| TfR.9.1B.39.27.L-35 | EYAMH (SEQ | GIAPGTGGTSYNQKFKG (SEQ | QGWLLRGGDY (SEQ ID |
| | ID NO: 62) | ID NO: 65) | NO: 64) |
| TfR.9.1B.39.38.L-21 | EYAMH (SEQ | GIAPNTGGTSYNQKFKG (SEQ | QGWLLRGGDY (SEQ ID |
| | ID NO: 62) | ID NO: 63) | NO: 64) |
| TfR.9.1B.39.38.L-6 | EYAMH (SEQ | GIAPNTGGTSYNQKFKG (SEQ | QGWLLRGGDY (SEQ ID |
| | ID NO: 62) | ID NO: 63) | NO: 64) |
| TfR.9.1B.39.27.L-19 | EYAMH (SEQ | GIAPGTGGTSYNQKFKG (SEQ | QGWLLRGGDY (SEQ ID |
| | ID NO: 62) | ID NO: 65) | NO: 64) |
| TfR.15.WH8.1 | SYYMH (SEQ | NIYPGSGSSNYAQKFQG (SEQ | SSYYGNFDTMDN (SEQ |
| | ID NO: 56) | ID NO: 57) | ID NO: 59) |
| TfR.9.1B.35 | EYAMH (SEQ | GINPNDGGTSYNQKFKG (SEQ | QGWLLRGGDY (SEQ ID |
| | ID NO: 62) | ID NO: 275) | NO: 64) |
| TfR.9.1B.39 | EYAMH (SEQ | GINPNDGGTSYNQKFKG (SEQ | QGWLLRGGDY (SEQ ID |
| | ID NO: 62) | ID NO: 275) | NO: 64) |

TABLE 7
Light Chain CDR Sequences of Anti-TfR Antigen Binding Domains

| Antibody | CDR-L1 | CDR-L2 | CDR-L3 |
|---|---|---|---|
| TfR.15.WH8.1.24A | RASSSIRYMN (SEQ ID | DTSNRAT (SEQ ID | HQRSSYPWT (SEQ |
| | NO: 66) | NO: 67) | ID NO: 68) |
| TfR.15.WH8.1.24A.42Q | RASSSIRQMN (SEQ ID | DTSNRAT (SEQ ID | HQRSSYPWT (SEQ |
| | NO: 69) | NO: 67) | ID NO: 68) |
| TfR.15.WH8.1.42Q | RASSSIRQMN (SEQ ID | DTSNRAT (SEQ ID | HQRSSYPWT (SEQ |
| | NO: 69) | NO: 67) | ID NO: 68) |
| TfR.15.WH8.1.42Q.H6-4 | RASSSIRQMN (SEQ ID | DTSNRAT (SEQ ID | HQRSSYPWT (SEQ |
| | NO: 69) | NO: 67) | ID NO: 68) |
| TfR.15.WH8.1.42Q.L7-2 | RASSSIDQMN (SEQ ID | DTSNRAT (SEQ ID | HQRSSYPWT (SEQ |
| | NO: 70) | NO: 67) | ID NO: 68) |
| TfR.15.WH8.1.42Q.L10-1 | RASSSIRQMA (SEQ ID | DTSNRAT (SEQ ID | HQRSSYPWT (SEQ |
| | NO: 71) | NO: 67) | ID NO: 68) |
| TfR.15.WH8.1.42Q.L10-8 | RASSSIRQMS (SEQ ID | DTSNRAT (SEQ ID | HQRSSYPWT (SEQ |
| | NO: 72) | NO: 67) | ID NO: 68) |
| TfR.15.WH8.1.42Q.L10-16 | RASSSIRQMH (SEQ ID | DTSNRAT (SEQ ID | HQRSSYPWT (SEQ |
| | NO: 73) | NO: 67) | ID NO: 68) |
| TfR.15.WH8.1.42Q.H3-7 | RASSSIRQMN (SEQ ID | DTSNRAT (SEQ ID | HQRSSYPWT (SEQ |
| | NO: 69) | NO: 67) | ID NO: 68) |
| TfR.9.1B.39.38 | KASQSLLDEGGKTYLN | LVSKLDS (SEQ ID | WQGTHFPQT (SEQ |
| | (SEQ ID NO: 74) | NO: 75) | ID NO: 76) |
| TfR.9.1B.39.27.L-35 | KASQSLLDEDGKTYLN | LVSKLDS (SEQ ID | WQGTHFPQT (SEQ |
| | (SEQ ID NO: 77) | NO: 75) | ID NO: 76) |
| TfR.9.1B.39.38.L-21 | KASQSLLDEDNKTYLN | LVSKLDS (SEQ ID | WQGTHFPQT (SEQ |
| | (SEQ ID NO: 78) | NO: 75) | ID NO: 76) |
| TfR.9.1B.39.38.L-6 | KASQSLLDEDKKTYLN | LVSKLDS (SEQ ID | WQGTHFPQT (SEQ |
| | (SEQ ID NO: 79) | NO: 75) | ID NO: 76) |
| TfR.9.1B.39.27.L-19 | KASQSLLDEDAKTYLN | LVSKLDS (SEQ ID | WQGTHFPQT (SEQ |
| | (SEQ ID NO: 80) | NO: 75) | ID NO: 76) |
| TfR.15.WH8.1 | RASSSIRYMN (SEQ ID | DTSNRAT (SEQ ID | HQRSSYPWT (SEQ |
| | NO: 66) | NO: 67) | ID NO: 68) |
| TfR.9.1B.35 | KSSQSLLDSDGKTYLN | LVSKLDS (SEQ ID | WQGTHFPQS (SEQ |
| | (SEQ ID NO: 276) | NO: 75) | ID NO: 277) |
| TfR.9.1B.39 | KASQSLLDEDGKTYLN | LVSKLDS (SEQ ID | WQGTHFPQT (SEQ |
| | (SEQ ID NO: 77) | NO: 75) | ID NO: 76) |

TABLE 8
VH and VL Sequences of Anti-TfR Antigen Binding Domains

| Antibody | VH sequence | VL sequence |
|---|---|---|
| TfR. 15.WH8.1.24A | QVQLVQSGAEVKKPGASVKVSC | ASSSIRYMNWYQQKPGQAPRRLI |
| | KASGYTFTSYYMHWVRQAPGQG | EIVLTQSPATLSLSPGERATLSCR |
| | LEWMGNIYPGSGSSNYAQKFQG | YDTSNRATGIPARFSGSGSGTDFT |
| | RVTMTRDTSTSTVYMELSSLRSE | LTISSLEPEDFAVYYCHQRSSYPW |
| | DTAVYYCTRSAYYGNFDTMDN | TFGGGTKVEIK (SEQ ID NO: 82) |
| | WGQGTTVTVSS (SEQ ID NO: 81) | |
| TfR.15.WH8.1.24A.42Q | QVQLVQSGAEVKKPGASVKVSC | EIVLTQSPATLSLSPGERATLSCR |
| | KASGYTFTSYYMHWVRQAPGQG | ASSSIRQMNWYQQKPGQAPRRLI |
| | LEWMGNIYPGSGSSNYAQKFQG | YDTSNRATGIPARFSGSGSGTDFT |
| | RVTMTRDTSTSTVYMELSSLRSE | LTISSLEPEDFAVYYCHQRSSYPW |
| | DTAVYYCTRSAYYGNFDTMDN | TFGGGTKVEIK (SEQ ID NO: 83) |
| | WGQGTTVTVSS (SEQ ID NO: 81) | |
| TfR.15.WH8.1.42Q | QVQLVQSGAEVKKPGASVKVSC | EIVLTQSPATLSLSPGERATLSCR |
| | KASGYTFTSYYMHWVRQAPGQG | ASSSIRQMNWYQQKPGQAPRRLI |
| | LEWMGNIYPGSGSSNYAQKFQG | YDTSNRATGIPARFSGSGSGTDFT |
| | RVTMTRDTSTSTVYMELSSLRSE | LTISSLEPEDFAVYYCHQRSSYPW |
| | DTAVYYCTRSSYYGNFDTMDNW | TFGGGTKVEIK (SEQ ID NO: 83) |
| | GQGTTVTVSS (SEQ ID NO: 84) | |
| TfR.15.WH8.1.42Q.H6-4 | QVQLVQSGAEVKKPGASVKVSC | EIVLTQSPATLSLSPGERATLSCR |
| | KASGYTFTSYYMHWVRQAPGQG | ASSSIRQMNWYQQKPGQAPRRLI |
| | LEWMGNIYPGSGSSNYAQKFQG | YDTSNRATGIPARFSGSGSGTDFT |
| | RVTMTRDTSTSTVYMELSSLRSE | LTISSLEPEDFAVYYCHQRSSYPW |
| | DTAVYYCTRSSYYGRFDTMDNW | TFGGGTKVEIK (SEQ ID NO: 83) |
| | GQGTTVTVSS (SEQ ID NO: 85) | |
| TfR. 15.WH8.1.42Q.L7-2 | QVQLVQSGAEVKKPGASVKVSC | EIVLTQSPATLSLSPGERATLSCR |
| | KASGYTFTSYYMHWVRQAPGQG | ASSSIDQMNWYQQKPGQAPRRLI |
| | LEWMGNIYPGSGSSNYAQKFQG | YDTSNRATGIPARFSGSGSGTDFT |
| | RVTMTRDTSTSTVYMELSSLRSE | LTISSLEPEDFAVYYCHQRSSYPW |
| | DTAVYYCTRSSYYGNFDTMDNW | TFGGGTKVEIK (SEQ ID NO: 86) |
| | GQGTTVTVSS (SEQ ID NO: 84) | |
| TfR. 15.WH8.1.42Q.L10-1 | QVQLVQSGAEVKKPGASVKVSC | EIVLTQSPATLSLSPGERATLSCR |
| | KASGYTFTSYYMHWVRQAPGQG | ASSSIRQMAWYQQKPGQAPRRLI |
| | LEWMGNIYPGSGSSNYAQKFQG | YDTSNRATGIPARFSGSGSGTDFT |
| | RVTMTRDTSTSTVYMELSSLRSE | LTISSLEPEDFAVYYCHQRSSYPW |
| | DTAVYYCTRSSYYGNFDTMDNW | TFGGGTKVEIK (SEQ ID NO: 87) |
| | GQGTTVTVSS (SEQ ID NO: 84) | |
| TfR.15.WH8.1.42Q.L10-8 | QVQLVQSGAEVKKPGASVKVSC | EIVLTQSPATLSLSPGERATLSCR |
| | KASGYTFTSYYMHWVRQAPGQG | ASSSIRQMSWYQQKPGQAPRRLI |
| | LEWMGNIYPGSGSSNYAQKFQG | YDTSNRATGIPARFSGSGSGTDFT |
| | RVTMTRDTSTSTVYMELSSLRSE | LTISSLEPEDFAVYYCHQRSSYPW |
| | DTAVYYCTRSSYYGNFDTMDNW | TFGGGTKVEIK (SEQ ID NO: 88) |
| | GQGTTVTVSS (SEQ ID NO: 84) | |
| TfR.15.WH8.1.42Q.L10-16 | QVQLVQSGAEVKKPGASVKVSC | EIVLTQSPATLSLSPGERATLSCR |
| | KASGYTFTSYYMHWVRQAPGQG | ASSSIRQMHWYQQKPGQAPRRLI |
| | LEWMGNIYPGSGSSNYAQKFQG | YDTSNRATGIPARFSGSGSGTDFT |
| | RVTMTRDTSTSTVYMELSSLRSE | LTISSLEPEDFAVYYCHQRSSYPW |
| | DTAVYYCTRSSYYGNFDTMDNW | TFGGGTKVEIK (SEQ ID NO: 89) |
| | GQGTTVTVSS (SEQ ID NO: 84) | |
| TfR.15.WH8.1.42Q.H3-7 | QVQLVQSGAEVKKPGASVKVSC | EIVLTQSPATLSLSPGERATLSCR |
| | KASGYTFTSYYMHWVRQAPGQG | ASSSIRQMNWYQQKPGQAPRRLI |
| | LEWMGNIYPGSGSSNYAQKFQG | YDTSNRATGIPARFSGSGSGTDFT |
| | RVTMTRDTSTSTVYMELSSLRSE | LTISSLEPEDFAVYYCHQRSSYPW |
| | DTAVYYCTRSSNYGNFDTMDNW | TFGGGTKVEIK (SEQ ID NO: 83) |
| | GQGTTVTVSS (SEQ ID NO: 90) | |
| TfR.9.1B.39.38 | QVQLVQSGAEVKKPGASVKVSC | DVVMTQSPLSLPVTLGQPASISCK |
| | KASGYTFTEYAMHWVRQAPGQG | ASQSLLDEGGKTYLNWLQQRPG |
| | LEWMGGIAPNTGGTSYNQKFKG | QSPRRLIYLVSKLDSGVPDRFSGS |
| | RVTMTVDTSTSTAYMELSSLRSE | GSGTDFTLKISRVEAEDVGVYYC |
| | DTAVYYCASQGWLLRGGDYWG | WQGTHFPQTFGGGTKVEIK (SEQ |
| | QGTLVTVSS (SEQ ID NO: 91) | ID NO: 92) |
| TfR.9.1B.39.27.L-35 | QVQLVQSGAEVKKPGASVKVSC | DVVMTQSPLSLPVTLGQPASISCK |
| | KASGYTFTEYAMHWVRQAPGQG | ASQSLLDEDGKTYLNWLQQRPG |
| | LEWMGGIAPGTGGTSYNQKFKG | QSPRRLIYLVSKLDSGVPDRFSGS |
| | RVTMTVDTSTSTAYMELSSLRSE | GSGTDFTLKISRVEAEDVGVYYC |
| | DTAVYYCASQGWLLRGGDYWG | WQGTHFPQTFGGGTKVEIK (SEQ |
| | QGTLVTVSS (SEQ ID NO: 93) | ID NO: 94) |
| TfR.9.1B.39.38.L-21 | QVQLVQSGAEVKKPGASVKVSC | DVVMTQSPLSLPVTLGQPASISCK |
| | KASGYTFTEYAMHWVRQAPGQG | ASQSLLDEDNKTYLNWLQQRPG |
| | LEWMGGIAPNTGGTSYNQKFKG | QSPRRLIYLVSKLDSGVPDRFSGS |
| | RVTMTVDTSTSTAYMELSSLRSE | GSGTDFTLKISRVEAEDVGVYYC |
| | DTAVYYCASQGWLLRGGDYWG | WQGTHFPQTFGGGTKVEIK (SEQ |
| | QGTLVTVSS (SEQ ID NO: 91) | ID NO: 95) |
| TfR.9.1B.39.38.L-6 | QVQLVQSGAEVKKPGASVKVSC | DVVMTQSPLSLPVTLGQPASISCK |
| | KASGYTFTEYAMHWVRQAPGQG | ASQSLLDEDKKTYLNWLQQRPG |
| | LEWMGGIAPNTGGTSYNQKFKG | QSPRRLIYLVSKLDSGVPDRFSGS |
| | RVTMTVDTSTSTAYMELSSLRSE | GSGTDFTLKISRVEAEDVGVYYC |
| | DTAVYYCASQGWLLRGGDYWG | WQGTHFPQTFGGGTKVEIK (SEQ |
| | QGTLVTVSS (SEQ ID NO: 91) | ID NO: 96) |
| TfR.9.1B.39.27.L-19 | QVQLVQSGAEVKKPGASVKVSC | DVVMTQSPLSLPVTLGQPASISCK |
| | KASGYTFTEYAMHWVRQAPGQG | ASQSLLDEDAKTYLNWLQQRPG |
| | LEWMGGIAPGTGGTSYNQKFKG | QSPRRLIYLVSKLDSGVPDRFSGS |
| | RVTMTVDTSTSTAYMELSSLRSE | GSGTDFTLKISRVEAEDVGVYYC |
| | DTAVYYCASQGWLLRGGDYWG | WQGTHFPQTFGGGTKVEIK (SEQ |
| | QGTLVTVSS (SEQ ID NO: 93) | ID NO: 97) |
| TfR.15.WH8.1 | QVQLVQSGAEVKKPGASVKVSC | EIVLTQSPATLSLSPGERATLSCR |
| | KASGYTFTSYYMHWVRQAPGQG | ASSSIRYMNWYQQKPGQAPRRLI |
| | LEWMGNIYPGSGSSNYAQKFQG | YDTSNRATGIPARFSGSGSGTDFT |
| | RVTMTRDTSTSTVYMELSSLRSE | LTISSLEPEDFAVYYCHQRSSYPW |
| | DTAVYYCTRSSYYGNFDTMDNW | TFGGGTKVEIK (SEQ ID NO: 270) |
| | GQGTTVTVSS (SEQ ID NO: 269) | |
| TfR.9.1B.35 | QVQLVQSGAEVKKPGASVKVSC | DVVMTQSPLSLPVTLGQPASISCK |
| | KASGYTFTEYAMHWVRQAPGQG | SSQSLLDSDGKTYLNWLQQRPGQ |
| | LEWMGGINPNDGGTSYNQKFKG | SPRRLIYLVSKLDSGVPDRFSGSG |
| | RVTMTVDTSTSTAYMELSSLRSE | SGTDFTLKISRVEAEDVGVYHCW |
| | DTAVYYCASQGWLLRGGDYWG | QGTHFPQSFGGGTKVEIK (SEQ ID |
| | QGTLVTVSS (SEQ ID NO: 271) | NO: 272) |
| TfR.9.1B.39 | QVQLVQSGAEVKKPGASVKVSC | DVVMTQSPLSLPVTLGQPASISCK |
| | KASGYTFTEYAMHWVRQAPGQG | ASQSLLDEDGKTYLNWLQQRPG |
| | LEWMGGINPNDGGTSYNQKFKG | QSPRRLIYLVSKLDSGVPDRFSGS |
| | RVTMTVDTSTSTAYMELSSLRSE | GSGTDFTLKISRVEAEDVGVYYC |
| | DTAVYYCASQGWLLRGGDYWG | WQGTHFPQTFGGGTKVEIK (SEQ |
| | QGTLVTVSS (SEQ ID NO: 273) | ID NO: 274) |

In the present disclosure, “TfRL10-16” or “TfR10-16” or “10-16” refers to TfR. 15.WH8.1.42Q.L10-16;
“TfRL10-8” or “TfR10-8” or “10-8” refer to TfR.15.WH8.1.42Q.L10-8;
“TfR7-2” or “TfRL7-2” or “L7-2” refers to TfR.15.WH8.1.42Q.L7-2;
“TfR42Q” or “42Q” refers to TfR.15.WH8.1.42Q;
“TfR39.38” or “39.38” refers to TfR.9.1B0.39.38; and
“TfR24A” or “24A” refers to TfR.15.WH8.1.24A.

In some aspects, the CDRs of an antigen-binding domain that specifically binds to human TfR can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In some aspects, provided herein are antigen-binding domains that specifically bind to human TfR and comprise VH and VL CDRs of an antibody listed in Tables 6, 7, and 8 as determined by the method in MacCallum R M et al.

In some aspects, an antigen-binding domain that specifically binds to human TfR comprises the six CDRs of an antibody listed in Tables 6, 7, and 8 above (i.e. the three V_{H} CDRs of the antibody listed in Table 6 and the three VL CDRs of the same antibody listed in Table 7) as determined by Kabat numbering. In some aspects, an antigen-binding domain that specifically binds to human TfR comprises the six Chothia CDRs of an antibody listed in Table 8.

In some aspects, the CDRs of an antigen-binding domain that specifically binds to human TfR can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.). In some aspects, provided herein are antigen-binding domains that specifically bind to human TfR and comprise VH and VL CDRs of an antibody listed in Table 8 as determined by the AbM numbering scheme.

In some aspects, an antigen-binding domain that specifically binds to human TfR comprises the six IMGT CDRs of an antibody listed in listed in Table 8 according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

In some aspects, an antigen-binding domain that specifically binds to human TfR comprises the VH of an antibody listed in Table 8.

In some aspects, an antigen-binding domain that specifically binds to human TfR comprises the VL of antibody listed in Table 8.

In some aspects, an antigen-binding domain that specifically binds to human TfR comprises the VH and the VL of an antibody listed in Table 8.

In some aspects, an antigen-binding domain of the present disclosure that specifically binds to human TfR comprises: (a) HVR-H1 comprising the amino acid sequence of SEQ ID NO:56; (b) HVR-H2 comprising the amino acid sequence of SEQ ID NO:57; (c) HVR-H3 comprising the amino acid sequence of SEQ ID NO:58; (d) HVR-L1 comprising the amino acid sequence of SEQ ID NO:66; (e) HVR-L2 comprising the amino acid sequence of SEQ ID NO:67; and (f) HVR-L3 comprising the amino acid sequence of SEQ ID NO:68; (a) HVR-H1 comprising the amino acid sequence of SEQ ID NO:56; (b) HVR-H2 comprising the amino acid sequence of SEQ ID NO:57; (c) HVR-H3 comprising the amino acid sequence of SEQ ID NO:58; (d) HVR-L1 comprising the amino acid sequence of SEQ ID NO:69; (e) HVR-L2 comprising the amino acid sequence of SEQ ID NO:67; and (f) HVR-L3 comprising the amino acid sequence of SEQ ID NO:68; (a) HVR-H1 comprising the amino acid sequence of SEQ ID NO:56; (b) HVR-H2 comprising the amino acid sequence of SEQ ID NO:57; (c) HVR-H3 comprising the amino acid sequence of SEQ ID NO:59; (d) HVR-L1 comprising the amino acid sequence of SEQ ID NO:69; (e) HVR-L2 comprising the amino acid sequence of SEQ ID NO:67; and (f) HVR-L3 comprising the amino acid sequence of SEQ ID NO:68; (a) HVR-H1 comprising the amino acid sequence of SEQ ID NO:56; (b) HVR-H2 comprising the amino acid sequence of SEQ ID NO:57; (c) HVR-H3 comprising the amino acid sequence of SEQ ID NO:60; (d) HVR-L1 comprising the amino acid sequence of SEQ ID NO:69; (e) HVR-L2 comprising the amino acid sequence of SEQ ID NO:67; and (f) HVR-L3 comprising the amino acid sequence of SEQ ID NO:68; (a) HVR-H1 comprising the amino acid sequence of SEQ ID NO:56; (b) HVR-H2 comprising the amino acid sequence of SEQ ID NO:57; (c) HVR-H3 comprising the amino acid sequence of SEQ ID NO:59; (d) HVR-L1 comprising the amino acid sequence of SEQ ID NO:70; (e) HVR-L2 comprising the amino acid sequence of SEQ ID NO:67; and (f) HVR-L3 comprising the amino acid sequence of SEQ ID NO:68; (a) HVR-H1 comprising the amino acid sequence of SEQ ID NO:56; (b) HVR-H2 comprising the amino acid sequence of SEQ ID NO:57; (c) HVR-H3 comprising the amino acid sequence of SEQ ID NO:59; (d) HVR-L1 comprising the amino acid sequence of SEQ ID NO:71; (e) HVR-L2 comprising the amino acid sequence of SEQ ID NO:67; and (f) HVR-L3 comprising the amino acid sequence of SEQ ID NO:68; (a) HVR-H1 comprising the amino acid sequence of SEQ ID NO:56; (b) HVR-H2 comprising the amino acid sequence of SEQ ID NO:57; (c) HVR-H3 comprising the amino acid sequence of SEQ ID NO:59; (d) HVR-L1 comprising the amino acid sequence of SEQ ID NO:72; (e) HVR-L2 comprising the amino acid sequence of SEQ ID NO:67; and (f) HVR-L3 comprising the amino acid sequence of SEQ ID NO:68; (a) HVR-H1 comprising the amino acid sequence of SEQ ID NO:56; (b) HVR-H2 comprising the amino acid sequence of SEQ ID NO:57; (c) HVR-H3 comprising the amino acid sequence of SEQ ID NO:59; (d) HVR-L1 comprising the amino acid sequence of SEQ ID NO:73; (e) HVR-L2 comprising the amino acid sequence of SEQ ID NO:67; and (f) HVR-L3 comprising the amino acid sequence of SEQ ID NO:68; (a) HVR-H1 comprising the amino acid sequence of SEQ ID NO:56; (b) HVR-H2 comprising the amino acid sequence of SEQ ID NO:57; (c) HVR-H3 comprising the amino acid sequence of SEQ ID NO:61; (d) HVR-L1 comprising the amino acid sequence of SEQ ID NO:69; (e) HVR-L2 comprising the amino acid sequence of SEQ ID NO:67; and (f) HVR-L3 comprising the amino acid sequence of SEQ ID NO:68; (a) HVR-H1 comprising the amino acid sequence of SEQ ID NO:62; (b) HVR-H2 comprising the amino acid sequence of SEQ ID NO:63; (c) HVR-H3 comprising the amino acid sequence of SEQ ID NO:64; (d) HVR-L1 comprising the amino acid sequence of SEQ ID NO:74; (e) HVR-L2 comprising the amino acid sequence of SEQ ID NO:75; and (f) HVR-L3 comprising the amino acid sequence of SEQ ID NO:76; (a) HVR-H1 comprising the amino acid sequence of SEQ ID NO:62; (b) HVR-H2 comprising the amino acid sequence of SEQ ID NO:65; (c) HVR-H3 comprising the amino acid sequence of SEQ ID NO:64; (d) HVR-L1 comprising the amino acid sequence of SEQ ID NO:77; (e) HVR-L2 comprising the amino acid sequence of SEQ ID NO:75; and (f) HVR-L3 comprising the amino acid sequence of SEQ ID NO:76; (a) HVR-H1 comprising the amino acid sequence of SEQ ID NO:62; (b) HVR-H2 comprising the amino acid sequence of SEQ ID NO:63; (c) HVR-H3 comprising the amino acid sequence of SEQ ID NO:64; (d) HVR-L1 comprising the amino acid sequence of SEQ ID NO:78; (e) HVR-L2 comprising the amino acid sequence of SEQ ID NO:75; and (f) HVR-L3 comprising the amino acid sequence of SEQ ID NO:76; (a) HVR-H1 comprising the amino acid sequence of SEQ ID NO:62; (b) HVR-H2 comprising the amino acid sequence of SEQ ID NO:63; (c) HVR-H3 comprising the amino acid sequence of SEQ ID NO:64; (d) HVR-L1 comprising the amino acid sequence of SEQ ID NO:79; (e) HVR-L2 comprising the amino acid sequence of SEQ ID NO:75; and (f) HVR-L3 comprising the amino acid sequence of SEQ ID NO:76; and (a) HVR-H1 comprising the amino acid sequence of SEQ ID NO:62; (b) HVR-H2 comprising the amino acid sequence of SEQ ID NO:65; (c) HVR-H3 comprising the amino acid sequence of SEQ ID NO:64; (d) HVR-L1 comprising the amino acid sequence of SEQ ID NO:80; (e) HVR-L2 comprising the amino acid sequence of SEQ ID NO:75; and (f) HVR-L3 comprising the amino acid sequence of SEQ ID NO:76.

In some aspects, an antigen-binding domain of the present disclosure that specifically binds to human TfR comprises: (a) HVR-H1 comprising the amino acid sequence of SEQ ID NO:56; (b) HVR-H2 comprising the amino acid sequence of SEQ ID NO:57; (c) HVR-H3 comprising the amino acid sequence of SEQ ID NO:59; (d) HVR-L1 comprising the amino acid sequence of SEQ ID NO:66; (e) HVR-L2 comprising the amino acid sequence of SEQ ID NO:67; and (f) HVR-L3 comprising the amino acid sequence of SEQ ID NO:68; (a) HVR-H1 comprising the amino acid sequence of SEQ ID NO:62; (b) HVR-H2 comprising the amino acid sequence of SEQ ID NO:275; (c) HVR-H3 comprising the amino acid sequence of SEQ ID NO:64; (d) HVR-L1 comprising the amino acid sequence of SEQ ID NO:276; (e) HVR-L2 comprising the amino acid sequence of SEQ ID NO:75; and (f) HVR-L3 comprising the amino acid sequence of SEQ ID NO:277; or (a) HVR-H1 comprising the amino acid sequence of SEQ ID NO:62; (b) HVR-H2 comprising the amino acid sequence of SEQ ID NO:275; (c) HVR-H3 comprising the amino acid sequence of SEQ ID NO:64; (d) HVR-L1 comprising the amino acid sequence of SEQ ID NO:77; (e) HVR-L2 comprising the amino acid sequence of SEQ ID NO:75; and (f) HVR-L3 comprising the amino acid sequence of SEQ ID NO:76.

In another aspect, an antigen-binding domain of the present disclosure that specifically binds to human TfR comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs:81, 84, 85, 90, 91, and 93. In certain aspects, a V_{H} sequence having at least 90%, 91%, 92%, 93%, 94%, 95%, 96%, 97%, 98%, 99%, or 100% identity to an amino acid sequence selected from the group consisting of SEQ ID NOs: 81, 84, 85, 90, 91, and 93 contains substitutions (e.g., conservative substitutions), insertions, or deletions relative to the reference sequence, but an antigen-binding domain that specifically binds to human TfR comprising that sequence retains the ability to bind to human TfR. In certain aspects, a total of 1 to 10 amino acids have been substituted, inserted, and/or deleted in SEQ ID NO: 81, 84, 85, 90, 91, or 93. In certain aspects, a total of 1 to 5 amino acids have been substituted, inserted and/or deleted in SEQ ID NO: 81, 84, 85, 90, 91, or 93. In certain aspects, substitutions, insertions, or deletions occur in regions outside the HVRs (i.e., in the FRs). Optionally, the antigen-binding domain that specifically binds to human TfR comprises the V_{H} sequence of SEQ ID NO: 81, 84, 85, 90, 91, and 93, including post-translational modifications of that sequence. In a particular aspect, the V_{H} comprises one, two or three HVRs selected from: (a) HVR-H1 comprising an amino acid sequence selected from the group consisting of SEQ ID NOs:56 and 62; (b) HVR-H2 comprising an amino acid sequence selected from the group consisting of SEQ ID NOs:57, 63, and 65; and (c) HVR-H3 comprising an amino acid sequence selected from the group consisting of SEQ ID NOs:58, 59, 60, 61, and 64.

In another aspect, an antigen-binding domain of the present disclosure that specifically binds to human TfR comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs: 269, 271, and 273. In certain aspects, a V_{H} sequence having at least 90%, 91%, 92%, 93%, 94%, 95%, 96%, 97%, 98%, 99%, or 100% identity to an amino acid sequence selected from the group consisting of SEQ ID NOs: 269, 271, and 273 contains substitutions (e.g., conservative substitutions), insertions, or deletions relative to the reference sequence, but an antigen-binding domain that specifically binds to human TfR comprising that sequence retains the ability to bind to human TfR. In certain aspects, a total of 1 to 10 amino acids have been substituted, inserted, and/or deleted in SEQ ID NO: 269, 271, or 273. In certain aspects, a total of 1 to 5 amino acids have been substituted, inserted and/or deleted in SEQ ID NO: 269, 271, or 273. In certain aspects, substitutions, insertions, or deletions occur in regions outside the HVRs (i.e., in the FRs). Optionally, the antigen-binding domain that specifically binds to human TfR comprises the V_{H} sequence of SEQ ID NO: 269, 271, and 273 including post-translational modifications of that sequence. In a particular aspect, the V_{H} comprises one, two or three HVRs selected from: (a) HVR-H1 comprising an amino acid sequence selected from the group consisting of SEQ ID NOs:56 and 62; (b) HVR-H2 comprising an amino acid sequence selected from the group consisting of SEQ ID NOs:57, 63, 65, and 275; and (c) HVR-H3 comprising an amino acid sequence selected from the group consisting of SEQ ID NOs:58, 59, 60, 61, and 64.

In another aspect, an antigen-binding domain of the present disclosure that specifically binds to human TfR comprises comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs:82, 83, 86, 87, 88, 89, 92, 94, 95, 96, and 97. In certain aspects, a V_{L} sequence having at least 90%, 91%, 92%, 93%, 94%, 95%, 96%, 97%, 98%, 99%, or 100% identity to an amino acid sequence selected from the group consisting of SEQ ID NOs: 82, 83, 86, 87, 88, 89, 92, 94, 95, 96, and 97, and contains substitutions (e.g., conservative substitutions), insertions, or deletions relative to the reference sequence, but an antigen-binding domain of the present disclosure that specifically binds to human TfR comprising that sequence retains the ability to bind to human TfR. In some aspects, a total of 1 to 10 amino acids have been substituted, inserted and/or deleted in SEQ ID NO: 82, 83, 86, 87, 88, 89, 92, 94, 95, 96, or 97. In certain aspects, a total of 1 to 5 amino acids have been substituted, inserted and/or deleted in SEQ ID NO: 82, 83, 86, 87, 88, 89, 92, 94, 95, 96, or 97. In certain aspects, the substitutions, insertions, or deletions occur in regions outside the HVRs (i.e., in the FRs). Optionally, the antigen-binding domain of the present disclosure that specifically binds to human TfR comprises the V_{L} sequence of SEQ ID NO: 82, 83, 86, 87, 88, 89, 92, 94, 95, 96, and 97, including post-translational modifications of that sequence. In a particular aspect, the V_{L} comprises one, two or three HVRs selected from (a) HVR-L1 comprising an amino acid sequence selected from the group consisting of SEQ ID NOs:66, 69, 70, 71, 72, 73, 74, 77, 78, 79, and 80; (b) HVR-L2 comprising an amino acid sequence selected from the group consisting of SEQ ID NOs:67 and 75; and (c) HVR-L3 comprising an amino acid sequence selected from the group consisting of SEQ ID NOs:68 and 76.

In another aspect, an antigen-binding domain of the present disclosure that specifically binds to human TfR comprises comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs:270, 272, and 274. In certain aspects, a V_{L} sequence having at least 90%, 91%, 92%, 93%, 94%, 95%, 96%, 97%, 98%, 99%, or 100% identity to an amino acid sequence selected from the group consisting of SEQ ID NOs: 270, 272, and 274, and contains substitutions (e.g., conservative substitutions), insertions, or deletions relative to the reference sequence, but an antigen-binding domain of the present disclosure that specifically binds to human TfR comprising that sequence retains the ability to bind to human TfR. In some aspects, a total of 1 to 10 amino acids have been substituted, inserted and/or deleted in SEQ ID NO: 270, 272, or 274. In certain aspects, a total of 1 to 5 amino acids have been substituted, inserted and/or deleted in SEQ ID NO: 270, 272, or 274. In certain aspects, the substitutions, insertions, or deletions occur in regions outside the HVRs (i.e., in the FRs). Optionally, the antigen-binding domain of the present disclosure that specifically binds to human TfR comprises the V_{L} sequence of SEQ ID NO: 270, 272 and 274, including post-translational modifications of that sequence. In a particular aspect, the V_{L} comprises one, two or three HVRs selected from (a) HVR-L1 comprising an amino acid sequence selected from the group consisting of SEQ ID NOs:66, 69, 70, 71, 72, 73, 74, 77, 78, 79, 80, and 276; (b) HVR-L2 comprising an amino acid sequence selected from the group consisting of SEQ ID NOs:67 and 75; and (c) HVR-L3 comprising an amino acid sequence selected from the group consisting of SEQ ID NOs:68, 76 and 277.

In some aspects, an antigen-binding domain of the present disclosure that specifically binds to human TfR is provided, wherein the antibody comprises a V_{H} as in any of the aspects provided above, and a V_{L} as in any of the aspects provided above. In some aspects, the antigen-binding domain comprises the V_{H} and V_{L} sequences in SEQ ID NOs: 81, 84, 85, 90, 91, and 93, and SEQ ID NOs: 82, 83, 86, 87, 88, 89, 92, 94, 95, 96, and 97, respectively, including post-translational modifications of those sequences.

In some aspects, an antigen-binding domain of the present disclosure that specifically binds to human TfR is provided, wherein the antibody comprises a V_{H} as in any of the aspects provided above, and a V_{L} as in any of the aspects provided above. In some aspects, the antigen-binding domain comprises the V_{H} and V_{L} sequences in SEQ ID NOs: 81, 84, 85, 90, 91, 93, 269, 271, and 273 and SEQ ID NOs: 82, 83, 86, 87, 88, 89, 92, 94, 95, 96, 97, 270, 272, and 274, respectively, including post-translational modifications of those sequences.

In some aspects, provided herein are antigen-binding domains that specifically bind to human TfR comprising a heavy chain variable domain (V_{H}) and a light chain variable domain (V_{L}), wherein the V_{H} and V_{L} are selected from the group consisting of: V_{H} comprising the amino acid sequence of SEQ ID NO:81 and V_{L} comprising the amino acid sequence of SEQ ID NO:82; V_{H} comprising the amino acid sequence of SEQ ID NO:81 and V_{L} comprising the amino acid sequence of SEQ ID NO:83; V_{H} comprising the amino acid sequence of SEQ ID NO:84 and V_{L} comprising the amino acid sequence of SEQ ID NO:83; V_{H} comprising the amino acid sequence of SEQ ID NO:85 and V_{L} comprising the amino acid sequence of SEQ ID NO:83; V_{H} comprising the amino acid sequence of SEQ ID NO:84 and V_{L} comprising the amino acid sequence of SEQ ID NO:86; V_{H} comprising the amino acid sequence of SEQ ID NO:84 and V_{L} comprising the amino acid sequence of SEQ ID NO:87; V_{H} comprising the amino acid sequence of SEQ ID NO:84 and V_{L} comprising the amino acid sequence of SEQ ID NO:88; V_{H} comprising the amino acid sequence of SEQ ID NO:84 and V_{L} comprising the amino acid sequence of SEQ ID NO:89; V_{H} comprising the amino acid sequence of SEQ ID NO:90 and V_{L} comprising the amino acid sequence of SEQ ID NO:83; V_{H} comprising the amino acid sequence of SEQ ID NO:91 and V_{L} comprising the amino acid sequence of SEQ ID NO:92; V_{H} comprising the amino acid sequence of SEQ ID NO:93 and V_{L} comprising the amino acid sequence of SEQ ID NO:94; V_{H} comprising the amino acid sequence of SEQ ID NO:91 and V_{L} comprising the amino acid sequence of SEQ ID NO:95; V_{H} comprising the amino acid sequence of SEQ ID NO:91 and V_{L} comprising the amino acid sequence of SEQ ID NO:96; and V_{H} comprising the amino acid sequence of SEQ ID NO:93 and V_{L} comprising the amino acid sequence of SEQ ID NO:97.

In some aspects, provided herein are antigen-binding domains that specifically bind to human TfR comprising a heavy chain variable domain (V_{H}) and a light chain variable domain (V_{L}), wherein the V_{H} and V_{L} are selected from the group consisting of: V_{H} comprising the amino acid sequence of SEQ ID NO:269 and V_{L} comprising the amino acid sequence of SEQ ID NO:270; V_{H} comprising the amino acid sequence of SEQ ID NO:271 and V_{L} comprising the amino acid sequence of SEQ ID NO:272; and V_{H} comprising the amino acid sequence of SEQ ID NO:273 and V_{L} comprising the amino acid sequence of SEQ ID NO:274.In some aspects, provided herein is an antigen-binding domain that competitively inhibits binding to human TfR of an antibody comprising a V_{H} amino acid sequence of an antibody in Table 8 and a V_{L} amino acid sequence of the same antibody in Table 8.

In some aspects, an antigen-binding domain that specifically binds to human TfR comprises a VH and a VL on a single polypeptide chain (e.g., a VH and VL in Table 8).

In some aspects, an antigen-binding domain that specifically binds to human TfR comprises an scFv. In some aspects, an scFv comprises a VH and a VL of an antibody listed in Table 8. The scFv can comprise a VH that is N-terminal to a VL or a VL that is N-terminal to a VH. The scFv can comprise a linker, e.g., between a VH and a VL. Accordingly, the scFv can be in the orientation VH-linker-VL or VL-linker-VH. Such a linker can be about 5 to about 25 amino acids in length. Such a linker can be about 5 to about 20 amino acids in length. Such a linker can be about 10 to about 25 amino acids in length. Such a linker can be about 10 to about 20 amino acids in length. Such a linker can be, e.g., a glycine linker, a glycine-rich linker, or a glycine-serine linker. Such a linker can comprise the amino acid sequence of GGSEGKSSGSGSESKSTGGS (SEQ ID NO: 209). Such a linker can comprise the amino acid sequence of GGGGSGGGGSGGGGSGGGGS (SEQ ID NO: 210).

In some aspects, an antigen-binding domain that specifically binds to human TfR comprises a VH on a first polypeptide and a VL on a second polypeptide (e.g., a Fab). The Fab comprises one constant domain, one VH, and one VL. In some aspects, the antigen-binding domain comprises a Fab comprising a constant domain, a VH of an antibody listed in Table 8 and a VL of the same antibody listed in Table 8. In some aspects, the antigen-binding domain that comprises a Fab comprises the CDRs of the antibody in Tables 6, 7, and 8 (e.g., the non-identical amino acids in the VH and/or VL are outside of the CDRs).

In some aspects, an antigen-binding domain that specifically binds to human TfR comprises the antigen-binding fragment of a heavy chain only antibody (e.g., a VHH or nanobody). In some aspects, an antigen-binding domain comprises a VHH comprising the VH of an antibody listed in Table 8. In other aspects, the antigen-binding domain comprises a VHH comprises the heavy chain CDRs of an antibody listed in Tables 6 and 8.

In some aspects, an antigen-binding domain that specifically binds to human TfR is a murine antigen-binding domain. In some aspects, an antigen-binding domain that specifically binds to human TfR is a chimeric antigen-binding domain. In some aspects, an antigen-binding domain that specifically binds to human TfR is a humanized antigen-binding domain. In some aspects, an antigen-binding domain that specifically binds to human TfR is a human antigen-binding domain.

In some aspects, an antigen-binding domain provided herein that specifically binds to human TfR also binds to cynomolgus monkey TfR.

In some aspects, an anti-TfR antigen-binding domain of the present disclosure comprises a heavy chain variable region (VH) complementarity determining region (CDR) 1, VH CDR2, VH CDR3, and a light chain variable region (VL) CDR1, VL CDR2, and VL CDR3 sequences comprising the amino acid sequences of SEQ ID NOs:56, 57, 59, 70, 67, and 68, respectively.

In some aspects, the anti-TfR antigen-binding domain comprises a VH and a VL comprising the amino acid sequences of SEQ ID NOs:84 and 86, respectively.

In some aspects, the anti-TfR antigen-binding domain comprises a heavy chain variable region (VH) complementarity determining region (CDR) 1, VH CDR2, VH CDR3 and a light chain variable region (VL) CDR1, CDR2, and CDR3 sequences comprising the amino acid sequences of SEQ ID NOs:56, 57, 59, 73, 67, and 68, respectively.

In some aspects, the anti-TfR antigen-binding domain comprises a VH and a VL comprising the amino acid sequences of SEQ ID NOs:84 and 89, respectively.

In some aspects, the anti-TfR antigen-binding domain comprises a heavy chain variable region (VH) complementarity determining region (CDR) 1, VH CDR2, VH CDR3 and a light chain variable region (VL) CDR1, CDR2, and CDR3 sequences comprising the amino acid sequences of SEQ ID NOs:62, 63, 64, 74, 75, and 76, respectively.

In some aspects, the anti-TfR antigen-binding domain comprises a VH and a VL comprising the amino acid sequences of SEQ ID NOs:91 and 92, respectively.

In some aspects, the antigen-binding domain provided herein binds human TfR with an affinity between about 500 nM and about 10,000 nM. In some aspects, the antigen-binding domain binds human TfR with an affinity between about 500 nM and about 5,000 nM, between about 500 nM and about 2,500 nM, between about 500 nM and about 1,000 nM, between about 500 nM and about 750 nM, between about 1,000 nM and about 2,5000 nM, between about 2,500 nM and about 5,000 nM, between about 2,500 nM and about 10,000 nM, between about 5,000 nM and about 10,000 nM, and values and ranges there between.

In some aspects, an antigen-binding domain provided herein binds human TfR with an affinity between about 10 nM and about 500 nM. In some aspects, the antigen-binding domain binds human TfR with an affinity between about 10 nM and about 250 nM, between about 10 nM and about 100 nM, between about 10 nM and about 50 nM, between about 50 nM and about 500 nM, between about 50 nM and about 250 nM, between about 100 nM and about 250 nM, between about 250 nM and about 500 nM, and values and ranges there between.

In some aspects, the antigen-binding domain binds human TfR with an affinity of less than about 10 nM. In some aspects, the antigen-binding domain binds human TfR with an affinity between about 1 nM and about 10 nM, between about 1 nM and about 5 nM, between about 0.1 nM and about 1 nM, between about 0.01 nM and about 0.1 nM, and values and ranges there between.

In some aspects, the affinity of anti-TfR antibody TfR.15.WH8.1.42Q.L10-16 (i.e., anti-TfRL10-16) to human TfR is approximately 2010 nM. In some aspects, the affinity of anti-TfR antibody TfR.15.WH8.1.42Q.L7-2 (i.e., anti-TfRL7-2) is approximately 309 nM. In some aspects, the affinity of anti-TfR antibody TfR.9.1B.39.38 (i.e., anti-TfR39.38) is approximately 3810 nM. Without being bound by this theory, use of an anti-TfR antibody in the multispecific binding proteins described herein, wherein the anti-TfR antibody displays lower affinity binding to human TfR (e.g., ~2000 nM), minimizes interference with iron uptake by reticulocytes, thereby reducing the likelihood of anemia. In some aspects, administration of a composition comprising an anti-pyroGlu3 Aβ antigen-binding domain and an anti-TfR antigen binding domain of the present disclosure does not result in anemia.

In some aspects, the binding of an antigen-binding domain provided herein that specifically binds to human TfR is measured using surface plasmon resonance. Surface plasmon resonance can be measured, e.g., using the Carterra LSA platform or BIACORE®. In some aspects, an antigen-binding domain provided herein that specifically binds to human TfR binds to human TfR as measured by, for example, radioimmunoassay (RIA), Western blot, or ELISA OD₄₅₀.

Also provided herein are antigen-binding domains that bind to the same epitope of TfR as a TfR antigen-binding domain provided herein. Also provided herein are antigen-binding domains that competitively inhibit binding to TfR of TfR antigen-binding domain provided herein.

### Antigen-Binding Domains That Bind to CD98hc

Provided herein are antigen-binding domains that specifically bind to human CD98hc.

Such antigen-binding domains can be capable of crossing the blood brain barrier (BBB) and capable of transporting other agents (e.g., therapeutically active agents) associated with the antigen-binding domain across the blood brain barrier. Accordingly, in some aspects, provided herein are antigen-binding domains that specifically bind to human CD98hc that are capable of being internalized in blood brain barrier epithelial cells.

Antibodies (e.g., antigen-binding domains) to human CD98hc were obtained, characterized, and recombinantly expressed as described previously in WO2024/026471 (filed Jul. 28, 2023) and in WO2025166042 (International Application Serial No. PCT/US2025/013843; filed Jan. 30, 2025), both of which are incorporated herein by reference in their entireties.

Table 9 below lists the heavy chain CDR amino acid sequences (according to Kabat numbering) of anti-CD98hc antigen-binding domains of the present disclosure. Table 10 below lists the light chain CDR amino acid sequences (according to Kabat numbering) of anti-CD98hc antigen-binding domains of the present disclosure. Table 11 below lists the heavy chain variable region and light chain variable region amino acid sequences of anti-CD98hc antigen-binding domains of the present disclosure.

TABLE 9
Heavy Chain CDR Sequences of Anti-CD98hc Antibodies

| Antibody | CDR-H1 | CDR-H2 | CDR-H3 |
|---|---|---|---|
| CD98hc.04.048.WH1 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.15 | AYDMS | YISSGGGSTYYADEVKG | QGGLPAWFVY |
| | (SEQ ID NO: 98) | (SEQ ID NO: 101) | (SEQ ID NO: 100) |
| CD98hc.04.048.WH1.20 | AYDMS | YISSGGGSTYYADFVKG | QGGLPAWFVY |
| | (SEQ ID NO: 98) | (SEQ ID NO: 102) | (SEQ ID NO: 100) |
| CD98hc.04.048.WH1.23 | AYDMS | YISSGGGSTYYADYVKG | QGGLPAWFVY |
| | (SEQ ID NO: 98) | (SEQ ID NO: 103) | (SEQ ID NO: 100) |
| CD98hc.04.048.WH1.078 | AYDMS | YKSSGGGSTYYADSVKG | QGGLPAWFVY |
| | (SEQ ID NO: 98) | (SEQ ID NO: 104) | (SEQ ID NO: 100) |
| CD98hc.04.048.WH1.083 | AYDMS | YRSSGGGSTYYADSVKG | QGGLPAWFVY |
| | (SEQ ID NO: 98) | (SEQ ID NO: 105) | (SEQ ID NO: 100) |
| CD98hc.04.048.WH1.105 | AYDMS | YISSQGGSTYYADSVKG | QGGLPAWFVY |
| | (SEQ ID NO: 98) | (SEQ ID NO: 106) | (SEQ ID NO: 100) |
| CD98hc.04.048.WH1.118 | AYDMS | YISSGKGSTYYADSVKG | QGGLPAWFVY |
| | (SEQ ID NO: 98) | (SEQ ID NO: 107) | (SEQ ID NO: 100) |
| CD98hc.04.048.WH1.119 | AYDMS | YISSGHGSTYYADSVKG | QGGLPAWFVY |
| | (SEQ ID NO: 98) | (SEQ ID NO: 108) | (SEQ ID NO: 100) |
| CD98hc.04.048.WH1.121 | AYDMS | YISSGRGSTYYADSVKG | QGGLPAWFVY |
| | (SEQ ID NO: 98) | (SEQ ID NO: 109) | (SEQ ID NO: 100) |
| CD98hc.04.048.WH1.133 | AYDMS | YISSGGGHTYYADSVKG | QGGLPAWFVY |
| | (SEQ ID NO: 98) | (SEQ ID NO: 110) | (SEQ ID NO: 100) |
| CD98hc.04.048.WH1.193 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.217 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.218 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.222 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.224 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.232 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.236 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.296 | AYDMS | YISSQGGSTYYADSVKG | QGGLPAWFVY |
| | (SEQ ID NO: 98) | (SEQ ID NO: 106) | (SEQ ID NO: 100) |
| CD98hc.04.048.WH1.151 | AYDMS | YISSGGGSDYYADSVKG | QGGLPAWFVY |
| | (SEQ ID NO: 98) | (SEQ ID NO: 111) | (SEQ ID NO: 100) |
| CD98hc.04.048.WH1.158 | AYDMS | YISSGGGSTYYADSVKG | QGSLPAWFVY |
| | (SEQ ID NO: 98) | (SEQ ID NO: 99) | (SEQ ID NO: 112) |
| CD98hc.04.048.WH1.162 | AYDMS | YISSGGGSTYYADSVKG | QGGHPAWFVY |
| | (SEQ ID NO: 98) | (SEQ ID NO: 99) | (SEQ ID NO: 113) |
| CD98hc.04.048.WH1.165 | AYDMS | YISSGGGSTYYADSVKG | QGGAPAWFVY |
| | (SEQ ID NO: 98) | (SEQ ID NO: 99) | (SEQ ID NO: 114) |
| CD98hc.04.048.WH1.166 | AYDMS | YISSGGGSTYYADSVKG | QGGTPAWFVY |
| | (SEQ ID NO: 98) | (SEQ ID NO: 99) | (SEQ ID NO: 115) |
| CD98hc.04.048.WH1.167 | AYDMS | YISSGGGSTYYADSVKG | QGGQPAWFVY |
| | (SEQ ID NO: 98) | (SEQ ID NO: 99) | (SEQ ID NO: 116) |
| CD98hc.04.048.WH1.168 | AYDMS | YISSGGGSTYYADSVKG | QGGLPALFVY |
| | (SEQ ID NO: 98) | (SEQ ID NO: 99) | (SEQ ID NO: 117) |
| CD98hc.04.048.WH1.170 | AYDMS | YISSGGGSTYYADSVKG | QGGLPAKFVY |
| | (SEQ ID NO: 98) | (SEQ ID NO: 99) | (SEQ ID NO: 118) |
| CD98hc.04.048.WH1.171 | AYDMS | YISSGGGSTYYADSVKG | QGGLPATFVY |
| | (SEQ ID NO: 98) | (SEQ ID NO: 99) | (SEQ ID NO: 119) |
| CD98hc.04.048.WH1.174 | AYDMS | YISSGGGSTYYADSVKG | QGGLPARFVY |
| | (SEQ ID NO: 98) | (SEQ ID NO: 99) | (SEQ ID NO: 120) |
| CD98hc.04.048.WH1.185 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.208 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.231 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.235 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.250 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.253 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.262 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.269 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.271 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.274 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.276 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.279 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.281 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.282 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.283 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.39 | AYDMS | YISSGGGSTYYADSVKG | QGGLPAWFVY |
| | (SEQ ID NO: 98) | (SEQ ID NO: 99) | (SEQ ID NO: 100) |
| CD98hc.04.048.WH1.55 | AYAMS | YISSGGGSTYYADSVKG | QGGLPAWFVY |
| | (SEQ ID NO: 121) | (SEQ ID NO: 99) | (SEQ ID NO: 100) |
| CD98hc.04.048.WH1.76 | AYAMS | YISSGGGSTYYADSVKG | QGGLPAWFVY |
| | (SEQ ID NO: 121) | (SEQ ID NO: 99) | (SEQ ID NO: 100) |

TABLE 10
Heavy Chain CDR Sequences of Anti-CD98hc Antibodies

| Antibody | CDR-L1 | CDR-L2 | CDR-L3 |
|---|---|---|---|
| CD98hc.04.048.WH1 | KSSQSLLYSNTQKNYLA | WASTRES (SEQ | QQYSYYPPT |
| | (SEQ ID NO: 122) | ID NO: 123) | (SEQ ID NO: 124) |
| CD98hc.04.048.WH1.15 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.20 | KSSQSLLYSNTQKNYLA | WASTRES | QQYSYYPPT |
| | (SEQ ID NO: 122) | (SEQ ID NO | (SEQ ID NO: 124) |
| | | 123) | |
| CD98hc.04.048.WH1.23 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.078 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.083 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.105 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.118 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.119 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.121 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.133 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.193 | KSSQSLKYSNTQKNYLA | WASTRES | QQYSYYPPT |
| | (SEQ ID NO: 125) | (SEQ ID NO: | (SEQ ID NO: 124) |
| | | 123) | |
| CD98hc.04.048.WH1.217 | KSSQSLLYQNTQKNYLA | WASTRES | QQYSYYPPT |
| | (SEQ ID NO: 126) | (SEQ ID NO: | (SEQ ID NO: 124) |
| | | 123) | |
| CD98hc.04.048.WH1.218 | KSSQSLLYENTQKNYLA | WASTRES | QQYSYYPPT |
| | (SEQ ID NO: 127) | (SEQ ID NO: | (SEQ ID NO: 124) |
| | | 123) | |
| CD98hc.04.048.WH1.222 | KSSQSLLYSHTQKNYLA | WASTRES | QQYSYYPPT |
| | (SEQ ID NO: 128) | (SEQ ID NO: | (SEQ ID NO: 124) |
| | | 123) | |
| CD98hc.04.048.WH1.224 | KSSQSLLYSTTQKNYLA | WASTRES | QQYSYYPPT |
| | (SEQ ID NO: 129) | (SEQ ID NO: | (SEQ ID NO: 124) |
| | | 123) | |
| CD98hc.04.048.WH1.232 | KSSQSLLYSNSQKNYLA | WASTRES | QQYSYYPPT |
| | (SEQ ID NO: 130) | (SEQ ID NO: | (SEQ ID NO: 124) |
| | | 123) | |
| CD98hc.04.048.WH1.236 | KSSQSLLYSNKQKNYLA | WASTRES | QQYSYYPPT |
| | (SEQ ID NO: 131) | (SEQ ID NO: | (SEQ ID NO: 124) |
| | | 123) | |
| CD98hc.04.048.WH1.296 | KSSQSLLYENTQKNYLA | WASTRES | QQYSYYPPT |
| | (SEQ ID NO: 127) | (SEQ ID NO: | (SEQ ID NO: 124) |
| | | 123) | |
| CD98hc.04.048.WH1.151 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.158 | KSSQSLLYSNTQKNYLA | WASTRES | QQYSYYPPT |
| | (SEQ ID NO: 122) | (SEQ ID NO: | (SEQ ID NO: 124) |
| | | 123 | |
| CD98hc.04.048.WH1.162 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.165 | KSSQSLLYSNTQKNYLA | WASTRES | QQYSYYPPT |
| | (SEQ ID NO: 122) | (SEQ ID NO: | (SEQ ID NO: 124) |
| | | 123 | |
| CD98hc.04.048.WH1.166 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.167 | KSSQSLLYSNTQKNYLA | WASTRES | QQYSYYPPT |
| | (SEQ ID NO: 122) | (SEQ ID NO: | (SEQ ID NO: 124) |
| | | 123 | |
| CD98hc.04.048.WH1.168 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.170 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.171 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.174 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.185 | KSSQSSLYSNTQKNYLA | WASTRES | QQYSYYPPT |
| | (SEQ ID NO: 132) | (SEQ ID NO: | (SEQ ID NO: 124) |
| | | 123) | |
| CD98hc.04.048.WH1.208 | KSSQSLLGSNTQKNYLA | WASTRES | QQYSYYPPT |
| | (SEQ ID NO: 133) | (SEQ ID NO: | (SEQ ID NO: 124) |
| | | 123) | |
| CD98hc.04.048.WH1.231 | KSSQSLLYSNIQKNYLA | WASTRES | QQYSYYPPT |
| | (SEQ ID NO: 134) | (SEQ ID NO: | (SEQ ID NO: 124) |
| | | 123) | |
| CD98hc.04.048.WH1.235 | KSSQSLLYSNHQKNYLA | WASTRES | QQYSYYPPT |
| | (SEQ ID NO: 135) | (SEQ ID NO: | (SEQ ID NO: 124) |
| | | 123) | |
| CD98hc.04.048.WH1.250 | KSSQSLLYSNTQDNYLA | WASTRES | QQYSYYPPT |
| | (SEQ ID NO: 136) | (SEQ ID NO: | (SEQ ID NO: 124) |
| | | 123) | |
| CD98hc.04.048.WH1.253 | KSSQSLLYSNTQGNYLA | WASTRES | QQYSYYPPT |
| | (SEQ ID NO: 137) | (SEQ NO: | (SEQ ID NO: 124) |
| | | 123) | |
| CD98hc.04.048.WH1.262 | KSSQSLLYSNTQKNYLA | WASTRES | QQKSYYPPT |
| | (SEQ ID NO: 122) | (SEQ ID NO: | (SEQ ID NO: 138) |
| | | 123) | |
| CD98hc.04.048.WH1.269 | KSSQSLLYSNTQKNYLA | WASTRES | QQYKYYPPT |
| | (SEQ ID NO: 122) | (SEQ ID NO: | (SEQ ID NO: 139) |
| | | 123) | |
| CD98hc.04.048.WH1.271 | KSSQSLLYSNTQKNYLA | WASTRES | QQYTYYPPT |
| | (SEQ ID NO: 122) | (SEQ ID NO: | (SEQ ID NO: 140) |
| | | 123 | |
| CD98hc.04.048.WH1.274 | KSSQSLLYSNTQKNYLA | WASTRES | QQYEYYPPT |
| | (SEQ ID NO: 122) | (SEQ ID NO: | (SEQ ID NO: 141) |
| | | 123) | |
| CD98hc.04.048.WH1.276 | KSSQSLLYSNTQKNYLA | WASTRES | QQYIYYPPT |
| | (SEQ ID NO: 122) | (SEQ ID NO: | (SEQ ID NO: 142) |
| | | 123) | |
| CD98hc.04.048.WH1.279 | KSSQSLLYSNTQKNYLA | WASTRES | QQYSVYPPT |
| | (SEQ ID NO: 122) | (SEQ ID NO: | (SEQ ID NO: 143) |
| | | 123) | |
| CD98hc.04.048.WH1.281 | KSSQSLLYSNTQKNYLA | WASTRES | QQYSEYPPT |
| | (SEQ ID NO: 122) | (SEQ ID NO: | (SEQ ID NO: 144) |
| | | 123) | |
| CD98hc.04.048.WH1.282 | KSSQSLLYSNTQKNYLA | WASTRES | QQYSLYPPT |
| | (SEQ ID NO: 122) | (SEQ ID NO: | (SEQ ID NO: 145) |
| | | 123) | |
| CD98hc.04.048.WH1.283 | KSSQSLLYSNTQKNYLA | WASTRES | QQYSQYPPT |
| | (SEQ ID NO: 122) | (SEQ ID NO: | (SEQ ID NO: 146) |
| | | 123) | |
| CD98hc.04.048.WH1.39 | KSSQSLLYSNTQKNYLA | WASTRES | AQYSYYPPT |
| | (SEQ ID NO: 122) | (SEQ ID NO: | (SEQ ID NO: 147) |
| | | 123) | |
| CD98hc.04.048.WH1.55 | KSSQSLLYSNTQKNYLA | WASTRES | QQYSYYPPT |
| | (SEQ ID NO: 122) | (SEQ ID NO: | (SEQ ID NO: 124) |
| | | 123) | |
| CD98hc.04.048.WH1.76 | KSSQSLLYSNTQKNYLA | WASTRES | AQYSYYPPT |
| | (SEQ ID NO: 122) | (SEQ ID NO: | (SEQ ID NO: 147) |
| | | 123) | |

TABLE 11
VH and VL Sequences of Anti-CD98hc Antibodies

| Antibody | VH sequence | VL sequence |
|---|---|---|
| CD98hc.04.048. | EVQLLESGGGLVQPGGSLRLSCAA | DIVMTQSPDSLAVSLGERATINCKSS |
| WH1 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 148) | |
| CD98hc.04.048. | EVQLLESGGGLVQPGGSLRLSCAA | DIVMTQSPDSLAVSLGERATINCKSS |
| WH1.15 | SGFGISAYDMSWVRQAPGKGLEW | QSLLYSNTQKNYLAWYQQKPGQPP |
| | VSYISSGGGSTYYADEVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 150) | |
| CD98hc.04.048. | EVQLLESGGGLVQPGGSLRLSCAA | DIVMTQSPDSLAVSLGERATINCKSS |
| WH1.20 | SGFGISAYDMSWVRQAPGKGLEW | QSLLYSNTQKNYLAWYQQKPGQPP |
| | VSYISSGGGSTYYADFVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 151) | |
| CD98hc.04.048. | EVQLLESGGGLVQPGGSLRLSCAA | DIVMTQSPDSLAVSLGERATINCKSS |
| WH1.23 | SGFGISAYDMSWVRQAPGKGLEW | QSLLYSNTQKNYLAWYQQKPGQPP |
| | VSYISSGGGSTYYADYVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 152) | |
| CD98hc.04.048. | EVQLLESGGGLVQPGGSLRLSCAA | DIVMTQSPDSLAVSLGERATINCKSS |
| WH1.078 | SGFGISAYDMSWVRQAPGKGLEW | QSLLYSNTQKNYLAWYQQKPGQPP |
| | VSYKSSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 153) | |
| CD98hc.04.048. | EVQLLESGGGLVQPGGSLRLSCAA | DIVMTQSPDSLAVSLGERATINCKSS |
| WH1.083 | SGFGISAYDMSWVRQAPGKGLEW | QSLLYSNTQKNYLAWYQQKPGQPP |
| | VSYRSSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 154) | |
| CD98hc.04.048. | EVQLLESGGGLVQPGGSLRLSCAA | DIVMTQSPDSLAVSLGERATINCKSS |
| WH1.105 | SGFGISAYDMSWVRQAPGKGLEW | QSLLYSNTQKNYLAWYQQKPGQPP |
| | VSYISSQGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 155) | |
| CD98hc.04.048. | EVQLLESGGGLVQPGGSLRLSCAA | DIVMTQSPDSLAVSLGERATINCKSS |
| WH1.118 | SGFGISAYDMSWVRQAPGKGLEW | QSLLYSNTQKNYLAWYQQKPGQPP |
| | VSYISSGKGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 156) | |
| CD98hc.04.048. | EVQLLESGGGLVQPGGSLRLSCAA | DIVMTQSPDSLAVSLGERATINCKSS |
| WH1.119 | SGFGISAYDMSWVRQAPGKGLEW | QSLLYSNTQKNYLAWYQQKPGQPP |
| | VSYISSGHGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 157) | |
| CD98hc.04.048. | EVQLLESGGGLVQPGGSLRLSCAA | DIVMTQSPDSLAVSLGERATINCKSS |
| WH1.121 | SGFGISAYDMSWVRQAPGKGLEW | QSLLYSNTQKNYLAWYQQKPGQPP |
| | VSYISSGRGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 158) | |
| CD98hc.04.048. | EVQLLESGGGLVQPGGSLRLSCAA | DIVMTQSPDSLAVSLGERATINCKSS |
| WH1.133 | SGFGISAYDMSWVRQAPGKGLEW | QSLLYSNTQKNYLAWYQQKPGQPP |
| | VSYISSGGGHTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 159) | |
| CD98hc.04.048. | EVQLLESGGGLVQPGGSLRLSCAA | DIVMTQSPDSLAVSLGERATINCKSS |
| WH1.193 | SGFGISAYDMSWVRQAPGKGLEW | QSLKYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 160) |
| | S (SEQ ID NO: 148) | |
| CD98hc.04.048. | EVQLLESGGGLVQPGGSLRLSCAA | DIVMTQSPDSLAVSLGERATINCKSS |
| WH1.217 | SGFGISAYDMSWVRQAPGKGLEW | QSLLYQNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 161) |
| | S (SEQ ID NO: 148) | |
| CD98hc.04.048. | EVQLLESGGGLVQPGGSLRLSCAA | DIVMTQSPDSLAVSLGERATINCKSS |
| WH1.218 | SGFGISAYDMSWVRQAPGKGLEW | QSLLYENTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | |
| | CARQGGLPAWFVYWGQGTLVTVS | DFTLTISSLQAEDVAVYYCQQYSYY |
| | S (SEQ ID NO: 148) | PPTFGGGTKVEIK (SEQ ID NO: 162) |
| CD98hc.04.048. | EVQLLESGGGLVQPGGSLRLSCAA | DIVMTQSPDSLAVSLGERATINCKSS |
| WH1.222 | SGFGISAYDMSWVRQAPGKGLEW | QSLLYSHTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 163) |
| | S (SEQ ID NO: 148) | |
| CD98hc.04.048. | EVQLLESGGGLVQPGGSLRLSCAA | DIVMTQSPDSLAVSLGERATINCKSS |
| WH1.224 | SGFGISAYDMSWVRQAPGKGLEW | QSLLYSTTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 164) |
| | S (SEQ ID NO: 148) | |
| CD98hc.04.048. | EVQLLESGGGLVQPGGSLRLSCAA | DIVMTQSPDSLAVSLGERATINCKSS |
| WH1.232 | SGFGISAYDMSWVRQAPGKGLEW | QSLLYSNSQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 165) |
| | S (SEQ ID NO: 148) | |
| CD98hc.04.048. | EVQLLESGGGLVQPGGSLRLSCAA | DIVMTQSPDSLAVSLGERATINCKSS |
| WH1.236 | SGFGISAYDMSWVRQAPGKGLEW | QSLLYSNKQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 166) |
| | S (SEQ ID NO: 148) | |
| CD98hc.04.048. | EVQLLESGGGLVQPGGSLRLSCAA | DIVMTQSPDSLAVSLGERATINCKSS |
| WH1.296 | SGFGISAYDMSWVRQAPGKGLEW | QSLLYENTQKNYLAWYQQKPGQPP |
| | VSYISSQGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 162) |
| | S (SEQ ID NO: 155) | |
| CD98hc.04.048. | EVQLLESGGGLVQPGGSLRLSCAA | DIVMTQSPDSLAVSLGERATINCKSS |
| WH1.151 | SGFGISAYDMSWVRQAPGKGLEW | QSLLYSNTQKNYLAWYQQKPGQPP |
| | VSYISSGGGSDYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 149) |
| | S (SEQ ID NO: 167) | |
| CD98hc.04.048. | EVQLLESGGGLVQPGGSLRLSCAA | DIVMTQSPDSLAVSLGERATINCKSS |
| WH1.158 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGSLPAWFVYWGQGTLVTVS | PPTFGGGTKVEIK (SEQ ID NO: 149) |
| | S (SEQ ID NO: 168) | |
| CD98hc.04.048. | EVQLLESGGGLVQPGGSLRLSCAA | DIVMTQSPDSLAVSLGERATINCKSS |
| WH1.162 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGHPAWFVYWGQGTLVTVS | PPTFGGGTKVEIK (SEQ ID NO: 149) |
| | S (SEQ ID NO: 169) | |
| CD98hc.04.048. | EVQLLESGGGLVQPGGSLRLSCAA | DIVMTQSPDSLAVSLGERATINCKSS |
| WH1.165 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGAPAWFVYWGQGTLVTVS | PPTFGGGTKVEIK (SEQ ID NO: 149) |
| | S (SEQ ID NO: 170) | |
| CD98hc.04.048. | EVQLLESGGGLVQPGGSLRLSCAA | DIVMTQSPDSLAVSLGERATINCKSS |
| WH1.166 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGTPAWFVYWGQGTLVTVS | PPTFGGGTKVEIK (SEQ ID NO: 149) |
| | S (SEQ ID NO: 171) | |
| CD98hc.04.048. | EVQLLESGGGLVQPGGSLRLSCAA | DIVMTQSPDSLAVSLGERATINCKSS |
| WH1.167 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGQPAWFVYWGQGTLVTVS | PPTFGGGTKVEIK (SEQ ID NO: 149) |
| | S (SEQ ID NO: 172) | |
| CD98hc.04.048. | EVQLLESGGGLVQPGGSLRLSCAA | DIVMTQSPDSLAVSLGERATINCKSS |
| WH1.168 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPALFVYWGQGTLVTVS | PPTFGGGTKVEIK (SEQ ID NO: 149) |
| | S (SEQ ID NO: 173) | |
| CD98hc.04.048. | EVQLLESGGGLVQPGGSLRLSCAA | DIVMTQSPDSLAVSLGERATINCKSS |
| WH1.170 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAKFVYWGQGTLVTVS | PPTFGGGTKVEIK (SEQ ID NO: 149) |
| | S (SEQ ID NO: 174) | |
| CD98hc.04.048. | EVQLLESGGGLVQPGGSLRLSCAA | DIVMTQSPDSLAVSLGERATINCKSS |
| WH1.171 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPATFVYWGQGTLVTVS | PPTFGGGTKVEIK (SEQ ID NO: 149) |
| | S (SEQ ID NO: 175) | |
| CD98hc.04.048. | EVQLLESGGGLVQPGGSLRLSCAA | DIVMTQSPDSLAVSLGERATINCKSS |
| WH1.174 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPARFVYWGQGTLVTVS | PPTFGGGTKVEIK (SEQ ID NO: 149) |
| | S (SEQ ID NO: 176) | |
| CD98hc.04.048. | EVQLLESGGGLVQPGGSLRLSCAA | DIVMTQSPDSLAVSLGERATINCKSS |
| WH1.185 | SGFGISAYDMSWVRQAPGKGLEW | QSSLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 177) |
| | S (SEQ ID NO: 148) | |
| CD98hc.04.048. | EVQLLESGGGLVQPGGSLRLSCAA | DIVMTQSPDSLAVSLGERATINCKSS |
| WH1.208 | SGFGISAYDMSWVRQAPGKGLEW | QSLLGSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 178) |
| | S (SEQ ID NO: 148) | |
| CD98hc.04.048. | EVQLLESGGGLVQPGGSLRLSCAA | DIVMTQSPDSLAVSLGERATINCKSS |
| WH1.231 | SGFGISAYDMSWVRQAPGKGLEW | QSLLYSNIQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 179) |
| | S (SEQ ID NO: 148) | |
| CD98hc.04.048. | EVQLLESGGGLVQPGGSLRLSCAA | DIVMTQSPDSLAVSLGERATINCKSS |
| WH1.235 | SGFGISAYDMSWVRQAPGKGLEW | QSLLYSNHQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 180) |
| | S (SEQ ID NO: 148) | |
| CD98hc.04.048. | EVQLLESGGGLVQPGGSLRLSCAA | DIVMTQSPDSLAVSLGERATINCKSS |
| WH1.250 | SGFGISAYDMSWVRQAPGKGLEW | QSLLYSNTQDNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 181) |
| | S (SEQ ID NO: 148) | |
| CD98hc.04.048. | EVQLLESGGGLVQPGGSLRLSCAA | DIVMTQSPDSLAVSLGERATINCKSS |
| WH1.253 | SGFGISAYDMSWVRQAPGKGLEW | QSLLYSNTQGNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSYY |
| | CARQGGLPAWFVYWGQGTLVTVS | PPTFGGGTKVEIK (SEQ ID NO: 182) |
| | S (SEQ ID NO: 148) | |
| CD98hc.04.048. | EVQLLESGGGLVQPGGSLRLSCAA | DIVMTQSPDSLAVSLGERATINCKSS |
| WH1.262 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQKSYY |
| | CARQGGLPAWFVYWGQGTLVTVS | PPTFGGGTKVEIK (SEQ ID NO: 183) |
| | S (SEQ ID NO: 148) | |
| CD98hc.04.048. | EVQLLESGGGLVQPGGSLRLSCAA | DIVMTQSPDSLAVSLGERATINCKSS |
| WH1.269 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYKYY |
| | CARQGGLPAWFVYWGQGTLVTVS | PPTFGGGTKVEIK (SEQ ID NO: 184) |
| | S (SEQ ID NO: 148) | |
| CD98hc.04.048. | EVQLLESGGGLVQPGGSLRLSCAA | DIVMTQSPDSLAVSLGERATINCKSS |
| WH1.271 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYTYY |
| | CARQGGLPAWFVYWGQGTLVTVS | PPTFGGGTKVEIK (SEQ ID NO: 185) |
| | S (SEQ ID NO: 148) | |
| CD98hc.04.048. | EVQLLESGGGLVQPGGSLRLSCAA | DIVMTQSPDSLAVSLGERATINCKSS |
| WH1.274 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYEYY |
| | CARQGGLPAWFVYWGQGTLVTVS | PPTFGGGTKVEIK (SEQ ID NO: 186) |
| | S (SEQ ID NO: 148) | |
| CD98hc.04.048. | EVQLLESGGGLVQPGGSLRLSCAA | DIVMTQSPDSLAVSLGERATINCKSS |
| WH1.276 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYIYY |
| | CARQGGLPAWFVYWGQGTLVTVS | PPTFGGGTKVEIK (SEQ ID NO: 187) |
| | S (SEQ ID NO: 148) | |
| CD98hc.04.048. | EVQLLESGGGLVQPGGSLRLSCAA | DIVMTQSPDSLAVSLGERATINCKSS |
| WH1.279 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSVY |
| | CARQGGLPAWFVYWGQGTLVTVS | PPTFGGGTKVEIK (SEQ ID NO: 188) |
| | S (SEQ ID NO: 148) | |
| CD98hc.04.048. | EVQLLESGGGLVQPGGSLRLSCAA | DIVMTQSPDSLAVSLGERATINCKSS |
| WH1.281 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSEY |
| | CARQGGLPAWFVYWGQGTLVTVS | PPTFGGGTKVEIK (SEQ ID NO: 189) |
| | S (SEQ ID NO: 148) | |
| CD98hc.04.048. | EVQLLESGGGLVQPGGSLRLSCAA | DIVMTQSPDSLAVSLGERATINCKSS |
| WH1.282 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSLY |
| | CARQGGLPAWFVYWGQGTLVTVS | PPTFGGGTKVEIK (SEQ ID NO: 190) |
| | S (SEQ ID NO: 148) | |
| CD98hc.04.048. | EVQLLESGGGLVQPGGSLRLSCAA | DIVMTQSPDSLAVSLGERATINCKSS |
| WH1.283 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCQQYSQY |
| | CARQGGLPAWFVYWGQGTLVTVS | PPTFGGGTKVEIK (SEQ ID NO: 191) |
| | S (SEQ ID NO: 148) | |
| CD98hc.04.048. | EVQLLESGGGLVQPGGSLRLSCAA | DIVMTQSPDSLAVSLGERATINCKSS |
| WH1.39 | SGFGISAYDMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCAQYSYY |
| | CARQGGLPAWFVYWGQGTLVTVS | PPTFGGGTKVEIK (SEQ ID NO: 192) |
| | S (SEQ ID NO: 148) | |
| CD98hc.04.048. | EVQLLESGGGLVQPGGSLRLSCAA | DIVMSQSPSSLAVSVGEKVTMSCKS |
| WH1.55 | SGFGISAYAMSWVRQAPGKGLEW | SQSLLYSNTQKNYLAWYQQKPGQS |
| | VSYISSGGGSTYYADSVKGRFTISR | PKLLIYWASTRESGVPDRFTGSGSG |
| | DNSKNTLYLQMNSLRAEDTAVYY | TDFTLTISSVKAEDLAIYYCQQYSY |
| | CARQGGLPAWFVYWGQGTLVTVS | YPPTFGGGTKLEIK (SEQ ID NO: 194) |
| | S (SEQ ID NO: 193) | |
| CD98hc.04.048. | EVQLLESGGGLVQPGGSLRLSCAA | DIVMTQSPDSLAVSLGERATINCKSS |
| WH1.76 | SGFGISAYAMSWVRQAPGKGLEW | QSLLYSNTQKNYLAWYQQKPGQPP |
| | VSYISSGGGSTYYADSVKGRFTISR | KLLIYWASTRESGVPDRFSGSGSGT |
| | DNSKNTLYLQMNSLRAEDTAVYY | DFTLTISSLQAEDVAVYYCAQYSYY |
| | CARQGGLPAWFVYWGQGTLVTVS | PPTFGGGTKVEIK (SEQ ID NO: 192) |
| | S (SEQ ID NO: 193) | |

In some aspects, an antigen-binding domain that specifically binds to human CD98hc comprises the six CDRs of an antibody listed in Tables 9, 10, and 11 (i.e. the three VH CDRs of the antibody listed in Table 9 and the three VL CDRs of the same antibody listed in Table 10). In some aspects, an antigen-binding domain that specifically binds to human CD98hc comprises the six CDRs of an antibody listed in Tables 9, 10, and 11 as determined by Kabat numbering. In some aspects, an antigen-binding domain that specifically binds to human CD98hc comprises the six Chothia CDRs of an antibody listed in Table 11.

In some aspects, the CDRs of an antigen-binding domain that specifically binds to human CD98hc can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dubel, eds., Chapter 31, pp. 422-439, Springer-Verlag, Berlin (2001). In some aspects, provided herein are antigen-binding domains that specifically bind to human CD98hc and comprise VH and VL CDRs of an antibody listed in Table 11 as determined by the method in MacCallum R M et al.

In some aspects, the CDRs of an antigen-binding domain that specifically binds to human CD98hc can be determined according to the AbM numbering scheme (as described above). In some aspects, provided herein are antigen-binding domains that specifically bind to human CD98hc and comprise VH and VL CDRs of an antibody listed in Table 11 as determined by the AbM numbering scheme.

In some aspects, an antigen-binding domain that specifically binds to human CD98hc comprises the six IMGT CDRs of an antibody listed in Table 11 according to the IMGT numbering system as described above.

In some aspects, an antigen-binding domain that specifically binds to human CD98hc provided herein is described by its VL domain alone, or its VH domain alone, or by its 3 VL CDRs alone, or its 3 VH CDRs alone.

In some aspects, an antigen-binding domain that specifically binds to human CD98hc comprises the VH of an antibody listed in Table 11.

In some aspects, an antigen-binding domain that specifically binds to human CD98hc comprises the VL of antibody listed in Table 11.

In some aspects, an antigen-binding domain that specifically binds to human CD98hc comprises the VH and the VL of an antibody listed in Table 11.

In another aspect, an antigen-binding domain of the present disclosure that specifically binds to human CD98hc comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs:148, 150, 151, 152, 153, 154, 155, 156, 157, 158, 159, 167, 168, 169, 170, 171, 172, 173, 174, 175, 176, and 193. In certain aspects, a V_{H} sequence having at least 90%, 91%, 92%, 93%, 94%, 95%, 96%, 97%, 98%, 99%, or 100% identity to an amino acid sequence selected from the group consisting of SEQ ID NOs: 148, 150, 151, 152, 153, 154, 155, 156, 157, 158, 159, 167, 168, 169, 170, 171, 172, 173, 174, 175, 176, and 193 contains substitutions (e.g., conservative substitutions), insertions, or deletions relative to the reference sequence, but an antigen-binding domain that specifically binds to human CD98hc comprising that sequence retains the ability to bind to human CD98hc. In certain aspects, a total of 1 to 10 amino acids have been substituted, inserted, and/or deleted in SEQ ID NO: 148, 150, 151, 152, 153, 154, 155, 156, 157, 158, 159, 167, 168, 169, 170, 171, 172, 173, 174, 175, 176, or 193. In certain aspects, a total of 1 to 5 amino acids have been substituted, inserted and/or deleted in SEQ ID NO: 148, 150, 151, 152, 153, 154, 155, 156, 157, 158, 159, 167, 168, 169, 170, 171, 172, 173, 174, 175, 176, or 193. In certain aspects, substitutions, insertions, or deletions occur in regions outside the HVRs (i.e., in the FRs). Optionally, the antigen-binding domain that specifically binds to human CD98hc comprises the V_{H} sequence of SEQ ID NO: 148, 150, 151, 152, 153, 154, 155, 156, 157, 158, 159, 167, 168, 169, 170, 171, 172, 173, 174, 175, 176, and 193, including post-translational modifications of that sequence.

In another aspect, an antigen-binding domain of the present disclosure that specifically binds to human CD98hc comprises comprises a light chain variable domain (V_{L}) having at least 90%, 91%, 92%, 93%, 94%, 95%, 96%, 97%, 98%, 99%, or 100% sequence identity to an amino acid sequence selected from the group consisting of SEQ ID NOs:149, 160, 161, 162, 163, 164, 165, 166, 177, 178, 179, 180, 181, 182, 183, 184, 185, 186, 187, 188, 189, 190, 191, 192, and 194. In certain aspects, a V_{L} sequence having at least 90%, 91%, 92%, 93%, 94%, 95%, 96%, 97%, 98%, 99%, or 100% identity to an amino acid sequence selected from the group consisting of SEQ ID NOs: 149, 160, 161, 162, 163, 164, 165, 166, 177, 178, 179, 180, 181, 182, 183, 184, 185, 186, 187, 188, 189, 190, 191, 192, and 194 and contains substitutions (e.g., conservative substitutions), insertions, or deletions relative to the reference sequence, but an antigen-binding domain of the present disclosure that specifically binds to human CD98hc comprising that sequence retains the ability to bind to human CD98hc. In some aspects, a total of 1 to 10 amino acids have been substituted, inserted and/or deleted in SEQ ID NO: 149, 160, 161, 162, 163, 164, 165, 166, 177, 178, 179, 180, 181, 182, 183, 184, 185, 186, 187, 188, 189, 190, 191, 192, or 194. In certain aspects, a total of 1 to 5 amino acids have been substituted, inserted and/or deleted in SEQ ID NO: 149, 160, 161, 162, 163, 164, 165, 166, 177, 178, 179, 180, 181, 182, 183, 184, 185, 186, 187, 188, 189, 190, 191, 192, or 194. In certain aspects, the substitutions, insertions, or deletions occur in regions outside the HVRs (i.e., in the FRs). Optionally, the antigen-binding domain of the present disclosure that specifically binds to human CD98hc comprises the V_{L} sequence of SEQ ID NO: 149, 160, 161, 162, 163, 164, 165, 166, 177, 178, 179, 180, 181, 182, 183, 184, 185, 186, 187, 188, 189, 190, 191, 192, and 194, including post-translational modifications of that sequence.

In some aspects, an antigen-binding domain of the present disclosure that specifically binds to human CD98hc is provided, wherein the antibody comprises a V_{H} as in any of the aspects provided above, and a V_{L} as in any of the aspects provided above. In some aspects, the antigen-binding domain comprises the V_{H} and V_{L} sequences in SEQ ID NOs: 148, 150, 151, 152, 153, 154, 155, 156, 157, 158, 159, 167, 168, 169, 170, 171, 172, 173, 174, 175, 176, and 193, and SEQ ID NOs: 149, 160, 161, 162, 163, 164, 165, 166, 177, 178, 179, 180, 181, 182, 183, 184, 185, 186, 187, 188, 189, 190, 191, 192, and 194, respectively, including post-translational modifications of those sequences.

In some aspects, provided herein are antigen-binding domains that specifically bind to human TfR comprising a heavy chain variable domain (V_{H}) and a light chain variable domain (V_{L}), wherein the V_{H} and V_{L} are selected from the group consisting of: V_{H} comprising the amino acid sequence of SEQ ID NO:148 and V_{L} comprising the amino acid sequence of SEQ ID NO:149; V_{H} comprising the amino acid sequence of SEQ ID NO:150 and V_{L} comprising the amino acid sequence of SEQ ID NO:149; V_{H} comprising the amino acid sequence of SEQ ID NO:151 and V_{L} comprising the amino acid sequence of SEQ ID NO:149; V_{H} comprising the amino acid sequence of SEQ ID NO:152 and V_{L} comprising the amino acid sequence of SEQ ID NO:149; V_{H} comprising the amino acid sequence of SEQ ID NO:153 and V_{L} comprising the amino acid sequence of SEQ ID NO:149; V_{H} comprising the amino acid sequence of SEQ ID NO:154 and V_{L} comprising the amino acid sequence of SEQ ID NO:149; V_{H} comprising the amino acid sequence of SEQ ID NO:155 and V_{L} comprising the amino acid sequence of SEQ ID NO:149; V_{H} comprising the amino acid sequence of SEQ ID NO:156 and V_{L} comprising the amino acid sequence of SEQ ID NO:149; V_{H} comprising the amino acid sequence of SEQ ID NO:157 and V_{L} comprising the amino acid sequence of SEQ ID NO:149; V_{H} comprising the amino acid sequence of SEQ ID NO:158 and V_{L} comprising the amino acid sequence of SEQ ID NO:149; V_{H} comprising the amino acid sequence of SEQ ID NO:159 and V_{L} comprising the amino acid sequence of SEQ ID NO:149; V_{H} comprising the amino acid sequence of SEQ ID NO:148 and V_{L} comprising the amino acid sequence of SEQ ID NO:160; V_{H} comprising the amino acid sequence of SEQ ID NO:148 and V_{L} comprising the amino acid sequence of SEQ ID NO:161; V_{H} comprising the amino acid sequence of SEQ ID NO:148 and V_{L} comprising the amino acid sequence of SEQ ID NO:162; V_{H} comprising the amino acid sequence of SEQ ID NO:148 and V_{L} comprising the amino acid sequence of SEQ ID NO:163; V_{H} comprising the amino acid sequence of SEQ ID NO:148 and V_{L} comprising the amino acid sequence of SEQ ID NO:164; V_{H} comprising the amino acid sequence of SEQ ID NO:148 and V_{L} comprising the amino acid sequence of SEQ ID NO:165; V_{H} comprising the amino acid sequence of SEQ ID NO:148 and V_{L} comprising the amino acid sequence of SEQ ID NO:166; V_{H} comprising the amino acid sequence of SEQ ID NO:155 and V_{L} comprising the amino acid sequence of SEQ ID NO:162; V_{H} comprising the amino acid sequence of SEQ ID NO:167 and V_{L} comprising the amino acid sequence of SEQ ID NO:149; V_{H} comprising the amino acid sequence of SEQ ID NO:168 and V_{L} comprising the amino acid sequence of SEQ ID NO:149; V_{H} comprising the amino acid sequence of SEQ ID NO:169 and V_{L} comprising the amino acid sequence of SEQ ID NO:149; V_{H} comprising the amino acid sequence of SEQ ID NO:170 and V_{L} comprising the amino acid sequence of SEQ ID NO:149; V_{H} comprising the amino acid sequence of SEQ ID NO:171 and V_{L} comprising the amino acid sequence of SEQ ID NO:149; V_{H} comprising the amino acid sequence of SEQ ID NO:172 and V_{L} comprising the amino acid sequence of SEQ ID NO:149; V_{H} comprising the amino acid sequence of SEQ ID NO:173 and V_{L} comprising the amino acid sequence of SEQ ID NO:149; V_{H} comprising the amino acid sequence of SEQ ID NO:174 and V_{L} comprising the amino acid sequence of SEQ ID NO:149; V_{H} comprising the amino acid sequence of SEQ ID NO:175 and V_{L} comprising the amino acid sequence of SEQ ID NO:149; V_{H} comprising the amino acid sequence of SEQ ID NO:176 and V_{L} comprising the amino acid sequence of SEQ ID NO:149; V_{H} comprising the amino acid sequence of SEQ ID NO:148 and V_{L} comprising the amino acid sequence of SEQ ID NO:177; V_{H} comprising the amino acid sequence of SEQ ID NO:148 and V_{L} comprising the amino acid sequence of SEQ ID NO:178; V_{H} comprising the amino acid sequence of SEQ ID NO:148 and V_{L} comprising the amino acid sequence of SEQ ID NO:179; V_{H} comprising the amino acid sequence of SEQ ID NO:148 and V_{L} comprising the amino acid sequence of SEQ ID NO:180; V_{H} comprising the amino acid sequence of SEQ ID NO:148 and V_{L} comprising the amino acid sequence of SEQ ID NO:181; V_{H} comprising the amino acid sequence of SEQ ID NO:148 and V_{L} comprising the amino acid sequence of SEQ ID NO:182; V_{H} comprising the amino acid sequence of SEQ ID NO:148 and V_{L} comprising the amino acid sequence of SEQ ID NO:183; V_{H} comprising the amino acid sequence of SEQ ID NO:148 and V_{L} comprising the amino acid sequence of SEQ ID NO:184; V_{H} comprising the amino acid sequence of SEQ ID NO:148 and V_{L} comprising the amino acid sequence of SEQ ID NO:185; V_{H} comprising the amino acid sequence of SEQ ID NO:148 and V_{L} comprising the amino acid sequence of SEQ ID NO:186; V_{H} comprising the amino acid sequence of SEQ ID NO:148 and V_{L} comprising the amino acid sequence of SEQ ID NO:187; V_{H} comprising the amino acid sequence of SEQ ID NO:148 and V_{L} comprising the amino acid sequence of SEQ ID NO:188; V_{H} comprising the amino acid sequence of SEQ ID NO:148 and V_{L} comprising the amino acid sequence of SEQ ID NO:189; V_{H} comprising the amino acid sequence of SEQ ID NO:148 and V_{L} comprising the amino acid sequence of SEQ ID NO:190; V_{H} comprising the amino acid sequence of SEQ ID NO:148 and V_{L} comprising the amino acid sequence of SEQ ID NO:191; V_{H} comprising the amino acid sequence of SEQ ID NO:148 and V_{L} comprising the amino acid sequence of SEQ ID NO:192; V_{H} comprising the amino acid sequence of SEQ ID NO:193 and V_{L} comprising the amino acid sequence of SEQ ID NO:194; and V_{H} comprising the amino acid sequence of SEQ ID NO:193 and V_{L} comprising the amino acid sequence of SEQ ID NO:192.

In some aspects, provided herein is an antigen-binding domain that competitively inhibits binding to CD98hc as an antibody comprising a VdH amino acid sequence of an antibody in Table 11 and comprising a VL amino acid sequence of the same antibody in Table 11.

In some aspects, an antigen-binding domain that specifically binds to human CD98hc comprises a VH and a VL on a single polypeptide chain (e.g., a VH and VL in Table 11). In some aspects, the antigen-binding domain comprises an scFv. The scFv can comprise a VH that is N-terminal to a VL or a VL that is N-terminal to a VH. The scFv can comprise a linker, e.g., between a VH and a VL. Accordingly, the scFv can be in the orientation VH-linker-VL or VL-linker-VH. Such a linker can be about 5 to about 25 amino acids in length. Such a linker can be about 5 to about 20 amino acids in length. Such a linker can be about 10 to about 25 amino acids in length. Such a linker can be about 10 to about 20 amino acids in length. Such a linker can be, e.g., a glycine linker, a glycine-rich linker, or a glycine-serine linker. Such a linker can comprise the amino acid sequence of GGSEGKSSGSGSESKSTGGS (SEQ ID NO: 209). Such a linker can comprise the amino acid sequence of GGGGSGGGGSGGGGSGGGGS (SEQ ID NO: 210).

In some aspects, an antigen-binding domain that specifically binds to human CD98hc comprises a VH on a first polypeptide and a VL on a second polypeptide (e.g., a Fab).

In some aspects, an antigen-binding domain that specifically binds to human CD98hc comprises the antigen-binding fragment of a heavy chain only antibody (e.g., a VHH or nanobody).

In some aspects, an antigen-binding domain that specifically binds to human CD98hc is a murine antigen-binding domain. In some aspects, an antigen-binding domain that specifically binds to human CD98hc is a chimeric antigen-binding domain. In some aspects, an antigen-binding domain that specifically binds to human CD98hc is a humanized antigen-binding domain. In some aspects, an antigen-binding domain that specifically binds to human CD98hc is a human antigen-binding domain.

In some aspects, an antigen-binding domain provided herein that specifically binds to human CD98hc also binds to cynomolgus monkey CD98hc.

In some aspects, the antigen-binding domain provided herein binds human CD98hc with an affinity between about 500 nM and about 10,000 nM. In some aspects, the antigen-binding domain binds human CD98hc with an affinity between about 500 nM and about 5,000 nM, between about 500 nM and about 2,500 nM, between about 500 nM and about 1,000 nM, between about 500 nM and about 750 nM, between about 1,000 nM and about 2,5000 nM, between about 2,500 nM and about 5,000 nM, between about 2,500 nM and about 10,000 nM, between about 5,000 nM and about 10,000 nM, and values and ranges there between.

In some aspects, an antigen-binding domain provided herein binds human CD98hc with an affinity between about 10 nM and about 500 nM. In some aspects, the antigen-binding domain binds human CD98hc with an affinity between about 10 nM and about 250 nM, between about 10 nM and about 100 nM, between about 10 nM and about 50 nM, between about 50 nM and about 500 nM, between about 50 nM and about 250 nM, between about 100 nM and about 250 nM, between about 250 nM and about 500 nM, and values and ranges there between.

In some aspects, the antigen-binding domain binds human CD98hc with an affinity less than about 10 nM. In some aspects, the antigen-binding domain binds human CD98hc with an affinity between about 1 nM and about 10 nM, between about 1 nM and about 5 nM, between about 0.1 nM and about 1 nM, between about 0.01 nM and about 0.1 nM, and values and ranges there between.

In some aspects, an antigen-binding domain provided herein that specifically binds to human CD98hc binds to human CD98hc is measured using surface plasmon resonance. Surface plasmon resonance can be measured, e.g., using the Carterra LSA platform. In some aspects, an antigen-binding domain provided herein that specifically binds to human CD98hc binds to human CD98hc as measured by, for example, radioimmunoassay (RIA), Western blot, or ELISA 0D450.

Also provided herein are antigen-binding domains that bind to the same epitope of CD98hc as a CD98hc antigen-binding domain provided herein. Also provided herein are antigen-binding domains that competitively inhibit binding to CD98hc of CD98hc antigen-binding domain provided herein.

Multi-Specific Binding Proteins that Comprise an Anti-pyroGlu3 Abeta (Aβ) Antigen-Binding Domain and an Antigen-Binding Domain that Binds to Human TfR

Provided herein are multi-specific binding proteins that comprise an antigen-binding domain that specifically binds to human anti-pyroGlu3 Abeta (Aβ) and an antigen-binding domain that specifically binds to human TfR.

In some aspects, an antibody or antigen-binding fragment thereof provided herein comprises an antigen-binding domain that specifically binds to human TfR. In some aspects, an antibody or antigen-binding fragment thereof comprises an antigen-binding domain that specifically binds to human TfR linked to a second antigen-binding domain that specifically binds to human pyroGlu3 Abeta (Aβ). Also provided herein are antibodies or antigen-binding fragments thereof that bind to the same epitope of TfR as a TfR antigen-binding domain provided herein. Also provided herein are antibodies or antigen-binding fragments thereof that competitively inhibit binding to TfR of a TfR antigen-binding domain provided herein.

In some aspects, a multi-specific binding protein provided herein comprises a first antigen-binding domain that specifically binds to human TfR linked to a second antigen-binding domain that specifically binds to human pyroGlu3 Abeta (Aβ). The first antigen-binding domain that specifically binds to human TfR can be any antigen-binding domain that binds to human TfR provided herein. The second antigen-binding domain can be any antigen-binding domain that specifically binds to human pyroGlu3 Abeta (Aβ) provided herein.

In some aspects, a multi-specific binding protein provided herein comprises an antigen-binding domain that specifically binds to human TfR that is linked to a second antibody or antigen-binding fragment thereof. The second antibody or antigen-binding fragment thereof specifically binds to human pyroGlu3 Abeta (Aβ). In some aspects, the second antibody or antigen-binding fragment does not bind to TfR.

In some aspects, a multi-specific binding protein provided herein is bi-specific. In some aspects, a multi-specific binding protein provided herein is bivalent, trivalent, or tetravalent.

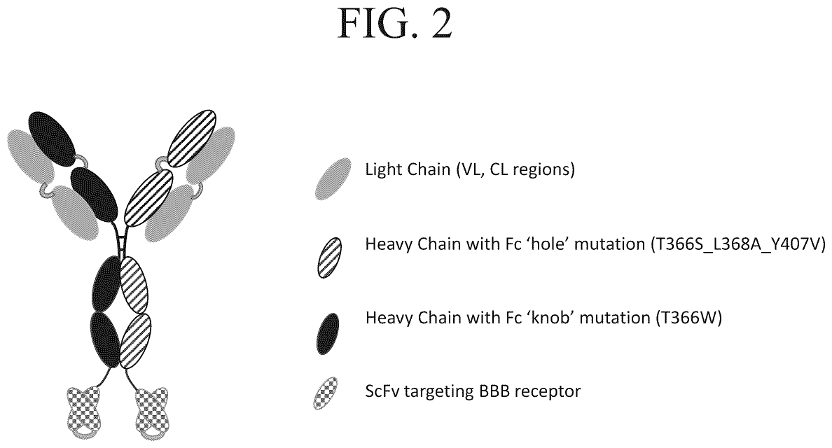

In some aspects, a multi-specific binding protein provided herein comprises a 2+1 multi-specific protein format (as shown in FIG. 1A and FIG. 1B) or a 2+2 multispecific-protein format (as shown in FIG. 2). As used herein, “2+1” refers to the multi-specific protein format in FIG. 1A or FIG. 1B.

In some aspects, a multi-specific binding protein provided herein comprises a TfR antigen-binding domain that is an scFv linked to an antibody that binds to human pyroGlu3 Abeta (Aβ), wherein the antibody that binds to human pyroGlu3 Abeta (Aβ) comprises two heavy chains and two light chains. In some aspects, the scFv is linked to the C-terminus of one of the two antibody heavy chains, e.g., via a protein linker.

In some aspects, the multi-specific binding protein comprises 1) an antigen-binding domain that specifically binds TfR, 2) a second antigen-binding domain that specifically binds to human pyroGlu3 Abeta (Aβ), and 3) an Fc region, wherein the TfR antigen-binding domain and the second antigen-binding domain are connected or linked to the Fc region of the multi-specific binding protein. In other aspects, the multi-specific binding protein comprises 1) an antigen-binding domain that comprises a heavy chain variable region and specifically binds TfR, 2) a second antigen binding domain that comprises a heavy-chain variable region and specifically binds to human pyroGlu3 Abeta (Aβ), and 3) an Fc region, wherein the TfR antigen-binding domain and the second antigen-binding domain are connected or linked to the Fc region of the multi-specific binding protein.

In some aspects, the multi-specific binding protein comprises 1) an antigen-binding domain that specifically binds TfR, 2) a second antigen-binding domain that binds to human pyroGlu3 Abeta (Aβ), and 3) an Fc region, wherein the antigen-binding domain that specifically binds TfR and the second antigen-binding domain are both connected or linked to the N-terminus of the Fc portion of the multi-specific binding protein. In other aspects, the antigen-binding domain that specifically binds TfR is connected or linked to the N-terminus of an Fc portion of the multi-specific protein and the second antigen-binding domain is linked to the C-terminus of the Fc portion of the multi-specific binding protein. In other aspects, the antigen-binding domain that specifically binds TfR is connected or linked to the C-terminus of an Fc portion of the multi-specific binding protein and the second antigen-binding domain is linked to the N-terminus of the Fc portion of the multi-specific binding protein.

In some aspects, a multi-specific binding protein provided herein comprises two scFv molecules that comprise antigen-binding domains that specifically bind TfR and an antibody that binds to human pyroGlu3 Abeta (Aβ), wherein the antibody comprises two heavy chains and two light chains, and wherein one of the two scFv molecules is linked to the C-terminus of one of the antibody heavy chains, and wherein the other scFv molecule is linked to the C-terminus of the other antibody heavy chain. In some aspects, the scFvs are linked to the heavy chains of the antibody via a protein linker.

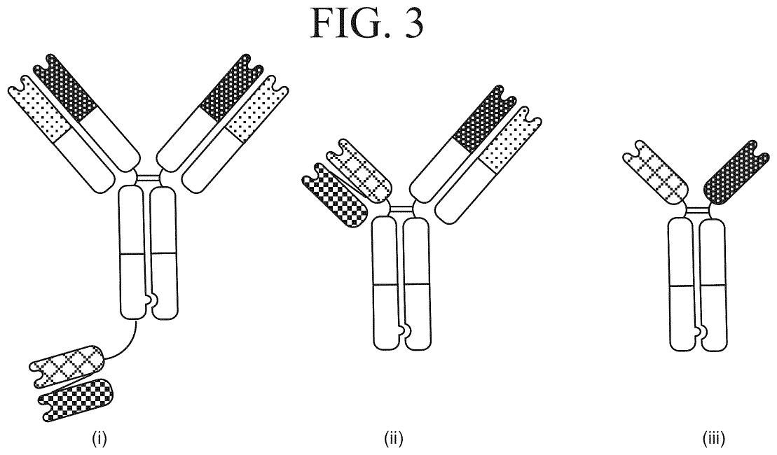

In some aspects, disclosed herein is a trivalent, bi-specific protein comprising (i) a single scFv, Fab or VHH antigen-binding domain that specifically binds to human TfR and (ii) an antibody that specifically binds to human pyroGlu3 Abeta (Aβ), wherein the antibody comprises two heavy chains and two light chains, and wherein the single scFv, Fab or VHH antigen-binding domain that specifically binds to human TfR is linked to the C-terminus or N-terminus of one of the two antibody heavy chains. The Fc may be a heterodimeric Fc, such as knob and hole Fc. An example of this 2+1 format with a knob-hole Fc is shown as (i) in FIG. 3.

In some aspects, disclosed herein is a tetravalent, bi-specific protein comprising (i) two scFv, Fab or VHH antigen-binding domains that specifically bind to human TfR and (ii) an antibody that binds to human pyroGlu3 Abeta (Aβ), wherein the antibody comprises two heavy chains and two light chains, and wherein one of the scFv, Fab, or VHH antigen-binding domains that specifically binds to human TfR is linked to the C-terminus of one of the two antibody heavy chains, and the other scFv, Fab, or VHH antigen-binding domain that specifically binds to human TfR is linked to the C-terminus or N-terminus of the other of the two antibody heavy chains. In some aspects, the two scFv, Fab or VHH antigen-binding domains that bind to human TfR can comprise the same amino acid sequence. In some aspects, the Fc is a heterodimeric Fc, such as an Fc region with knob and hole mutations.

In some aspects, disclosed herein is a bivalent, bispecific protein comprising (i) a single scFv, VHH, or Fab antigen-binding domain that specifically binds to human TfR linked to the N-terminus of an Fc domain of an Fc dimer, and (ii) a second antigen binding domain that binds to human pyroGlu3 Abeta (Aβ), wherein the second antigen binding domain is linked to the N-terminus of the second Fc domain of an Fc dimer. An example of this format is shown as (iii) in FIG. 3.

In some aspects, disclosed herein is a bivalent, bi-specific protein comprising (i) a single scFv, VHH, or Fab antigen-binding domain that specifically binds to human TfR linked to the N-terminus or C-terminus of an Fc domain of an Fc dimer and (ii) a second antigen binding domain, wherein the second antigen binding domain binds to human pyroGlu3 Abeta (Aβ) and comprises a single scFv, VHH or Fab linked to N-terminus of the Fc. In some aspects, disclosed herein is a bivalent, bi-specific protein comprising (i) a single scFv, VHH, or Fab antigen-binding domain that specifically binds to human TfR linked to the N-terminus or C-terminus of an Fc domain of an Fe dimer and (ii) a second antigen binding domain, wherein the second antigen binding domain binds to human pyroGlu3 Abeta (Aβ) and comprises a single scFv, VHH or Fab linked to the C-terminus of the second Fc domain of the Fe dimer. In some aspects, the Fe is a heterodimeric Fe, such as an Fc domain with knob and hole mutations. An example of this format is shown as (ii) in FIG. 3, wherein a single scFv is linked to the N terminus of a first Fc domain of the Fc region and the second antigen binding domain is linked to the N terminus of the second Fc domain of the Fc region.

In some aspects, disclosed herein is a bivalent, bispecific protein comprising (i) a single scFv, VHH, or Fab antigen-binding domain that specifically binds to human TfR and is linked to the N-terminus of an Fc domain of an Fe dimer, and (ii) a second antigen binding domain that binds to human pyroGlu3 Abeta (Aβ) and is linked to the N-terminus of the second Fc domain of an Fe dimer. An example of this format is shown as (iii) in FIG. 3.

In some aspects, disclosed herein is a bivalent, bi-specific protein comprising (i) a single scFv, VHH, or Fab antigen-binding domain that binds to human TfR and is linked to the N-terminus or C-terminus of an Fc domain of an Fe dimer and (ii) a second antigen binding domain, wherein the second antigen binding domain binds to human pyroGlu3 Abeta (Aβ) and comprises a single scFv, VHH or Fab linked to N-terminus of the Fe or C-terminus of a second Fc domain of the Fe dimer. In some aspects, the Fe is a heterodimeric Fe, such as an Fc domain with knob and hole mutations. An example of this 1+1 format is shown as (ii) in FIG. 3, where a single scFv is linked to the N terminus of an Fc domain of an Fc region and a second antigen binding domain is linked to the N terminus of a second Fc domain of the Fc region.

As provided herein, an antibody or antigen-binding fragment thereof, or multi-specific binding protein provided herein can be multi-specific, e.g., bi-specific. Many different formats and uses of bi-specific binding molecules are known in the art (reviewed in, e.g., Kontermann; Drug Discov Today, 2015 July; 20(7):838-47; MAbs, 2012 March-April; 4(2):182-97). A bispecific protein according to the present disclosure is not limited to any particular bispecific format or method of producing it. Accordingly, a bispecific protein of the present disclosure can include various configurations having a first antigen-binding domain that binds to human TfR and a second antigen-binding domain or antibody or antigen-binding fragment thereof that binds to human pyroGlu3 Aβ.

Bispecific molecules include, e.g., a kappa-lambda body, a dual-affinity re-targeting molecule (DART), a knob-in-hole antibody, a strand-exchange engineered domain body (SEEDbody), and a DuoBody. In some aspects, a bispecific antibody or antigen-binding fragment thereof, or multi-specific protein provided herein comprises a knob mutation and a hole mutation. In some aspects, the knob mutation comprises the amino acid substitution T366W according to EU numbering. In some aspects, the hole mutation comprises the amino acids substitutions T366S, L368A, and Y407V according to EU numbering. Examples of bispecific molecules that can be used in the present disclosure include (i) a single antibody that has two arms comprising different antigen-binding domains; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig.TM.) Molecule, In: Antibody Engineering, Springer Berlin Heidelberg (2010)); (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody. Other examples of antibody structures are described in WO2019/246288, which is incorporated by reference.

In some aspects, a bispecific protein provided herein is selected from one of the following formats: CrossMab, DAF (two-in-one), DAF (four-in-one), DutaMab, DT-IgG, Charge pair, Fab-arm exchange, Triomab, LUZ-Y, Fcab, kappalambda-body, Orthogonal Fab, DVD-IgG, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG (four-in-one), Nanobody, Nanbody-HAS, BiTE, TandAb, scDiabody-CH3, Diabody-CH3, Triple Body, miniantibody, minibody, TriBi minibody, scFv-CH3 KIH, Fab-scFv, scFV-CH-CL-scFV, F(ab′)2, F(ab′)2-scFv2, scFV-KIH, Fab-scFv-Fc, Tetravalent HCAb, scDiabody-Fc, Diabody-Fe, Tandem scFv-Fc, Intrabody, ImmTAC, HSAbody, scDiabody-HAS, Tandem scFv-toxin, IgG-IgG, Cov-X-body, and scFv1-PEG-scFv2, as described in Spiess, C., et al., Alternative molecular formats and therapeutic applications for bispecific antibodies. Mol. Immunol. (2015), which is incorporated by reference herein.

In some aspects, a multi-specific binding protein provided herein is bivalent. In some aspects, a multi-specific binding protein provided herein is multivalent (e.g., bivalent). In some aspects, an antibody or antigen-binding fragment thereof, or multi-specific binding protein provided herein is trivalent (e.g., in a 2+1 format). In some aspects, a trivalent format comprises a single TfR antigen-binding domain provided herein and two antigen-binding domains that bind to N3pE Abeta (Aβ). The two antigen-binding domains that bind to a CNS antigen or a brain antigen can comprise the same amino acid sequence or can comprise different amino acid sequences. In some aspects, the TfR antigen-binding domain is an scFv. In some aspects, the TfR antigen-binding domain is a VHH. In some aspects, the TfR antigen-binding domain is a Fab.

In some aspects, a multi-specific binding protein provided herein is tetravalent (e.g., in a 2+2 format). In some aspects, a tetravalent format comprises two TfR antigen-binding domains provided herein and two antigen-binding domains that bind to a CNS antigen or a brain antigen.

The two TfR antigen-binding domains can comprise the same amino acid sequence or can comprise different amino acid sequences. In some aspects, the two TfR antigen-binding domains comprise the same amino acid sequence. In some aspects, one or both of the TfR antigen-binding domains is an scFv. The two antigen-binding domains that bind to a N3pE Abeta (Aβ) antigen can comprise the same amino acid sequence or can comprise different amino acid sequences

An antibody or antigen-binding fragment thereof, or multi-specific binding protein provided herein can comprise a linker. The linker can be e.g., a glycine linker, a glycine-rich linker, or a glycine-serine linker. The linker can comprise the amino acid sequence (GGGGS)×3 (SEQ ID NO: 207). The linker can comprise the amino acid sequence (GGSGG)×3 (SEQ ID NO: 208).

An antibody or antigen-binding fragment thereof, or multi-specific binding protein provided herein can comprise a constant region. In some aspects, a TfR antigen-binding domain provided herein is linked to the constant region, e.g., the C-terminus of the constant region. In some aspects, a constant domain is a human constant domain. In some aspects, a constant domain is a murine, rat, rabbit, or monkey (e.g., cynomolgus) constant domain. The constant region can be a heavy chain constant region. The constant region can be a human constant region. The constant region can be a human heavy chain constant region. The constant region can be an IgG constant region. The constant region can be an IgG1 constant region. The constant region can be an IgG2 constant region. The constant region can be an IgG4 constant region. The constant region can be a human IgG constant region. The constant region can be a human IgG1 constant region. The constant region can be a human IgG2 constant region. The constant region can be a human IgG4 constant region. The linker can comprise the amino acid sequence GGSGG (no repeats) (SEQ ID NO: 214). The linker may be 1 to 20 amino acids in length.

In some aspects, an antibody or antigen-binding fragment thereof, or multi-specific binding protein provided herein comprises a heavy chain and a light chain. With respect to the heavy chain, in some aspects, the heavy chain of an antigen-binding protein described herein can be an alpha (α), delta (δ), epsilon (ε), gamma (γ) or mu (μ) heavy chain. In some aspects, the heavy chain can comprise a human alpha (α), delta (δ), epsilon (ε), gamma (γ) or mu (μ) heavy chain. In some aspects, the heavy chain comprises a human gamma (γ) heavy chain constant region. In some aspects, the heavy chain of comprises the amino acid sequence of an IgG1 heavy chain constant region. In some aspects, the heavy chain comprises the amino acid sequence of an IgG2 (e.g., IgG2a or IgG2b) heavy chain constant region. In some aspects, the heavy chain comprises the amino acid sequence of an IgG4 heavy chain constant region. With respect to the light chain, in some aspects, the light chain is a kappa light chain. In some aspects, the light chain is a lambda light chain. In some aspects, the light chain is a human kappa light chain or a human lambda light chain.

In some aspects, an antibody or antigen-binding fragment thereof, or multi-specific binding protein provided herein comprises constant regions comprising the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In some aspects, an antibody or antigen-binding fragment thereof, or multi-specific binding protein provided herein comprises constant regions comprising the amino acid sequences of the constant regions of an IgG, IgE, IgM, IgD, IgA, or IgY immunoglobulin molecule, any class (e.g., IgG1, IgG2, IgG3, IgG4, IgAQ1, and IgA2), or any subclass (e.g., IgG2a and IgG2b) of immunoglobulin molecule. In some aspects, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgAQ1, and IgA2), or any subclass (e.g., IgG2a and IgG2b) of immunoglobulin molecule.

Non-limiting examples of human constant region sequences have been described in e.g., U.S. Pat. No. 5,693,780 and Kabat E A et al., (1991) Sequences of Proteins of Immunological Interest, Fifth Edition, U.S. Department of Health and Human Services, NIH Publication No. 91-3242).

In some aspects, a constant region provided herein comprises a knob mutation. In some aspects, a constant region provided herein comprises a hole mutation. Accordingly, in some aspects, an antibody or antigen-binding fragment thereof, or multi-specific binding protein provided herein can comprise a constant region comprising a knob mutation and a constant region comprising a hole mutation. Description of knob and hole mutations are provided herein.

Variations in Linker Length Between the C-Terminus of an Anti-pyroGlu3 Abeta (Aβ) Antibody Heavy Chain Fc and an Antigen-Binding Domain that Binds to TfR

As described herein, certain anti-TfR antibodies can block binding of transferrin to its receptor (TfR1) on the surface of cells, interfering with iron transport. Disruption in iron transport into and uptake by erythroid progenitor cells (reticulocytes) can lead to a reduction in hematocrit, hemoglobin levels, and red blood cell count, resulting in anemia. Both antibody dependent cellular cytotoxicity (ADCC) and complement dependent cytotoxicity (CDC) have been described to also be involved in these adverse hemotologic processes due to Fc-mediated cytotoxic effects driven by Fc gamma receptor (FcγR) binding to the Fc region of the antibody. Eliminating or reducing the Fc effector function of an antibody (such as by mutating certain amino acid residues within the Fc region of the antibody) in order to reduce or eliminate ADCC and/or CDC is a potential mitigation strategy for the development of TfR1-based therapies designed to cross the blood-brain barrier in order to reduce such hematologic cytotoxic and adverse effects (Couch et al (2013), Sci Transl Med 5:183ra57).

However, changes in certain amino acid residues within the Fc region of an anti-Ap antibody in order to reduce ADCC and/or CDC can reduce or eliminate the effector function of the antibody when it is bound to its target. As such, Fc mutations resulting in reduced ADCC and/or CDC have the risk of reducing the full therapeutic efficacy of an anti-Abeta (Aβ) antibody. For instance, one mechanism of brain clearance of Abeta (Aβ) by therapeutic anti-Abeta (Aβ) antibodies is associated with the Fe effector function of the antibody, enabling recruitment of myeloid cells in the brain (e.g., microglial cells), which express Fc gamma receptors (FcγR), to aid in effective and efficient removal of Abeta (Aβ) plaques (See, e.g., Condello et al, 2015, Nat Commun 6:6176). Accordingly, in certain uses of anti-Abeta (Aβ) antibodies for treatment of various Abeta (Aβ)-associated brain disorders, such as Alzheimer's disease, an antibody having Fc-mediated effector function is desirable in order to achieve the beneficial clinical effects.

Based on these observations, it is therefor desirable to have an effective anti-pyroGlu3 Abeta (Aβ) antibody comprising an antigen-binding domain that binds TfR to facilitate transport across the BBB that reduces hematologic adverse events mediated by the Fc effector function of the antibody, while still maintaining Fc effector function of the anti-pyroGlu3 Abeta (Aβ) antibody in the brain. Without wishing to be bound by this theory, reducing the length of the linker located between the C-terminal end of the anti-pyroGlu3 Abeta (Aβ) antibody heavy chain (e.g., antibody heavy chain constant domain or antibody heavy chain Fc) and the anti-TfR antigen binding domain (e.g., scFv in 2+1 format) is contemplated to position the anti-pyroGlu3 Abeta (Aβ) antibody Fc region further into the TfR binding site upon anti-TfR scFv binding (or by reducing the flexibility/increasing the rigidity of the molecule), thereby preventing its ability to bind to FcγRs on immune cells when bound to, e.g., reticulocytes, thus reducing or eliminating the ADCC-mediated adverse hematologic events. In this way, the interaction between the anti-Abeta (Aβ) antibody Fc domain and FcγRs, which is necessary for the Fc-mediated ADCC response to occur, is prevented or reduced when the anti-Abeta (Aβ)/TfR composition is bound to reticulocytes. Additionally, it is contemplated that removal of the linker located between the C-terminus of the heavy chain (e.g., heavy chain constant domain or antibody heavy chain Fc) of the anti-pyroGlu3 Abeta (Aβ) antibody and the N-terminus of the anti-TfR antigen binding domain (e.g., scFv in 2+1 format) can create a more rigid, less flexible molecule that further helps reduce or eliminate ADCC-mediated adverse hematologic events associated with binding to FcγRs on immune cells.

Accordingly, in certain aspects, the present disclosure provides compositions comprising an anti-pyroGlu3 Abeta (Aβ) antibody having a fully active Fc region (e.g., a human IgG1 having a fully active Fc region) and a TfR antigen binding domain that can be transported across the blood brain barrier, wherein the TfR antigen binding domain is linked to the C-terminal region of the anti-pyroGlu3 Abeta (Aβ) antibody heavy chain (e.g., antibody heavy chain constant domain or antibody heavy chain Fc), wherein the composition reduces or eliminates ADCC-mediated adverse hematologic events associated with binding to FcγRs on immune cells when the composition is bound to reticulocytes. (See Example 29, Tables 23 and 24.)

In certain anti-pyroGlu3 Abeta (Aβ) antigen-binding domain/anti-TfR antigen binding domain compositions of the present disclosure, the linker located between the C-terminus of the heavy chain (e.g., heavy chain constant domain or antibody heavy chain Fc) of the anti-pyroGlu3 Abeta (Aβ) antibody and the N-terminus of the anti-TfR antigen binding domain scFv comprises the 5 amino acid linker GGSGG (SEQ ID NO: 214). In some aspects, the linker located between the C-terminus of the heavy chain (e.g., heavy chain constant domain or antibody heavy chain Fc) of the anti-pyroGlu3 Abeta (Aβ) antibody and the N-terminus of the anti-TfR antigen binding domain scFv comprises SEQ ID NO: 280. In some aspects, the linker located between the C-terminus of the heavy chain (e.g., heavy chain constant domain or antibody heavy chain Fc) of the anti-pyroGlu3 Abeta (Aβ) antibody and the N-terminus of the anti-TfR antigen binding domain scFv is shortened to 1-4 amino acids. In certain aspects, there is no linker located between the C-terminus of the heavy chain (e.g., heavy chain constant domain or antibody heavy chain Fc) of the anti-pyroGlu3 Abeta (Aβ) antibody and the N-terminus of the anti-TfR antigen binding domain scFv (e.g., SEQ ID NO: 281) (e.g., the C-terminus of the heavy chain Fc of the anti-pyroGlu3 Abeta (Aβ) antibody and the N-terminus of the anti-TfR antigen binding domain scFv are directly linked). In certain aspects, the N-terminal amino acid residues QV of the anti-TfR antigen binding domain scFv are removed, further reducing the length between the C-terminus of the heavy chain (e.g., heavy chain constant domain or antibody heavy chain Fc) of the anti-pyroGlu3 Abeta (Aβ) antibody and the N-terminus of the anti-TfR antigen binding domain scFv.

In certain aspects, the amino acid linker between the C-terminus of the heavy chain (e.g., heavy chain constant domain or antibody heavy chain Fc) of the anti-pyroGlu3 Abeta (Aβ) antibody and the N-terminus of the anti-TfR antigen binding domain scFv may be shortened, for example, to 1-5 amino acids. To further increase the rigidity, the C-terminus of the anti-pyroGlu3 Abeta (Aβ) antibody heavy chain (e.g., heavy chain constant domain or antibody heavy chain Fc) may be truncated and the N-terminus of the anti-TfR antigen binding domain may be truncated. For example, the amino acid sequence PGK may be removed from the C-terminus of the heavy chain (e.g., heavy chain constant domain or antibody heavy chain Fc) of the anti-pyroGlu3 Abeta (Aβ) antibody, and the amino acid sequence QV may be removed from the N-terminus of the anti-TfR antigen binding domain (e.g., SEQ ID NO:282).

Multi-Specific Binding Proteins that Comprise an Anti-pyroGlu3 Abeta (a$) Antigen-Binding Domain and an Antigen-Binding Domain that Binds to Human CD98hc

Provided herein are multi-specific binding proteins that comprise an antigen-binding domain that specifically binds to human anti-pyroGlu3 Abeta (Aβ) and an antigen-binding domain that specifically binds to human CD98hc.

In some aspects, an antibody or antigen-binding fragment thereof provided herein comprises an antigen-binding domain that specifically binds to human CD98hc. In some aspects, an antibody or antigen-binding fragment thereof comprises an antigen-binding domain that specifically binds to human CD98hc linked to a second antigen-binding domain that specifically binds to human pyroGlu3 Abeta (Aβ). Also provided herein are antibodies or antigen-binding fragments thereof that bind to the same epitope of CD98hc as a CD98hc antigen-binding domain provided herein. Also provided herein are antibodies or antigen-binding fragments thereof that competitively inhibit binding to CD98hc of a CD98hc antigen-binding domain provided herein.

In some aspects, a multi-specific binding protein provided herein comprises a first antigen-binding domain that specifically binds to human CD98hc linked to a second antigen-binding domain that specifically binds to human pyroGlu3 Abeta (Aβ). The first antigen-binding domain that specifically binds to human CD98hc can be any antigen-binding domain that binds to human CD98hc provided herein. The second antigen-binding domain can be any antigen-binding domain that specifically binds to human pyroGlu3 Abeta (Aβ) provided herein.

In some aspects, a multi-specific binding protein provided herein comprises an antigen-binding domain that specifically binds to human CD98hc that is linked to a second antibody or antigen-binding fragment thereof. The second antibody or antigen-binding fragment thereof specifically binds to human pyroGlu3 Abeta (Aβ). In some aspects, the second antibody or antigen-binding fragment does not bind to CD98hc.

In some aspects, a multi-specific binding protein provided herein comprises a 2+1 multi-specific protein format (as shown in FIG. 1A and FIG. 1B) or a 2+2 multi-specific protein format (as shown in FIG. 2).

In some aspects, a multi-specific binding protein provided herein comprises a CD98hc antigen-binding domain that is an scFv linked to an antibody that binds to Abeta (pyroGlu3 Aβ), wherein the antibody comprises two heavy chains and two light chains. In some aspects, the scFv is linked to the C-terminus of one of the two antibody heavy chains, e.g., via a protein linker.

In some aspects, the multi-specific binding protein comprises 1) an antigen-binding domain that specifically binds CD98hc, 2) a second antigen-binding domain that specifically binds to human pyroGlu3 Abeta (Aβ), and 3) an Fc region, wherein the CD98hc antigen-binding domain and the second antigen-binding domain are connected or linked to the Fc region of the multi-specific protein. In other aspects, the multi-specific protein comprises 1) an antigen-binding domain that comprises a heavy chain variable region and specifically binds CD98hc, 2) a second antigen binding domain that comprises a heavy-chain variable region and binds to human pyroGlu3 Abeta (Aβ), and 3) an Fc region, wherein the CD98hc antigen-binding domain and the second antigen-binding domain are connected or linked to the Fc region of the multi-specific protein.

In some aspects, the multi-specific binding protein comprises 1) an antigen-binding domain that specifically binds CD98hc, 2) a second antigen-binding domain that binds to human pyroGlu3 Abeta (Aβ), and 3) an Fc region, wherein the antigen-binding domain that specifically binds CD98hc and the second antigen-binding domain are both connected or linked to the N-terminus of the Fc portion of the multi-specific protein. In other aspects, the antigen-binding domain that specifically binds CD98hc is connected or linked to the N-terminus of an Fc portion of the multi-specific protein and the second antigen-binding domain is linked to the C-terminus of the Fc portion of the multi-specific protein. In other aspects, the antigen-binding domain that specifically binds CD98hc is connected or linked to the C-terminus of an Fc portion of the multi-specific protein and the second antigen-binding domain is linked to the N-terminus of the Fc portion of the multi-specific protein.

In some aspects, a multi-specific binding protein provided herein comprises two scFv molecules that comprise antigen-binding domains that specifically bind CD98hc and an antibody that binds to human pyroGlu3 Abeta (Aβ), wherein the antibody comprises two heavy chains and two light chains, and wherein one of the two scFv molecules is linked to the C-terminus of one of the antibody heavy chains, and wherein the other scFv molecule is linked to the C-terminus of the other antibody heavy chain. In some aspects, the scFvs are linked to the heavy chains of the antibody via a protein linker.

In some aspects, disclosed herein is a trivalent, bi-specific protein comprising (i) a single scFv, Fab or VHH antigen-binding domain that specifically binds to human CD98hc and (ii) an antibody that specifically binds to human pyroGlu3 Abeta (Aβ), wherein the antibody comprises two heavy chains and two light chains, and wherein the single scFv, Fab or VHH antigen-binding domain that specifically binds to human CD98hc is linked to the C-terminus or N-terminus of one of the two antibody heavy chains. The Fc may be a heterodimeric Fc, such as knob and hole Fc. An example of this 2+1 format with a knob-hole Fc is shown as (i) in FIG. 3.

In some aspects, disclosed herein is a tetravalent, bi-specific protein comprising (i) two scFv, Fab or VHH antigen-binding domains that specifically bind to human CD98hc and (ii) an antibody that binds to human pyroGlu3 Abeta (Aβ), wherein the antibody comprises two heavy chains and two light chains, and wherein one of the scFv, Fab, or VHH antigen-binding domains that specifically binds to human CD98hc is linked to the C-terminus of one of the two antibody heavy chains, and the other scFv, Fab, or VHH antigen-binding domain that specifically binds to human CD98hc is linked to the C-terminus or N-terminus of the other of the two antibody heavy chains. In some aspects, the two scFv, Fab or VHH antigen-binding domains that bind to human CD98hc can comprise the same amino acid sequence. In some aspects, the Fc is a heterodimeric Fc, such as an Fc region with knob and hole mutations.

In some aspects, disclosed herein is a bivalent, bispecific protein comprising (i) a single scFv, VHH, or Fab antigen-binding domain that specifically binds to human CD98hc linked to the N-terminus of an Fc domain of an Fc dimer, and (ii) a second antigen binding domain that binds to human pyroGlu3 Abeta (Aβ), wherein the second antigen binding domain is linked to the N-terminus of the second Fc domain of an Fc dimer. An example of this 1+1 format is shown as (iii) in FIG. 3.

In some aspects, disclosed herein is a bivalent, bi-specific protein comprising (i) a single scFv, VHH, or Fab antigen-binding domain that specifically binds to human CD98hc linked to the N-terminus or C-terminus of an Fc domain of an Fc dimer and (ii) a second antigen binding domain, wherein the second antigen binding domain binds to human pyroGlu3 Abeta (Aβ) and comprises a single scFv, VHH or Fab linked to N-terminus of the Fc. In some aspects, disclosed herein is a bivalent, bi-specific protein comprising (i) a single scFv, VHH, or Fab antigen-binding domain that specifically binds to human CD98hc linked to the N-terminus or C-terminus of an Fc domain of an Fc dimer and (ii) a second antigen binding domain, wherein the second antigen binding domain binds to human pyroGlu3 Abeta (Aβ) and comprises a single scFv, VHH or Fab linked to the C-terminus of the second Fc domain of the Fc dimer. In some aspects, the Fc is a heterodimeric Fc, such as an Fc region with knob and hole mutations. An example of this 1+1 format is shown as (ii) in FIG. 3, wherein a single scFv is linked to the N terminus of a first Fc domain of the Fc region and the second antigen binding domain is linked to the N terminus of the second Fc domain of the Fc region.

In some aspects, disclosed herein is a bivalent, bispecific protein comprising (i) a single scFv, VHH, or Fab antigen-binding domain that specifically binds to human CD98hc and is linked to the N-terminus of an Fc domain of an Fc dimer, and (ii) a second antigen binding domain that binds to human pyroGlu3 Abeta (Aβ) and is linked to the N-terminus of the second Fc domain of an Fc dimer. An example of this 1+1 format is shown as (iii) in FIG. 3.

In some aspects, disclosed herein is a bivalent, bi-specific protein comprising (i) a single scFv, VHH, or Fab antigen-binding domain that binds to human CD98hc and is linked to the N-terminus or C-terminus of an Fc domain of an Fc dimer and (ii) a second antigen binding domain, wherein the second antigen binding domain binds to human pyroGlu3 Abeta (Aβ) and comprises a single scFv, VHH or Fab linked to N-terminus of the Fc or C-terminus of a second Fc domain of the Fc dimer. In some aspects, the Fc is a heterodimeric Fc, such as an Fc domain with knob and hole mutations. An example of this 1+1 format is shown as (ii) in FIG. 3, where a single scFv is linked to the N terminus of an Fc domain of an Fc region and a second antigen binding domain is linked to the N terminus of a second Fc domain of the Fc region.

As provided herein, an antibody or antigen-binding fragment thereof, or multi-specific protein provided herein can be multi-specific, e.g., bi-specific. As described above, a bispecific protein of the present disclosure can include various configurations having a first antigen-binding domain that binds to human CD98hc and a second antigen-binding domain that binds to human pyroGlu3 Abeta (Aβ).

In some aspects, an antibody or antigen-binding fragment thereof, or multi-specific binding protein provided herein comprises a CD98hc binding domain and a knob mutation and a hole mutation. Knob and hole mutations are described in detail herein and similar apply to agents comprising CD98hc binding domains. In some aspects, the knob mutation comprises the amino acid substitution T366W according to EU numbering. In some aspects, the hole mutation comprises the amino acids substitutions T366S, L368A, and Y407V according to EU numbering. Examples of bispecific molecules that can be used are described herein.

In some aspects, bispecific protein formats are described above. In some aspects, an antibody or antigen-binding fragment thereof or multi-specific binding protein comprising a CD98hc binding domain and provided herein is bivalent or tetravalent. Formats comprising two CD98hc antigen-binding domains can comprise the same amino acid sequence or can comprise different amino acid sequences. In some aspects, the two CD98hc antigen-binding domains comprise the same amino acid sequence. In some aspects, one or both of the CD98hc antigen-binding domains is an scFv. The two antigen-binding domains that bind to a N3pE Abeta (Aβ) antigen can comprise the same amino acid sequence or can comprise different amino acid sequences.

An antibody or antigen-binding fragment thereof, or multi-specific binding protein comprising a CD98hc binding domain and provided herein can comprise a linker. The linker can be e.g., a glycine linker, a glycine-rich linker, or a glycine-serine linker. The linker can comprise the amino acid sequence (GGGGS)×3 (SEQ ID NO: 207). The linker can comprise the amino acid sequence (GGSGG)×3 (SEQ ID NO: 208).

An antibody or antigen-binding fragment thereof, or multi-specific binding protein comprising a CD98hc binding domain and provided herein can comprise a constant region, as described herein.

In some aspects, an antibody or antigen-binding fragment thereof, or multi-specific binding protein comprising a CD98hc binding domain as provided herein comprises a heavy chain and a light chain, as described herein.

In some aspects, an antibody or antigen-binding fragment thereof, or multi-specific protein comprising a CD98hc binding domain as provided herein comprises constant regions comprising the amino acid sequences of the constant regions of an IgG, IgE, IgM, IgD, IgA, or IgY immunoglobulin molecule, or a human IgG, IgE, IgM, IgD, IgA, or IgY immunoglobulin molecule, or any class (e.g., IgG1, IgG2, IgG3, IgG4, IgAQ1, and IgA2), or any subclass (e.g., IgG2a and IgG2b) of immunoglobulin molecule. Non-limiting examples of human constant regions and human constant region sequences are described herein.

Multi-Specific Binding Proteins that Comprise an Antigen-Binding Domain that Binds to PyroGlu3 Abeta (Aβ) and an Antigen-Binding Domain that Binds to Blood-Brain Barrier Receptors or Proteins

Provided herein are compositions that specifically bind to human receptors or proteins of the blood-brain barrier.

In some aspects, the composition comprises a multispecific protein comprising (i) an antigen-binding domain that specifically binds to human transferrin receptor (TfR) or human CD98 heavy chain (CD98hc) and (ii) an isolated anti-pyroGlu3 Abeta (Aβ) antibody or antigen binding fragment thereof.

In some aspects, provided herein are multispecific proteins comprising (i) an antigen-binding domain that specifically binds to a blood brain barrier (BBB) target selected from human transferrin receptor (TfR) or human CD98 heavy chain (CD98hc) and (ii) an antigen-binding domain that specifically binds to pyroGlu3 Abeta (Aβ).

In some aspects, the anti-pyroGlu3 Abeta (Aβ) antigen-binding domain is an antibody. In some aspects, the antibody is a full-length antibody.

In some aspects, the antibody is an IgG antibody. In some aspects, the IgG antibody is an IgG1 antibody or an IgG4 antibody.

In some aspects, the anti-pyroGlu3 Abeta (Aβ) antibody comprises a heavy chain with a Fc hole mutation.

In some aspects, the anti-pyroGlu3 Abeta (Aβ) antibody comprises a heavy chain with a Fc knob mutation.

In some aspects, the anti-BBB antigen-binding domain is a scFv.

In some aspects, the anti-BBB scFv is linked to the C-terminus of the anti-pyroGlu3 Aβ antibody heavy chain with a Fc hole mutation.

In some aspects, the anti-BBB scFv is linked to the C-terminus of the anti-pyroGlu3 Aβ antibody heavy chain with a Fc knob mutation.

In some aspects, the multispecific protein comprises two antigen-binding domains that specifically bind to a BBB target selected from human TfR and human CD98hc. In some aspects, both of the anti-BBB antigen-binding domains are scFvs.

In some aspects, the first anti-BBB scFv is linked to the C-terminus of the anti-pyroGlu3 Abeta (Aβ) antibody heavy chain with a Fc hole mutation. In some aspects, the second anti-BBB scFv is linked to the C-terminus of the anti-pyroGlu3 Abeta (Aβ) antibody heavy chain with a Fc knob mutation.

In some aspects, the anti-BBB antigen-binding domain is an antibody. In some aspects, the antibody is a full-length antibody.

In some aspects, the antibody is an IgG antibody. In some aspects, the IgG antibody is an IgG1 antibody or an IgG4 antibody.

In some aspects, the anti-BBB antibody comprises a heavy chain with a Fc hole mutation.

In some aspects, the anti-BBB antibody comprises a heavy chain with a Fc knob mutation.

In some aspects, the anti-pyroGlu3 Abeta (Aβ) antigen-binding domain is a scFv.

In some aspects, the anti-pyroGlu3 Abeta (Aβ) scFv is linked to the C-terminus of the anti-BBB antibody heavy chain with a Fc hole mutation.

In some aspects, the anti-pyroGlu3 Abeta (Aβ) scFv is linked to the C-terminus of the anti-BBB antibody heavy chain with a Fc knob mutation.

In some aspects, the multispecific protein comprises two antigen-binding domains that specifically bind to a pyroGlu3 Abeta (Aβ), wherein both of the anti-pyroGlu3 Abeta (Aβ) antigen-binding domains are scFvs.

In some aspects, the first anti-pyroGlu3 Abeta (Aβ) scFv is linked to the C-terminus of the anti-BBB antibody heavy chain with a Fc hole mutation. In some aspects, the second anti-pyroGlu3 Abeta (Aβ) scFv is linked to the C-terminus of the anti-BBB antibody heavy chain with a Fc knob mutation.

In some aspects, the antibody is a monoclonal antibody.

In some aspects, the antibody is a humanized antibody.

In some aspects, the anti-pyroGlu3 Abeta (Aβ) antigen-binding domain is selected from the group consisting of a VHH, a Fab, a Fab′, a Fab′-SH, a F(ab′)2, a Fv, or a scFv.

In some aspects, the anti-BBB antigen-binding domain is a VHH, a Fab, a Fab′, a Fab′-SH, a F(ab′)2, a Fv, or a scFv.

In some aspects, the multispecific protein further comprises iii) an Fc region.

In some aspects, the Fc region is fully active. In some aspects, a multispecific protein of the present disclosure has a fully active Fc region, and thus, enables recruitment of myeloid cells in the brain (e.g., microglial cells) to aid in the effective and efficient removal of Abeta (Aβ) plaques.

In some aspects, the Fc region is partially active.

In some aspects, the Fc region is not silent.

In some aspects, the anti-pyroGlu3 Abeta (Aβ) antigen-binding domain is a VHH.

In some aspects, the anti-BBB antigen-binding domain is a VHH.

In some aspects, the multispecific protein comprises an IgG Fc region, the anti-BBB antigen-binding domain is a VHH that is N-terminal to one protein fragment of the Fc region, and the anti pyroGlu3 Abeta (Aβ) antigen-binding domain is a VHH that is N-terminal to the other protein fragment of the Fc region.

In some aspects, the anti pyroGlu3 Abeta (Aβ) antigen-binding domain is a Fab.

In some aspects, the anti-BBB antigen-binding domain is a Fab.

In some aspects, the multispecific protein comprises an IgG Fc region, the anti-BBB antigen-binding domain is a Fab that is N-terminal to one protein fragment of the Fc region, and the anti-pyroGlu3 Abeta (Aβ) antigen-binding domain is a Fab that is N-terminal to the other protein fragment of the Fc region.

In some aspects, the anti-BBB antigen-binding domain is a scFv.

In some aspects, the anti-pyroGlu3 Abeta (Aβ) antigen-binding domain is a scFv.

In some aspects, the multispecific protein comprises an IgG Fc region, the anti-BBB antigen-binding domain is a scFv that is N-terminal to one protein fragment of the Fc region, and the anti-pyroGlu3 Abeta (Aβ) antigen-binding domain is a scFv that is N-terminal to the other protein fragment of the Fc region.

In some aspects, the multispecific protein comprises an IgG Fc region, the anti-BBB antigen-binding domain is a scFv that is N-terminal to one protein fragment of the Fc region, and the anti-pyroGlu3 Abeta (Aβ) antigen-binding domain is a Fab that is N-terminal to the other protein fragment of the Fc region.

In some aspects, the multispecific protein comprises an IgG Fc region, the anti-BBB antigen-binding domain is a Fab that is N-terminal to one protein fragment of the Fc region, and the anti-pyroGlu3 Abeta (Aβ) antigen-binding domain is a Fab that is N-terminal to the other protein fragment of the Fc region

In some aspects, the IgG Fc region is an IgG1 Fc region or an IgG4 Fc region.

In some aspects, the IgG Fc region comprises knob and hole mutations.

In some aspects, the multispecific protein is bivalent.

In some aspects, the multispecific protein is trivalent.

In some aspects, the trivalent protein comprises one antigen-binding domain that binds to the BBB target and two antigen-binding domains that bind to pyroGlu3 Abeta (Aβ).

In some aspects, the trivalent protein comprises two antigen-binding domains that bind to the BBB target and one antigen-binding domain that binds to pyroGlu3 Abeta (Aβ).

In some aspects, the multispecific protein is tetravalent, optionally wherein the tetravalent protein comprises two of antigen-binding domains that bind to the BBB target and two antigen-binding domains that bind to pyroGlu3 Abeta (Aβ).

In some aspects, the multispecific protein is a bispecific T-cell engager (BiTE), dual-affinity re-targeting protein (DARTs), or Tandem diabody (TandAb).

In some aspects, the anti-BBB antigen-binding domain and the anti-pyroGlu3 Abeta (Aβ) antigen-binding domain are contained within a single polypeptide chain. In some aspects, the single polypeptide chain further comprises an Fc domain.

In some aspects, the anti-pyroGlu3 Abeta (Aβ) antigen-binding domain is N-terminal to the anti-BBB antigen-binding domain.

In some aspects, the anti-pyroGlu3 Abeta (Aβ) antigen-binding domain thereof is C-terminal to the anti-BBB antigen-binding domain.

In some aspects, the anti-pyroGlu3 Abeta (Aβ) antigen-binding domain and the anti-BBB antigen-binding domain are directly connected via a peptide bond.

In some aspects, the anti-pyroGlu3 Abeta (Aβ) antigen-binding domain and the anti-BBB antigen-binding domain are connected via a linker.

In some aspects, the linker is a peptide linker.

In some aspects, the anti-BBB antigen-binding domain and the anti-pyroGlu3 Abeta (Aβ) antigen-binding domain are N-terminal to the Fc domain.

In some aspects, the anti-BBB antigen-binding domain and the anti-pyroGlu3 Abeta (Aβ) antigen-binding domain are C-terminal to the Fc domain.

In some aspects, the anti-BBB antigen-binding domain is N-terminal to the Fc domain and the anti-pyroGlu3 Abeta (Aβ) antigen-binding domain is C-terminal to the Fc domain.

In some aspects, the anti-pyroGlu3 Abeta (Aβ) antigen-binding domain is N-terminal to the Fc domain and the anti-BBB antigen-binding domain is C-terminal to the Fc domain.

In some aspects, the multispecific protein comprises two copies of the anti-pyroGlu3 Abeta (Aβ) antigen-binding domain and/or two copies of the anti-BBB antigen-binding domain.

In some aspects, an anti-pyroGlu3 Abeta (Aβ) antigen-binding domain of the present disclosure is an antagonist to pyroGlu3 Abeta (Aβ) activity or pathology.

In some aspects, an anti-pyroGlu3 Abeta (Aβ) antigen-binding domain of the present disclosure reduces pyroGlu3 Abeta (Aβ) levels in the brain.

In some aspects, an anti-pyroGlu3 Abeta (Aβ) antigen-binding domain of the present disclosure competes with one or more antibodies selected from anti-pyroGlu3 Abeta (Aβ) antibody AB-1, AB-2, AB-3, AB-4, AB-5, AB-6, AB-7, AB-8, AB-9, AB-10, and any combination thereof for binding to pyroGlu3 Abeta (Aβ).

In some aspects, the anti-pyroGlu3 Abeta (Aβ) antigen-binding domain binds human pyroGlu3 Abeta (Aβ), binds mouse Abeta (Aβ), binds cynomolgus Abeta (Aβ), binds both human and mouse Abeta (Aβ), or binds human, mouse, and cynomolgus Abeta (Aβ).

In some aspects, the anti-pyroGlu3 Abeta (Aβ) antigen-binding domain binds human pyroGlu3 Abeta (Aβ) with an affinity of about 1 nM to about 100 nM, of about 5 nM to about 100 nM from about 7 nM to about 88 nM. In some aspects, the anti-pyroGlu3 Abeta (Aβ) antigen-binding domain binds human pyroGlu3 Abeta (Aβ) with an affinity of about 10 nM to about 50 nM.

In some aspects, the antigen-binding domain is an anti-TfR antigen-binding domain.

In some aspects, the anti-pyroGlu3 Abeta (Aβ) antigen-binding domain in the multispecific protein is N-terminal to the anti-TfR antigen-binding domain.

In some aspects, the anti-pyroGlu3 Abeta (Aβ) antigen-binding domain is C-terminal to the anti-TfR antigen-binding domain.

In some aspects, the anti-pyroGlu3 Abeta (Aβ) antigen-binding domain and the anti-TfR antigen-binding domain are directly connected via a peptide bond.

In some aspects, the anti-pyroGlu3 Abeta (Aβ) antigen-binding domain and the anti-TfR antigen-binding domain are connected via a linker.

In some aspects, the linker is a peptide linker.

In some aspects, the multispecific protein further comprises iii) an Fc region.

In some aspects, the Fc region is fully active. In some aspects, a multispecific protein of the present disclosure has a fully active Fc region, and thus, enables recruitment of myeloid cells in the brain (e.g., microglial cells) to aid in the effective and efficient removal of Abeta (Aβ) plaques.

In some aspects, the Fc region is partially active.

In some aspects, the Fc region is not silent.

In some aspects, the anti-TfR antigen-binding domain and the anti-pyroGlu3 Abeta (Aβ) antigen-binding domain are linked to the N-terminus of the Fc portion of the multi-specific binding protein.

In some aspects, the anti-TfR antigen-binding domain and the anti-pyroGlu3 Abeta (Aβ) antigen-binding domain are both linked to the C-terminus of the Fc portion of the multi-specific binding protein.

In some aspects, the anti-TfR antigen-binding domain is linked to the N-terminus of the Fc portion and the anti-pyroGlu3 Abeta (Aβ) antigen-binding domain is linked to the C-terminus of the Fc portion of the multispecific protein.

In some aspects, the anti-pyroGlu3 Abeta (Aβ) antigen-binding domain is linked to the N-terminus of the Fc portion of the multi-specific binding protein and the anti-TfR antigen-binding domain is linked to the C-terminus of the Fc portion.

In some aspects, the anti-TfR antigen-binding domain is a single scFv, or a VHH, or a Fab.

In some aspects, the anti-TfR antigen-binding domain binds to a TfR domain with a binding affinity of about 1 nM to about 100 nM, about 1 nM to about 1000 nM, about 1 nM to about 2000 nM, or about 40 nM to about 2200 nM.

In some aspects, the multi-specific binding protein comprises two copies of the anti-pyroGlu3 Abeta (Aβ) antigen-binding domain.

In some aspects, the multi-specific binding protein comprises an Fc domain or Fc region.

In some aspects, the single scFv, Fab or VHH is linked to the C-terminus of the Fc domain or Fc region, wherein the two copies of the anti-pyroGlu3 Abeta (Aβ) antigen-binding domain are linked to the N-terminus of the Fc domain or Fc region.

In some aspects, the single scFv, Fab or VHH is linked to the N-terminus of the Fc domain or Fc region, wherein the two copies of the anti-pyroGlu3 Abeta (Aβ) antigen-binding domain are linked to the C-terminus of the Fc domain or Fc region.

In some aspects, the anti-TfR antigen binding domain is an anti-TfR antibody, and wherein the antibody comprises two heavy chains and two light chains.

In some aspects, two copies of an anti-pyroGlu3 Abeta (Aβ) antigen-binding domain are linked to the C-terminus of the two anti-TfR antibody heavy chains.

In some aspects, the anti-TfR antigen binding domain comprises two scFv, Fab, or VHH domains linked to the C-terminus of the heavy chain.

In some aspects, two copies of the anti-pyroGlu3 Abeta (Aβ) antigen-binding domain are linked to the N-terminus of the Fc domain or Fc region.

In some aspects, the anti-TfR antigen binding domain comprises two scFv, Fab, or VHH antigen-binding domains that specifically bind to human TfR linked to the N-terminus of the heavy chain.

In some aspects, two copies of the anti-pyroGlu3 Abeta (Aβ) antigen-binding domain are linked to the C-terminus of the Fc domain or Fc region.

In some aspects, the anti-TfR antigen binding domain comprises a single scFv, VHH, or Fab antigen-binding domain.

In some aspects, the multi-specific binding protein further comprises (ii) an Fc domain or Fc region, and (iii) a single copy of the anti-pyroGlu3 Abeta (Aβ) antigen-binding domain.

In some aspects, the single scFv, VHH, or Fab antigen-binding domain is linked to the C-terminus of the Fc domain or Fc region and the anti-pyroGlu3 Abeta (Aβ) antigen-binding domain is linked to N-terminus of the Fc domain or Fc region.

In some aspects, the single scFv, VHH, or Fab antigen-binding domain is linked to the N-terminus of the Fc domain or Fc region and the anti-pyroGlu3 Abeta (Aβ) antigen-binding domain is linked to C-terminus of the Fc domain or Fc region.

In some aspects, the single scFv, VHH, or Fab antigen-binding domain and the anti-pyroGlu3 Abeta (Aβ) antigen-binding domain are both linked to the N-terminus of the Fc domain or Fc region.

In some aspects, the single scFv, VHH, or Fab antigen-binding domain and the anti-pyroGlu3 Abeta (Aβ) antigen-binding domain are both linked to the C-terminus of the Fc domain or Fc region.

In some aspects, the Fe is a single chain, engineered monovalent Fc domain.

In some aspects, the multi-specific binding protein is a bispecific protein.

In some aspects, provided herein is a composition comprising any of the multi-specific binding proteins disclosed herein.

### Polynucleotides and Methods of Making the Same

In some aspects, provided herein is a nucleotide sequence encoding an antigen-binding domain that specifically binds to a human blood brain barrier protein or receptor and an antigen-binding domain that specifically binds to a human pyroGlu3 Abeta (Aβ) peptide. In some aspects, provided herein are polynucleotides encoding an antigen-binding domain that specifically binds to a human blood brain barrier protein or receptor and encoding an antigen-binding thereof domain that specifically binds to a human pyroGlu3 Abeta (Aβ) peptide. In some aspects, the human blood brain barrier protein or receptor is TfR. In some aspects, the human blood brain barrier protein or receptor is CD98hc.

Provided herein are polynucleotides comprising a nucleotide sequence encoding the compositions described herein, or a domain thereof described herein, that are optimized, e.g., by codon/RNA optimization, replacement with heterologous signal sequences, and/or elimination of mRNA instability elements. Methods to generate optimized nucleic acids for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly.

A polynucleotide comprising a nucleotide sequence encoding the compositions described herein, or a domain thereof described herein, can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising, e.g., the sequence encoding the light chain and/or heavy chain of an antigen-binding domain, antibody, or antigen-binding fragment thereof. The amplified nucleic acids can be cloned into vectors for expression in host cells and for further cloning, for example, to generate an antigen-binding domain that specifically bind to human TfR, fusion protein, complex, antibody, antigen-binding fragment thereof, or multi-specific protein described herein, or a domain thereof described herein.

Polynucleotides provided herein can be in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure.

In some aspects, polynucleotides provided herein are in the form of RNA. In some aspects, polynucleotides provided herein are in the form of RNA encoding a fusion protein or complex provided herein. In some aspects, a polynucleotide provided herein is a synthetic messenger RNA (mRNA). In some aspects, the synthetic mRNA has at least one nucleoside modification. In some aspects, the at least one nucleoside modification is selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In certain aspects, provided herein are vectors (e.g., expression vectors) comprising polynucleotides comprising nucleotide sequences encoding the compositions described herein, or a domain thereof described herein, for recombinant expression in a host cell, e.g., in a mammalian host cell or in an E. coli cell. A vector for the production of the compositions described herein, or a domain thereof described herein, can be produced, e.g., by recombinant DNA technology using techniques well known in the art. These methods include, for example, in vitro recombinant DNA techniques, synthetic techniques, and in vivo genetic recombination. Also provided are replicable vectors comprising a nucleotide sequence encoding the compositions described herein, or a domain thereof described herein, operably linked to a promoter. Such vectors can, for example, include the nucleotide sequence encoding the constant region of an antigen-binding domain, antibody or antigen-binding fragment thereof (see, e.g., International Publication Nos. WO 86/05807 and WO 89/01036; and U.S. Pat. No. 5,122,464), and variable domains of the antigen-binding domain, antibody or antigen-binding fragment thereof can be cloned into such a vector for expression of the entire heavy, the entire light chain, or both the entire heavy and light chains. In some aspects, the vector is gene therapy vector (e.g., an AAV or lentiviral vector).

In certain aspects, provided herein are expression systems comprising polynucleotides comprising nucleotide sequences encoding the compositions described herein, or a domain thereof described herein and an antibody to pyroGlu3 Abeta (Aβ) or antigen-binding domain thereof. An expression system can be included on a vector. An expression system can also be integrated into a host cell chromosome. In some aspects, an expression system is a cell free expression system. In some aspects, an expressions system comprises a host cell comprising a polynucleotide and/or vector provided herein.

Accordingly, also provided herein are cells, e.g., host cells, comprising polynucleotides and/or vectors for recombinantly expressing the compositions described herein and an antibody to pyroGlu3 Abeta (Aβ) or antigen-binding domain thereof. In some aspects, for the expression of double-chained antigen-binding proteins, vectors encoding both the heavy and light chains, individually, can be co-expressed in the host cell for expression of the entire immunoglobulin. In some aspects, a host cell contains two different vectors, a first vector comprising a polynucleotide encoding a heavy chain of an antigen-binding protein described herein, and a second vector comprising a polynucleotide encoding a light chain of the an antigen-binding protein. In some aspects, a first host cell comprises a first vector comprising a polynucleotide encoding a heavy chain, and a second host cell comprises a second vector comprising a polynucleotide encoding a light chain. In some aspects, provided herein is a population of host cells comprising such first host cell and such second host cell.

In some aspects, provided herein are methods for producing the compositions described herein, or a domain thereof and an antibody to pyroGlu3 Abeta (Aβ) or antigen-binding domain thereof described herein in a host cell.

An expression vector can be transferred to a cell (e.g., host cell) by conventional techniques, and the resulting cells can then be cultured by conventional techniques to produce the compositions described herein, or a domain thereof described herein.

A variety of host-expression vector systems can be utilized to the compositions described herein, or a domain thereof described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express the compositions described herein, or a domain thereof described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing coding sequences; or mammalian cell systems (e.g., COS (e.g., COS1 or COS), CHO, BHK, MDCK, HEK 293, NS0, PER.C6, VERO, CRL7030, HsS78Bst, HeLa, and NIH3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing the compositions described herein, or a domain thereof described herein are CHO cells, for example CHO cells from the CHO GS System™ (Lonza). In some aspects, cells for expressing the compositions described herein, or a domain thereof described herein as described herein are human cells, e.g., human cell lines. In some aspects, a mammalian expression vector is pOptiVEC™ or pcDNA3.3. In some aspects, bacterial cells, such as Escherichia coli, or eukaryotic cells (e.g., mammalian cells) are used for the expression of the compositions described herein, or a domain thereof described herein. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45: 101-105; and Cockett M I et al., (1990) Biotechnology 8: 662-667). In some aspects, the compositions described herein, or a domain thereof described herein is produced by CHO cells or NS0 cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Once the compositions described herein, or a domain thereof described herein has been produced by recombinant expression, it can be purified by any method known in the art for purification, for example, by chromatography (e.g., ion exchange, affinity, particularly by affinity for the specific antigen after Protein A, and sizing column chromatography), centrifugation, differential solubility, or by any other standard technique for the purification of proteins. Further, the compositions described herein, or a domain thereof described herein can be fused to heterologous polypeptide sequences to facilitate purification.

In some aspects, the compositions described herein are isolated or purified. Generally, an isolated or purified composition described herein is one that is substantially free of other proteins. For example, in some aspects, a preparation of the compositions described herein is substantially free of cellular material and/or chemical precursors.

Polynucleotides Encoding Agents Comprising an Antibody to PyroGlu3 Abeta (Aβ) or Antigen-Binding Domain Thereof and Antigen Binding Domains that Bind TfR and Methods of Making the Same

In some aspects, provided herein are polynucleotides comprising a nucleotide sequence encoding a composition comprising an antigen binding domain that specifically binds to human TfR, as described herein and an antigen binding domain that specifically binds to pyroGlu3 Abeta (Aβ). In some aspects, provided herein are polynucleotides comprising a first nucleotide sequence encoding an antigen-binding domain that specifically bind to human TfR, as described herein and a second nucleotide sequence encoding an antibody to pyroGlu3 Abeta (Aβ) or antigen-binding domain thereof. In some aspects, the polynucleotide is DNA. In some aspects, the polynucleotide is RNA or mRNA.

In some aspects, provided herein are polynucleotides comprising a nucleotide sequence encoding an antigen-binding domain that specifically bind to human TfR, a fusion protein, a complex, an antibody, an antigen-binding fragment thereof, or a multi-specific binding protein described herein or a domain thereof described herein.

In some aspects, a polynucleotide provided herein comprises a nucleic acid molecule encoding the heavy chain of an antigen-binding domain that specifically binds to human TfR provided herein. In some aspects, a polynucleotide provided herein comprises a nucleic acid molecule encoding the light chain of an antigen-binding domain that specifically binds to human TfR provided herein. In some aspects, a polynucleotide provided herein comprises a nucleic acid molecule encoding the heavy chain of an antigen-binding domain that specifically binds to human TfR provided herein and a nucleic acid molecule encoding the light chain of an antigen-binding domain that specifically binds to human TfR provided herein.

In some aspects, a polynucleotide provided herein comprises a nucleic acid molecule encoding the heavy chain of an antigen-binding domain that specifically binds to pyroGlu3 Abeta (Aβ) provided herein. In some aspects, a polynucleotide provided herein comprises a nucleic acid molecule encoding the light chain of an antigen-binding domain that specifically binds to pyroGlu3 Abeta (Aβ) provided herein. In some aspects, a polynucleotide provided herein comprises a nucleic acid molecule encoding the heavy chain of an antigen-binding domain that specifically binds to pyroGlu3 Abeta (Aβ) provided herein and a nucleic acid molecule encoding the light chain of an antigen-binding domain that specifically binds to pyroGlu3 Aβ provided herein.

In some aspects, combinations or compositions of polynucleotides are provided herein. In some aspects, a combination or composition comprises a first polynucleotide, a second polynucleotide, and a third polynucleotide, wherein the first, second, and third polynucleotides encode a multi-specific protein provided herein, e.g., wherein the first polynucleotide encodes a first heavy chain, the second polynucleotide encodes a second heavy chain and an antigen-binding domain that specifically binds to human TfR provided herein, and the third polynucleotide encodes a light chain. In some aspects, the antigen-binding domains that bind to human TfR is a scFv. In some aspects, the first heavy chain comprises a knob mutation and the second heavy chain comprises a hole mutation. In some aspects, the first heavy chain comprises a hole mutation and the second heavy chain comprises a knob mutation.

In some aspects, a combination or composition comprises a first polynucleotide, a second polynucleotide, and a third polynucleotide, wherein the first, second, and third polynucleotides encode a multi-specific protein provided herein, wherein the first polynucleotide encodes a first heavy chain and a first antigen-binding domain that specifically binds to human TfR, the second polynucleotide encodes a second heavy chain and a second antigen-binding domain that specifically binds to human TfR, and the third polynucleotide encodes a light chain. In some aspects, the first and second antigen-binding domains that bind to human TfR comprise the same amino acid sequence. In some aspects, the first and second antigen-binding domains that bind to human TfR comprise different amino acid sequences. In some aspects, the first and/or second antigen-binding domains that bind to human TfR are scFvs. In some aspects, the first heavy chain comprises a knob mutation and the second heavy chain comprises a hole mutation. In some aspects, the first heavy chain comprises a hole mutation and the second heavy chain comprises a knob mutation.

In some aspects, a combination or composition comprises a first polynucleotide and a second polynucleotide, wherein the first and second polynucleotides encode a multi-specific protein provided herein, wherein the first polynucleotide encodes a heavy chain and an antigen-binding domain that bind to human TfR provided herein, and wherein the second polynucleotide encodes a light chain.

In certain aspects, provided herein are vectors (e.g., expression vectors) comprising polynucleotides comprising nucleotide sequences encoding an antigen-binding domain that specifically bind to human TfR, antibody, antigen-binding fragment thereof, or multi-specific binding protein described herein, or a domain thereof described herein, for recombinant expression in a host cell, e.g., in a mammalian host cell.

In certain aspects, provided herein are expression systems comprising polynucleotides comprising nucleotide sequences encoding the compositions described herein, or a domain thereof described herein and an antibody to pyroGlu3 Abeta (Aβ) or antigen-binding domain thereof.

Accordingly, also provided herein are cells, e.g., host cells, comprising polynucleotides and/or vectors for recombinantly expressing an antigen-binding domain that specifically bind to human TfR, fusion protein, complex, antibody, antigen-binding fragment thereof, or multi-specific binding protein described herein, or a domain thereof described herein and an antigen-binding domain that specifically binds to pyroGlu3 Abeta (Aβ).

In some aspects, provided herein are methods for producing an antigen-binding domain that specifically binds to human TfR, fusion protein, complex, antibody, antigen-binding fragment thereof, or multi-specific protein described herein, or a domain thereof and an antigen-binding domain that specifically binds to pyroGlu3 Abeta (Aβ) described herein in a host cell.

In some aspects, provided herein are methods for producing a single chain antigen-binding domain that specifically binds to human TfR, a linker, an Fc domain, and a second antigen-binding domain, as described herein.

In some aspects, a composition that binds to human TfR as described herein is isolated or purified (e.g., is one that is substantially free of other proteins, and/or is substantially free of cellular material and/or chemical precursors).

### Polynucleotides Encoding Agents Comprising an Antibody to PyroGlu3 Abeta (Aβ) or Antigen-Binding Domain Thereof and Antigen Binding Domains That Bind CD98hc and Methods of Making the Same

In some aspects, provided herein are polynucleotides comprising a nucleotide sequence encoding a composition comprising an antigen binding domain that specifically binds to human CD98hc described herein or a domain thereof described herein, and an antigen-binding domain that specifically binds to pyroGlu3 Abeta (Aβ).

In some aspects, provided herein are polynucleotides comprising a first nucleotide sequence encoding an antigen-binding domain that specifically bind to human CD98hc, as described herein and a second nucleotide sequence encoding an antigen-binding domain that specifically binds to pyroGlu3 Abeta (Aβ). In some aspects, the polynucleotide is DNA. In some aspects, the polynucleotide is RNA or mRNA.

In some aspects, provided herein are polynucleotides comprising a nucleotide sequence encoding compositions that specifically bind to human CD98hc described herein or a domain thereof described herein.

In some aspects, a polynucleotide provided herein comprises a nucleic acid molecule encoding the heavy chain of an antigen-binding domain that specifically binds to human CD98hc provided herein. In some aspects, a polynucleotide provided herein comprises a nucleic acid molecule encoding the light chain of an antigen-binding domain that specifically binds to human CD98hc provided herein. In some aspects, a polynucleotide provided herein comprises a nucleic acid molecule encoding the heavy chain of an antigen-binding domain that specifically binds to human CD98hc provided herein and a nucleic acid molecule encoding the light chain of an antigen-binding domain that specifically binds to human CD98hc provided herein.

In some aspects, a polynucleotide provided herein comprises a nucleic acid molecule encoding the heavy chain of an antigen-binding domain that specifically binds to pyroGlu3 Abeta (Aβ) provided herein. In some aspects, a polynucleotide provided herein comprises a nucleic acid molecule encoding the light chain of an antigen-binding domain that specifically binds to pyroGlu3 Abeta (Aβ) provided herein. In some aspects, a polynucleotide provided herein comprises a nucleic acid molecule encoding the heavy chain of an antigen-binding domain that specifically binds to pyroGlu3 Abeta (Aβ) provided herein and a nucleic acid molecule encoding the light chain of an antigen-binding domain that specifically binds to pyroGlu3 Abeta (Aβ) provided herein.

In some aspects, combinations or compositions of polynucleotides are provided herein. In some aspects, a combination or composition comprises a first polynucleotide, a second polynucleotide, and a third polynucleotide, wherein the first, second, and third polynucleotides encode a multi-specific protein provided herein, e.g., wherein the first polynucleotide encodes a first heavy chain, the second polynucleotide encodes a second heavy chain and an antigen-binding domain that specifically binds to human CD98hc provided herein, and the third polynucleotide encodes a light chain. In some aspects, the antigen-binding domains that bind to human CD98hc is a scFv. In some aspects, the first heavy chain comprises a knob mutation and the second heavy chain comprises a hole mutation. In some aspects, the first heavy chain comprises a hole mutation and the second heavy chain comprises a knob mutation.

In some aspects, a combination or composition comprises a first polynucleotide, a second polynucleotide, and a third polynucleotide, wherein the first, second, and third polynucleotides encode a multi-specific protein provided herein, wherein the first polynucleotide encodes a first heavy chain and a first antigen-binding domain that specifically binds to human CD98hc, the second polynucleotide encodes a second heavy chain and a second antigen-binding domain that specifically binds to human CD98hc, and the third polynucleotide encodes a light chain. In some aspects, the first and second antigen-binding domains that bind to human CD98hc comprise the same amino acid sequence. In some aspects, the first and second antigen-binding domains that bind to human CD98hc comprise different amino acid sequences. In some aspects, the first and/or second antigen-binding domains that bind to human CD98hc are scFvs. In some aspects, the first heavy chain comprises a “knob” mutation and the second heavy chain comprises a “hole” mutation. In some aspects, the first heavy chain comprises a “hole” mutation and the second heavy chain comprises a “knob” mutation.

In some aspects, a combination or composition comprises a first polynucleotide and a second polynucleotide, wherein the first and second polynucleotides encode a multi-specific protein provided herein, wherein the first polynucleotide encodes a heavy chain and an antigen-binding domain that bind to human CD98hc provided herein, and wherein the second polynucleotide encodes a light chain.

In certain aspects, provided herein are vectors (e.g., expression vectors) comprising polynucleotides comprising the compositions that bind CD98hc described herein, or a domain thereof described herein, for recombinant expression in a host cell, e.g., in a mammalian host cell.

In certain aspects, provided herein are expression systems comprising polynucleotides comprising nucleotide sequences encoding the compositions that bind CD98hc described herein, or a domain thereof described herein and an antigen-binding domain that specifically binds to pyroGlu3 Abeta (Aβ). Accordingly, also provided herein are cells, e.g., host cells, comprising polynucleotides and/or vectors for recombinantly expressing the compositions that bind CD98hc described herein, or a domain thereof described herein and an antigen-binding domain that specifically binds to pyroGlu3 Abeta (Aβ).

In some aspects, provided herein are methods for producing a composition that binds to human CD98hc as described herein, or a domain thereof, and an antigen-binding domain that specifically binds to pyroGlu3 Abeta (Aβ) described herein in a host cell.

In some aspects, provided herein are methods for producing a single chain antigen-binding domain that specifically binds to human CD98hc, a linker, an Fc domain, and an antigen-binding domain that specifically binds to pyroGlu3 Abeta (Aβ).

In some aspects, provided herein are methods for producing a single chain antigen-binding domain that specifically binds to human CD98hc, a linker, an Fc domain, and a second antigen-binding domain, as described herein.

A variety of host-expression vector systems and host cells are described herein.

In some aspects, a composition that binds to human CD98hc as described herein is isolated or purified (e.g., is one that is substantially free of other proteins, and/or is substantially free of cellular material and/or chemical precursors).

### Pharmaceutical Compositions

Provided herein are pharmaceutical compositions comprising an antigen-binding domain that specifically binds to pyroGlu3 Abeta (Aβ). Also provided herein are pharmaceutical compositions comprising an anti-pyroGlu3 Abeta (Aβ) antibody, antigen-binding domain, antigen-binding fragment thereof that specifically binds to pyroGlu3 Abeta (Aβ) a pharmaceutically acceptable carrier, excipient or stabilizer.

Provided herein are pharmaceutical compositions comprising an antigen-binding domain that specifically binds to pyroGlu3 Abeta (Aβ) and comprising compositions that that bind to human TfR or to human CD98hc as described herein and a pharmaceutically acceptable carrier, excipient or stabilizer (Remington's Pharmaceutical Sciences (1990) Mack Publishing Co., Easton, PA). Also provided herein are pharmaceutical compositions comprising an antigen-binding domain that specifically binds to pyroGlu3 Abeta (Aβ) and comprising compositions that bind to human CD98hc as described herein and a pharmaceutically acceptable carrier, excipient or stabilizer. Acceptable carriers, excipients, or stabilizers are nontoxic to recipients at the dosages and concentrations employed. Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some aspects, a pharmaceutical composition comprising an antigen-binding domain that specifically binds to pyroGlu3 Abeta (Aβ) herein are pharmaceutical compositions comprising an antigen-binding domain that specifically binds to pyroGlu3 Abeta (Aβ) and compositions that bind to human TfR as described herein and a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). In some aspects, a pharmaceutical composition comprises an antigen-binding domain that specifically binds to herein are pharmaceutical compositions comprising an antigen-binding domain that specifically binds to pyroGlu3 Abeta (Aβ) and compositions that bind to human CD98hc as described herein and a pharmaceutically acceptable carrier. Pharmaceutical compositions described herein are, in some aspects, for use as a medicament. The compositions to be used for in vivo administration can be sterile. This is readily accomplished by filtration through, e.g., sterile filtration membranes.

In some aspects, provided herein are pharmaceutical compositions comprising a polynucleotide encoding an antigen-binding domain that specifically binds to herein are pharmaceutical compositions comprising an antigen-binding domain that specifically binds to Abeta (Aβ) and a composition that binds to human TfR as described herein. Also provided herein are pharmaceutical compositions comprising a polynucleotide encoding an antigen-binding domain that specifically binds to herein are pharmaceutical compositions comprising an antigen-binding domain that specifically binds to Abeta (Aβ) and a composition that binds to human CD98hc as described herein. In some aspects, the polynucleotide is RNA. In some aspects, the polynucleotide is a synthetic mRNA. In some aspects, the polynucleotide is a modified mRNA. In some aspects, the pharmaceutical composition comprising a polynucleotide further comprises a lipid-based transfection reagent.

A pharmaceutical composition described herein can be used to exert a biological effect(s) in vivo or in vitro. For example, a pharmaceutical composition described herein can be used to cross a blood brain barrier, e.g., in a subject.

In some aspects, a pharmaceutical composition provided herein is used to treat diseases or conditions such as a neurodegenerative disease. In some aspects, a pharmaceutical composition provided herein is used to treat diseases or conditions such as dementia, Alzheimer's disease (AD), amyloidosis, and other diseases and disorders associated with Abeta (Aβ).

In some aspects, a pharmaceutical composition provided herein is administered in combination with another compound or composition. In some aspects, the combination is used to treat diseases or conditions such as neurodegenerative diseases, dementia, Alzheimer's disease (AD), amyloidosis, and other diseases and disorders associated with Abeta.

In some aspects, a pharmaceutical composition provided herein is administered in combination with another agent useful in the treatment of Alzheimer's disease. Exemplary treatments for Alzheimer's disease are known in the art and include those discussed in Cummings, J. L., et al., Translational Research & Clinical Interventions 11:e70098 (2025), which is herein incorporated by reference in its entirety. Other exemplary treatments for Alzheimer's disease include, e.g., 2-HOBA, 50561, ABBV-916, ACI-24.060, ACI-35, AL002, AL101, Aldesleukin, ASN51, Astragalus, Baricitinib, BCG vaccine, Benfotiamine, Bepranemab, BIIB080, BMS-986446, Brexanolone, Brivaracetam, Bumetanide, Cannabidiol, Ciprofloxacin+Celecoxib, Contraloid acetate, CORT108297, CT1812, DAOIB+AO, Dasatinib+Quercetin, Dexmedetomidine, Dimebon, E2814+Lecanemab, Edaravone, Enrupatinib, EX039, Exercised plasma, Fasudil, Foralumab, IGC-AD1, Insulin, ITI-1284, IVL3003, JNJ-63733657, Lecanemab, Lemborexant, Lenalidomide, Leuprolide, Levetiracetam, LY3372689, Memantine, Mevidalen, MIB-626, MK-1167, Nicotinamide riboside, Nicotine transdermal patch, Nicotinic Acid, NPO3, Polypill, Rifaximin, R07269162, RQC, Sabirnetug, Sargramostim, Senicapoc, SHR-1707, Simufilam, Siponimod, Sirolimus, SPG302, Suvorexant, Tertomotide, Trazodone, Trontinemab, UE2343, Valaciclovir, Valiltramiprosate, XProl595, and Yangxue Qingnao.

In some aspects, a pharmaceutical composition provided herein is administered in combination with a compound or composition that targets tau. The term “tau,” as used herein, refers to the microtubule-associated protein and includes translational modifications, which include but are not limited to phosphorylation. In some aspects, the tau-targeting compound reduces abnormal accumulation and formation of tau. In some aspects, the tau-targeting compound comprises an antibody or antigen-binding fragment thereof, a peptide mimetic, a small molecule, an RNA-based molecule (e.g., RNAi, siRNA, or ASO), or a gene therapy. In some aspects, the tau-targeting compound comprises an antibody that bind to tau. In some aspects, the antibody that bind to tau comprises one or more of posdinemab (Johnson & Johnson), VY7523 (Voyager), etalanetug (Eisai) and BMS-986446 (Bristol Myers Squibb). In some aspects, the tau-targeting compound comprises an RNA interference (RNAi) compound, a small interfering RNA (siRNA), or an antisense oligonucleotide (ASO) such as ARO-MAPT(Arrowhead) or LY3954068 (Lilly) or IONIS-MAPTRx (BIIB080) (Biogen/Ionis).

In some aspects, a pharmaceutical composition provided herein is administered in combination with another compound or composition that targets apolipoprotein E receptor 2 (ApoER2), e.g., the splicing, expression, signaling, or any combination thereof of ApoER2. In some aspects, the ApoER2-targeting compound comprises an antibody or antigen-binding fragment thereof, a peptide mimetic, a small molecule, an RNA-based molecule (e.g., RNAi, siRNA, or ASO), or a gene therapy.

In some aspects, a pharmaceutical composition provided herein is administered in combination with another compound or composition that targets apolipoprotein E4 (ApoE4), e.g., the gene expression and/or protein levels or ApoE4. In some aspects, the ApoE4-targeting compound comprises an antibody or antigen-binding fragment thereof, a peptide mimetic, a small molecule, an RNA-based molecule (e.g., RNAi, siRNA, or ASO), or a gene therapy. In some aspects, the compound or composition that targets ApoE4 comprises TRACER™ capsid (Voyager) (Poblano J, Castillo-Tobias I, Berlanga L, et al. Drugs targeting ApoE4 that regulate beta-amyloid aggregation in the brain: Therapeutic potential for Alzheimer's disease. Basic Clin Pharmacol Toxicol. 2024; 135(3): 237-249. doi:10.1111/bcpt.14055).

In some aspects, a pharmaceutical composition provided herein is administered in combination with another compound or composition that targets apolipoprotein E2 (ApoE2), e.g., the expression and/or protein levels of ApoE2. In some aspects, the ApoE2-targeting compound comprises an antibody or antigen-binding fragment thereof, a peptide mimetic, a small molecule, an RNA-based molecule (e.g., RNAi, siRNA, or ASO), or a gene therapy. In some aspects, the ApoE2-targeting compound comprises a TRACER™ capsid (Voyager) or LX1001 (Lexeo).

In some aspects, a pharmaceutical composition provided herein is administered in combination with another compound or composition that targets nucleotide-binding oligomerization domain-like receptor pyrin domain-containing 3 (NLRP3). In some aspects, the NLRP3-targeting compound comprises ACI-19764 and/or NMRA-215. In some aspects, the NLRP3-targeting compound comprises an antibody or antigen-binding fragment thereof, a peptide mimetic, a small molecule, an RNA-based molecule (e.g., RNAi, siRNA, or ASO), or a gene therapy. In some aspects, the compound or composition reduces activation of the NLRP3 inflammasome and/or comprises dapansutrile (OLT1177), NT-0796, and/or VTX3232.

In some aspects, a pharmaceutical composition provided herein is administered in combination with another compound or composition that targets glycogen synthase kinase-3 beta (GSK3B). In some aspects, the GSK3B-targeting compound comprises an antibody or antigen-binding fragment thereof, a peptide mimetic, a small molecule, an RNA-based molecule (e.g., RNAi, siRNA, or ASO), or a gene therapy. In some aspects, the compound or composition that targets GSK3B comprises SAR50225.

In some aspects, a pharmaceutical composition provided herein is administered in combination with another compound or composition that targets (e.g., agonizes) glucagon-like peptide-1 (GLP-1). In some aspects, the GLP-1-targeting compound comprises an antibody or antigen-binding fragment thereof, a peptide mimetic, a small molecule, an RNA-based molecule (e.g., RNAi, siRNA, or ASO), or a gene therapy. In some aspects, the GLP-1 agonists comprises exendin, lixisenatide, dulaglutide, liraglutide, semaglutide, tirzepatide, or combinations thereof.

In some aspects, a pharmaceutical composition provided herein is formulated for intravenous administration. In some aspects, a pharmaceutical composition provided herein is formulated for subcutaneous administration.

Methods of Using Agents Comprising Antigen-Binding Domains that Bind PyroGlu3 Abeta (Aβ)

As disclosed herein, anti-pyroGlu3 Abeta (Aβ) antibodies of the present disclosure may be used for treating various diseases, disorders, and conditions. In some embodiments, the present disclosure provides methods for treating an individual having a neurodegenerative disorder, such as, for example, Alzheimer's disease, comprising administering to the individual a therapeutically effective amount of an anti-pyroGlu3 Abeta (Aβ) antibody of the present disclosure.

In some embodiments, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some embodiments, the subject or individual is a human.

Methods of Using Agents Comprising Antigen-Binding Domains that Bind PyroGlu3 Abeta (Aβ) and Antigen-Binding Domains that Bind to Blood-Brain Barrier Receptors or Proteins

Compositions provided herein comprise (i) an anti-TfR or an anti-CD98hc antigen-binding domain and (ii) an anti pyroGlu3 Abeta (Aβ) antigen-binding domain that can advantageously be transported across a blood brain barrier.

Accordingly, provided herein are methods of administering or transporting compositions comprising (i) an antigen-binding protein that specifically binds to human TfR or human CD98hc and (ii) an antigen-binding domain the specifically binds to pyroGlu3 Abeta (Aβ) across the blood brain barrier of a subject comprising administering to the subject such compositions.

In view of the ability of compositions provided herein to specifically bind to human TfR or human CD98hc and be transported across a blood brain barrier, these compositions can be used to treat a neurological disease or disorder. In some aspects, a method of treating a neurological disease or disorder in a subject comprises administering to the subject a composition comprising (i) an antigen-binding protein that specifically binds to human TfR or human CD98hc and (ii) an antigen-binding domain that specifically binds to pyroGlu3 Abeta (Aβ).

The neurological disease or disorder can be, for example, a neuropathy disorder, a neurodegenerative disease, or amyloidosis. The neurological disease or disorder can be, for example, a neurodegenerative disease dementia or Alzheimer's disease (AD). In some aspects, the neurological disease or disorder is dementia. In some aspects, the neurological disease or disorder is mild cognitive impairment. The terms “mild cognitive impairment” and “MCI,” used interchangeably herein, refer to an early stage of memory loss, or other cognitive ability loss (such as language or visual/spatial perception) in individuals who maintain the ability to independently perform most activities of daily living.

In some aspects, the neurological disease or disorder is Alzheimer's disease. In some aspects, the Alzheimer's disease is preclinical Alzheimer's disease. As used herein, “preclinical Alzheimer's disease” refers to a disease stage in which the subject is amyloid-positive but has no symptoms. In some aspects, the Alzheimer's disease is early Alzheimer's disease. As used herein, “early Alzheimer's disease” refers to a disease stage in which the subject is amyloid-positive and has mild symptoms (as observed in MCI or mild dementia). In some aspects, the Alzheimer's disease is late Alzheimer's disease. As used herein, “late Alzheimer's disease” refers to a disease stage in which the subject has moderate-severe dementia.

In some aspects, provided herein is a method of administering or transporting compositions comprising administering to the subject any of the multispecific proteins disclosed herein, any of the compositions disclosed herein, any of the pharmaceutical compositions disclosed herein, or any of the nucleotide sequences disclosed herein.

In some aspects, provided herein is a method of treating a lysosomal storage disease or disorder in a subject comprising administering to the subject any of the multispecific proteins disclosed herein, any of the compositions disclosed herein, any of the pharmaceutical compositions disclosed herein, or any of the nucleotide sequences disclosed herein.

In some aspects, provided herein is a method of treating a CNS disease or disorder in a subject comprising administering to the subject any of the multispecific proteins disclosed herein, any of the compositions disclosed herein, any of the pharmaceutical compositions disclosed herein, or any of the nucleotide sequences disclosed herein.

In some aspects, the CNS disease or disorder is Alzheimer's disease.

As described herein, compositions comprising an anti-Abeta (pyroGlu3 Aβ) antigen-binding domain and an anti-TfR antigen binding domain of the present disclosure are useful for treating a neurological disease or disorder, such as, for example, Alzheimer's disease. In some embodiments, the composition comprises AB-6-TfRL10-16. In some embodiments, the composition comprises AB-7-TfRL10-16. In some embodiments, the composition comprises AB-8-TfRL10-16. In some embodiments, the composition comprises AB-8-TfRL10-8. In some embodiments, the composition comprises AB-8-TfR42Q. Compositions of the present disclosure and uses thereof have a number of desirable properties and provide certain advantages for safety and efficacy in the treatment of neurological diseases and disorders, including Alzheimer's disease, and other diseases and disorders associated with the deposition of amyloid-beta.

As known by one of skill in the art, anti-TfR antibodies can block the binding of transferrin to its receptor (TfR1) on the surface of cells, thus interfering with iron transport. Disruption in iron transport into and uptake by erythroid progenitor cells (reticulocytes) can lead to a reduction in hematocrit, hemoglobin levels, and red blood cell count, resulting in anemia. Without being bound by this theory, use of an anti-TfR antibody in the multispecific binding proteins described herein, wherein the anti-TfR antibody displays lower affinity binding to human TfR (e.g., ~2000 nM), minimizes interference with iron uptake by reticulocytes, thereby reducing the likelihood of anemia. In some aspects, administration of a composition comprising an anti-pyroGlu3 Abeta (Aβ) antigen-binding domain and an anti-TfR antigen binding domain of the present disclosure does not result in anemia.

In some aspects, a composition comprising an anti-pyroGlu3 Aβ antigen-binding domain and an anti-TfR antigen binding domain of the present disclosure is associated with minimal hematologic adverse events when administered to a subject. In some aspects, administration of a composition comprising an anti-pyroGlu3 Abeta (Aβ) antigen-binding domain and an anti-TfR antigen binding domain of the present disclosure to a subject is associated with no adverse or long-term reduction in reticulocyte counts in the subject. In some aspects, administration of a composition comprising an anti-pyroGlu3 Abeta (Aβ) antigen-binding domain and an anti-TfR antigen binding domain of the present disclosure to a subject is associated with no adverse or significant reduction in red blood cell counts in the subject. In some aspects, administration of a composition comprising an anti-pyroGlu3 Abeta (Aβ) antigen-binding domain and an anti-TfR antigen binding domain of the present disclosure to a subject is associated with no adverse or significant reduction in hematocrit in the subject. In some aspects, administration of a composition comprising an anti-pyroGlu3 Abeta (Aβ) antigen-binding domain and an anti-TfR antigen binding domain of the present disclosure to a subject is associated with no adverse or significant reduction in hemoglobin levels in the subject. In some embodiments, the composition comprises AB-6-TfRL10-16. In some embodiments, the composition comprises AB-7-TfRL10-16. In some embodiments, the composition comprises AB-8-TfRL10-16. In some embodiments, the composition comprises AB-8-TfRL10-8. In some embodiments, the composition comprises AB-8-TfR42Q.

In some aspects, administration of a composition comprising an anti-pyroGlu3 Abeta (Aβ) antigen-binding domain and an anti-TfR antigen binding domain of the present disclosure to a subject results in rapid clearance or reduction in Abeta (Aβ) levels in the brain. In some aspects, administration of a composition comprising an anti-pyroGlu3 Abeta (Aβ) antigen-binding domain and an anti-TfR antigen binding domain of the present disclosure to a subject requires less frequent MRI monitoring or no monitoring of Abeta (Aβ) levels in the brain compared to administration of other anti-Abeta (Aβ) antibody therapies. In some aspects, administration of a composition comprising an anti-pyroGlu3 Abeta (Aβ) antigen-binding domain and an anti-TfR antigen binding domain of the present disclosure to a subject does not require APOE genotyping prior to administration to determine if the subject is homozygous or heterozygous for APOE4. In some aspects, administration of a composition comprising an anti-pyroGlu3 Abeta (Aβ) antigen-binding domain and an anti-TfR antigen binding domain of the present disclosure to a subject is associated with reduced occurrence of Amyloid-Related Imaging Abnormalities (ARIA) compared to administration of other anti-Abeta (Aβ) antibody therapies. In some aspects, a therapeutically effective dose of a composition comprising an anti-pyroGlu3 Abeta (Aβ) antigen-binding domain and an anti-TfR antigen binding domain of the present disclosure is a lower therapeutically effective dose than that of other anti-pyroGlu3 Abeta (Aβ) antibodies.

In some aspects, the methods provided herein comprise administering an an anti-pyroGlu3 Abeta (Aβ) antigen-binding domain and an anti-BBB (e.g., TfR or CD98hc) antigen binding domain and administering another compound or composition, e.g., another agent useful for the treatment of Alzheimer's disease, a tau-targeting agent, an ApoER2-targeting agent, an ApoE4-targeting agent, an ApoE2-targeting agent, a NLRP3-targeting agent, a GSK3B targeting agent, a GLP-1 targeting agent, etc as discussed above. Compositions provided herein can be used to detect an antigen (e.g., a CNS antigen or a brain antigen). In some aspects, the compositions provided herein can be labeled. Exemplary labels include, for example, radioisotopes (e.g., ⁶⁴CU) and fluorescent labels. Accordingly, methods of detecting the compositions provided herein are provided. In some aspects, a method of detecting an antigen in the CNS (e.g., brain) of a subject comprises administering the compositions provided herein in the CNS (e.g., brain). Such methods can further comprise, e.g., performing Positron emission tomography (PET) imaging on the subject. In some aspects, disclosed herein is a method of detecting a CNS antigen in vitro, comprising contacting an in vitro sample with a composition disclosed herein and locating the imaging agent within the sample.

Compositions provided herein can be used for prognostic, diagnostic, monitoring, and/or screening applications, including in vivo applications well known and standard to the skilled artisan and based on the present description. In some aspects, provided herein is a composition for use as a diagnostic. In some aspects, the compositions provided herein comprise a detectable label.

All references cited herein, including patent applications and publications, are hereby incorporated by reference in their entirety.

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Antigen-Binding Domains Against Human Transferrin Receptor

Antibodies to human transferrin receptor were obtained, characterized, and recombinantly expressed as described previously in WO2024/026472 (filed Jul. 28, 2023) and WO2025/166045 (International Application No. PCT/US2025/013846; filed Jan. 30, 2025), which is incorporated herein by reference in its entirety.

### Example 2: Antigen-Binding Domains Against Human CD98hc

Antibodies to human CD98hc were obtained, characterized, and recombinantly expressed as described previously in WO2024/026471 (filed Jul. 28, 2023) and in WO2025166042 (International Application Serial No. PCT/US2025/013843; filed Jan. 30, 2025), both of which are incorporated herein by reference in their entireties.

### Example 3: Synthesis of Unconjugated and Carrier-Conjugated Abeta Peptides

Synthetic peptides of human N-terminally truncated pyroglutamate-modified (pyroGlu) and non-modified (i.e., non-pyroglutamate-modified) Abeta fragments for use in the generation and characterization of anti-pyroGlu3 Abeta (Aβ) antibodies of the present disclosure were generated as follows. Nucleic acids encoding human pyroGlu3-16 Abeta (Aβ), human pyroGlu3-24 Abeta (Aβ), human pyroGlul 1-24 Abeta (Aβ), and nitrotyrosine 10 Aβ3-14. See Table 12 below for amino acid sequences of these polypeptides. Abeta (Aβ) fragments were chemically synthesized with a C-terminal cysteine residue and conjugated to keyhole limpet hemocyanin (KLH) or bovine serum albumin (BSA) for use as immunogens (YenZym, Brisbane, CA and LifeTein, Somerset, NJ).

These polypeptides along with nucleic acids encoding human non-modified 3-16 Abeta (Aβ) fragment and human non-modified 3-24 Abeta (Aβ) fragment were chemically synthesized with a C-terminal cysteine residue and either biotinylated or left unconjugated for use as screening reagents (YenZym, Brisbane, CA and Genscript, Piscataway NJ). Additional polypeptides—human non-modified Abeta (Aβ) 1-40, human Abeta (Aβ) 1-42, and human Abeta (Aβ 3-40)—were obtained commercially (AnaSpec, Fremont, CA). All polypeptides were reconstituted according to manufacturer's instructions prior to use.

Amino acid sequences of human pyroGlu3-16 Abeta (Aβ) fragment, human non-modified 3-16 Abeta (Aβ) fragment, human pyroGlu3-24 Abeta (Aβ) fragment, human non-modified 3-24 Abeta (Aβ) fragment, human non-modified Abeta (Aβ) 1-40, human Abeta (Aβ) 1-42 and human Abeta (Aβ) 3-40 are set forth below in Table 12.

TABLE 12

| Abeta (Aβ) | | |
|---|---|---|
| Polypeptide | Amino Acid Sequence | SEQ ID NO: |
| Human Pyroglutamated | [pyr]-EFRHDSGYEVHHQK | SEQ ID NO: 215 |
| Glu3-16 | | |
| Human Non-modified | EFRHDSGYEVHHQK | SEQ ID NO: 216 |
| 3-16 Abeta | | |
| Human Pyroglutamated | [pyr]-EFRHDSGYEVHHQKLVFFAEDV | SEQ ID NO: 217 |
| Glu3-24 Abeta | | |
| Human Non-modified | EFRHDSGYEVHHQKLVFFAEDV | SEQ ID NO: 218 |
| 3-24 Abeta | | |
| Human Non-modified | DAEFRHDSGYEVHHQKLVFFAEDVGSNKG | SEQ ID NO: 219 |
| 1-40 Abeta | AIIGLMVGGVV | |
| Human Non-modified | DAEFRHDSGYEVHHQKLVFFAEDVGSNKG | SEQ ID NO: 1 |
| 1-42 Abeta | AIIGLMVGGVVIA | |
| Human Non-modified | EFRHDSGYEVHHQKLVFFAEDVGSNKGAII | SEQ ID NO: 220 |
| 3-40 | GLMVGGVV | |
| Human Pyroglutamated | [pyr]-EVHHQKLVFFAEDV | SEQ ID NO: 221 |
| Glu11-24 Abeta | | |
| Human Nitrosylated | EFRHDSG-[3NT]Y-EVHH | SEQ ID NO: 222 |
| Tyr10 Abeta 3-14 | | |

### Example 4: Generation of Anti-Pyroglu3 Abeta Hybridoma Antibodies

Alloy ATX-GKH hyperimmune strain mice (Alloy Therapeutics, Waltham, MA) were immunized twice a week by subcutaneous or intraperitoneal injections of alternating KLH- or BSA-conjugated peptides of human pyroGlu3-16, pyroGlu3-24, pyroGlul 1-24 and/or nitrotyrosine 10-Aβ3-14 Abeta (Aβ) fragments (with or without adjuvant). After a total of 8 injections and three days following the final injection boost, lymph nodes and spleens were harvested from the mice for hybridoma cell line generation. Sera from the animals were analyzed for reactivity to human pyroGlu3 moiety by ELISA against the pyroGlu-modified and non-modified polypeptides described above in Example 3.

Lymphocytes from animals whose sera demonstrated strong binding reactivity to human pyroGlu3-24 Abeta (Aβ) peptide by ELISA were isolated and fused with SP2/mIL-6 (CRL-2016, American Type Culture Collection, Rockville, MD) mouse myeloma cells via electrofusion. The fused cells were mixed with anti-mouse IgG Fc-FITC and plated into methylcellulose-based ClonaCell-HY Medium D (Stemcell Technologies, Cat #03804) containing HAT components. Fluorescent IgG positive colonies were selected and transferred by a ClonePix 2 system (Molecular Devices, Sunnyvale, CA) into 96-well plates. In total, ~4800 hybridoma clones were isolated and screened for binding to human pyroGlu3-24 Abeta (Aβ) polypedptide fragments as described below.

### Example 5: Screening of Hybridoma Supernatants by ELISA

Supernatants from the hybridomas obtained as described above were screened for their ability to differentially bind human pyroGlu3-24 Abeta (Aβ) polypeptides as compared to non-pyroglutamate-modified Abeta (Aβ) polypeptides by ELISA. Briefly, 96-well polystyrene plates were coated with streptavidin, washed, and blocked with ELISA diluent (PBS+0.5% BSA+0.05% Tween20). The plates were then incubated with biotinylated Abeta (Aβ) polypeptides, washed, and incubated with undiluted hybridoma cell culture supernatants. After another round of washing, the plates were incubated with anti-mouse IgG Fc-HRP (Jackson Immunoresearch, Cat #115-035-071). After a final round of washing, the plates were developed with TOMB substrate, quenched with sulfuric acid, and read on a SpectraMax M5 for absorbance at 450 nm. After multiple rounds of ELISA screening, a total of 280 anti-pyroGlu3 Abeta (Aβ) hybridoma clones were identified that displayed greater than 10-fold difference in binding to pyroGlu3-24 Abeta (Aβ) polypeptides over background binding levels. Anti-pyroGlu3 Abeta (Aβ) antibodies identified using this screen were further characterized as described below.

### Example 6: Screening Hybridoma Supernatants by Immunohistochemical Staining

A total of 96 anti-pyroGlu3 Abeta (Aβ) hybridoma supernatants obtained as described above were screened for their ability to bind to pyroGlu3 Abeta (Aβ) expressed in amyloid plaques by immunohistochemical (IHC) staining. Briefly, 20 m sagittal sections from non-fixed 5XFAD mouse brains were washed three times in PBS for 5 min, and then permeabilized with PBST (PBS+0.3% Triton X-100) for 15 min. Brain sections were washed twice in distilled water for 5 min and blocked with 2.5% BSA (MilliporeSigma, Cat #A7030) and 2.5% donkey serum (Jackson Immunoresearch, Cat #017000121) in PBS containing 0.3% Triton X-100 for 1 h at RT. Hybridoma supernatant antibody titers were determined using a Gator Bio-Layer Interferometry (Palo Alto, CA) system, and supernatants were normalized to 20 μg/mL. Tissue sections were incubated with 20 μg/mL hybridoma antibody supernatants and 2 μg/mL anti-Abeta (−Aβ) (6E10) rabbit antibody (Novus Biologicals, Cat #NBP2-62566) in PBS containing 0.75% BSA, 0.75% donkey serum and 0.3% Triton X-100 overnight at 4 °C. The tissue sections were washed three times in PBS for 10 min and incubated in the dark with a secondary antibody solution consisting of both 0.2% Alexa fluor 647 donkey anti-rabbit IgG (Jackson Immunoresearch, Cat #711-605-152) and 0.2% Alexa fluor 488 donkey anti-human IgG (Jackson Immunoresearch, Cat #711-605-152) in PBS containing 0.75% BSA, 0.75% donkey serum and 0.3% Triton X-100 for 2 h at RT. Brain sections were then washed three times in PBS for 10 min and rinsed in distilled water. Slides were coverslipped with VECTASHIELD HardSet Antifade Mounting Medium with DAPI (Vector Laboratories, Cat #H150010). Slides were imaged using ZEISS Axioscan. A total of 21 anti-pyroGlu3 Abeta (Aβ) hybridoma antibodies were identified that displayed binding to amyloid plaques with a similar staining pattern as that observed using either anti-pyroGlu 3 antibody donanemab or remternetug. Donanemab (brand name Kisunla™) is an FDA-approved, Eli Lilly-developed intravenous immunotherapy for early symptomatic Alzheimer's disease. Remternetug (LY3372993) is an investigational anti-amyloid monoclonal antibody developed by Eli Lilly for early Alzheimer's disease.

### Example 7: Screening of Hybridoma Supernatants by SPR

Supernatants from hybridomas obtained as described above were further screened for binding to human pyro-Glu3 Abeta (Aβ) polypeptides (pE3-16, pE3-24, pE3-40, pE3-42) and non-modified Abeta (Aβ) polypeptides (3-40, 1-42) using a Carterra LSA instrument (Carterra, Salt Lake City, UT). Hybridoma supernatants were diluted 1:4 with running buffer HBS-EP+(Teknova) with 0.5 mg/ml BSA (MP Biomedicals). An anti-multispecies Fc capture lawn was prepared using a SAD200M sensor chip (Carterra) by coupling a mixture of 20 μg/mL of multi-species Fc VHH (ThermoFisher Scientific). Antibodies were injected over the capture lawn for 10 minutes each. PyroGlu3 Abeta (Aβ) polypeptides analytes were diluted with running buffer, in a series of three, five-fold serial dilutions and two, ten-fold serial dilutions, starting at 2500 nM. Each analyte injection cycle consisted of a single analyte injected non-regeneratively, from low to high concentrations, for three minutes, and dissociation was followed for five minutes. At the end of each injection cycle, the VHH capture lawn was regenerated with 10 mM glycine-HCl pH 1.7 buffer (Carterra). Data were processed and analyzed using NextGenKIT high-throughput kinetics analysis software (Carterra). Antibodies were selected for further characterization based on specific or preferential binding to pyroGlu3 Abeta (Aβ) polypeptides compared to binding to non-modified Abeta (Aβ) polypeptides.

### Example 8: Molecular Cloning of Anti-pyroGlu3 Aβ Antibodies

Anti-pyroGlu3 Abeta (Aβ) antibodies of the present disclosure derived from hybridoma clones as described above were sequenced via Reptor next-generation hybridoma sequencing platform (Abterra Bio, San Diego, CA).

Amino acid sequences of the variable heavy chains and variable light chains of the anti-pyroGlu3 Abeta (Aβ) antibodies of the present disclosure were subjected to in silico analysis for identification of possible high-risk liabilities and potential immunogenicity that are known to be inherent risks affecting antibody stability, efficacy, safety, and manufacturability. Amino acids identified as high-risk liabilities (e.g., unpaired Cys, N-linked glycosylation, Asn deamidation, or Asp isomerization) were modified to amino acids having lower risk for these liabilities. Amino acids that were identified as potentially immunogenic when compared to known human germline amino acid sequences were modified to amino acids having less predicted immunogenicity.

Amino acid sequences of the Kabat heavy chain CDR amino acid sequences, Kabat light chain CDR amino acid sequences, and heavy chain variable region and light chain variable region amino acid sequences of the anti-pyroGlu3 Abeta (Aβ) antibodies of the present disclosure are provided above in Tables 3, 4, and 5 above, corresponding to anti-pyroGlu3 Abeta (Aβ) antibodies AB-1, AB-2, AB-3, AB-4, and AB-5 and to liability-removed anti-pyroGlu3 Abeta (Aβ) antibody variants AB-6, AB-7, AB-8, AB-9, and AB-10.

### Example 9: Production of Recombinant Anti-pyroGlu3 Aβ Antibodies

Anti-pyroGlu3 Abeta (Aβ) antibody variable gene regions of the present disclosure were synthesized and subcloned into mammalian expression vectors encoding human IgG1 and IgK to express the anti-pyroGlu3 Abeta (Aβ) antibodies in Expi293 or ExpiCHO cells (Invitrogen) using standard procedures. Cell culture supernatants containing secreted recombinant anti-pyroGlu3 Abeta (Aβ) antibodies were harvested at 5 days post transfection and purified using protein A chromatography using standard procedures.

### Example 10: Screening of Purified Anti-pyroGlu3 Abeta Antibodies by ELISA

Purified recombinant anti-pyroGlu3 Abeta (Aβ) antibodies of the present disclosure were screened by ELISA as described above for their ability to bind human pyroGlu3 Abeta (Aβ) polypeptides as compared their binding to isotype and reference anti-Abeta antibody control antibodies donanemab, remternetug, and bapineuzumab. The absorbance values as a ratio of polypeptide binding over background values were calculated and are summarized in Table 13 below. All 5 recombinant anti-pyroGlu3 Abeta (Aβ) antibodies tested in this assay (AB-1, AB-2, AB-3, AB-4, and AB-5) showed high levels of binding to human pyroGlu3-24 Abeta (Aβ) polypeptide, displaying much higher binding to human pyro-Glu3-24 Aβ polypeptide than that observed against human non-modified E3-24 Abeta (Aβ) polypeptide. Anti-Abeta (Aβ) antibodies donanemab and remternetug showed similar binding to both modified and non-modified Abeta (Aβ) 3-24 polypeptides. None of the recombinant anti-pyroGlu3 Abeta (Aβ) antibodies of the present disclosure showed binding to a longer-length human non-modified Abeta (Aβ) 3-40 polypeptide; however, both donanemab and remternetug showed low levels of binding to human non-modified Abeta (Aβ) 3-40 polypeptide. Additionally, anti-AP antibody bapineuzumab showed high levels of binding to human non-modified Abeta (Aβ) 3-40 polypeptide.

TABLE 13

| | ELISA binding ratio | | |
|---|---|---|---|
| | (fold-change over background) | | |
| | | Human | Human |
| | Human | non-modified | non-modified |
| | pyroGln3-24 | E3-24 | E3-40 |
| Antibody | Abeta (Aβ) | Abeta (Aβ) | Abeta (Aβ) |
| AB-1 | 58.1 | 16.9 | 1.3 |
| AB-2 | 63.0 | 13.7 | 1.5 |
| AB-3 | 66.0 | 15.1 | 1.3 |
| AB-4 | 64.8 | 27.0 | 1.6 |
| AB-5 | 66.7 | 30.8 | 1.5 |
| Isotype control | 1.6 | 1.0 | 1.3 |
| Donanemab | 42.9 | 55.4 | 5.9 |
| Remternetug | 65.6 | 46.0 | 3.6 |
| Bapineuzumab | 3.4 | 3.7 | 66.6 |

### Example 11: Screening of Purified Anti-pyroGlu3 Abeta (Aβ) Antibodies By Immunohistochemical Staining

Purified anti-pyroGlu3 Abeta (Aβ) antibodies of the present disclosure were examined for their ability to bind to pyroGlu3 Abeta (Aβ) expressed in amyloid plaques in vivo by immunohistochemical staining, as described above. All 5 anti-pyroGlu3 Abeta (Aβ) antibodies (AB-1 through AB-5) of the present disclosure showed a similar staining pattern to amyloid plaques in unfixed 5XFAD mouse brain sections as observed with anti-Abeta (Aβ) antibodies donanemab and remternetug (data not shown). These results indicated that anti-pyroGlu3 Abeta (Aβ) antibodies of the present disclosure had similar binding patterns to amyloid plaques in vivo in unfixed 5XFAD mouse brains as two other previously-described anti-Abeta (Aβ) antibodies donanemab and remternetug.

### Example 12: Assessment of Anti-pyroGlu3 ABeta (Aβ) Antibodies by Biological Function Assays

Purified anti-pyroGlu3 Abeta (Aβ) antibodies of the present disclosure were further characterized for their biological activities in an ex vivo amyloid plaque phagocytosis assay as follows. Frozen brain tissue from 5XFAD mice were sectioned to 20 μm and mounted on poly-D-lysine-coated cover glass (15 mm) and placed in 24-well tissue culture plates. Consecutive sections were positioned in the same order as sectioned and were incubated with or without antibodies (0.1-10 μg/ml, 500 ml, 1 hr, room temperature). Primary human macrophages or iPSC-derived microglia (5×10⁵ cells per well) were then added to sections and incubated for 24 h at 37 °C. At the end of incubation, media were removed, brain sections and cells were homogenized with 150 μL guanidine HCL lysis buffer (5.5M guanidine+50 mM Tris+1X protease inhibitor cocktail), and Abeta (Aβ)1-42 concentration quantified by ELISA. Briefly, 96-well polystyrene plates were coated with 10 μg/ml capture antibody 21F12 (Thermo Fisher Scientific, Catalog #MA5-51771), washed and blocked with ELISA diluent (PBS+2% BSA+0.05% Tween20). The plates were incubated with cell lysates, washed, and then incubated with 1 μg/ml biotinylated anti-Abeta AR) antibody 3D6 (Alector in-house reagent). After another round of washes, the plates were incubated with streptavidin-HRP (1:4000 dilution, Thermo Fisher Scientific, Cat #21130), followed with color development with TMB substrate. The level of residual Abeta (Aβ) after phagocytosis was measured on a SpectraMax M5 for absorbance at 450 nm. The level of residual Abeta (Aβ) was normalized to that observed in isotype control group.

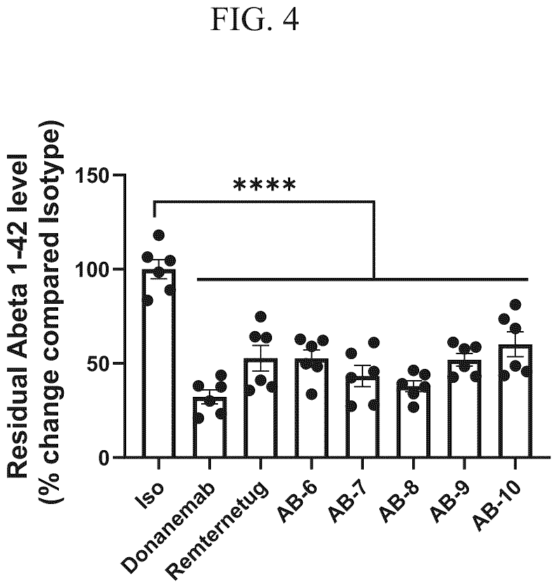

The ability of anti-pyroGlu3 Abeta (Aβ) antibodies of the present disclosure to promote plaque phagocytosis in this assay was compared to that observed with anti-Abeta (Aβ) antibodies donanemab and remternetug. As shown in FIG. 4, anti-pyroGlu3 Abeta (Aβ) antibodies at 0.1 μg/ml showed ~50% plaque clearance compared to that observed with isotype control antibody. Additionally, the extent of plaque phagocytosis observed in this assay with anti-pyroGlu3 Abeta (Aβ) antibodies of the present disclosure was similar to that observed with reference anti-Abeta (Aβ) antibodies donanemab or remternetug.

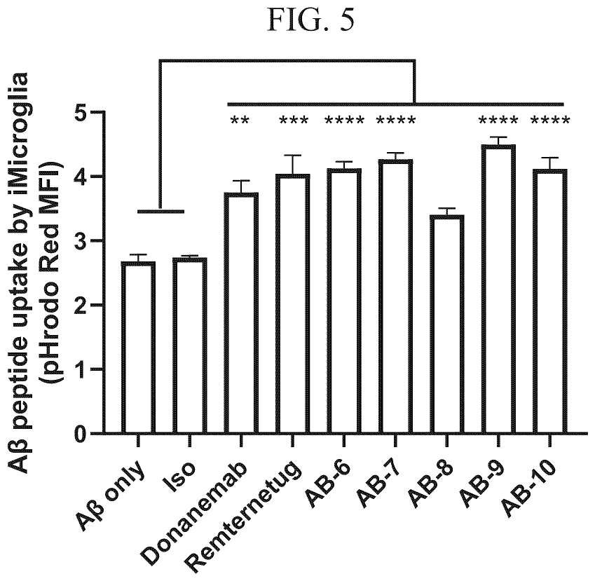

To investigate whether anti-pyroGlu3 Abeta (Aβ) antibodies of the present disclosure promoted uptake of pyroGlu3 Abeta (Aβ) by microglia in the CNS, a human CNS triple culture cell model was developed as follows. iPSC-derived microglia precursors expressing GFP were grown in parallel with iPSC-derived neuron and human fetal astrocyte co-cultures, and matured to form a genetically and functionally relevant human CNS triple culture cell system. CNS triple culture cells were simultaneously treated with anti-pyroGlu3 Abeta (Aβ) hybridoma antibodies of the present disclosure (0.01-10 μg/ml) and pyroGlu3-24 Abeta (Aβ) polypeptides (1 μM) labeled with fluorescent pH-sensitive pHrodo-Red dye for 24h. Uptake of pyroGlu3-24 Abeta (Aβ) by microglia in this human CNS triple culture cell system was monitored by IncuCyte in real time. The fluorescence intensity of pHrodo-Red signal was normalized to GFP tagged microglia to evaluate the effect of microglial pyroGlu3-24 Abeta (Aβ) phagocytosis for antibody candidate screening. As shown in FIG. 5, anti-pyroGlu3 Abeta (Aβ) antibodies of the present disclosure (0.1 μg/ml) showed similar increases in microglial pyroGlu3 Abeta (Aβ) phagocytosis as that observed with anti-Abeta (Aβ) antibodies donanemab or remternetug. These data indicated that anti-pyroGlu3 Abeta (Aβ) antibodies of the present disclosure were effective at phagocytosis of amyloid plaques.

### Example 13: In Vivo Screening of Purified Anti-pyroGlu3 Abeta (Aβ)Human IgG Antibodies

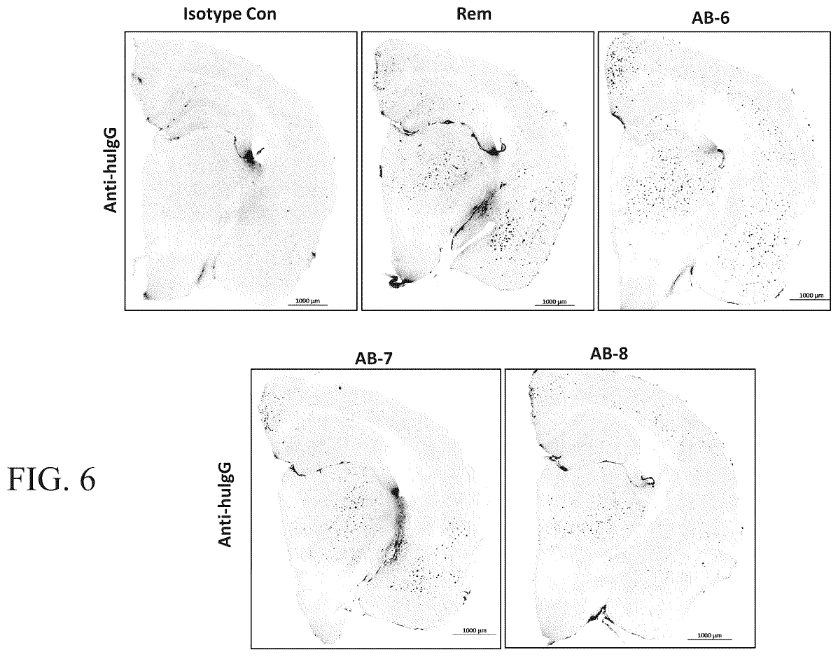

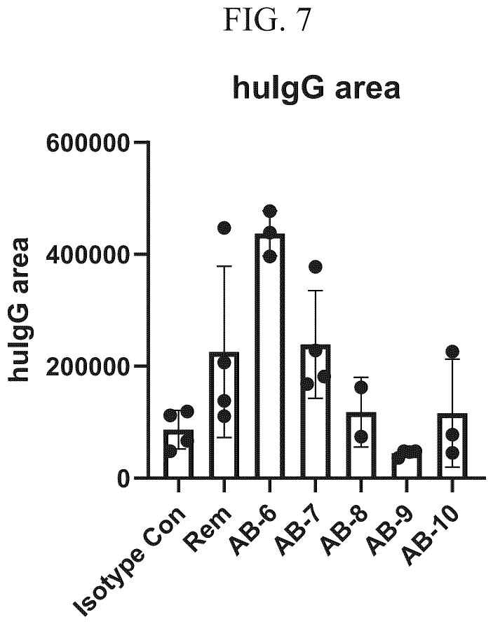

The extent of in vivo target engagement via plaque binding of peripherally administered anti-pyroGlu Abeta (Aβ) antibodies of the present disclosure was assessed as follows. 5XFAD mice aged to 6 months old were dosed with anti-pyroGlu3 Abeta (Aβ) antibodies of the present disclosure at 50 mg/kg intravenously via the tail vein; serum and brain samples were assessed 4 days after dosing. Fixed brains were processed for immunofluorescent staining and dosed anti-pyroGlu3 Abeta (Aβ) antibodies of the present disclosure and anti-Abeta (Aβ) antibody remternetug were detected by fluorescently labeled secondary antibodies against human IgG (FIG. 6). Anti-pyroGlu3 Abeta (Aβ) antibodies of the present disclosure bound to plaques that were also labeled by anti-Abeta (Aβ) antibody 6E10 (Novus Biologicals, Cat #NBP2-62566) (data not shown). Total area of antibody signal at plaques were quantified and averaged for each mouse (FIG. 7).

In this assay, anti-pyroGlu3 Aβ antibodies of the present disclosure showed varying degrees of amyloid plaque binding: anti-pyroGlu3 Abeta (Aβ) antibodies AB-7, AB-8, and AB-10 showed comparable amyloid plaque binding as that observed with anti-Abeta (Aβ) antibody remternetug. Anti-pyroGlu3 Abeta (Aβ) antibody AB-6 showed greater amyloid plaque binding compared to that observed with anti-Abeta (Aβ antibody remternetug.

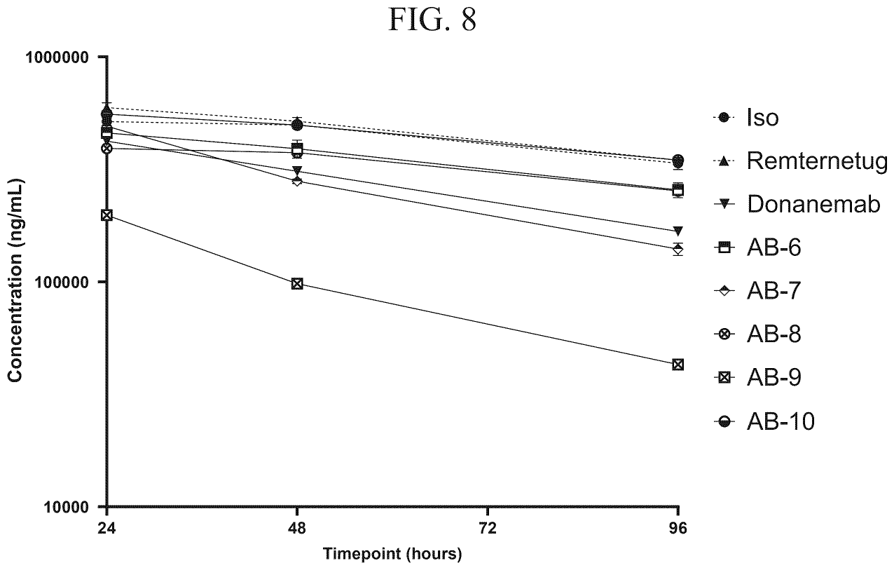

Serum samples were also collected from the animals on day 1 and day 2 to assess peripheral antibody levels (FIG. 8). Anti-pyroGlu3 Abeta (Aβ) antibodies AB-6, AB-8, and AB-10 showed a slower peripheral clearance profile than anti-pyroGlu3 Abeta (Aβ) antibodies AB-7 and AB-9 in this assay.

### Example 14: SPR Binding of Anti-pyroGlu3 Aβ huIgG Antibodies

Binding kinetics of the recombinant supernatants to human pyroGlu3 Abeta (Aβ) polypeptides (pE3-42, pE3-40, pE3-24, pE3-16) and non-modified Abeta (Aβ) polypeptides (1-42, 3-40, 3-16, 1-40). Abeta (Aβ) and non-modified Abeta (Aβ) peptides were evaluated using a Carterra LSA instrument (Carterra, Salt Lake City, UT) as follows. Anti-pytoGlu3 Abeta (Aβ) huIgG1 recombinant supernatants were diluted 1:5 with running buffer HBS-EP+(Teknova) with 0.5 mg/ml BSA (MP Biomedicals). An anti-huFc and anti-human CH1 capture lawn was prepared using a SAD200M sensor chip (Carterra) by coupling a mixture of 20 μg/mL of biotinylated anti-human Fc VHH (ThermoFisher Scientific) and 20 μg/mL of biotinylated anti-human CH1 VHH (ThermoFisher Scientific). The antibodies were injected over the capture lawn for 10 minutes each. Spots that yielded less than 20 RU of analyte binding were excluded from further analysis.

Various Abeta (Aβ) polypeptide analytes listed above were diluted with running buffer, in a series of eight, five-fold serial dilutions starting from 2500 nM. Each Abeta (Aβ) polypeptide analyte injection cycle consisted of a single analyte injected non-regeneratively, from low to high concentrations, for three minutes, and dissociation was followed for five minutes. At the end of each injection cycle, the VHH capture lawn was regenerated with 10 mM glycine-HCl pH 1.7 buffer (Carterra). Data were processed and analyzed using NextGenKIT high-throughput kinetics analysis software (Carterra).

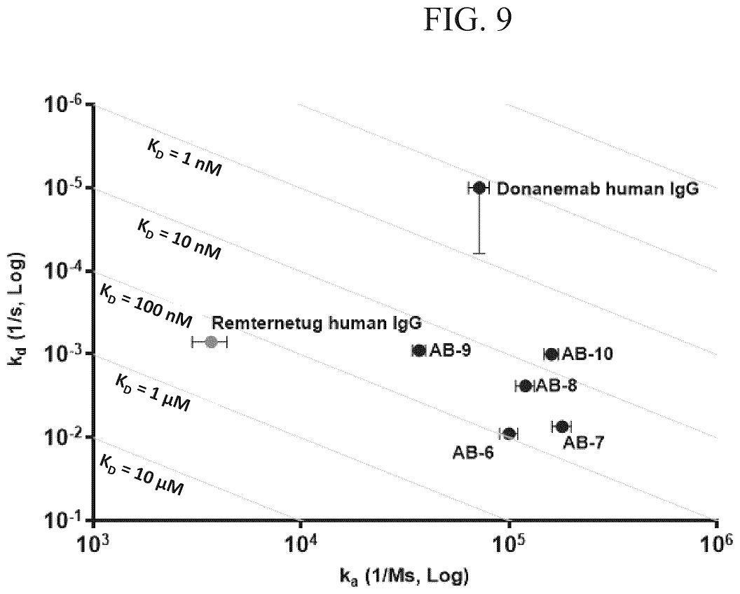

Equilibrium dissociation constants (K_{D}) were calculated from the fitted association and dissociation rate constant analysis (k-on and k-off) of each anti-pyroGlu3 Abeta (Aβ) antibody of the present disclosure binding to human pyroGlu3-24 Abeta (Aβ). These results are summarized in Table 14 below. Three anti-pyroGlu3 Abeta (Aβ) antibodies of the present disclosure (AB-6, AB-7, and AB-8) and anti-Abeta (Aβ) antibody remternetug were shown to bind specifically to pyroGlu3 pE3-24 Abeta (Aβ). These anti-Abeta (Aβ) antibodies did not show binding non-pyroglutamate-modified Abeta (Aβ) peptides. Two anti-pyroGlu3 Abeta (Aβ) antibodies of the present disclosure (AB-9, AB-10) and anti-Abeta (Aβ) antibody donanemab showed preferential binding to pyroGlu3 modified Abeta (Aβ) polypeptides compared to that observed with non-pyroglutamate-modified Abeta (Aβ) peptides. Taken together, these results indicated the specificity of anti-pyroGlu3 Abeta (Aβ) antibodies of the present disclosure bind to human N-terminally truncated pyroglutamate-modified form(s) of Abeta (Aβ), further displaying affinity to human N-terminally truncated pyroglutamate-modified form(s) of Abeta (Aβ) in the single- and double-digit nM range. FIG. 9 provides isoaffinity plots showing equilibrium dissociation constants (K_{D}), as a function of the association and dissociation rate constants (k-on and k-off), of anti-pyroGlu3 Abeta (Aβ) huIgG antibodies of the present disclosure to pyroGlu3 Abeta (Aβ) peptide (pE3-24).

TABLE 14

Rate constants to pE3-24 by Carterra (SPR)

| | kₐ | k_{d} | K_{D} |
|---|---|---|---|
| Antibody | (1/M s) | (1/s) | (nM) |
| AB-6 | 1E+05 ± 1E+04 | 9E−03 ± 4E−04 | 88 |
| AB-7 | 2E+05 ± 2E+04 | 7E−03 ± 3E−04 | 41 |
| AB-8 | 1E+05 ± 1E+04 | 2E−03 ± 9E−05 | 20 |
| AB-9 | 4E+04 ± 3E+03 | 9E−04 ± 4E−05 | 24 |
| AB-10 | 2E+05 ± 1E+04 | 1E−03 ± 4E−05 | 7 |
| donanemab | 7E+04 ± 8E+03 | 1E−05 ± 5E−05 | <1 |
| remternetug | 4E+03 ± 7E+02 | 7E−04 ± 6E−05 | 195 |

| | |
|---|---|
| bapineuzumab | No binding |

### Example 15: In Vivo Serum PK, Brain Uptake, and Safety of Anti-TfR scFvs

Two in vivo studies were performed to select anti-TfR scFvs with favorable serum PK, brain PK, and safety profiles to determine an optimal anti-TfR scFv to pair with anti-pyroGlu3 Abeta (Aβ) antibodies of the present disclosure. Three anti-TfR scFvs (TfRL7-2, TfRL10-16, and TfR39.38) were tested in the 2+1 format as described herein. The antibodies were generated with a human IgG1 WT Fc backbone due to the requirement of binding to Fc Receptors (e.g., such as FcγRIIa) in order to enhance phagocytic activity of the anti-pyroGlu3 Abeta (Aβ) antibodies. Due to known hematological safety liabilities of anti-TfR BBB shuttles with active Fc regions due to high expression of TfR in reticulocytes (see Couch et al., 2013, Sci Transl Med, 5(183):183ra57, 1-12) a key component of this screening was to look at the effect of these anti-TfR antibodies on reticulocyte levels in serum. Dose levels of antibodies comprising an anti-TfR binding moiety can affect reticulocyte depletion (Couch et al., 2013). Accordingly, a dose range of 3 mg/kg, 10 mg/kg, and 30 mg/kg was selected to assess the relationship between reticulocytes depletion and antibody dose.

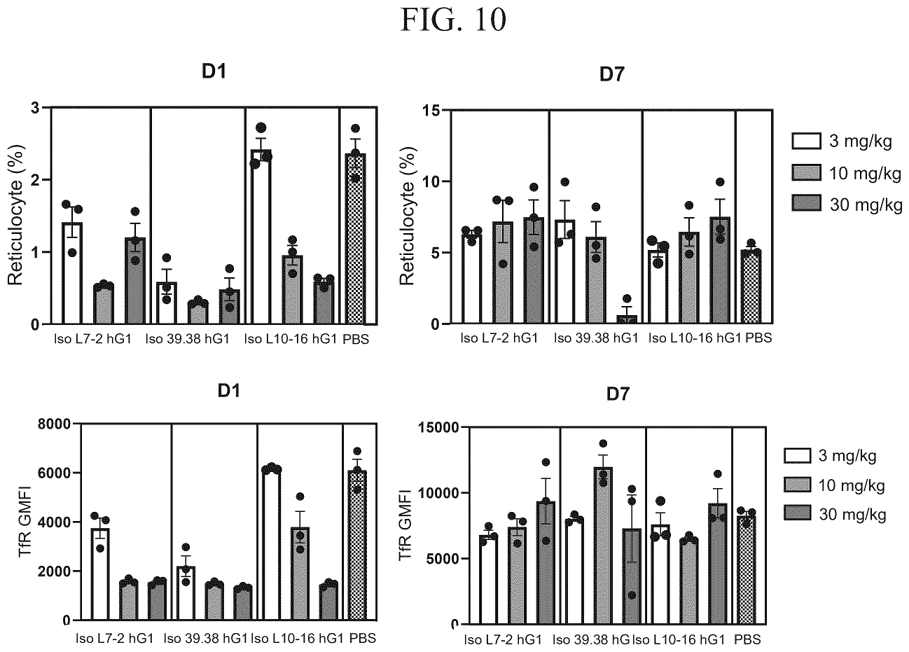

To evaluate the safety and dose relationship of anti-TfR antibody candidates for use as described herein, an in vivo study using 2+1 format antibodies comprising an isotype control cargo and a hIgG1 WT Fc was performed in hu TfR KI mice to measure changes in reticulocyte counts, TfR levels on reticulocytes, and serum antibody levels. Mice were weighed and dosed via intravenous injection of the tail vein with PBS or 2+1 format antibodies containing 3 different anti-TfR scFvs (Iso-TfRL7-2 hIgG1 WT, Iso-TfRL10-16 hIgG1 WT, Iso-TfR39.38-hIgG1 WT) at 3 mg/kg, 10 mg/kg, and 30 mg/kg. Whole blood samples were obtained from the mice 1 day and 7 days post-dose, and reticulocyte counts and TfR levels on reticulocytes were measured by flow cytometry (FIG. 10).

At day 1 post-dose, at all antibody dose levels tested, mice administered Iso-TfRL7-2 hIgG1 WT and Iso-TfR39.38 hIgG1 WT displayed reduced reticulocyte counts and reduced TfR expression levels on reticulocytes compared to that observed in the control PBS group. However, mice administered Iso-TfRL10-16 did not show a reduction in reticulocyte counts or changes in TfR expression levels in reticulocytes in animals administered the antibody at a dose 3 mg/kg. By day 7 post-dose, all animal groups that showed reduced reticulocyte counts and reduced TfR expression levels recovered, except for the 30 mg/kg Iso-TfR39.38 hIgG1 WT group.

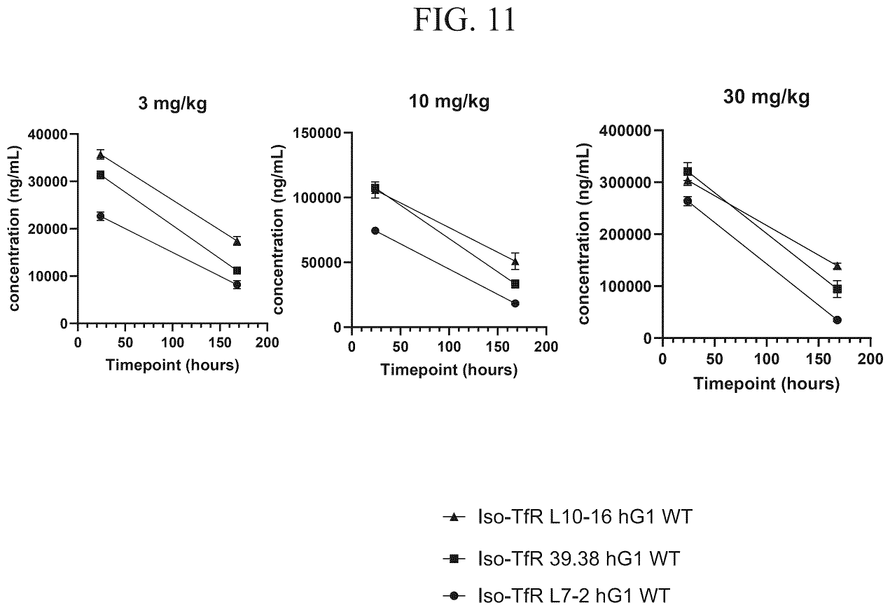

Animals were bled by sub-mandibular veins at 1 day and 7 days post-dosing to determine serum antibody concentrations (FIG. 11). Iso-TfRL10-16 showed slower peripheral clearance compared to that observed with either Iso-TfRL7-2 hIgG1 WT or Iso-TfR39.38 hIgG1 WT at all three dose levels tested. These results indicated that, based on this study, anti-TfRL10-16 scFv showed favorable serum antibody levels and safety profiles.

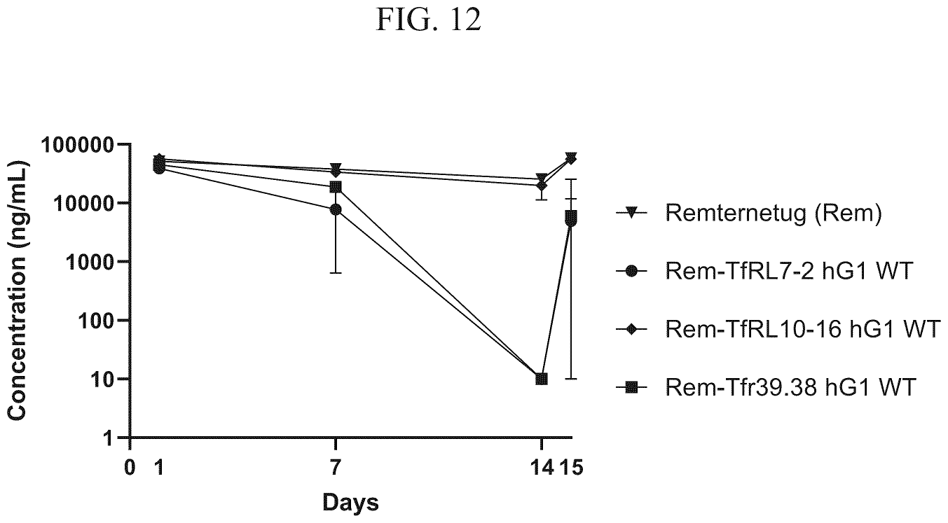

Another in vivo study was conducted to assess serum PK and brain uptake of anti-pyroGlu3 Abeta (Aβ)-TfR 2+1 antibodies of the present disclosure at low dose (3 mg/kg) in human TfR knock-in (huTfR KI) mice. Eight- to twelve-week-old female homozygous huTfR KI mice were weighed and dosed via intravenous injection of the tail vein with anti-Abeta (Aβ) antibody remternetug IgG control and 3 remternetug-TfR 2+1 antibodies (Rem-TfRL7-2 hIgG1 WT, Rem-TfRL10-16 hIgG1 WT, Rem-TfR39.38-hIgG1 WT) at 3 mg/kg with two doses 14 days apart. Animals were bled by sub-mandibular vein at 1 day, 7 days, and 14 days following the first dose, as well as 24 hours following the second dose, to determine serum PK profiles (FIG. 12). Remternetug-TfRL10-16 hIgG1 WT showed a serum PK in an expected range typical 2+1 TfR antibodies. Remternetug-TfRL7-2 hIgG1 WT and Remternetug-TfR39.38 hIgG1 WT displayed faster peripheral clearance compared to Remternetug-TfRL10-16 hIgG1 WT, possibly due to anti-drug antibody development (ADA) in these mice.

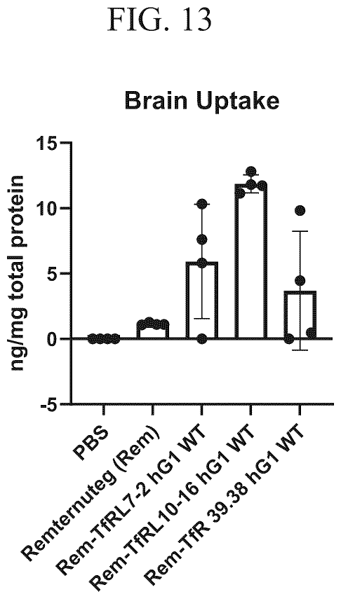

To determine brain penetration of 2+1 anti-TfR antibodies coupled with anti-Ap antibody cargo, brains of the animals were collected 24 hours after the second dose to determine levels of brain uptake (FIG. 13). Analyses were performed using vessel-depleted brain lysates to measure IgG levels in the brain parenchyma, as described previously (See International Patent Publication No. WO2024/026472). Antibody concentrations were normalized to total protein concentration in the lysate and then graphed as antibody concentration (ng antibody/mg total protein). All Remternetug-TfR antibodies tested showed higher brain uptake compared to Remternetug without an anti-TfR antigen-binding component. Remternetug-TfR L10-16 hIgG1 WT group showed the highest and most consistent increase in brain concentration levels. Variable levels of brain concentration observed in the Rem-TfRL7-2 and Rem-TfR39.38 groups could possibly be due to the development of ADA that was observed from low serum PK at day 14 following administration of the first dose.

Taken together, these studies showed that anti-TfR antigen-binding domain antibodies of the present disclosure resulted in strong brain uptake of anti-Abeta (Aβ) antibodies at low dose (3 mg/kg). As anti-TfRL10-16 displayed good serum PK parameters, did not reduce reticulocyte counts, and did not reduce TfR levels on reticulocytes at low dose with hIgG1 WT Fc, use of this anti-TfR antigen-binding domain antibody paired with an anti-Abeta (Aβ) antibody (e.g., in a 2+1 format) antibody of the present disclosure should provide a means for increased CNS delivery of the anti-Abeta (Aβ) antibody together with a favorable safety profile.

### Example 16: In Vivo Murine Studies with Anti-pyroGlu3 Abeta (Aβ) Human TfR Antibodies

To assess brain uptake and safety of anti-pyroGlu3 Abeta (Aβ) huTfR antibodies of the present disclosure in a 2+1 format, a single dose acute study is conducted in human TfR KI mice. At 24 h post dosing, the blood, brain, spleen, and liver are harvested from the animals. Blood is measured for complete blood count analysis. Vessel depleted brain lysates and terminal serum samples are measured for antibody levels. Changes in TfR levels in brain, spleen, and liver area are also measured.

Similarly, a multi-dose study is conducted in human TfR KI mice administered three weekly doses of anti-pyroGlu3 Abeta (Aβ) huTfR antibodies in a 2+1 format of the present disclosure. Blood samples are collected in life at day 1, and 24 h prior to administration of each dose for reticulocyte measurement by FACs; serum antibody levels are also measured. At 24h post final dose, the animals are taken down and whole blood, brains, spleens, and livers are harvested and processed for similar measurements as the single dose acute study described above. ADA levels are also characterized for any antibody groups showing signs of non-linear pharmacokinetics.

Anti-pyroGlu3 Abeta (Aβ) human TfR antibodies in a 2+1 format are also assessed for nonspecific binding with a single dose PK study in wild-type mice. Sera are collected from the animals at 1 h, and at day 1, day 4, day 7, and 14 post dosing and analyzed for antibody levels and peripheral clearance, which are compared to that observed with control huIgG.

### Example 17: In Vivo Studies with Anti-pyroGlu3 Abeta (Aβ) Murine TfR IgG2a Surrogates in a 2+1Format

To investigate brain uptake and safety of murine surrogate anti-pyroGlu3 Abeta (Aβ) TfR IgG2a antibodies in a 2+1 format, a single low dose acute study is conducted in aged 5XFAD mice (expressing murine TfR surrogate). At 24 h post dosing, the blood, brain, spleen, and liver are harvested. Blood is measured for complete blood count analysis. Vessel-depleted brain lysates and serum samples are assessed for antibody levels. TfR levels in brain, spleen, and liver are measured by western blot. Target engagement on amyloid plaques is also assessed in brain sections of the animals.

Similarly, a multi-dose study is conducted in aged 5XFAD mice with 3 weekly low dose antibody administration. Blood samples are collected in life for reticulocyte measurements and serum PK analysis. At 24 h following the final dose, blood, brains, spleens, and livers are harvested and processed with similar measurements as the single dose acute study described above. A chronic multidose efficacy study is performed to assess the extent of plaque and amyloid beta reductions over 12 weekly doses. Brain tissue is harvested for Abeta (Aβ) plaque staining and lysates generated to measure changes in Abeta (Aβ) levels at study termination.

Additionally, murine dose-range finding studies are performed at low and high dose levels in aged 5XFAD mice to acquire the toxicokinetic profile of anti-pyroGlu3 Abeta (Aβ) antibodies of the present disclosure on a murine TfR ms IgG2a backbone. A separate toxicology cohort of mice is assessed for clinical, histological, and hematological pathology after receiving 5 weekly doses. Brains are collected and processed for anti-amyloid-beta staining and vessel depletion to measure uptake of dosed antibodies.

An additional study in aged 5XFAD is used to assess whether amyloid-related imaging abnormalities (ARIA) are observed or at a higher risk with anti-pyroGlu3 Abeta (Aβ) antibodies with TfR binding. Mice are pre-imaged using MRI to establish baseline images and at various intervals after receiving high and low doses of antibody chronically over the course of 12 weeks.

### Example 18: Non-Human Primate (NHP) Dose Range Finding (DRF) Study of Anti-pyroGlu3 Abeta (Aβ)-huTfR Antibodies

In this Example and all following Examples, the term non-human primate or NHP refers to cynomolgous fasicularis monkeys.

A PK study in non-human primates following weekly IV administration of anti-pyroGlu3 Abeta (Aβ) antibodies of the present disclosure is conducted to assess antibody levels in serum and safety of the dosed antibodies. Serum, CSF, peripheral, and brain tissues are collected. The study includes 2 dose levels of anti-pyroGlu3 Abeta (Aβ) antibodies (high and low) as well as a control, non-BBB targeting anti-pyroGlu3 Abeta (Aβ) antibodies. Standard safety assessments (e.g., mortality, clinical signs, body weight, clinical pathology, as well as macro- and microscopic evaluation) are performed.

### Example 19: Assessment of Characteristics for Manufacturing of Anti-PyroGlu3 Abeta (Aβ)-huTfR Antibodies in a 2+1 Format

The physiochemical and biochemical properties of a protein can impact protein stability and can pose potential risks during manufacturing. An assessment is conducted by testing selected anti-pyroGlu3 Abeta (Aβ)-huTfR antibodies in a 2+1 format, to determine and identify these characteristics. Several analytical techniques are utilized including hydrophobic interaction, freeze/thaw, heat stress, thermostability, concentratability, demonstrating that the properties of those selected antibodies met the criteria for further development.

### Example 20: Assessment of Characteristics of Disulfide Stapled TfR scFv

Single-chain fragment variable (scFv) antibody formats consist of 2 variable domains (VH and VL) that are linked by a flexible linker. Due to weak interaction between VH and VL domains, some scFvs may show increased risk of aggregation or may display reduced stability. To overcome this, two mutations are introduced into the VH and VL framework (VH44 and VL100) to introduce a disulfide bond (e.g., a disulfide staple) between the VH and VL domain (Reiter et al., 1994, Biochemistry, 33:5451-5459 and Wetherill et al., 2012, Protein Engineering, Design & Selection, 25:321-329). A “disulfide staple” refers to the engineering of one or more disulfide bonds to stabilize a molecule's structure, enhance its stability, or improve any functional properties compared to the molecule without the engineered disulfide staple. An example sequence of TfRL10-16 with disulfide stapled mutations in VH-VL and VL-VH orientation is listed below in Table 15, Table 16 and Table 17 provide CDR sequences of these constructs). Several properties of the disulfide stapled TfR scFvs are assessed, including affinity to human and cyno TfR apical domain, manufacturing characteristics (e.g., hydrophobic interaction, freeze/thaw, heat stress, thermostability, concentratability), and in vivo characteristics (e.g., brain uptake, reticulocytes count). Results from this study suggest that disulfide stapled TfRL10-16 scFvs show less risk of aggregation, maintain affinities in the desired range, exhibit good brain uptake, and display minimum reticulocyte reduction.

TABLE 15
VH and VL Sequences of disulfide stapled TfR scFv
(mutations to introduce a disulfide bond underlined)

| Antibody | Sequence |
|---|---|
| Disulfide stapled | QVQLVQSGAEVKKPGASVKVSCKASGYTFTSYYMH |
| TfR.15.WH8.1.42Q.L10-16 | WVRQAPGQCLEWMGNIYPGSGSSNYAQKFQGRVTM |
| Mutate AA to cysteines at | TRDTSTSTVYMELSSLRSEDTAVYYCTRSSYYGNFDT |
| VH44 and VL100 | MDNWGQGTTVTVSSGGSEGKSSGSGSESKSTGGSEIV |
| (in VH-VL orientation) | LTQSPATLSLSPGERATLSCRASSSIRQMHWYQQKPG |
| | QAPRRLIYDTSNRATGIPARFSGSGSGTDFTLTISSL |
| | EPEDFAVYYCHQRSSYPWTFGCGTKVEIK |
| | (SEQ ID NO: 202) |
| Disulfide stapled | EPEDFAVYYCHQRSSYPWTFGCGTKVEIKGGSEGKSS |
| TfR.15.WH8.1.42Q.L10-16 | EIVLTQSPATLSLSPGERATLSCRASSSIRQMHWYQQK |
| Mutate AA to cysteines at VH44 | PGQAPRRLIYDTSNRATGIPARFSGSGSGTDFTLTISS |
| VL100 (in VL-VH orientation) | LGSGSESKSTGGSQVQLVQSGAEVKKPGASVKVSCKAS |
| | GYTFTSYYMHWVRQAPGQCLEWMGNIYPGSGSSNY |
| | AQKFQGRVTMTRDTSTSTVYMELSSLRSEDTAVYYC |
| | TRSSYYGNFDTMDNWGQGTTVTVSS (SEQ ID |
| | NO: 203) |

TABLE 16
CDR sequences of disulfide stapled
TfR.15.WH8.1.42Q.L10-16 VH-VL scFv

| Antibody | CDR-H1 | CDR-H2 | CDR-H3 |
|---|---|---|---|
| TfR.15.WH8.1. | SYYMH | NIYPGSGSS | SSYYGNF |
| -42Q.L1016 HL | (SEQ ID | NYAQKFQG | DTMDN |
| scFv | NO: 56) | (SEQ ID | (SEQ ID |
| | | NO: 57) | NO: 59) |
| | CDR-L1 | CDR-L2 | CDR-L3 |
| | RASSSIRQM | DTSNRAT | HQRSSYPWT |
| | H (SEQ ID | (SEQ ID | (SEQ ID |
| | NO: 73) | NO: 67) | NO: 68) |

TABLE 17
CDR sequences of disulfide stapled
TfR.15.WH8.1.42Q.L10-16 VL-VH scFv

| Antibody | CDR-H1 | CDR-H2 | CDR-H3 |
|---|---|---|---|
| TfR.15.WH8. | SYYMH | NIYPGSGS | SSYYGNF |
| 1.42Q.L10 | (SEQ ID | SNYAQKFQ | DTMDN |
| | NO: 56) | G (SEQ ID | (SEQ ID |
| | | NO: 57) | NO: 59) |
| | CDR-L1 | CDR-L2 | CDR-L3 |
| | RASSSIRQM | DTSNRAT | HQRSSYPWT |
| | H (SEQ ID | (SEQ ID | (SEQ ID |
| | NO: 73) | NO: 67) | NO: 68) |

Provided below (Table 18) is the amino acid sequence of antibody 8D3v12 scFv, a mouse surrogate TfR scFv (VH-linker-VL format) for use in similar studies (does not include a linker between Fc and scFv (e.g., GGSGG (SEQ ID NO:214)).

(SEQ ID NO: 195)
EVQLVESGGGLVQPGNSLTLSCVASGFTFSNYGMHWIRQAPKKGLE
WIAAIYYDSSKMNYADTVKGRFTISRDNSKNTLYLEMNSLRSEDT
AMYYCAVPTSHYVVDVWGQGVSVTVSSGGSEGKSSGSGSESKSTG
GSDIQMTQSPASLSASLEEIVTITCQASQDIGNWLAWYQQKPGKS
PQLLIYGATSLADGVPSRFSGSRSGTQFSLKISRVQVEDIGIYYC
LQAYNTPWTFGGGTKLELK

TABLE 18

| Antibody | CDR-H1 | CDR-H2 | CDR-H3 |
|---|---|---|---|
| 8D3v12 scFv | NYGMH | AIYYDSSKM | PTSHYV |
| mouse surrogate | (SEQ ID | NYADTVKG | VDV |
| TfR scFv (VH- | NO: 196) | (SEQ ID | (SEQ ID |
| linker-VL format) | | NO: 197) | NO: 198) |
| | CDR-L1 | CDR-L2 | CDR-L3 |
| | QASQDIGNWLA | GATSLAD | LQAYNTPWT |
| | (SEQ ID | (SEQ ID | (SEQ ID |
| | NO: 199) | NO: 200) | NO: 201) |

### Example 21: Amino Acid Sequences of Select Anti-pyroGlu3 Abeta (Aβ)Antigen-Binding Domain and an Anti-TfR Antigen Binding Domain Compositions of the Present Disclosure

Provided below in Table 19 are amino acid sequences of select anti-pyroGlu3 Abeta (Aβ) antigen-binding domain/anti-TfRL10-16 scFv antigen binding domain compositions in 2+1 format. Provided below in Table 20 are amino acid sequences of murine anti-pyroGlu3 Aβ antibodies/anti-TfR surrogate scFvs (msTfR 8D3v12) in a 2+1 format.

TABLE 19

| | Anti-Abeta (Aβ) VH (WT | | Anti-Abeta (Aβ) |
|---|---|---|---|
| | huIgG1) + Knob Fc + | Anti-Abeta | VL + huKappa |
| | Linker + scFv | (Aβ) VH (WT | constant |
| | TfRL10-16 (2+1) | huIgG1) + Hole Fc | (LC) |
| AB-6/TfRL10-16 | QLQLQESGPGLVKPSET | QLQLQESGPGLVKPSETL | DIQMTQSPSSLS |
| | LSLTCTVSGGSISSGSYY | SLTCTVSGGSISSGSYYW | ASVGDRVTITC |
| | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | TYSTPITFGQGT |
| | GCLVKDYFPEPVTVSW | DYFPEPVTVSWNSGALT | RLEIKRTVAAPS |
| | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 225) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK | |
| | LSLSPGKGGSGGQVQLV | (SEQ ID NO: 224) | |
| | QSGAEVKKPGASVKVS | | |
| | CKASGYTFTSYYMHWV | | |
| | RQAPGQGLEWMGNIYP | | |
| | GSGSSNYAQKFQGRVT | | |
| | MTRDTSTSTVYMELSSL | | |
| | RSEDTAVYYCTRSSYYG | | |
| | NFDTMDNWGQGTTVT | | |
| | VSSGGSEGKSSGSGSES | | |
| | KSTGGSEIVLTQSPATLS | | |
| | LSPGERATLSCRASSSIR | | |
| | QMHWYQQKPGQAPRR | | |
| | LIYDTSNRATGIPARFSG | | |
| | SGSGTDFTLTISSLEPED | | |
| | FAVYYCHQRSSYPWTF | | |
| | GGGTKVEIK (SEQ ID | | |
| | NO: 223) | | |
| AB-7/TfRL10-16 | QLQLQESGPGLVKPSET | QLQLQESGPGLVKPSETL | DIQMTQSPSSLS |
| | LSLTCTVSGGSTSSSSYY | SLTCTVSGGSTSSSSYYW | ASVGDRVTITC |
| | WGWIRQPPGKGLEWIG | GWIRQPPGKGLEWIGSIY | RASQSISNYLN |
| | SIYFSGSTYYNPSLKSRV | FSGSTYYNPSLKSRVTIS | WYQQKPGKAP |
| | TISVDTSKNQFSLKLSSV | VDTSKNQFSLKLSSVTA | KLLIYAASSLQS |
| | TAADTAVYYCVRHREM | ADTAVYYCVRHREMATI | GVPSRFSGSGS |
| | ATIRDYHYYYMDVWG | RDYHYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | SYSTPLTFGGGT |
| | GCLVKDYFPEPVTVSW | DYFPEPVTVSWNSGALT | KVEIKRTVAAP |
| | NSGALTSGVHTFPAVLQ | SGVHTFPAVLQSSGLYSL | SVFIFPPSDEQL |
| | SSGLYSLSSVVTVPSSSL | SSVVTVPSSSLGTQTYIC | KSGTASVVCLL |
| | GTQTYICNVNHKPSNTK | NVNHKPSNTKVDKKVEP | NNFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 228) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK | |
| | LSLSPGKGGSGGQVQLV | (SEQ ID NO: 227) | |
| | QSGAEVKKPGASVKVS | | |
| | CKASGYTFTSYYMHWV | | |
| | RQAPGQGLEWMGNIYP | | |
| | GSGSSNYAQKFQGRVT | | |
| | MTRDTSTSTVYMELSSL | | |
| | RSEDTAVYYCTRSSYYG | | |
| | NFDTMDNWGQGTTVT | | |
| | VSSGGSEGKSSGSGSES | | |
| | KSTGGSEIVLTQSPATLS | | |
| | LSPGERATLSCRASSSIR | | |
| | QMHWYQQKPGQAPRR | | |
| | LIYDTSNRATGIPARFSG | | |
| | SGSGTDFTLTISSLEPED | | |
| | FAVYYCHQRSSYPWTF | | |
| | GGGTKVEIK (SEQ ID | | |
| | NO: 226) | | |
| AB-8/TfRL10-16 | EVQLVESGGGLIQPGGS | EVQLVESGGGLIQPGGSL | DIQMTQSPSSLS |
| | LRLSCAASGFTVSSNYM | RLSCAASGFTVSSNYMS | ASVGDRVTITC |
| | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK | |
| | KGGSGGQVQLVQSGAE | (SEQ ID NO: 230) | |
| | VKKPGASVKVSCKASG | | |
| | YTFTSYYMHWVRQAPG | | |
| | QGLEWMGNIYPGSGSS | | |
| | NYAQKFQGRVTMTRDT | | |
| | STSTVYMELSSLRSEDT | | |
| | AVYYCTRSSYYGNFDT | | |
| | MDNWGQGTTVTVSSGG | | |
| | SEGKSSGSGSESKSTGG | | |
| | SEIVLTQSPATLSLSPGE | | |
| | RATLSCRASSSIRQMHW | | |
| | YQQKPGQAPRRLIYDTS | | |
| | NRATGIPARFSGSGSGT | | |
| | DFTLTISSLEPEDFAVYY | | |
| | CHQRSSYPWTFGGGTK | | |
| | VEIK (SEQ ID NO: 229) | | |

TABLE 20

| | Anti-Abeta (Aβ) VH (WT | | |
|---|---|---|---|
| | huIgG1) + Knob Fc + | Anti-Abeta (Aβ) | Anti-Abeta (Aβ) |
| | Linker + scFv | VH (WT | VL + huKappa |
| | msTfR8D3v12 (2+1) | huIgG1) + Hole Fc | constant (LC) |
| AB-6/msTfR 8D3v12 | QLQLQESGPGLVKPSET | QLQLQESGPGLVKPSETL | DIQMTQSPSSLS |
| | LSLTCTVSGGSISSGSYY | SLTCTVSGGSISSGSYYW | ASVGDRVTITC |
| | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSAKTTAPSV | VTVSSAKTTAPSVYPLAP | PEDFATYYCQQ |
| | YPLAPVCGDTTGSSVTL | VCGDTTGSSVTLGCLVK | TYSTPITFGQGT |
| | GCLVKGYFPEPVTLTW | GYFPEPVTLTWNSGSLSS | RLEIKRADAAP |
| | NSGSLSSGVHTFPAVLQ | GVHTFPAVLQSDLYTLSS | TVSIFPPSSEQLT |
| | SDLYTLSSSVTVTSSTW | SVTVTSSTWPSQSITCNV | SGGASVVCFLN |
| | PSQSITCNVAHPASSTK | AHPASSTKVDKKIEPRGP | NFYPKDINVKW |
| | VDKKIEPRGPTIKPCPPC | TIKPCPPCKCPAPNLLGG | KIDGSERQNGV |
| | KCPAPNLLGGPSVFIFPP | PSVFIFPPKIKDVLMISLS | LNSWTDQDSK |
| | KIKDVLMISLSPIVTCVV | PIVTCVVVDVSEDDPDV | DSTYSMSSTLT |
| | VDVSEDDPDVQISWFV | QISWFVNNVEVHTAQTQ | LTKDEYERHNS |
| | NNVEVHTAQTQTHRED | THREDYNSTLRVVSALPI | YTCEATHKTST |
| | YNSTLRVVSALPIQHQD | QHQDWMSGKEFKCKVN | SPIVKSFNRNEC |
| | WMSGKEFKCKVNNKD | NKDLPAPIERTISKPKGS | (SEQ ID NO: 234) |
| | LPAPIERTISKPKGSVRA | VRAPQVYVLPPPEEEMT | |
| | PQVYVLPPPEEEMTKKQ | KKQVTLSCAVTDFMPED | |
| | VTLWCMVTDFMPEDIY | IYVEWTNNGKTELNYKN | |
| | VEWTNNGKTELNYKNT | TEPVLDSDGSYFMVSKL | |
| | EPVLDSDGSYFMYSKLR | RVEKKNWVERNSYSCSV | |
| | VEKKNWVERNSYSCSV | VHEGLHNHHTTKSFSRT | |
| | VHEGLHNHHTTKSFSRT | PGK (SEQ ID NO: 233) | |
| | PGKGGSGGEVQLVESG | | |
| | GGLVQPGNSLTLSCVAS | | |
| | GFTFSNYGMHWIRQAP | | |
| | KKGLEWIAAIYYDSSK | | |
| | MNYADTVKGRFTISRD | | |
| | NSKNTLYLEMNSLRSED | | |
| | TAMYYCAVPTSHYVVD | | |
| | VWGQGVSVTVSSGGSE | | |
| | GKSSGSGSESKSTGGSDI | | |
| | QMTQSPASLSASLEEIVT | | |
| | ITCQASQDIGNWLAWY | | |
| | QQKPGKSPQLLIYGATS | | |
| | LADGVPSRFSGSRSGTQ | | |
| | FSLKISRVQVEDIGIYYC | | |
| | LQAYNTPWTFGGGTKL | | |
| | ELK (SEQ ID NO: 232) | | |
| AB-7/msTfR 8D3v12 | QLQLQESGPGLVKPSET | QLQLQESGPGLVKPSETL | DIQMTQSPSSLS |
| | LSLTCTVSGGSTSSSSYY | SLTCTVSGGSTSSSSYYW | ASVGDRVTITC |
| | WGWIRQPPGKGLEWIG | GWIRQPPGKGLEWIGSIY | RASQSISNYLN |
| | SIYFSGSTYYNPSLKSRV | FSGSTYYNPSLKSRVTIS | WYQQKPGKAP |
| | TISVDTSKNQFSLKLSSV | VDTSKNQFSLKLSSVTA | KLLIYAASSLQS |
| | TAADTAVYYCVRHREM | ADTAVYYCVRHREMATI | GVPSRFSGSGS |
| | ATIRDYHYYYMDVWG | RDYHYYYMDVWGKGTT | GTDFTLTISSLQ |
| | KGTTVTVSSAKTTAPSV | VTVSSAKTTAPSVYPLAP | PEDFATYYCQQ |
| | YPLAPVCGDTTGSSVTL | VCGDTTGSSVTLGCLVK | SYSTPLTFGGGT |
| | GCLVKGYFPEPVTLTW | GYFPEPVTLTWNSGSLSS | KVEIKRADAAP |
| | NSGSLSSGVHTFPAVLQ | GVHTFPAVLQSDLYTLSS | TVSIFPPSSEQLT |
| | SDLYTLSSSVTVTSSTW | SVTVTSSTWPSQSITCNV | SGGASVVCFLN |
| | PSQSITCNVAHPASSTK | AHPASSTKVDKKIEPRGP | NFYPKDINVKW |
| | VDKKIEPRGPTIKPCPPC | TIKPCPPCKCPAPNLLGG | KIDGSERQNGV |
| | KCPAPNLLGGPSVFIFPP | PSVFIFPPKIKDVLMISLS | LNSWTDQDSK |
| | KIKDVLMISLSPIVTCVV | PIVTCVVVDVSEDDPDV | DSTYSMSSTLT |
| | VDVSEDDPDVQISWFV | QISWFVNNVEVHTAQTQ | LTKDEYERHNS |
| | NNVEVHTAQTQTHRED | THREDYNSTLRVVSALPI | YTCEATHKTST |
| | YNSTLRVVSALPIQHQD | QHQDWMSGKEFKCKVN | SPIVKSFNRNEC |
| | WMSGKEFKCKVNNKD | NKDLPAPIERTISKPKGS | (SEQ ID NO: 237) |
| | LPAPIERTISKPKGSVRA | VRAPQVYVLPPPEEEMT | |
| | PQVYVLPPPEEEMTKKQ | KKQVTLSCAVTDFMPED | |
| | VTLWCMVTDFMPEDIY | IYVEWTNNGKTELNYKN | |
| | VEWTNNGKTELNYKNT | TEPVLDSDGSYFMVSKL | |
| | EPVLDSDGSYFMYSKLR | RVEKKNWVERNSYSCSV | |
| | VEKKNWVERNSYSCSV | VHEGLHNHHTTKSFSRT | |
| | VHEGLHNHHTTKSFSRT | PGK (SEQ ID NO: 236) | |
| | PGKGGSGGEVQLVESG | | |
| | GGLVQPGNSLTLSCVAS | | |
| | GFTFSNYGMHWIRQAP | | |
| | KKGLEWIAAIYYDSSK | | |
| | MNYADTVKGRFTISRD | | |
| | NSKNTLYLEMNSLRSED | | |
| | TAMYYCAVPTSHYVVD | | |
| | VWGQGVSVTVSSGGSE | | |
| | GKSSGSGSESKSTGGSDI | | |
| | QMTQSPASLSASLEEIVT | | |
| | ITCQASQDIGNWLAWY | | |
| | QQKPGKSPQLLIYGATS | | |
| | LADGVPSRFSGSRSGTQ | | |
| | FSLKISRVQVEDIGIYYC | | |
| | LQAYNTPWTFGGGTKL | | |
| | ELK (SEQ ID NO: 235) | | |
| AB-8/msTfR 8D3v12 | EVQLVESGGGLIQPGGS | EVQLVESGGGLIQPGGSL | DIQMTQSPSSLS |
| | LRLSCAASGFTVSSNYM | RLSCAASGFTVSSNYMS | ASVGDRVTITC |
| | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSAKTTAPSVYPL | TVSSAKTTAPSVYPLAPV | PEDFATYYCQQ |
| | APVCGDTTGSSVTLGCL | CGDTTGSSVTLGCLVKG | SYSIPPTFGGGT |
| | VKGYFPEPVTLTWNSGS | YFPEPVTLTWNSGSLSSG | KVEIKRADAAP |
| | LSSGVHTFPAVLQSDLY | VHTFPAVLQSDLYTLSSS | TVSIFPPSSEQLT |
| | TLSSSVTVTSSTWPSQSI | VTVTSSTWPSQSITCNVA | SGGASVVCFLN |
| | TCNVAHPASSTKVDKKI | HPASSTKVDKKIEPRGPT | NFYPKDINVKW |
| | EPRGPTIKPCPPCKCPAP | IKPCPPCKCPAPNLLGGP | KIDGSERQNGV |
| | NLLGGPSVFIFPPKIKDV | SVFIFPPKIKDVLMISLSPI | LNSWTDQDSK |
| | LMISLSPIVTCVVVDVSE | VTCVVVDVSEDDPDVQI | DSTYSMSSTLT |
| | DDPDVQISWFVNNVEV | SWFVNNVEVHTAQTQT | LTKDEYERHNS |
| | HTAQTQTHREDYNSTL | HREDYNSTLRVVSALPIQ | YTCEATHKTST |
| | RVVSALPIQHQDWMSG | HQDWMSGKEFKCKVNN | SPIVKSFNRNEC |
| | KEFKCKVNNKDLPAPIE | KDLPAPIERTISKPKGSVR | (SEQ ID NO: |
| | RTISKPKGSVRAPQVYV | APQVYVLPPPEEEMTKK | 240) |
| | LPPPEEEMTKKQVTLW | QVTLSCAVTDFMPEDIY | |
| | CMVTDFMPEDIYVEWT | VEWTNNGKTELNYKNT | |
| | NNGKTELNYKNTEPVL | EPVLDSDGSYFMVSKLR | |
| | DSDGSYFMYSKLRVEK | VEKKNWVERNSYSCSVV | |
| | KNWVERNSYSCSVVHE | HEGLHNHHTTKSFSRTP | |
| | GLHNHHTTKSFSRTPGK | GK (SEQ ID NO: 239) | |
| | GGSGGEVQLVESGGGL | | |
| | VQPGNSLTLSCVASGFT | | |
| | FSNYGMHWIRQAPKKG | | |
| | LEWIAAIYYDSSKMNY | | |
| | ADTVKGRFTISRDNSKN | | |
| | TLYLEMNSLRSEDTAM | | |
| | YYCAVPTSHYVVDVWG | | |
| | QGVSVTVSSGGSEGKSS | | |
| | GSGSESKSTGGSDIQMT | | |
| | QSPASLSASLEEIVTITC | | |
| | QASQDIGNWLAWYQQ | | |
| | KPGKSPQLLIYGATSLA | | |
| | DGVPSRFSGSRSGTQFS | | |
| | LKISRVQVEDIGIYYCLQ | | |
| | AYNTPWTFGGGTKLEL | | |
| | K (SEQ ID NO: 238) | | |

### Example 22: Screening Anti-pyroGlu3 Abeta (Aβ)-huTfR Antibodies by Biological Functional Assays

Anti-pyroGlu3 Abeta (Aβ)-TfR antibodies of the present disclosure were further screened for their biological activities in an ex vivo amyloid plaque phagocytosis assay as follows. Frozen brain tissue from 5XFAD mice were sectioned to 20 μm and mounted on poly-D-lysine-coated cover glass (15 mm) and placed in 24-well tissue culture plates. Consecutive sections were positioned in the same order as sectioned and were incubated with or without antibodies (67 nM, 500 ml, 1 hr, room temperature). Primary human macrophages (5×10⁵ cells per well) were then added to the sections and incubated for 24 h at 37 °C. At the end of incubation, media were removed, brain sections and cells were homogenized with 150 μL guanidine HCL lysis buffer (5.5M guanidine+50 mM Tris+1X protease inhibitor cocktail), and Aβ₁₋₄₂ polypeptide concentration quantified by ELISA as described previously. The level of residual Abeta (Aβ) was normalized to that observed in isotype control group. The ability of anti-pyroGlu3 Abeta (Aβ) antibodies of the present disclosure to display plaque phagocytosis in this assay was compared to that observed with anti-Abeta (Aβ) antibodies donanemab and remternetug.

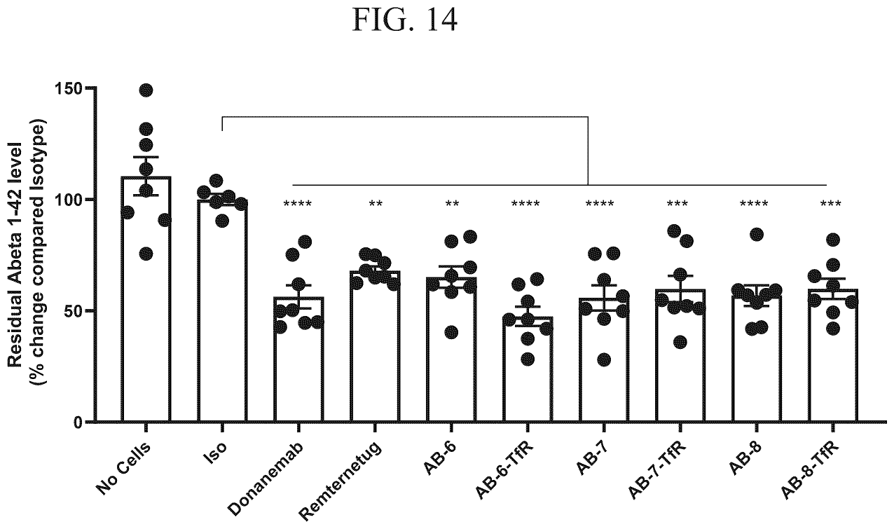

As shown in FIG. 14, anti-pyroGlu3 Abeta (Aβ)-huTfR antibodies at 67 nM showed ~50% Abeta (Aβ)₁₋₄₂ polypeptide clearance compared to that observed with isotype control. Additionally, the extent of Abeta (Aβ)₁₋₄₂ polypeptide phagocytosis (e.g., plaque clearance) observed with anti-pyroGlu3 Abeta (Aβ)-huTfR antibodies (AB-6/TfRL10-16, AB-7/TfRL10-16, and AB-8/TfRL10-16) of the present disclosure was similar to that observed with anti-pyroGlu3 Abeta (Aβ) antibodies (AB-6, AB-7 and AB-8) that did not comprise a TfR-binding moiety.

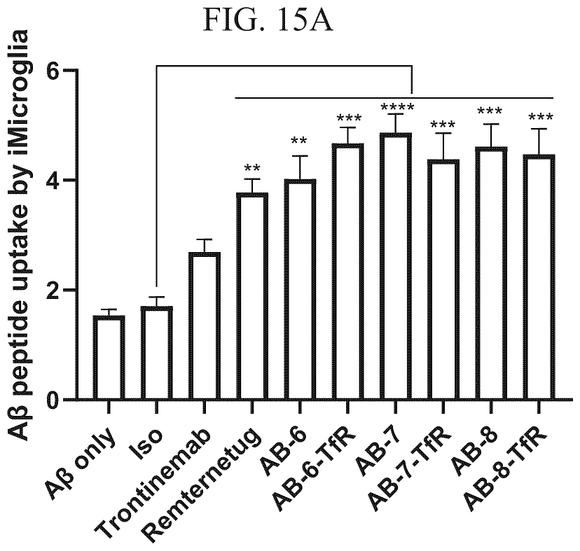

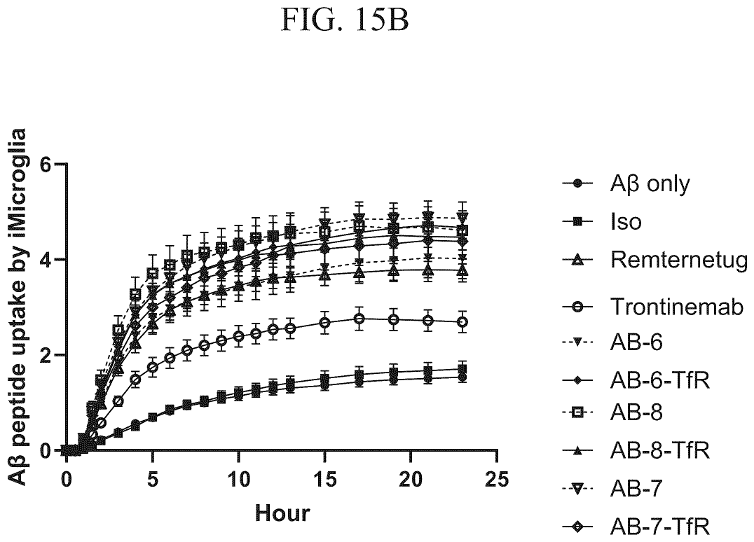

To investigate whether anti-pyroGlu3 Abeta (Aβ)-huTfR antibodies of the present disclosure promote uptake of pyroGlu3 Abeta (Aβ) by microglia in the CNS, a human CNS triple culture cell model was used. CNS triple culture cells were simultaneously incubated with anti-pyroGlu3 Abeta (Aβ) hybridoma antibodies of the present disclosure (6.7 nM) and pyroGlu3-24 Abeta (Aβ) polypeptides (1 μM) labeled with fluorescent pH-sensitive pHrodo-Red dye for 24h. Uptake of pyroGlu3-24 Abeta (Aβ) by microglia in this human CNS triple culture cell system was monitored by IncuCyte in real time. The fluorescence intensity of pHrodo-Red signal was normalized to GFP tagged microglia to evaluate the effect of microglial pyroGlu3-24 Abeta (Aβ) phagocytosis for antibody screening. FIG. 15A shows data from the 24 hr time point in this study (one way ANOVA with Tukey's multiple comparisons test. **: p<0.005; ***: p<0.001; **** p<0.0001). FIG. 15B shows the time course of Abeta (Aβ) peptide uptake in this study. As shown in FIG. 15A and FIG. 15B, anti-pyroGlu3 Abeta (Aβ) antibodies of the present disclosure (with or without a TfR-binding moiety) showed significant increases in microglial pyroGlu3 Abeta (Aβ) phagocytosis. As shown in FIG. 15A, anti-pyroGlu3 Abeta (Aβ) antibodies of the present disclosure (AB-7=p<0.0001; AB-8/TfRL10-16, AB-8, AB-7/TfRL10-16, and AB-6/TfRL10-16=p<0.001) demonstrated a higher increase in microglial pyroGlu3 Abeta (Aβ) phagocytosis than that observed with either anti-Abeta (Aβ) antibody trontinemab or remternetug when compared to the isotype control group. AB-6 performed similarly to remternetug (p<0.005). These data indicated that anti-pyroGlu3 Abeta (Aβ) antibodies of the present disclosure were effective at phagocytosis of Abeta (Aβ) plaques.

### Example 23: Cell Uptake Studies of Anti-pyroGlu3 Abeta (Aβ) Antibodies in hCMEC/D3 Cell Line

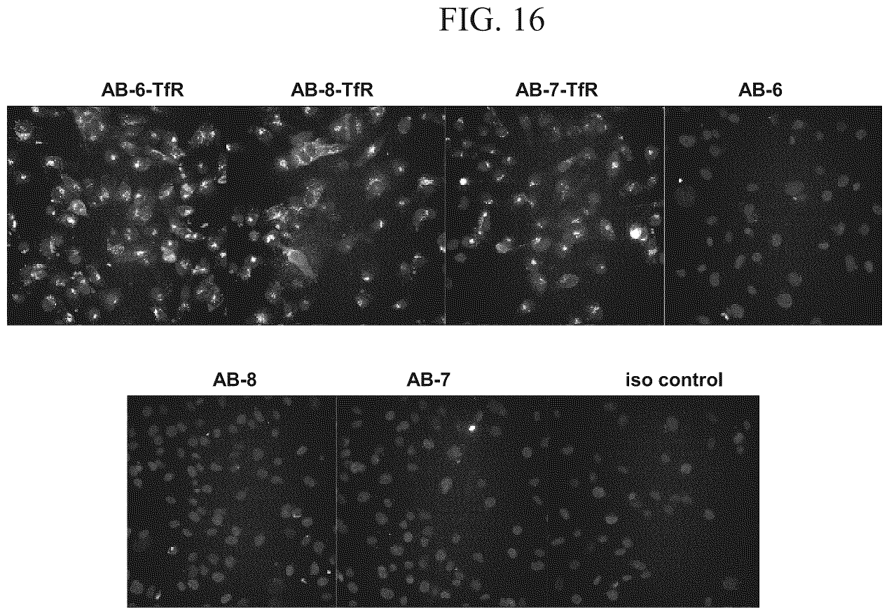

The extent of cellular uptake of anti-pyroGlu3 Abeta (Aβ) antibodies with or without an anti-human TfRL10-16 binding moiety was assessed using human brain endothelial-like cell line hCMEC/D3. An acute cell trafficking assay was established to quantify the uptake of antibodies after a 2 hr incubation at 37 °C HCMEC/D3 cells were seeded onto 96-well microtiter plates. After 24-72 hrs, the cells were incubated with various anti-pyroGlu3 Abeta (Aβ) antibodies of the present disclosure diluted into fresh media. After 2 hrs incubation, the cells were washed once in PBS, fixed in paraformaldehyde, and blocked in 1% BSA, 0.2% saponin diluted into PBS for 1 h. Anti-human IgG antibody labeled with Alexa-Fluor-488 was added and incubated overnight. Nuclei were labeled with DAPI. Images were obtained using the Operetta CLS (Revvity). FIG. 16 shows the extent of cellular uptake of anti-pyroGlu3 Abeta (Aβ) antibodies with and without the TfRL10-16 binding moiety compared to that of isotype controls. Antibodies having a TfR binding moiety exhibited the highest level of cellular uptake in hCMEC/D3 cells in this assay. No uptake was observed with non-TfR binding antibodies or the isotype control.

### Example 24: In Vivo Brain Uptake and Serum PK of Anti-pyroGlu3 Abeta (Aβ)-huTfR Antibodies

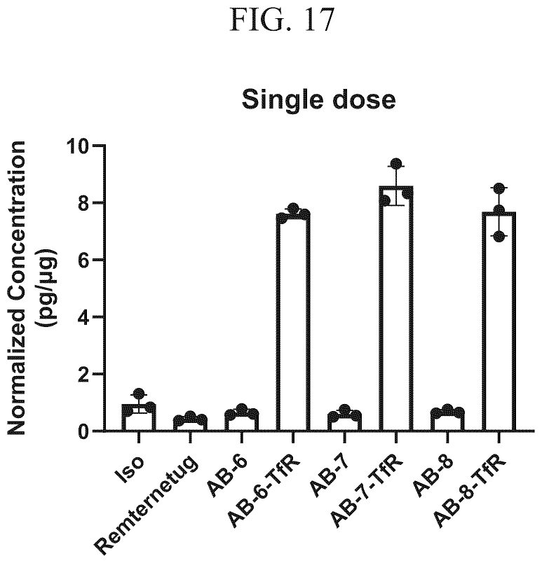

An in vivo study in hu-TfR KI mice was performed to investigate brain uptake and serum PK of anti-pyroGlu3 Abeta (Aβ)-huTfR antibodies of the present disclosure using a single dose regime. In this single dose study, eight- to twelve-week-old female homozygous huTfR KI mice were weighed and dosed via intravenous (i.v.) injection of the tail vein with 3 mg/kg of IgG control, anti-Abeta (Aβ) antibody remternetug, three anti-pyroGlu3 Abeta (Aβ) antibodies without a TfR binding moiety (AB-6, AB-7, and AB-8; also called “naked anti-pyroGlu3 Abeta (Aβ) antibodies”), and anti-pyroGlu3 Abeta (Aβ)-huTfR antibodies (AB-6/TfRL10-16, AB-7/TfRL10-16, and AB-8/TfRL10-16). Brains were collected 24 hours post-dose to determine brain uptake of the antibodies (FIG. 17). Analyses were performed using vessel-depleted brain lysates to measure IgG levels in the brain parenchyma, as described previously (WO2024026472A2). Antibody concentrations were normalized to the total protein concentration in the lysate and then graphed as antibody concentration (pg antibody/g total protein). Remternetug and anti-pyroGlu3 Abeta (Aβ) antibodies without a TfR binding moiety showed similar brain uptake as observed with isotype control. All three anti-pyroGlu3 Abeta (Aβ)-huTfR antibodies tested showed much higher brain uptake in the mice compared to that observed with anti-pyroGlu3 Abeta (Aβ) antibodies without a TfR binding moiety and compared to that observed with remternetug.

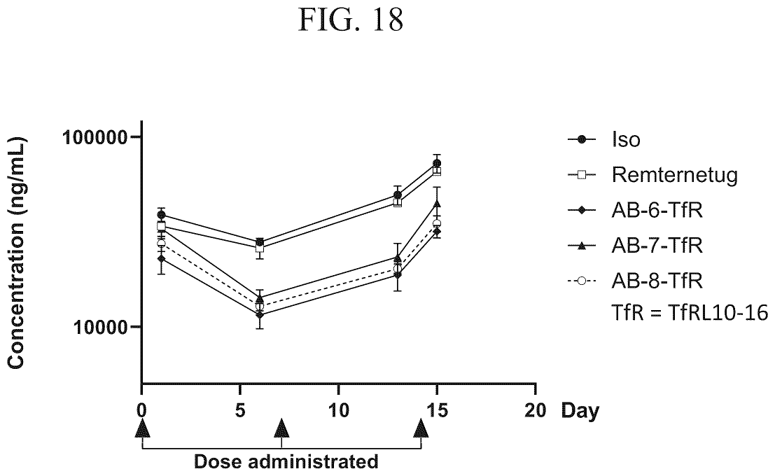

An in vivo multi-dose study was conducted to assess both serum PK and brain uptake of anti-pyroGlu3 Abeta (Aβ)-huTfR antibodies (AB-6/TfRL10-16, AB-7/TfRL10-16, and AB-8/TfRL10-16) in huTfR KI mice at 3 mg/kg with three intravenous doses on Day 0, Day 7, and Day 14. Animals were bled by sub-mandibular veins at Day 1, Day 6, Day 13, and Day 15 post-dosing to determine serum antibody levels (FIG. 18). All anti-pyroGlu3 Abeta (Aβ)-huTfR antibodies displayed similar serum PK profiles, showing faster peripheral clearance compared to that observed with isotype control or remternetug.

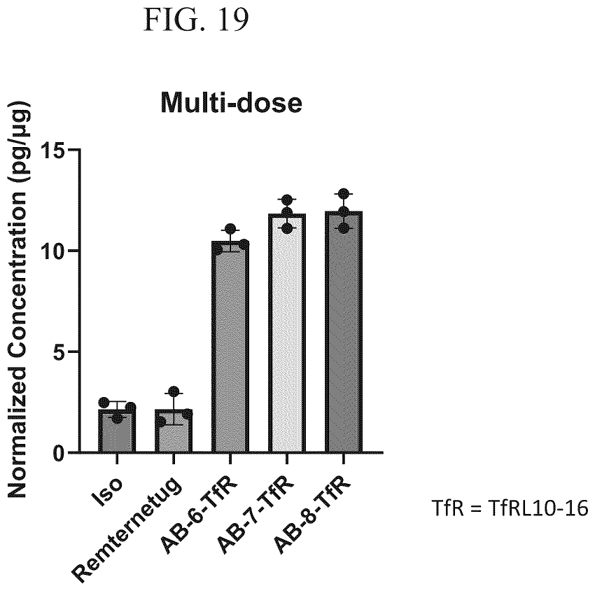

Additionally, brains were collected 24 hours following administration of the third dose of antibody to determine brain uptake in this multi-dose in vivo study. As shown in FIG. 19, all three anti-pyroGlu3 Abeta (Aβ)-huTfR antibodies (AB-6/TfRL10-16, AB-7/TfRL10-16, and AB-8/TfRL10-16) showed higher brain uptake compared to that observed with isotype control or remternetug.

### Example 25: In Vivo Brain Uptake and Serum PK of Anti-pyroGlu3 Abeta (Aβ)-msTfR Antibodies in Aged 5XFAD Mice

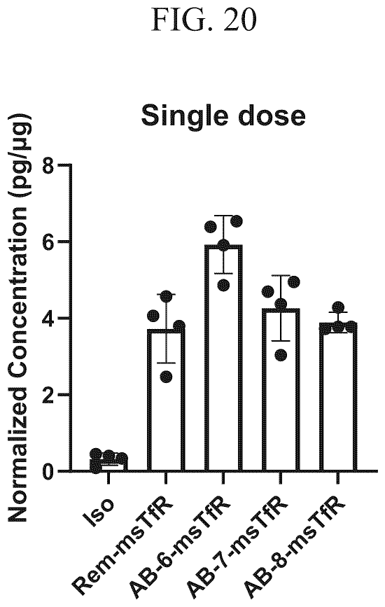

To investigate brain uptake and safety of anti-pyroGlu3 Abeta (Aβ) antibodies with a murine TfR surrogate (msTfR) in a 2+1 format, a single dose (3 mg/kg) study was conducted in aged (9-month old) 5XFAD mice, and brain samples were collected at 24 h post dosing for brain uptake measurements of the antibodies. The anti-pyroGlu3 Abeta (Aβ)-msTfR antibodies are shown in Table 20. Vessel-depleted brain lysates were assessed for antibody levels in the brain parenchyma. As shown in FIG. 20, all anti-pyroGlu3 Abeta (Aβ)-msTfR antibodies tested showed higher brain uptake compared to that observed with isotype control. AB-6-msTfR showed higher brain concentration levels compared to that observed with remternetug-msTfR. AB-7-msTfR and AB-8-msTfR showed similar brain uptake as observed with remternetug-msTfR.

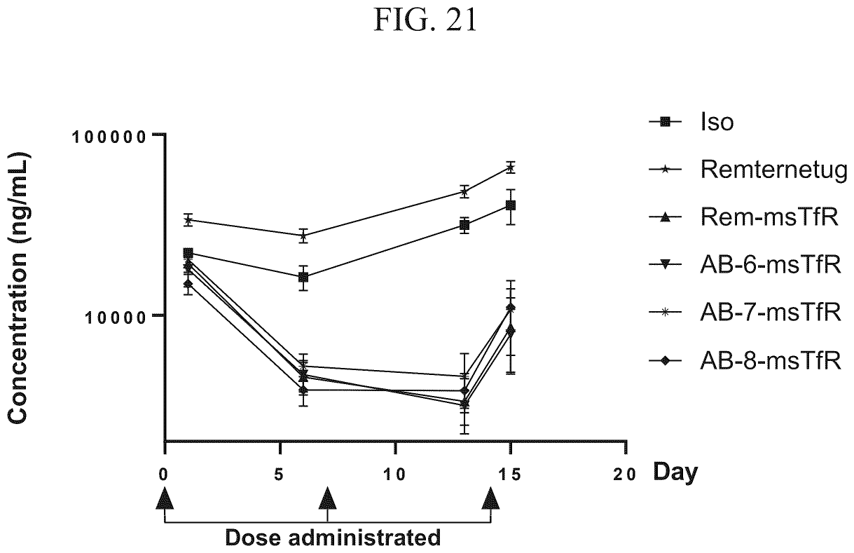

Similarly, a multi-dose study PK was conducted in aged 5XFAD mice with 3 weekly intravenous doses at 3 mg/kg on Day 0, Day 7, and Day 14. Blood samples are collected in life for serum PK analysis at Day 1, Day 6, Day 13, and Day 15 post-dosing. As shown in FIG. 21, all anti-pyroGlu3 Abeta (Aβ)-msTfR antibodies displayed similar serum PK profiles, showing faster peripheral clearance compared to that observed with isotype control.

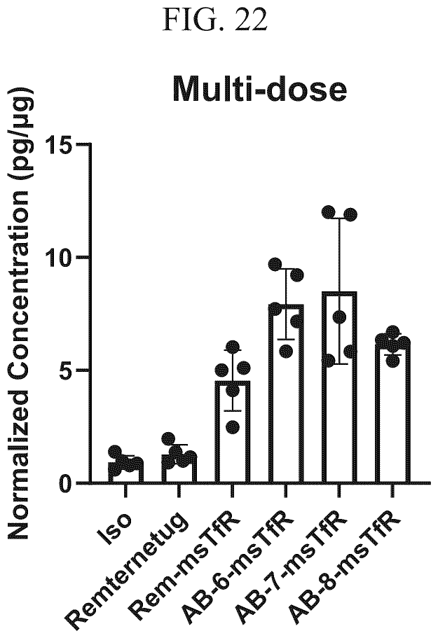

Anti-pyroGlu3 Abeta (Aβ)-msTfR antibodies tested in this study showed lower antibody levels in serum compared to that observed in isotype control animals after three doses. At 24 h post final dose, brains were harvested and processed as described above. As shown in FIG. 22, all anti-pyroGlu3 Abeta (Aβ)-msTfR antibodies tested showed higher brain uptake compared to that observed with Remternetug without an anti-TfR component. Additionally, AB-6-msTfR, AB-7-msTfR, and AB-8-msTfR showed higher brain concentration levels compared to that observed with remternetug-msTfR.

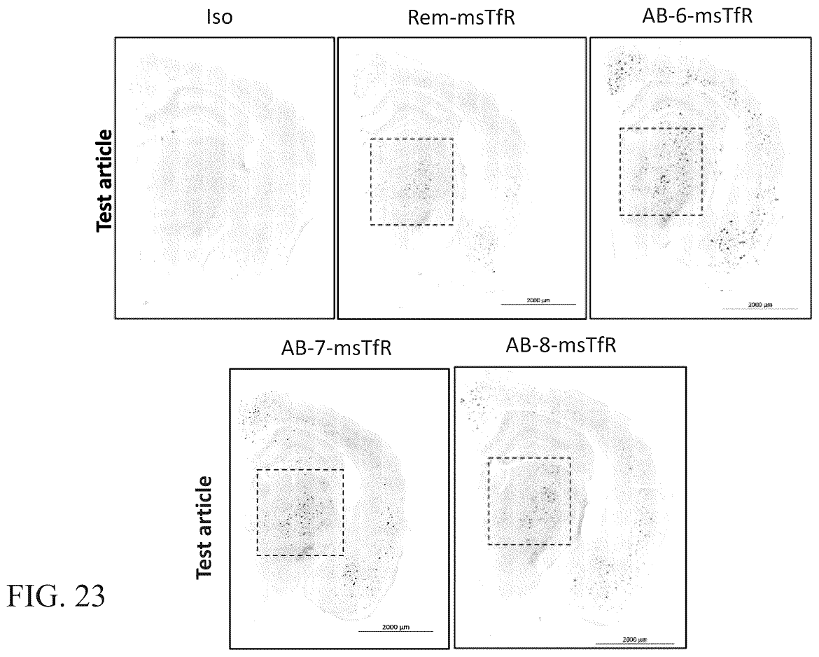

The extent of in vivo target engagement measured via plaque binding of anti-pyroGlu Abeta (Aβ) antibodies of the present disclosure was assessed as described above. Fixed brains were processed for immunofluorescent staining; dosed anti-pyroGlu3 Abeta (Aβ) antibodies of the present disclosure were detected by fluorescently labeled secondary antibodies against human IgG. As shown in FIG. 23, anti-pyroGlu3 Abeta (Aβ) antibodies of the present disclosure bound to plaques that were also labeled by anti-Abeta (Aβ) antibody 6E10 (Novus Biologicals, Cat #NBP2-62566) (data not shown). All anti-pyroGlu3 Abeta (Aβ)-msTfR antibodies of the present disclosure (AB-6-msTfR, AB-7-msTfR, and AB-8-msTfR) showed amyloid plaque binding. AB-6-msTfR, AB-7-msTfR, and AB-8-msTfR also showed higher amyloid plaque binding, as measured by staining in this assay, compared to that observed with remternetug-msTfR.

### Example 26: In Vivo Brain PK of Anti-pyroGlu3 Abeta (Aβ)-msTfR Antibodies in 5XFAD at 2 Dose Levels

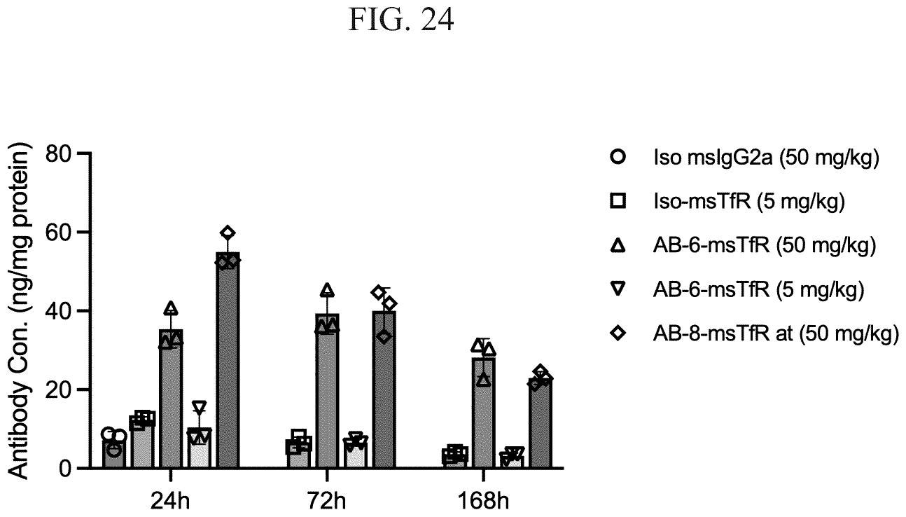

To investigate brain PK of murine TfR surrogate anti-pyroGlu3 Abeta (Aβ) 2+1 antibodies, a single dose (5 or 50 mg/kg) study was conducted in aged 5XFAD mice. Brain samples were collected at 24 h, 72h, and 168 h post antibody administration for brain uptake measurements. Vessel-depleted brain lysates of the animals were assessed for antibody levels in the brain parenchyma. As shown in FIG. 24, both AB-6-msTfR and AB-8-msTfR at 50 mg/kg showed more than 5-fold higher antibody levels in brain parenchyma compared to that observed with isotype control (50 mg/kg) at 24h, with the greatest increase observed at 24 hr for AB-8-msTfR at 50 mg/kg. AB-6-msTfR at 5 mg/kg showed a similar level of brain uptake as Iso-msTfR (5 mg/kg). All 50 mg/kg groups showed elevated antibody levels in brain parenchyma at 72 h and 168 h time points.

### Example 27: In Vivo Aβ Reduction by Anti-pyroGlu3 Abeta (Aβ)-msTfR Antibodies

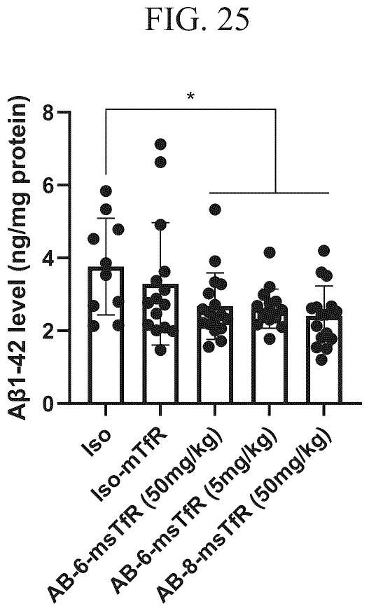

A chronic multidose efficacy study was performed to assess the extent of amyloid beta reductions following 4 weekly doses of anti-pyroGlu3 Abeta (Aβ)-msTfR antibodies of the present disclosure at 5 and 50 mg/kg. Brain tissue was harvested for plaque staining and lysates generated in 8M guanidine hydrochloride (GuHCl) to measure Abeta (Aβ) levels at study termination. An ELISA (as described in deMattaos et al, 2012, Neuron, 76:908-920) was used to determine the levels of Abeta (Aβ)₁₋₄₂ in the cortex of the mice after generation of whole brain lysates in GuHCl. As shown in FIG. 25, Abeta (Aβ)₁₋₄₂ levels were reduced by approximately ~30% in animals administered either 5 mg/kg or 50 mg/kg of AB-6-msTfR, and 50 mg/kg of AB-8-msTfR compared to that observed with isotype msGgG2a control. These results showed that in an in vivo animal model, anti-pyroGlu3 Abeta (Aβ)-msTfR antibodies of the present disclosure were effective at reducing Abeta (Aβ)₁₋₄₂ levels by approximately ~0.30%.

### Example 28: Amino Acid Sequences of Select Anti-pyroGlu3 Abeta (Aβ) Antigen-Binding Domain and an Antd-TfR Antigen Binding Domain Compositions of the Present Disclosure

Provided below in Table 21 are amino acid sequences of select anti-pyroGlu3 Abeta (Aβ) antigen-binding domain (AB-6)/anti-TfR scFv antigen binding domain compositions in 2+1 format. Provided below in Table 22 are amino acid sequences of select anti-pyroGlu3 Abeta (Aβ) antigen-binding domain (AB-8)/anti-TfR scFv antigen binding domain compositions in 2+1 format.

TABLE 21

| | Heavy Chain Knob | Heavy Chain Hole | Light Chain |
|---|---|---|---|
| AB-6 hIgG1 | QLQLQESGPGLVKPSET | QLQLQESGPGLVKPSETL | DIQMTQSPSSLS |
| TfR.15.WH8.1 scFv | LSLTCTVSGGSISSGSYY | SLTCTVSGGSISSGSYYW | ASVGDRVTITC |
| 2+1 | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | TYSTPITFGQGT |
| | GCLVKDYFPEPVTVSW | DYFPEPVTVSWNSGALT | RLEIKRTVAAPS |
| | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 225) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK (SEQ ID NO: 224) | |
| | LSLSPGKGGSGGQVQLV | | |
| | QSGAEVKKPGASVKVS | | |
| | CKASGYTFTSYYMHWV | | |
| | RQAPGQGLEWMGNIYP | | |
| | GSGSSNYAQKFQGRVT | | |
| | MTRDTSTSTVYMELSSL | | |
| | RSEDTAVYYCTRSSYYG | | |
| | NFDTMDNWGQGTTVT | | |
| | VSSGGSEGKSSGSGSES | | |
| | KSTGGSEIVLTQSPATLS | | |
| | LSPGERATLSCRASSSIR | | |
| | YMNWYQQKPGQAPRR | | |
| | LIYDTSNRATGIPARFSG | | |
| | SGSGTDFTLTISSLEPED | | |
| | FAVYYCHQRSSYPWTF | | |
| | GGGTKVEIK (SEQ ID | | |
| | NO: 241) | | |
| AB-6 hIgG1 | QLQLQESGPGLVKPSET | QLQLQESGPGLVKPSETL | DIQMTQSPSSLS |
| TfR.15.WH8.1.42Q | LSLTCTVSGGSISSGSYY | SLTCTVSGGSISSGSYYW | ASVGDRVTITC |
| scFv 2+1 | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | TYSTPITFGQGT |
| | GCLVKDYFPEPVTVSW | DYFPEPVTVSWNSGALT | RLEIKRTVAAPS |
| | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 225) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK (SEQ ID NO: 224) | |
| | LSLSPGKGGSGGQVQLV | | |
| | QSGAEVKKPGASVKVS | | |
| | CKASGYTFTSYYMHWV | | |
| | RQAPGQGLEWMGNIYP | | |
| | GSGSSNYAQKFQGRVT | | |
| | MTRDTSTSTVYMELSSL | | |
| | RSEDTAVYYCTRSSYYG | | |
| | NFDTMDNWGQGTTVT | | |
| | VSSGGSEGKSSGSGSES | | |
| | KSTGGSEIVLTQSPATLS | | |
| | LSPGERATLSCRASSSIR | | |
| | QMNWYQQKPGQAPRR | | |
| | LIYDTSNRATGIPARFSG | | |
| | SGSGTDFTLTISSLEPED | | |
| | FAVYYCHQRSSYPWTF | | |
| | GGGTKVEIK (SEQ ID | | |
| | NO: 242) | | |
| AB-6 hIgG1 | QLQLQESGPGLVKPSET | QLQLQESGPGLVKPSETL | DIQMTQSPSSLS |
| TfR.15.WH8.1.42Q. | LSLTCTVSGGSISSGSYY | SLTCTVSGGSISSGSYYW | ASVGDRVTITC |
| H6-4 scFv 2+1 | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | TYSTPITFGQGT |
| | GCLVKDYFPEPVTVSW | DYFPEPVTVSWNSGALT | RLEIKRTVAAPS |
| | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 225) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK (SEQ ID NO: 224) | |
| | LSLSPGKGGSGGQVQLV | | |
| | QSGAEVKKPGASVKVS | | |
| | CKASGYTFTSYYMHWV | | |
| | RQAPGQGLEWMGNIYP | | |
| | GSGSSNYAQKFQGRVT | | |
| | MTRDTSTSTVYMELSSL | | |
| | RSEDTAVYYCTRSSYYG | | |
| | RFDTMDNWGQGTTVTV | | |
| | SSGGSEGKSSGSGSESKS | | |
| | TGGSEIVLTQSPATLSLS | | |
| | PGERATLSCRASSSIRY | | |
| | MNWYQQKPGQAPRRLI | | |
| | YDTSNRATGIPARFSGS | | |
| | GSGTDFTLTISSLEPEDF | | |
| | AVYYCHQRSSYPWTFG | | |
| | GGTKVEIK (SEQ ID NO: | | |
| | 243) | | |
| AB-6 hIgG1 | QLQLQESGPGLVKPSET | QLQLQESGPGLVKPSETL | DIQMTQSPSSLS |
| TfR.15.WH8.1.42Q.L | LSLTCTVSGGSISSGSYY | SLTCTVSGGSISSGSYYW | ASVGDRVTITC |
| 10-1 scFv 2+1 | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | TYSTPITFGQGT |
| | GCLVKDYFPEPVTVSW | DYFPEPVTVSWNSGALT | RLEIKRTVAAPS |
| | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 225) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK | |
| | LSLSPGKGGSGGQVQLV | (SEQ ID NO: 224) | |
| | QSGAEVKKPGASVKVS | | |
| | CKASGYTFTSYYMHWV | | |
| | RQAPGQGLEWMGNIYP | | |
| | GSGSSNYAQKFQGRVT | | |
| | MTRDTSTSTVYMELSSL | | |
| | RSEDTAVYYCTRSSYYG | | |
| | NFDTMDNWGQGTTVT | | |
| | VSSGGSEGKSSGSGSES | | |
| | KSTGGSEIVLTQSPATLS | | |
| | LSPGERATLSCRASSSIR | | |
| | QMAWYQQKPGQAPRR | | |
| | LIYDTSNRATGIPARFSG | | |
| | SGSGTDFTLTISSLEPED | | |
| | FAVYYCHQRSSYPWTF | | |
| | GGGTKVEIK (SEQ ID | | |
| | NO: 244) | | |
| AB-6 hIgG1 | QLQLQESGPGLVKPSET | QLQLQESGPGLVKPSETL | DIQMTQSPSSLS |
| TfR.15.WH8.1.42Q.L | LSLTCTVSGGSISSGSYY | SLTCTVSGGSISSGSYYW | ASVGDRVTITC |
| 10-8 scFv 2+1 | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | TYSTPITFGQGT |
| | GCLVKDYFPEPVTVSW | DYFPEPVTVSWNSGALT | RLEIKRTVAAPS |
| | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 225) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK (SEQ ID NO: 224) | |
| | LSLSPGKGGSGGQVQLV | | |
| | QSGAEVKKPGASVKVS | | |
| | CKASGYTFTSYYMHWV | | |
| | RQAPGQGLEWMGNIYP | | |
| | GSGSSNYAQKFQGRVT | | |
| | MTRDTSTSTVYMELSSL | | |
| | RSEDTAVYYCTRSSYYG | | |
| | NFDTMDNWGQGTTVT | | |
| | VSSGGSEGKSSGSGSES | | |
| | KSTGGSEIVLTQSPATLS | | |
| | LSPGERATLSCRASSSIR | | |
| | QMSWYQQKPGQAPRRL | | |
| | IYDTSNRATGIPARFSGS | | |
| | GSGTDFTLTISSLEPEDF | | |
| | AVYYCHQRSSYPWTFG | | |
| | GGTKVEIK (SEQ ID NO: | | |
| | 245) | | |
| AB-6 hIgG1 | QLQLQESGPGLVKPSET | QLQLQESGPGLVKPSETL | DIQMTQSPSSLS |
| TfR.15.WH8.1.24A | LSLTCTVSGGSISSGSYY | SLTCTVSGGSISSGSYYW | ASVGDRVTITC |
| scFv 2+1 | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | TYSTPITFGQGT |
| | GCLVKDYFPEPVTVSW | DYFPEPVTVSWNSGALT | RLEIKRTVAAPS |
| | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 225) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK (SEQ ID NO: 224) | |
| | LSLSPGKGGSGGQVQLV | | |
| | QSGAEVKKPGASVKVS | | |
| | CKASGYTFTSYYMHWV | | |
| | RQAPGQGLEWMGNIYP | | |
| | GSGSSNYAQKFQGRVT | | |
| | MTRDTSTSTVYMELSSL | | |
| | RSEDTAVYYCTRSAYY | | |
| | GNFDTMDNWGQGTTV | | |
| | TVSSGGSEGKSSGSGSE | | |
| | SKSTGGSEIVLTQSPATL | | |
| | SLSPGERATLSCRASSSI | | |
| | RYMNWYQQKPGQAPR | | |
| | RLIYDTSNRATGIPARFS | | |
| | GSGSGTDFTLTISSLEPE | | |
| | DFAVYYCHQRSSYPWT | | |
| | FGGGTKVEIK (SEQ ID | | |
| | NO: 246) | | |
| AB-6 hIgG1 | QLQLQESGPGLVKPSET | QLQLQESGPGLVKPSETL | DIQMTQSPSSLS |
| TfR.15.WH8.1.42Q.L | LSLTCTVSGGSISSGSYY | SLTCTVSGGSISSGSYYW | ASVGDRVTITC |
| 7-2 scFv 2+1 | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | TYSTPITFGQGT |
| | GCLVKDYFPEPVTVSW | DYFPEPVTVSWNSGALT | RLEIKRTVAAPS |
| | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 225) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK (SEQ ID NO: 224) | |
| | LSLSPGKGGSGGQVQLV | | |
| | QSGAEVKKPGASVKVS | | |
| | CKASGYTFTSYYMHWV | | |
| | RQAPGQGLEWMGNIYP | | |
| | GSGSSNYAQKFQGRVT | | |
| | MTRDTSTSTVYMELSSL | | |
| | RSEDTAVYYCTRSSYYG | | |
| | NFDTMDNWGQGTTVT | | |
| | VSSGGSEGKSSGSGSES | | |
| | KSTGGSEIVLTQSPATLS | | |
| | LSPGERATLSCRASSSID | | |
| | QMNWYQQKPGQAPRR | | |
| | LIYDTSNRATGIPARFSG | | |
| | SGSGTDFTLTISSLEPED | | |
| | FAVYYCHQRSSYPWTF | | |
| | GGGTKVEIK (SEQ ID | | |
| | NO: 247) | | |
| AB-6 hIgG1 | QLQLQESGPGLVKPSET | QLQLQESGPGLVKPSETL | DIQMTQSPSSLS |
| TfR.9.1B.39.38 scFv | LSLTCTVSGGSISSGSYY | SLTCTVSGGSISSGSYYW | ASVGDRVTITC |
| 2+1 | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | TYSTPITFGQGT |
| | GCLVKDYFPEPVTVSW | DYFPEPVTVSWNSGALT | RLEIKRTVAAPS |
| | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 225) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK (SEQ ID NO: 224) | |
| | LSLSPGKGGSGGQVQLV | | |
| | QSGAEVKKPGASVKVS | | |
| | CKASGYTFTEYAMHWV | | |
| | RQAPGQGLEWMGGIAP | | |
| | NTGGTSYNQKFKGRVT | | |
| | MTVDTSTSTAYMELSSL | | |
| | RSEDTAVYYCASQGWL | | |
| | LRGGDYWGQGTLVTVS | | |
| | SGGSEGKSSGSGSESKS | | |
| | TGGSDVVMTQSPLSLPV | | |
| | TLGQPASISCKASQSLLD | | |
| | EGGKTYLNWLQQRPGQ | | |
| | SPRRLIYLVSKLDSGVP | | |
| | DRFSGSGSGTDFTLKISR | | |
| | VEAEDVGVYYCWQGT | | |
| | HFPQTFGGGTKVEIK | | |
| | (SEQ ID NO: 248) | | |
| AB-6 hIgG1 | QLQLQESGPGLVKPSET | QLQLQESGPGLVKPSETL | DIQMTQSPSSLS |
| TfR15.WH8.1.42Q..L | LSLTCTVSGGSISSGSYY | SLTCTVSGGSISSGSYYW | ASVGDRVTITC |
| 10-16.dsFvHL | WGWIRQPPGKGLEWIG | GWIRQPPGKGLEWIGSF | RASQSIRFYLN |
| Disulfide stapled | SFYYSGTIYYNPSLKSR | YYSGTIYYNPSLKSRVTI | WYQQKPGKAP |
| TfR. 15.WH8.1.42Q.L | VTISVDTSKNQFSLKLSS | SVDTSKNQFSLKLSSVTA | KVVIYGASSLQ |
| 10-16 (substitute AA | VTAADTAVYYCARQRE | ADTAVYYCARQREYSSS | SGVPSRFSGSGS |
| to cysteines at VH44 | YSSSSNYYYYYMDVWG | SNYYYYYMDVWGKGTT | GTDFTLTISSLQ |
| and VL100) | KGTTVTVSSASTKGPSV | VTVSSASTKGPSVFPLAP | PEDFATYYCQQ |
| (in VH-VL | FPLAPSSKSTSGGTAAL | SSKSTSGGTAALGCLVK | TYSTPITFGQGT |
| orientation) | GCLVKDYFPEPVTVSW | DYFPEPVTVSWNSGALT | RLEIKRTVAAPS |
| See Table 15 above | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 225) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK (SEQ ID NO: 224) | |
| | LSLSPGKGGSGGQVQLV | | |
| | QSGAEVKKPGASVKVS | | |
| | CKASGYTFTSYYMHWV | | |
| | RQAPGQCLEWMGNIYP | | |
| | GSGSSNYAQKFQGRVT | | |
| | MTRDTSTSTVYMELSSL | | |
| | RSEDTAVYYCTRSSYYG | | |
| | NFDTMDNWGQGTTVT | | |
| | VSSGGSEGKSSGSGSES | | |
| | KSTGGSEIVLTQSPATLS | | |
| | LSPGERATLSCRASSSIR | | |
| | QMHWYQQKPGQAPRR | | |
| | LIYDTSNRATGIPARFSG | | |
| | SGSGTDFTLTISSLEPED | | |
| | FAVYYCHQRSSYPWTF | | |
| | GCGTKVEIK (SEQ ID | | |
| | NO: 278) | | |
| AB-6 hIgG1 | QLQLQESGPGLVKPSET | QLQLQESGPGLVKPSETL | DIQMTQSPSSLS |
| TfR.9.1B.35 | LSLTCTVSGGSISSGSYY | SLTCTVSGGSISSGSYYW | ASVGDRVTITC |
| scFv 2+1 | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | TYSTPITFGQGT |
| | GCLVKDYFPEPVTVSW | DYFPEPVTVSWNSGALT | RLEIKRTVAAPS |
| | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C |
| | NKALPAPIEKTISKAKG | KAKGQPREPQVYTLPPS | (SEQ ID NO: |
| | QPREPQVYTLPPSRDEL | RDELTKNQVSLSCAVKG | 225) |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK (SEQ ID NO: 224) | |
| | LSLSPGKGGSGGQVQLV | | |
| | QSGAEVKKPGASVKVS | | |
| | CKASGYTFTEYAMHWV | | |
| | RQAPGQGLEWMGGINP | | |
| | NDGGTSYNQKFKGRVT | | |
| | MTVDTSTSTAYMELSSL | | |
| | RSEDTAVYYCASQGWL | | |
| | LRGGDYWGQGTLVTVS | | |
| | SGGSEGKSSGSGSESKS | | |
| | TGGSDVVMTQSPLSLPV | | |
| | TLGQPASISCKSSQSLLD | | |
| | SDGKTYLNWLQQRPGQ | | |
| | SPRRLIYLVSKLDSGVP | | |
| | DRFSGSGSGTDFTLKISR | | |
| | VEAEDVGVYHCWQGT | | |
| | HFPQSFGGGTKVEIK | | |
| | (SEQ ID NO: 249) | | |
| AB-6 hIgG1 | QLQLQESGPGLVKPSET | QLQLQESGPGLVKPSETL | DIQMTQSPSSLS |
| TfR.9.1B.39 | LSLTCTVSGGSISSGSYY | SLTCTVSGGSISSGSYYW | ASVGDRVTITC |
| scFv 2+1 | WGWIRQPPGKGLEWIG | GWIRQPPGKGLEWIGSF | RASQSIRFYLN |
| | SFYYSGTIYYNPSLKSR | YYSGTIYYNPSLKSRVTI | WYQQKPGKAP |
| | VTISVDTSKNQFSLKLSS | SVDTSKNQFSLKLSSVTA | KVVIYGASSLQ |
| | VTAADTAVYYCARQRE | ADTAVYYCARQREYSSS | SGVPSRFSGSGS |
| | YSSSSNYYYYYMDVWG | SNYYYYYMDVWGKGTT | GTDFTLTISSLQ |
| | KGTTVTVSSASTKGPSV | VTVSSASTKGPSVFPLAP | PEDFATYYCQQ |
| | FPLAPSSKSTSGGTAAL | SSKSTSGGTAALGCLVK | TYSTPITFGQGT |
| | GCLVKDYFPEPVTVSW | DYFPEPVTVSWNSGALT | RLEIKRTVAAPS |
| | NSGALTSGVHTFPAVLQ | SGVHTFPAVLQSSGLYSL | VFIFPPSDEQLK |
| | SSGLYSLSSVVTVPSSSL | SSVVTVPSSSLGTQTYIC | SGTASVVCLLN |
| | GTQTYICNVNHKPSNTK | NVNHKPSNTKVDKKVEP | NFYPREAKVQ |
| | VDKKVEPKSCDKTHTC | KSCDKTHTCPPCPAPELL | WKVDNALQSG |
| | PPCPAPELLGGPSVFLFP | GGPSVFLFPPKPKDTLMI | NSQESVTEQDS |
| | PKPKDTLMISRTPEVTC | SRTPEVTCVVVDVSHED | KDSTYSLSSTLT |
| | VVVDVSHEDPEVKFNW | PEVKFNWYVDGVEVHN | LSKADYEKHKV |
| | YVDGVEVHNAKTKPRE | AKTKPREEQYNSTYRVV | YACEVTHQGLS |
| | EQYNSTYRVVSVLTVL | SVLTVLHQDWLNGKEY | SPVTKSFNRGE |
| | HQDWLNGKEYKCKVS | KCKVSNKALPAPIEKTIS | C (SEQ ID NO: |
| | NKALPAPIEKTISKAKG | KAKGQPREPQVYTLPPS | 225) |
| | QPREPQVYTLPPSRDEL | RDELTKNQVSLSCAVKG | |
| | TKNQVSLWCLVKGFYP | FYPSDIAVEWESNGQPEN | |
| | SDIAVEWESNGQPENNY | NYKTTPPVLDSDGSFFLV | |
| | KTTPPVLDSDGSFFLYS | SKLTVDKSRWQQGNVFS | |
| | KLTVDKSRWQQGNVFS | CSVMHEALHNHYTQKSL | |
| | CSVMHEALHNHYTQKS | SLSPGK (SEQ ID NO: 224) | |
| | LSLSPGKGGSGGQVQLV | | |
| | QSGAEVKKPGASVKVS | | |
| | CKASGYTFTEYAMHWV | | |
| | RQAPGQGLEWMGGINP | | |
| | NDGGTSYNQKFKGRVT | | |
| | MTVDTSTSTAYMELSSL | | |
| | RSEDTAVYYCASQGWL | | |
| | LRGGDYWGQGTLVTVS | | |
| | SGGSEGKSSGSGSESKS | | |
| | TGGSDVVMTQSPLSLPV | | |
| | TLGQPASISCKASQSLLD | | |
| | EDGKTYLNWLQQRPGQ | | |
| | SPRRLIYLVSKLDSGVP | | |
| | DRFSGSGSGTDFTLKISR | | |
| | VEAEDVGVYYCWQGT | | |
| | HFPQTFGGGTKVEIK | | |
| | (SEQ ID NO: 250) | | |

TABLE 22

| | Heavy Chain Knob | Heavy Chain Hole | Light Chain |
|---|---|---|---|
| AB-8 hIgG1 | EVQLVESGGGLIQPGGS | EVQLVESGGGLIQPGGSL | DIQMTQSPSSLS |
| TfR.15.WH8.1 scFv | LRLSCAASGFTVSSNYM | RLSCAASGFTVSSNYMS | ASVGDRVTITC |
| 2+1 | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KGGSGGQVQLVQSGAE | | |
| | VKKPGASVKVSCKASG | | |
| | YTFTSYYMHWVRQAPG | | |
| | QGLEWMGNIYPGSGSS | | |
| | NYAQKFQGRVTMTRDT | | |
| | STSTVYMELSSLRSEDT | | |
| | AVYYCTRSSYYGNFDT | | |
| | MDNWGQGTTVTVSSGG | | |
| | SEGKSSGSGSESKSTGG | | |
| | SEIVLTQSPATLSLSPGE | | |
| | RATLSCRASSSIRYMNW | | |
| | YQQKPGQAPRRLIYDTS | | |
| | NRATGIPARFSGSGSGT | | |
| | DFTLTISSLEPEDFAVYY | | |
| | CHQRSSYPWTFGGGTK | | |
| | VEIK (SEQ ID NO: 251) | | |
| AB-8 hIgG1 | EVQLVESGGGLIQPGGS | EVQLVESGGGLIQPGGSL | DIQMTQSPSSLS |
| TfR.15.WH8.1.42Q | LRLSCAASGFTVSSNYM | RLSCAASGFTVSSNYMS | ASVGDRVTITC |
| scFv 2+1 | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KGGSGGQVQLVQSGAE | | |
| | VKKPGASVKVSCKASG | | |
| | YTFTSYYMHWVRQAPG | | |
| | QGLEWMGNIYPGSGSS | | |
| | NYAQKFQGRVTMTRDT | | |
| | STSTVYMELSSLRSEDT | | |
| | AVYYCTRSSYYGNFDT | | |
| | MDNWGQGTTVTVSSGG | | |
| | SEGKSSGSGSESKSTGG | | |
| | SEIVLTQSPATLSLSPGE | | |
| | RATLSCRASSSIRQMNW | | |
| | YQQKPGQAPRRLIYDTS | | |
| | NRATGIPARFSGSGSGT | | |
| | DFTLTISSLEPEDFAVYY | | |
| | CHQRSSYPWTFGGGTK | | |
| | VEIK (SEQ ID NO: 252) | | |
| AB-8 hIgG1 | EVQLVESGGGLIQPGGS | EVQLVESGGGLIQPGGSL | DIQMTQSPSSLS |
| TfR.15.WH8.1.42Q. | LRLSCAASGFTVSSNYM | RLSCAASGFTVSSNYMS | ASVGDRVTITC |
| H6-4 scFv 2+1 | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KGGSGGQVQLVQSGAE | | |
| | VKKPGASVKVSCKASG | | |
| | YTFTSYYMHWVRQAPG | | |
| | QGLEWMGNIYPGSGSS | | |
| | NYAQKFQGRVTMTRDT | | |
| | STSTVYMELSSLRSEDT | | |
| | AVYYCTRSSYYGRFDT | | |
| | MDNWGQGTTVTVSSGG | | |
| | SEGKSSGSGSESKSTGG | | |
| | SEIVLTQSPATLSLSPGE | | |
| | RATLSCRASSSIRYMNW | | |
| | YQQKPGQAPRRLIYDTS | | |
| | NRATGIPARFSGSGSGT | | |
| | DFTLTISSLEPEDFAVYY | | |
| | CHQRSSYPWTFGGGTK | | |
| | VEIK (SEQ ID NO: 253) | | |
| AB-8 hIgG1 | EVQLVESGGGLIQPGGS | EVQLVESGGGLIQPGGSL | DIQMTQSPSSLS |
| TfR.15.WH8.1.42Q.L | LRLSCAASGFTVSSNYM | RLSCAASGFTVSSNYMS | ASVGDRVTITC |
| 10-1 scFv 2+1 | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KGGSGGQVQLVQSGAE | | |
| | VKKPGASVKVSCKASG | | |
| | YTFTSYYMHWVRQAPG | | |
| | QGLEWMGNIYPGSGSS | | |
| | NYAQKFQGRVTMTRDT | | |
| | STSTVYMELSSLRSEDT | | |
| | AVYYCTRSSYYGNFDT | | |
| | MDNWGQGTTVTVSSGG | | |
| | SEGKSSGSGSESKSTGG | | |
| | SEIVLTQSPATLSLSPGE | | |
| | RATLSCRASSSIRQMAW | | |
| | YQQKPGQAPRRLIYDTS | | |
| | NRATGIPARFSGSGSGT | | |
| | DFTLTISSLEPEDFAVYY | | |
| | CHQRSSYPWTFGGGTK | | |
| | VEIK (SEQ ID NO: 254) | | |
| AB-8 hIgG1 | EVQLVESGGGLIQPGGS | EVQLVESGGGLIQPGGSL | DIQMTQSPSSLS |
| TfR.15.WH8.1.42Q.L | LRLSCAASGFTVSSNYM | RLSCAASGFTVSSNYMS | ASVGDRVTITC |
| 10-8 scFv 2+1 | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KGGSGGQVQLVQSGAE | | |
| | VKKPGASVKVSCKASG | | |
| | YTFTSYYMHWVRQAPG | | |
| | QGLEWMGNIYPGSGSS | | |
| | NYAQKFQGRVTMTRDT | | |
| | STSTVYMELSSLRSEDT | | |
| | AVYYCTRSSYYGNFDT | | |
| | MDNWGQGTTVTVSSGG | | |
| | SEGKSSGSGSESKSTGG | | |
| | SEIVLTQSPATLSLSPGE | | |
| | RATLSCRASSSIRQMSW | | |
| | YQQKPGQAPRRLIYDTS | | |
| | NRATGIPARFSGSGSGT | | |
| | DFTLTISSLEPEDFAVYY | | |
| | CHQRSSYPWTFGGGTK | | |
| | VEIK (SEQ ID NO: 255) | | |
| AB-8 hIgG1 | EVQLVESGGGLIQPGGS | EVQLVESGGGLIQPGGSL | DIQMTQSPSSLS |
| TfR.15.WH8.1.24A | LRLSCAASGFTVSSNYM | RLSCAASGFTVSSNYMS | ASVGDRVTITC |
| scFv 2+1 | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KGGSGGQVQLVQSGAE | | |
| | VKKPGASVKVSCKASG | | |
| | YTFTSYYMHWVRQAPG | | |
| | QGLEWMGNIYPGSGSS | | |
| | NYAQKFQGRVTMTRDT | | |
| | STSTVYMELSSLRSEDT | | |
| | AVYYCTRSAYYGNFDT | | |
| | MDNWGQGTTVTVSSGG | | |
| | SEGKSSGSGSESKSTGG | | |
| | SEIVLTQSPATLSLSPGE | | |
| | RATLSCRASSSIRYMNW | | |
| | YQQKPGQAPRRLIYDTS | | |
| | NRATGIPARFSGSGSGT | | |
| | DFTLTISSLEPEDFAVYY | | |
| | CHQRSSYPWTFGGGTK | | |
| | VEIK (SEQ ID NO: 256) | | |
| AB-8 hIgG1 | EVQLVESGGGLIQPGGS | EVQLVESGGGLIQPGGSL | DIQMTQSPSSLS |
| TfR.15.WH8.1.42Q.L | LRLSCAASGFTVSSNYM | RLSCAASGFTVSSNYMS | ASVGDRVTITC |
| 7-2 scFv 2+1 | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KGGSGGQVQLVQSGAE | | |
| | VKKPGASVKVSCKASG | | |
| | YTFTSYYMHWVRQAPG | | |
| | QGLEWMGNIYPGSGSS | | |
| | NYAQKFQGRVTMTRDT | | |
| | STSTVYMELSSLRSEDT | | |
| | AVYYCTRSSYYGNFDT | | |
| | MDNWGQGTTVTVSSGG | | |
| | SEGKSSGSGSESKSTGG | | |
| | SEIVLTQSPATLSLSPGE | | |
| | RATLSCRASSSIDQMNW | | |
| | YQQKPGQAPRRLIYDTS | | |
| | NRATGIPARFSGSGSGT | | |
| | DFTLTISSLEPEDFAVYY | | |
| | CHQRSSYPWTFGGGTK | | |
| | VEIK (SEQ ID NO: 257) | | |
| AB-8 hIgG1 | EVQLVESGGGLIQPGGS | EVQLVESGGGLIQPGGSL | DIQMTQSPSSLS |
| TfR9.1B.39.38 scFv | LRLSCAASGFTVSSNYM | RLSCAASGFTVSSNYMS | ASVGDRVTITC |
| 2+1 | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KGGSGGQVQLVQSGAE | | |
| | VKKPGASVKVSCKASG | | |
| | YTFTEYAMHWVRQAPG | | |
| | QGLEWMGGIAPNTGGT | | |
| | SYNQKFKGRVTMTVDT | | |
| | STSTAYMELSSLRSEDT | | |
| | AVYYCASQGWLLRGGD | | |
| | YWGQGTLVTVSSGGSE | | |
| | GKSSGSGSESKSTGGSD | | |
| | VVMTQSPLSLPVTLGQP | | |
| | ASISCKASQSLLDEGGK | | |
| | TYLNWLQQRPGQSPRR | | |
| | LIYLVSKLDSGVPDRFS | | |
| | GSGSGTDFTLKISRVEA | | |
| | EDVGVYYCWQGTHFPQ | | |
| | TFGGGTKVEIK (SEQ ID | | |
| | NO: 258) | | |
| AB-8 hIgG1 | EVQLVESGGGLIQPGGS | EVQLVESGGGLIQPGGSL | DIQMTQSPSSLS |
| TfR.15.WH8.1.42Q.L | LRLSCAASGFTVSSNYM | RLSCAASGFTVSSNYMS | ASVGDRVTITC |
| 10-16.dsFvHL | SWVRQAPGKGLEWVSV | WVRQAPGKGLEWVSVI | RASQSISNYLN |
| Disulfide stapled | IYSGGTKHYADSVKGRF | YSGGTKHYADSVKGRFT | WYQQKPGKAP |
| TfR.15.WH8.1.42Q.L | TISRDNSKNTLYLQMNS | ISRDNSKNTLYLQMNSL | KLLIYDAYSLQI |
| 10-16 (substitute AA | LRAEDTAVYYCARSYY | RAEDTAVYYCARSYYY | GVPSRFSGSGS |
| to cysteines at VH44 | YGSGSSDAFDIWGQGT | GSGSSDAFDIWGQGTMV | GTDFTLTISSLQ |
| and VL100) | MVTVSSASTKGPSVFPL | TVSSASTKGPSVFPLAPS | PEDFATYYCQQ |
| (in VH-VL | APSSKSTSGGTAALGCL | SKSTSGGTAALGCLVKD | SYSIPPTFGGGT |
| orientation) | VKDYFPEPVTVSWNSG | YFPEPVTVSWNSGALTS | KVEIKRTVAAP |
| See Table 15 above | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KGGSGGQVQLVQSGAE | | |
| | VKKPGASVKVSCKASG | | |
| | YTFTSYYMHWVRQAPG | | |
| | QCLEWMGNIYPGSGSS | | |
| | NYAQKFQGRVTMTRDT | | |
| | STSTVYMELSSLRSEDT | | |
| | AVYYCTRSSYYGNFDT | | |
| | MDNWGQGTTVTVSSGG | | |
| | SEGKSSGSGSESKSTGG | | |
| | SEIVLTQSPATLSLSPGE | | |
| | RATLSCRASSSIRQMHW | | |
| | YQQKPGQAPRRLIYDTS | | |
| | NRATGIPARFSGSGSGT | | |
| | DFTLTISSLEPEDFAVYY | | |
| | CHQRSSYPWTFGCGTK | | |
| | VEIK (SEQ ID NO: 279) | | |
| AB-8 hIgG1 | EVQLVESGGGLIQPGGS | EVQLVESGGGLIQPGGSL | DIQMTQSPSSLS |
| TfR.9.1B.35 scFv 2+1 | LRLSCAASGFTVSSNYM | RLSCAASGFTVSSNYMS | ASVGDRVTITC |
| | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KGGSGGQVQLVQSGAE | | |
| | VKKPGASVKVSCKASG | | |
| | YTFTEYAMHWVRQAPG | | |
| | QGLEWMGGINPNDGGT | | |
| | SYNQKFKGRVTMTVDT | | |
| | STSTAYMELSSLRSEDT | | |
| | AVYYCASQGWLLRGGD | | |
| | YWGQGTLVTVSSGGSE | | |
| | GKSSGSGSESKSTGGSD | | |
| | VVMTQSPLSLPVTLGQP | | |
| | ASISCKSSQSLLDSDGKT | | |
| | YLNWLQQRPGQSPRRLI | | |
| | YLVSKLDSGVPDRFSGS | | |
| | GSGTDFTLKISRVEAED | | |
| | VGVYHCWQGTHFPQSF | | |
| | GGGTKVEIK (SEQ ID | | |
| | NO: 259) | | |
| AB-8 hIgG1 | EVQLVESGGGLIQPGGS | EVQLVESGGGLIQPGGSL | DIQMTQSPSSLS |
| TfR.9.1B.39 scFv 2+1 | LRLSCAASGFTVSSNYM | RLSCAASGFTVSSNYMS | ASVGDRVTITC |
| | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KGGSGGQVQLVQSGAE | | |
| | VKKPGASVKVSCKASG | | |
| | YTFTEYAMHWVRQAPG | | |
| | QGLEWMGGINPNDGGT | | |
| | SYNQKFKGRVTMTVDT | | |
| | STSTAYMELSSLRSEDT | | |
| | AVYYCASQGWLLRGGD | | |
| | YWGQGTLVTVSSGGSE | | |
| | GKSSGSGSESKSTGGSD | | |
| | VVMTQSPLSLPVTLGQP | | |
| | ASISCKASQSLLDEDGK | | |
| | TYLNWLQQRPGQSPRR | | |
| | LIYLVSKLDSGVPDRFS | | |
| | GSGSGTDFTLKISRVEA | | |
| | EDVGVYYCWQGTHFPQ | | |
| | TFGGGTKVEIK (SEQ ID | | |
| | NO: 260) | | |

### Example 29: Amino Acid Sequences of Select Anti-pyroGlu3 Abeta (Aβ) Antigen-Binding Domain and Anti-TfR Antigen Binding Domain Compositions of the Present Disclosure Comprising Different Linkers of Varying Lengths

Provided below in Table 24 are amino acid sequences of select anti-pyroGlu3 Abeta (Aβ) antigen-binding domain (AB-8)/anti-TfR scFv antigen binding domain compositions in 2+1 format comprising different linkers of varying lengths (no linker, −5 linker, and no linker+disulfide staple).

These different linkers of varying lengths (no linker, −5 linker, and no linker+disulfide staple) correspond to the amino acid region located between the C-terminus of the anti-Abeta (Aβ) heavy chain Fc and the N-terminus of the TfR scFv. (See Table 23 below.) In certain anti-pyroGlu3 Abeta (Aβ) antigen-binding domain/anti-TfR antigen binding domain compositions of the present disclosure, the linker located between the anti-pyroGlu3 Abeta (Aβ) antibody heavy chain Fc and the N-terminus of the anti-TfR antigen binding domain scFv comprises the 5 amino acid linker GGSGG (SEQ ID NO: 214). In certain anti-pyroGlu3 Abeta (Aβ) antigen-binding domain/anti-TfR antigen binding domain compositions of the present disclosure, there is no linker located between the anti-pyroGlu3 Abeta (Aβ) antibody heavy chain Fc and the N-terminus of the anti-TfR antigen binding domain scFv. In certain anti-pyroGlu3 Abeta (Aβ) antigen-binding domain/anti-TfR antigen binding domain compositions of the present disclosure, the N-terminal amino acid residues QV of the anti-TfR scFv are removed, further reducing the length of the linker located between the anti-pyroGlu3 Abeta (Aβ) antibody heavy chain Fc and the N-terminus of the anti-TfR antigen binding domain scFv.

Provided in Table 24 are anti-pyroGlu3 Abeta (Aβ) antigen-binding domain/anti-TfR antigen binding domain compositions of the present disclosure comprising alternative linkers and linker lengths, including no linker, −5 linker, and no linker+disulfide staple.

TABLE 23

| | | |
|---|---|---|
| 5 amino acid | HEALHNHYTQKSLSLS | SEQ ID |
| GGSGG (SEQ | PGKGGSGGQVQLVQS | NO: 280 |
| ID | | |
| NO: 214) | | |
| linker | | |
| No linker | HEALHNHYTQKSLSLS | SEQ ID |
| | PGKQVQLVQS | NO: 281 |
| −5 linker | HEALHNHYTQKSLSLS | SEQ ID |
| | QLVQS | NO: 282 |

In italics: C-terminus of anti-Abeta (Aβ) antibody heavy chain Fc
Underlined GGSGG (SEQ ID NO: 214): Linker between c-terminus of anti- Abeta (Aβ) antibody heavy chain Fc and anti-TfR scFv
Bold: N-terminus of anti-TfR scFv

TABLE 24

| | Heavy Chain Knob | Heavy Chain Hole | Light Chain |
|---|---|---|---|
| AB-8 hIgG1 | EVQLVESGGGLIQPGGS | EVQLVESGGGLIQPGGSL | DIQMTQSPSSLS |
| TfR.15.WH8.1 scFv | LRLSCAASGFTVSSNYM | RLSCAASGFTVSSNYMS | ASVGDRVTITC |
| 2+1, no linker | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK | |
| | KQVQLVQSGAEVKKPG | (SEQ ID NO: 230) | |
| | ASVKVSCKASGYTFTSY | | |
| | YMHWVRQAPGQGLEW | | |
| | MGNIYPGSGSSNYAQKF | | |
| | QGRVTMTRDTSTSTVY | | |
| | MELSSLRSEDTAVYYCT | | |
| | RSSYYGNFDTMDNWGQ | | |
| | GTTVTVSSGGSEGKSSG | | |
| | SGSESKSTGGSEIVLTQS | | |
| | PATLSLSPGERATLSCR | | |
| | ASSSIRYMNWYQQKPG | | |
| | QAPRRLIYDTSNRATGIP | | |
| | ARFSGSGSGTDFTLTISS | | |
| | LEPEDFAVYYCHQRSSY | | |
| | PWTFGGGTKVEIK (SEQ | | |
| | ID NO: 261) | | |
| AB-8 hIgG1 | EVQLVESGGGLIQPGGS | EVQLVESGGGLIQPGGSL | DIQMTQSPSSLS |
| TfR.15.WH8.1 scFv | LRLSCAASGFTVSSNYM | RLSCAASGFTVSSNYMS | ASVGDRVTITC |
| 2+1, −5 linker | SWVRQAPGKGLEWVSV | WVRQAPGKGLEWVSVI | RASQSISNYLN |
| | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSQL | SLSPGK (SEQ ID NO: 230) | |
| | VQSGAEVKKPGASVKV | | |
| | SCKASGYTFTSYYMHW | | |
| | VRQAPGQGLEWMGNIY | | |
| | PGSGSSNYAQKFQGRVT | | |
| | MTRDTSTSTVYMELSSL | | |
| | RSEDTAVYYCTRSSYYG | | |
| | NFDTMDNWGQGTTVT | | |
| | VSSGGSEGKSSGSGSES | | |
| | KSTGGSEIVLTQSPATLS | | |
| | LSPGERATLSCRASSSIR | | |
| | YMNWYQQKPGQAPRR | | |
| | LIYDTSNRATGIPARFSG | | |
| | SGSGTDFTLTISSLEPED | | |
| | FAVYYCHQRSSYPWTF | | |
| | GGGTKVEIK (SEQ ID | | |
| | NO: 262) | | |
| AB-8 hIgG1 | EVQLVESGGGLIQPGGS | EVQLVESGGGLIQPGGSL | DIQMTQSPSSLS |
| TfR.15.WH8.1 scFv | LRLSCAASGFTVSSNYM | RLSCAASGFTVSSNYMS | ASVGDRVTITC |
| 2+1, no linker, | SWVRQAPGKGLEWVSV | WVRQAPGKGLEWVSVI | RASQSISNYLN |
| disulfide staple | IYSGGTKHYADSVKGRF | YSGGTKHYADSVKGRFT | WYQQKPGKAP |
| | TISRDNSKNTLYLQMNS | ISRDNSKNTLYLQMNSL | KLLIYDAYSLQI |
| | LRAEDTAVYYCARSYY | RAEDTAVYYCARSYYY | GVPSRFSGSGS |
| | YGSGSSDAFDIWGQGT | GSGSSDAFDIWGQGTMV | GTDFTLTISSLQ |
| | MVTVSSASTKGPSVFPL | TVSSASTKGPSVFPLAPS | PEDFATYYCQQ |
| | APSSKSTSGGTAALGCL | SKSTSGGTAALGCLVKD | SYSIPPTFGGGT |
| | VKDYFPEPVTVSWNSG | YFPEPVTVSWNSGALTS | KVEIKRTVAAP |
| | ALTSGVHTFPAVLQSSG | GVHTFPAVLQSSGLYSLS | SVFIFPPSDEQL |
| | LYSLSSVVTVPSSSLGTQ | SVVTVPSSSLGTQTYICN | KSGTASVVCLL |
| | TYICNVNHKPSNTKVDK | VNHKPSNTKVDKKVEPK | NNFYPREAKVQ |
| | KVEPKSCDKTHTCPPCP | SCDKTHTCPPCPAPELLG | WKVDNALQSG |
| | APELLGGPSVFLFPPKPK | GPSVFLFPPKPKDTLMIS | NSQESVTEQDS |
| | DTLMISRTPEVTCVVVD | RTPEVTCVVVDVSHEDP | KDSTYSLSSTLT |
| | VSHEDPEVKFNWYVDG | EVKFNWYVDGVEVHNA | LSKADYEKHKV |
| | VEVHNAKTKPREEQYN | KTKPREEQYNSTYRVVS | YACEVTHQGLS |
| | STYRVVSVLTVLHQDW | VLTVLHQDWLNGKEYK | SPVTKSFNRGE |
| | LNGKEYKCKVSNKALP | CKVSNKALPAPIEKTISK | C (SEQ ID NO: |
| | APIEKTISKAKGQPREPQ | AKGQPREPQVYTLPPSR | 231) |
| | VYTLPPSRDELTKNQVS | DELTKNQVSLSCAVKGF | |
| | LWCLVKGFYPSDIAVE | YPSDIAVEWESNGQPEN | |
| | WESNGQPENNYKTTPP | NYKTTPPVLDSDGSFFLV | |
| | VLDSDGSFFLYSKLTVD | SKLTVDKSRWQQGNVFS | |
| | KSRWQQGNVFSCSVMH | CSVMHEALHNHYTQKSL | |
| | EALHNHYTQKSLSLSPG | SLSPGK (SEQ ID NO: 230) | |
| | KQVQLVQSGAEVKKPG | | |
| | ASVKVSCKASGYTFTSY | | |
| | YMHWVRQAPGQCLEW | | |
| | MGNIYPGSGSSNYAQKF | | |
| | QGRVTMTRDTSTSTVY | | |
| | MELSSLRSEDTAVYYCT | | |
| | RSSYYGNFDTMDNWGQ | | |
| | GTTVTVSSGGSEGKSSG | | |
| | SGSESKSTGGSEIVLTQS | | |
| | PATLSLSPGERATLSCR | | |
| | ASSSIRYMNWYQQKPG | | |
| | QAPRRLIYDTSNRATGIP | | |
| | ARFSGSGSGTDFTLTISS | | |
| | LEPEDFAVYYCHQRSSY | | |
| | PWTFGCGTKVEIK (SEQ | | |
| | ID NO: 263) | | |
| AB-8 hIgG1 | EVQLVESGGGLIQPGGS | HC hole (Same as #12) | DIQMTQSPSSLS |
| TfR.15.WH8.1.42Q | LRLSCAASGFTVSSNYM | EVQLVESGGGLIQPGGSL | ASVGDRVTITC |
| scFv 2+1 no linker | SWVRQAPGKGLEWVSV | RLSCAASGFTVSSNYMS | RASQSISNYLN |
| | IYSGGTKHYADSVKGRF | WVRQAPGKGLEWVSVI | WYQQKPGKAP |
| | TISRDNSKNTLYLQMNS | YSGGTKHYADSVKGRFT | KLLIYDAYSLQI |
| | LRAEDTAVYYCARSYY | ISRDNSKNTLYLQMNSL | GVPSRFSGSGS |
| | YGSGSSDAFDIWGQGT | RAEDTAVYYCARSYYY | GTDFTLTISSLQ |
| | MVTVSSASTKGPSVFPL | GSGSSDAFDIWGQGTMV | PEDFATYYCQQ |
| | APSSKSTSGGTAALGCL | TVSSASTKGPSVFPLAPS | SYSIPPTFGGGT |
| | VKDYFPEPVTVSWNSG | SKSTSGGTAALGCLVKD | KVEIKRTVAAP |
| | ALTSGVHTFPAVLQSSG | YFPEPVTVSWNSGALTS | SVFIFPPSDEQL |
| | LYSLSSVVTVPSSSLGTQ | GVHTFPAVLQSSGLYSLS | KSGTASVVCLL |
| | TYICNVNHKPSNTKVDK | SVVTVPSSSLGTQTYICN | NNFYPREAKVQ |
| | KVEPKSCDKTHTCPPCP | VNHKPSNTKVDKKVEPK | WKVDNALQSG |
| | APELLGGPSVFLFPPKPK | SCDKTHTCPPCPAPELLG | NSQESVTEQDS |
| | DTLMISRTPEVTCVVVD | GPSVFLFPPKPKDTLMIS | KDSTYSLSSTLT |
| | VSHEDPEVKFNWYVDG | RTPEVTCVVVDVSHEDP | LSKADYEKHKV |
| | VEVHNAKTKPREEQYN | EVKFNWYVDGVEVHNA | YACEVTHQGLS |
| | STYRVVSVLTVLHQDW | KTKPREEQYNSTYRVVS | SPVTKSFNRGE |
| | LNGKEYKCKVSNKALP | VLTVLHQDWLNGKEYK | C (SEQ ID NO: |
| | APIEKTISKAKGQPREPQ | CKVSNKALPAPIEKTISK | 231) |
| | VYTLPPSRDELTKNQVS | AKGQPREPQVYTLPPSR | |
| | LWCLVKGFYPSDIAVE | DELTKNQVSLSCAVKGF | |
| | WESNGQPENNYKTTPP | YPSDIAVEWESNGQPEN | |
| | VLDSDGSFFLYSKLTVD | NYKTTPPVLDSDGSFFLV | |
| | KSRWQQGNVFSCSVMH | SKLTVDKSRWQQGNVFS | |
| | EALHNHYTQKSLSLSPG | CSVMHEALHNHYTQKSL | |
| | KQVQLVQSGAEVKKPG | SLSPGK (SEQ ID NO: 230) | |
| | ASVKVSCKASGYTFTSY | | |
| | YMHWVRQAPGQGLEW | | |
| | MGNIYPGSGSSNYAQKF | | |
| | QGRVTMTRDTSTSTVY | | |
| | MELSSLRSEDTAVYYCT | | |
| | RSSYYGNFDTMDNWGQ | | |
| | GTTVTVSSGGSEGKSSG | | |
| | SGSESKSTGGSEIVLTQS | | |
| | PATLSLSPGERATLSCR | | |
| | ASSSIRQMNWYQQKPG | | |
| | QAPRRLIYDTSNRATGIP | | |
| | ARFSGSGSGTDFTLTISS | | |
| | LEPEDFAVYYCHQRSSY | | |
| | PWTFGGGTKVEIK (SEQ | | |
| | ID NO: 264) | | |
| AB-8 hIgG1 | EVQLVESGGGLIQPGGS | HC hole (Same as #12) | DIQMTQSPSSLS |
| TfR.15.WH8.1.42Q | LRLSCAASGFTVSSNYM | EVQLVESGGGLIQPGGSL | ASVGDRVTITC |
| scFv 2+1, −5 linker | SWVRQAPGKGLEWVSV | RLSCAASGFTVSSNYMS | RASQSISNYLN |
| | IYSGGTKHYADSVKGRF | WVRQAPGKGLEWVSVI | WYQQKPGKAP |
| | TISRDNSKNTLYLQMNS | YSGGTKHYADSVKGRFT | KLLIYDAYSLQI |
| | LRAEDTAVYYCARSYY | ISRDNSKNTLYLQMNSL | GVPSRFSGSGS |
| | YGSGSSDAFDIWGQGT | RAEDTAVYYCARSYYY | GTDFTLTISSLQ |
| | MVTVSSASTKGPSVFPL | GSGSSDAFDIWGQGTMV | PEDFATYYCQQ |
| | APSSKSTSGGTAALGCL | TVSSASTKGPSVFPLAPS | SYSIPPTFGGGT |
| | VKDYFPEPVTVSWNSG | SKSTSGGTAALGCLVKD | KVEIKRTVAAP |
| | ALTSGVHTFPAVLQSSG | YFPEPVTVSWNSGALTS | SVFIFPPSDEQL |
| | LYSLSSVVTVPSSSLGTQ | GVHTFPAVLQSSGLYSLS | KSGTASVVCLL |
| | TYICNVNHKPSNTKVDK | SVVTVPSSSLGTQTYICN | NNFYPREAKVQ |
| | KVEPKSCDKTHTCPPCP | VNHKPSNTKVDKKVEPK | WKVDNALQSG |
| | APELLGGPSVFLFPPKPK | SCDKTHTCPPCPAPELLG | NSQESVTEQDS |
| | DTLMISRTPEVTCVVVD | GPSVFLFPPKPKDTLMIS | KDSTYSLSSTLT |
| | VSHEDPEVKFNWYVDG | RTPEVTCVVVDVSHEDP | LSKADYEKHKV |
| | VEVHNAKTKPREEQYN | EVKFNWYVDGVEVHNA | YACEVTHQGLS |
| | STYRVVSVLTVLHQDW | KTKPREEQYNSTYRVVS | SPVTKSFNRGE |
| | LNGKEYKCKVSNKALP | VLTVLHQDWLNGKEYK | C (SEQ ID NO: |
| | APIEKTISKAKGQPREPQ | CKVSNKALPAPIEKTISK | 231) |
| | VYTLPPSRDELTKNQVS | AKGQPREPQVYTLPPSR | |
| | LWCLVKGFYPSDIAVE | DELTKNQVSLSCAVKGF | |
| | WESNGQPENNYKTTPP | YPSDIAVEWESNGQPEN | |
| | VLDSDGSFFLYSKLTVD | NYKTTPPVLDSDGSFFLV | |
| | KSRWQQGNVFSCSVMH | SKLTVDKSRWQQGNVFS | |
| | EALHNHYTQKSLSLSQL | CSVMHEALHNHYTQKSL | |
| | VQSGAEVKKPGASVKV | SLSPGK (SEQ ID NO: 230) | |
| | SCKASGYTFTSYYMHW | | |
| | VRQAPGQGLEWMGNIY | | |
| | PGSGSSNYAQKFQGRVT | | |
| | MTRDTSTSTVYMELSSL | | |
| | RSEDTAVYYCTRSSYYG | | |
| | NFDTMDNWGQGTTVT | | |
| | VSSGGSEGKSSGSGSES | | |
| | KSTGGSEIVLTQSPATLS | | |
| | LSPGERATLSCRASSSIR | | |
| | QMNWYQQKPGQAPRR | | |
| | LIYDTSNRATGIPARFSG | | |
| | SGSGTDFTLTISSLEPED | | |
| | FAVYYCHQRSSYPWTF | | |
| | GGGTKVEIK (SEQ ID | | |
| | NO: 265) | | |
| AB-8 hIgG1 | EVQLVESGGGLIQPGGS | HC hole (Same as #12) | DIQMTQSPSSLS |
| TfR.15.WH8.1.42Q | LRLSCAASGFTVSSNYM | EVQLVESGGGLIQPGGSL | ASVGDRVTITC |
| scFv 2+1 no linker, | SWVRQAPGKGLEWVSV | RLSCAASGFTVSSNYMS | RASQSISNYLN |
| disulfide staple | IYSGGTKHYADSVKGRF | WVRQAPGKGLEWVSVI | WYQQKPGKAP |
| | TISRDNSKNTLYLQMNS | YSGGTKHYADSVKGRFT | KLLIYDAYSLQI |
| | LRAEDTAVYYCARSYY | ISRDNSKNTLYLQMNSL | GVPSRFSGSGS |
| | YGSGSSDAFDIWGQGT | RAEDTAVYYCARSYYY | GTDFTLTISSLQ |
| | MVTVSSASTKGPSVFPL | GSGSSDAFDIWGQGTMV | PEDFATYYCQQ |
| | APSSKSTSGGTAALGCL | TVSSASTKGPSVFPLAPS | SYSIPPTFGGGT |
| | VKDYFPEPVTVSWNSG | SKSTSGGTAALGCLVKD | KVEIKRTVAAP |
| | ALTSGVHTFPAVLQSSG | YFPEPVTVSWNSGALTS | SVFIFPPSDEQL |
| | LYSLSSVVTVPSSSLGTQ | GVHTFPAVLQSSGLYSLS | KSGTASVVCLL |
| | TYICNVNHKPSNTKVDK | SVVTVPSSSLGTQTYICN | NNFYPREAKVQ |
| | KVEPKSCDKTHTCPPCP | VNHKPSNTKVDKKVEPK | WKVDNALQSG |
| | APELLGGPSVFLFPPKPK | SCDKTHTCPPCPAPELLG | NSQESVTEQDS |
| | DTLMISRTPEVTCVVVD | GPSVFLFPPKPKDTLMIS | KDSTYSLSSTLT |
| | VSHEDPEVKFNWYVDG | RTPEVTCVVVDVSHEDP | LSKADYEKHKV |
| | VEVHNAKTKPREEQYN | EVKFNWYVDGVEVHNA | YACEVTHQGLS |
| | STYRVVSVLTVLHQDW | KTKPREEQYNSTYRVVS | SPVTKSFNRGE |
| | LNGKEYKCKVSNKALP | VLTVLHQDWLNGKEYK | C (SEQ ID NO: |
| | APIEKTISKAKGQPREPQ | CKVSNKALPAPIEKTISK | 231) |
| | VYTLPPSRDELTKNQVS | AKGQPREPQVYTLPPSR | |
| | LWCLVKGFYPSDIAVE | DELTKNQVSLSCAVKGF | |
| | WESNGQPENNYKTTPP | YPSDIAVEWESNGQPEN | |
| | VLDSDGSFFLYSKLTVD | NYKTTPPVLDSDGSFFLV | |
| | KSRWQQGNVFSCSVMH | SKLTVDKSRWQQGNVFS | |
| | EALHNHYTQKSLSLSPG | CSVMHEALHNHYTQKSL | |
| | KQVQLVQSGAEVKKPG | SLSPGK (SEQ ID NO: 230) | |
| | ASVKVSCKASGYTFTSY | | |
| | YMHWVRQAPGQCLEW | | |
| | MGNIYPGSGSSNYAQKF | | |
| | QGRVTMTRDTSTSTVY | | |
| | MELSSLRSEDTAVYYCT | | |
| | RSSYYGNFDTMDNWGQ | | |
| | GTTVTVSSGGSEGKSSG | | |
| | SGSESKSTGGSEIVLTQS | | |
| | PATLSLSPGERATLSCR | | |
| | ASSSIRQMNWYQQKPG | | |
| | QAPRRLIYDTSNRATGIP | | |
| | ARFSGSGSGTDFTLTISS | | |
| | LEPEDFAVYYCHQRSSY | | |
| | PWTFGCGTKVEIK (SEQ | | |
| | ID NO: 266) | | |

### Example 30: Binding Affinity of Anti-pyroGlu3 Abeta (Aβ)-huTfR Antibodies to pyroGlu3-24 Abeta (Aβ)Peptide and to Transferrin Receptor Extracellular Domain (TfR ECD)

As described above and throughout the Examples and Figures, TfRL10-16, TfR10-16 or 10-16 refers to TfR.15.WH8.1.42Q.L10-16; TfRL10-8 or TfR10-8 or 10-8 refers to TfR.15.WH8.1.42Q.L10-8; TfR7-2 or TfRL7-2, L7-2 or 7-2 refers to TfR.15.WH8.1.42Q.L7-2; TfR42Q or 42Q refers to TfR.15.WH8.1.42Q; TfR39.38 or 39.38 refers to TfR.9.1B.39.38; and TfR24A or 24A refers to TfR.15.WH8.1.24A.

The binding kinetics of anti-pyroGlu3 AP-huTfR antibodies of the present disclosure to human pyroGlu3-24 Aβ peptide were assessed using the Carterra LSA-XT. Binding kinetics to human and cynomolgus monkey TfR extracellular domains (ECDs) was performed using a Biacore T200. For pyroGlu3-24 Aβ pE3-24 peptide binding, antibodies were captured with a polyclonal anti-human Fc lawn on an HC200M sensor chip (Carterra), with peptide injected in a concentration series. Data were analyzed using NextGenKIT software. For human and cynomolgus TfR ECDs, biotinylated ECDs were immobilized on a Streptavidin coated Biacore sensor chip (Cytiva), and anti-pyroGlu3 AP-huTfR antibodies in a 2+1 format of the present disclosure were injected in a concentration series using a single-cycle kinetics method. Data were analyzed using BiaEvaluation software (Cytiva). Results from these studies are summarized in Table 25 below.

TABLE 25

| | Human | Human | Cyno |
|---|---|---|---|
| | pyroGlu3-24 | TfR ECD | TfR ECD |
| Antibody | Aβ peptide (nM) | (nM) | (nM) |
| AB-6-TfR42Q | 418 | 24 | 12 |
| AB-8-TfR42Q | 117 | 52 | 30 |
| AB-8-TfR7-2 | 127 | 427 | 737 |
| AB-8-TfR10-8 | 116 | 675 | 680 |
| AB-6-TfR24A | 526 | 330 | 644 |
| AB-8-TfR24A | 139 | 1210 | 2070 |
| AB-8-TfR9.1B.35 | 194 | 1270 | 4110 |
| AB-8-TfR39.38 | 131 | 1880 | 2610 |
| AB-8-TfR10-16 | 91 | N/A | 3770 |
| AB-6-TfR10-16 | 227 | 725 | 1325 |

As shown in Table 25, anti-Aβ Fab components of various AB-8-TfR antibodies of the present disclosure in a 2+1 format bound to pyroGlu-Aβ peptide pE3-24 with affinities ranging from 91 nM to 194 nM. Anti-Aβ Fab components of various AB-6-TfR antibodies in a 2+1 format of the present disclosure bound to pyroGlu-Aβ peptide pE3-24 with affinities ranging from 227 nM to 526 nM. These measured values were within experimental error for this method.

Anti-TfR scFv components of various AB-8-TfR antibodies in a 2+1 format bound to both human and cynomolgus TfR ECDs with affinities ranging from 52-1880 nM and 30-4110 nM, respectively, as shown in Table 25. In particular, the TfR scFv component of the AB-8-TfR10-16 antibody in a 2+1 format bound to both human and cynomolgus TfR ECD with affinities of approximately 1,000 nM, while that of the AB-6-TfR10-16 antibody in a 2+1 format bound to cynomolgus TfR ECD with approximately 3-fold lower affinity. Additional affinity measurements of the binding of these two antibodies to human and cynomolgus TfR apical domains showed comparable affinities of approximately 3 μM and 6-7 μM, respectively (see Example 31 and Table 26).

Binding Affinities of AB-8-TfR42Q, AB-8-TfR10-8, AB-8-TfR10-16, AB-6-TfR10-16 to pyroGlu3-24 Abeta (Aβ) Peptide

To confirm the binding interactions in the above experiments, binding affinity and kinetics of purified anti-pyroGlu3 Abeta (Aβ)-huTfR antibodies in a 2+1 format were further tested in an additional assay. This assay used a sensor chip with slightly different surface chemistry and was selected to optimize the kinetic quality and minimize the chance for mass-transport limitation. This assay also used an independently generated pyroglutamate-modified Abeta (Aβ) polypeptide (pE3-24; LifeTein) and a previously tested, non-modified Abeta (Aβ) polypeptide as a negative control (Aβ3-40; Yenzym). Binding kinetics and affinity were determined using a Carterra LSA instrument (Carterra, Salt Lake City, UT).

Antibodies were diluted to concentrations of 30 μg/mL and 6 μg/mL in multiple replicates using running buffer consisting of 1×HBS-EP+(Teknova, #H8022) supplemented with 0.5 mg/mL bovine serum albumin (BSA; Millipore, #82-045-1). A capture lawn was prepared on an SAH30M sensor chip (Carterra, #4279) by coupling a mixture of biotinylated anti-human Fc VHH (20 μg/mL; Thermo Fisher Scientific, #7103262100) and biotinylated anti-human CH1 VHH (20 μg/mL; Thermo Fisher Scientific, #7103202100). Antibody replicates were printed as a single array over the capture lawn, with each print step conducted for 10 minutes.

Pyroglutamate-modified Abeta (Aβ) (pE3-24) and non-modified Abeta (Aβ) (3-40) peptide analytes were diluted in running buffer and prepared as a concentration series comprising six three-fold serial dilutions followed by two ten-fold dilutions, starting from 2500 nM (0.1, 1.0, 10, 31, 93, 278, 833, and 2500 nM). Each analyte injection cycle consisted of a single, non-regenerative injection of analyte over the captured antibody array, performed sequentially from the lowest to highest concentration. Analytes were injected for 3 minutes, followed by a dissociation phase of 5 minutes in running buffer. At the conclusion of each injection cycle, the VHH capture lawn was regenerated using 10 mM glycine-HCl, pH 1.7. Binding data were processed and analyzed using NextGenKIT high-throughput kinetics analysis software (Carterra).

As shown in Table 26, anti-Abeta (Aβ) Fab components of AB-8-TfR antibodies in a 2+1 format bound to pyroglutamate-modified Abeta (Aβ) peptide pE3-24 with equilibrium dissociation constants (K_{D}) ranging from approximately 6 nM to 13 nM. Anti-Abeta (Aβ) Fab components of AB-6-TfR antibodies in a 2+1 format bound to the same pyroglutamate-modified Abeta (Aβ) peptide with affinities of approximately 45 nM. As expected, no significant binding was observed to the negative control unmodified E3-40 peptide (data not shown). The values in Table 26 provided additional information for use in further development of the anti-pyroGlu3 Abeta (Aβ)-huTfR antibodies.

TABLE 26

| | Human |
|---|---|
| | pyroGlu3-24 |
| | Abeta (Aβ) |
| Antibody | peptide (nM) |
| AB-8-TfR42Q (N = 8) | 10 ± 3 |
| AB-8-TfR10-8 (N = 8) | 8 ± 2 |
| AB-8-TfR10-16 (N = 4) | 10 ± 0.5 |
| AB-6-TfR10-16 (N = 4) | 45 ± 3 |

### Binding Kinetics of AB-8-TfR42Q, AB-8-TfR10-8, AB-8-TfR10-16, AB-6-TfR10-16, and AB-8-TfR39.38 to Human and Cynomolgus TfR ECD

To confirm the binding interactions from the previous binding kinetics experiment using human and cyno TfR ECD, an additional assay was performed. The kinetics of the anti-pyroGlu3 Abeta (Aβ)-huTfR antibodies in a 2+1 format binding to human and cynomolgus monkey TfR ECD were tested using the Biacore T200 (Cytiva) and employing a method designed to evaluate rapid binding kinetics. This analysis used TfR ECDs produced from constructs that included N-terminal His/Avi tags for capture and detection. A capture chip was prepared using rabbit anti-Avi-tag (R&D Systems MAB10546) immobilized on a CM-4 sensor chip (Cytiva) by amine coupling using a running buffer without BSA (HBS-EP+, cat #8020, Teknova, Hollister, CA). Purified, tagged ECDs were captured at low densities on the chip (~8-14 RUs), and anti-pyroGlu3 Aβ-huTfR antibodies in a 2+1 format were diluted in Running Buffer (HBS-EP+ with 0.5 mg/mL BSA, Roche cat #3117332001) and injected at a fast flow rate of 75 μL/min using a single-cycle kinetics method. Five concentrations were injected in series starting from low to high concentrations, with the range adjusted for each antibody's kinetics that had been estimated during earlier screening analyses.

The analyte range for AB-8-TfR.42Q was 300 nM to 0.5 nM. For AB-8-TfR.L10-8, the range was 4 uM to 6.4 nM, and for the other antibodies, the range was 8 μM to 12.8 nM; all were done in 3-fold serial dilutions. The analytes were injected for 10 seconds, followed by 10 minutes of dissociation in Running Buffer after the final, highest concentration injection. After the injection series of each test antibody, the chip surface was regenerated with 10 mM Glycine pH2.0. Data were analyzed using BiaEvaluation software (Cytiva).

The anti-TfR scFv components of the various anti-pyroGlu3 Abeta (Aβ)-huTfR antibodies bound to both human and cyno TfR ECDs with affinities ranging from double-digit nM to single digit μM affinities, as shown in Table 27 and Table 28, respectively. AB-8-TfR42Q bound human and cyno TfR ECD at approximately 45 nM and 23 nM, respectively. AB-8-TfR10-8 bound human and cyno TfR ECD at approximately 732 nM and 661 nM, respectively. AB-8-TfR10-16 and AB-6-TfR10-16, which share the same TfR scFv component, bound human and cyno TfR ECD at approximately 2 μM. AB-8-TfR39.38 also bound human TfR ECD at approximately 2 μM, and bound cyno TfR ECD at approximately 4.6 μM. The values in Tables 27 and 28 provided additional information on the binding kinetics (on- and off-rates) for use in further development of the anti-pyroGlu3 Abeta (Aβ)-huTfR antibodies.

TABLE 27
Binding kinetics and affinity of anti-pyroGlu3
Abeta (Aβ)-huTfR antibodies to human TfR ECD

| | kₐ | k_{d} | K_{D} |
|---|---|---|---|
| | (1/Ms) | (1/s) | (nM) |
| AB-8-TfR42Q | 6.7e+5 | 3.0e−2 | 45 |
| AB-8-TfR10-8 | 1.1e+5 | 8.2e−2 | 732 |
| AB-8-TfR10-16 | 7.4e+4 | 1.5e−1 | 1990 |
| AB-6-TfR10-16 | 6.9e+4 | 1.5e−1 | 2110 |
| AB-8-TfR39.38 | 5.3e+4 | 1.0e−1 | 1912 |

TABLE 28
Binding kinetics and affinity of anti-pyroGlu3
Abeta (Aβ)-huTfR antibodies to cyno TfR ECD

| | kₐ | k_{d} | K_{D} |
|---|---|---|---|
| | (1/Ms) | (1/s) | (nM) |
| AB-8-TfR42Q | 5.6e+5 | 1.3e−2 | 23 |
| AB-8-TfR10-8 | 6.6e+4 | 4.4e−2 | 661 |
| AB-8-TfR10-16 | 2.7e+4 | 6.6e−2 | 2460 |
| AB-6-TfR10-16 | 3.1e+4 | 5.7e−2 | 1860 |
| AB-8-TfR39.38 | 9.1e+4 | 4.2e−1 | 4600 |

### Example 31: Binding Affinity of AB-6-TfR10-16 and AB-8-TfR10-16 to Human and Cynomolgus TfR Apical Domain

The affinities of AB-6-TfR10-16 binding to both the human and cynomolgus TfR apical domains were also measured and were comparable to AB-8-TfR10-16 as measured using the Carterra LSA-XT. Antibodies were captured with an anti-human Fc and CH1 surface on a Carterra sensor chip, and a concentration series of human or cynomolgus TfR apical domain was injected in a single-cycle kinetics method. Data were analyzed using NextGenKIT software (Carterra). These values are shown in Table 29.

TABLE 29

| | Human TfR | Cynomolgus TfR |
|---|---|---|
| | apical domain | apical domain |
| Antibody | (nM) | (nM) |
| AB-8-TfR10-16 | 3500 | 7000 |
| AB-6-TfR10-16 | 3300 | 6100 |

### Example 32: Enhanced Uptake of pyroGlu3 Abeta (Aβ) by Microglia Using a Human CNS Triple Culture Cell Model

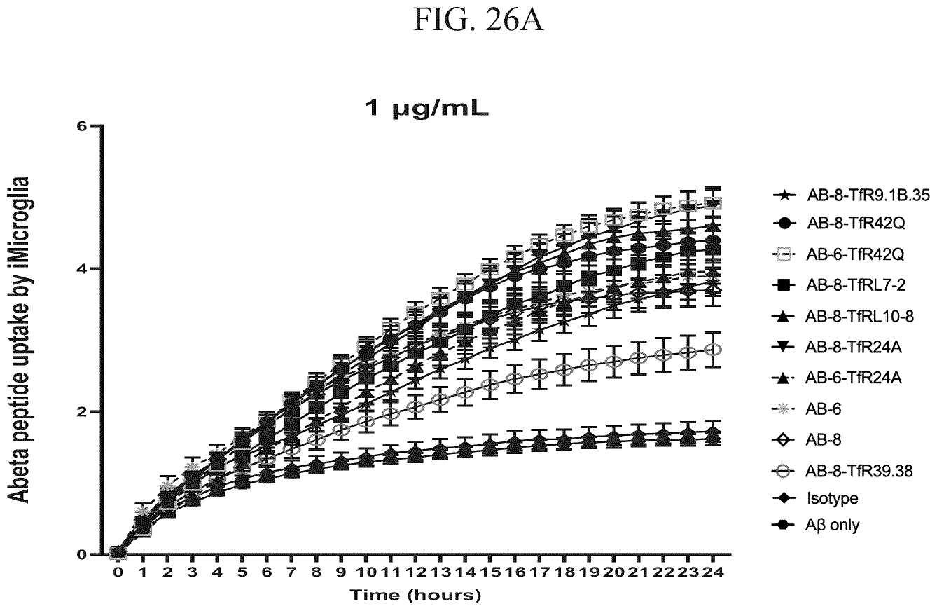

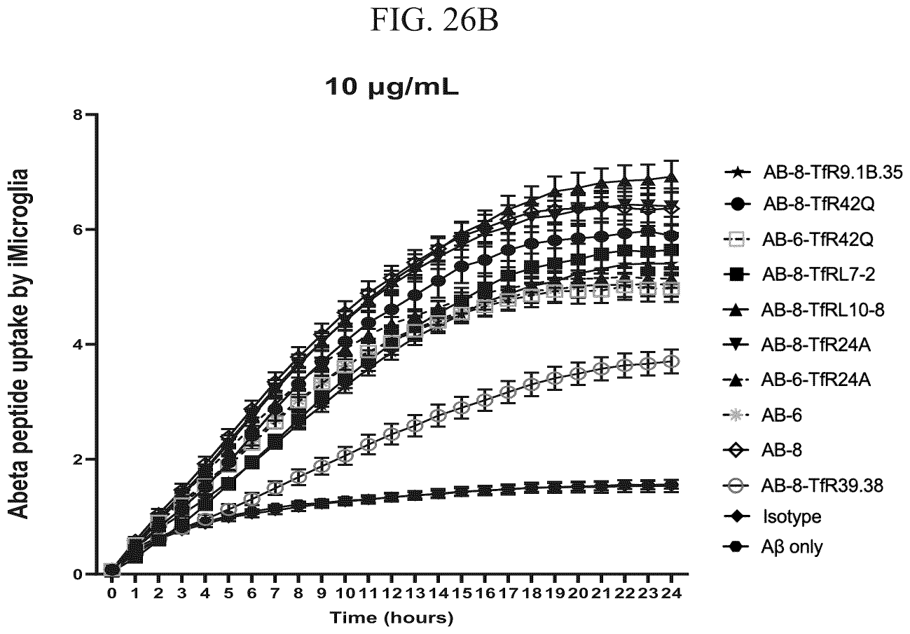

Anti-pyroGlu3 Abeta (Aβ) antibodies of the present disclosure were assessed for their ability to promote the uptake of pyroGlu3 Abeta (Aβ) by microglia using a human CNS triple culture cell model. iPSC-derived microglia precursors expressing GFP were grown in parallel with iPSC-derived neuron and human fetal astrocyte co-cultures, and matured to form a genetically and functionally relevant human CNS triple culture cell system. CNS triple culture cells were simultaneously treated with anti-pyroGlu3 Abeta (Aβ) antibodies (1 and 10 μg/mL) and pyroGlu3-24 Aβ polypeptides (1 μM) labeled with fluorescent pH-sensitive pHrodo-Red dye for 24 hours. Uptake of pyroGlu3-24 Abeta (Aβ) by microglia in this human CNS triple culture cell system was monitored by IncuCyte in real time. The fluorescence intensity of pHrodo-Red signal was normalized to GFP-tagged microglia to evaluate the effect of microglial pyroGlu3-24 Aβ phagocytosis. As shown in FIG. 26A and FIG. 26B, anti-pyroGlu3 Abeta (Aβ)-huTfR antibodies showed similar increases in microglial pyroGlu3 Aβ phagocytosis as that observed with naked anti-pyroGlu3 Abeta (Aβ) antibodies (AB-6 and AB-8) at either 1 ag/ml or 10 μg/ml. These data indicated that anti-pyroGlu3 Abeta (Aβ)-huTfR antibodies of the present disclosure (e.g., AB-8-TfR42Q) are effective at phagocytosis of pyroGlu3 Aβ.

### Example 33: Enhanced Uptake of pyroGlu3 Abeta (Aβ) by Anti-pyroGlu3 Aβ Antibody with Active Fc in a Human CNS Triple Culture Cell Model

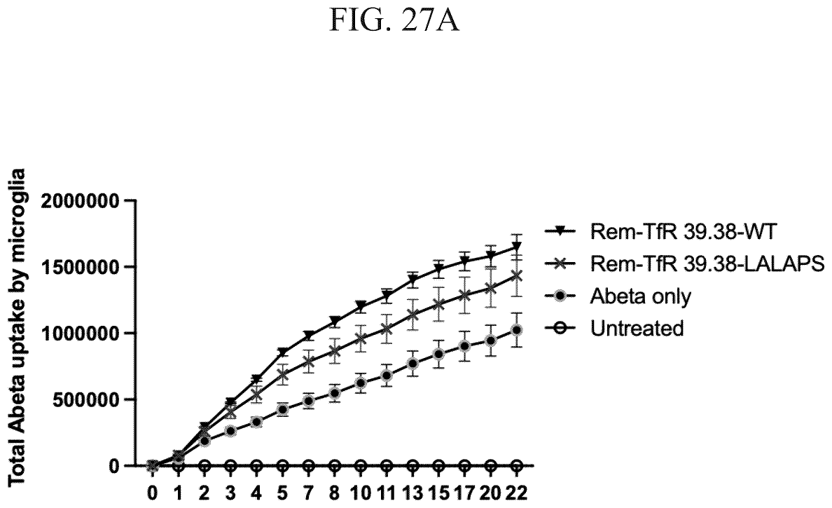

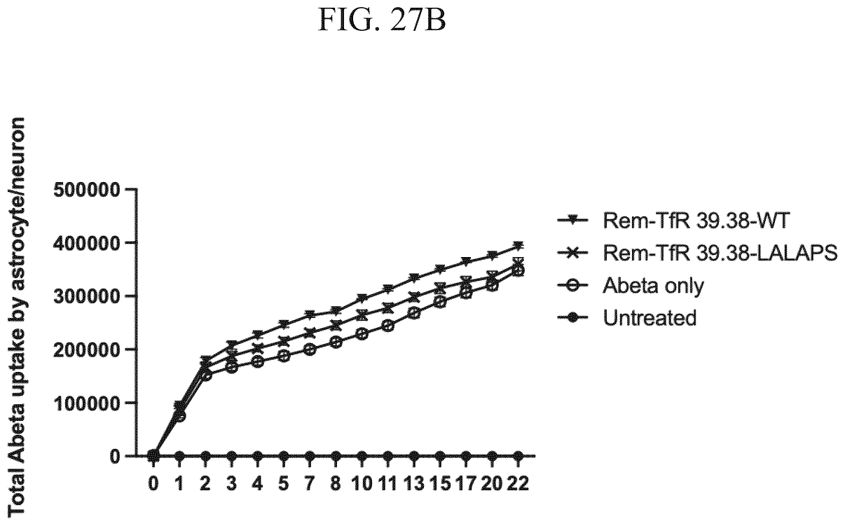

Anti-pyroGlu3 Abeta (Aβ) antibodies of the present disclosure formatted with different Fc variants were assessed for their ability to promote the uptake of pyroGlu3 Abeta (Aβ) using a human CNS triple culture cell model. iPSC-derived microglia precursors expressing GFP were grown in parallel with iPSC-derived neuron and human fetal astrocyte co-cultures. CNS triple culture cells were simultaneously treated with anti-pyroGlu3 Abeta (Aβ) antibodies (67 nM) and pyroGlu3-24 Abeta (Aβ) polypeptides (1 μM) labeled with fluorescent pH-sensitive pHrodo-Red dye for 24 hours. Uptake of pyroGlu3-24 Abeta (Aβ) by microglia or astrocyte/neuron in this human CNS triple culture cell system was monitored by IncuCyte in real time. The fluorescence intensity of pHrodo-Red signal was normalized to GFP tagged microglia or non-GFP tagged astrocyte and neuron to evaluate the effect of pyroGlu3-24 Abeta (Aβ) phagocytosis. As shown in FIG. 27A and FIG. 27B, an anti-pyroGlu3 Abeta (Aβ)-huTfR with WT IgG antibody Fc (“remternetug-TfR 39.38-WT”) showed higher levels of pyroGlu3 Aβ in both microglia and astrocyte/neuron, respectively, compared to that observed with an anti-pyroGlu3 Abeta (Aβ)-huTfR with Fc variant LALAPS (which has reduced effector function) (“remternetug-TfR 39.38-LALAPS”). These data indicated that an active Fc in these antibody formats resulted in increased levels of phagocytosis of pyroGlu3 Abeta (Aβ) by anti-pyroGlu3 Abeta (Aβ) antibody.

### Example 34. Transcytosis of Anti-pyroGlu3 Abeta (Aβ)-TfR Antibodies Across Human or Cynomolgus Monkey Brain Endothelial Cells

A human cerebral endothelial cell line (hCMEC/D3) obtained from Sigma/Millipore (Cat #SCC066; passages 5-20) was grown on rat tail collagen type I (Cat #Sigma-Aldrich 08-115, 60 g/mL) coated plates in endothelia cell medium MV (Cat #PromoCell C-22220). Cynomolgus monkey primary brain microvascular endothelial cells obtained from Cell Biologics (passages 2-5) were grown on 0.2% gelatin coated plates in monkey endothelial cell medium (Cat #Cell Biologics MIK 168). Both human and cynomolgus monkey brain endothelial cells were seeded in 65 mm transwells with 0.4 μm pore polyester membrane inserts (Cat #Costar 3470, 1.5×10⁵ cells/cm2) and cultured for 4 days for brain endothelial monolayer formation. Anti-pyroGlu3 Abeta (Aβ)-huTfR antibodies of the present disclosure (500 nM) were added in the apical chamber and pulse-incubated with brain endothelia cells for 2 hours at 37 °C and 5% CO₂. Transwells were then apically (150 μL) and basolaterally (700 μL) washed with culture medium three times. Fresh medium was added to apical (100 μL) and basolateral (600 μL) chambers and the cultures were incubated at 37 °C and 5% CO₂ for 4 hours, upon which time entire apical and basolateral supernatants were collected to assess the recycling and transcytosis of the antibodies into the apical and basolateral chambers, respectively. The cell lysate of brain endothelial monolayer was prepared with RIPA buffer and collected.

Antibody concentrations in media samples and cell lysates from the transcytosis assay were measured by Mesoscale Discovery (MSD) analysis. A streptavidin multi-array 384-well plate was coated with 0.1 μg/ml biotin-Goat anti-Human IgG (Cat #SouthernBiotech 2049-08) diluted in Tris assay buffer (1% BSA in TBST) and incubated at room temperature for 1 hour. After washing with wash buffer (0.05% Tween-20 in TBS) three times, 25 μl/well of apical and basolateral samples and cell lysates in Tris assay buffer were added and incubated at room temperature for 2 hours, followed with 3×washing. The plate was incubated with 0.25 μg/mL ru-Goat anti-Human IgG working solution (Cat #MSD R32AJ-5) in 0.1% mouse serum in Tris assay buffer for 1 hour. Following a final wash cycle, the wells were developed with MSD Read Buffer T (Cat #MSD R92TD-1) at room temperature according to the manufacturer's recommendations. Electrochemiluminescence signals were measured by ultra-sensitive plate imager (Cat #MESO SECTOR S 600MM).

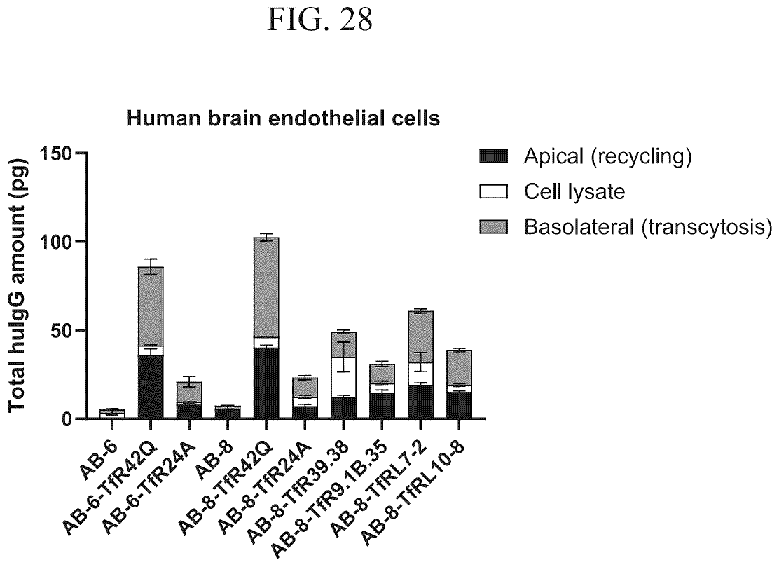

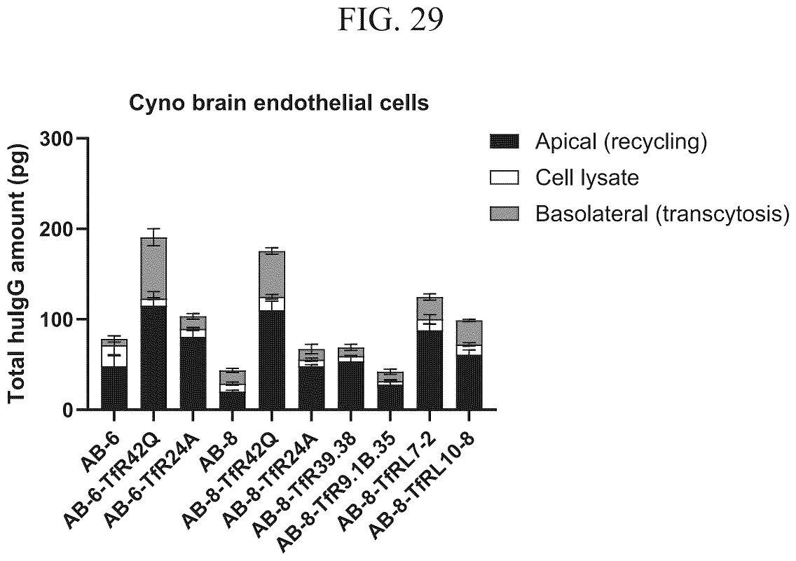

As shown in FIG. 28 and FIG. 29, a significant increase in apical recycling and basolateral transcytosis of anti-pyroGlu3 Abeta (Aβ)-huTfR antibodies was observed compared to that with anti-pyroGlu3 Abeta (Aβ) antibodies lacking a TfR binding moiety in both human (FIG. 28) and cynomolgus monkey (FIG. 29) cerebral endothelial cells. The greatest transcytosis of anti-pyroGlu3 Abeta (Aβ)-TfR antibodies across human brain endothelial cell line hCMEC/D3 was observed for AB-8-TfR42Q. These results indicated that anti-pyroGlu3 Abeta (Aβ)-huTfR antibodies of the present disclosure were effective at crossing the brain endothelia monolayer via TfR-mediated transport pathways.

### Example 35: Anti-pyroGlu3 Abeta (Aβ)-huTfR Antibodies Show Reduced Antibody Dependent Cellular Cytotoxicity (ADCC) In Vitro in CHO Cells Overexpressing huTfR

In addition to its expression by brain endothelial cells, TfR is also expressed at high levels in immature red blood cells (reticulocytes) in the periphery. Safety liabilities associated with anti-transferrin receptor antibodies with active Fc effector function (e.g., a wild type human IgG1 Fc) have been previously reported. In huTfR knock-in mice treated with certain anti-TfR antibodies, acute clinical signs and reductions in reticulocytes were observed after dosing (Couch et al., 2013, Science Translation Medicine, 5(183)). This observation has been linked to Fc effector function of the antibodies, and antibody dependent cellular cytotoxicity (ADCC) and complement dependent cytotoxicity (CDC) have been described to be involved in these processes.

ADCC activity of anti-pyroGlu3 Abeta (Aβ)-TfR antibodies of the present disclosure were thus assessed using an effector cell line (engineered Jurkat cells stably expressing FcγRIIIa V158 variant) (Promega G7010). The engineered effector cell contains a nuclear factor of activated T-cells (NFAT) response element that drives expression of firefly luciferease upon signaling through FcγRIIIa. For target cells, human transferrin receptor (huTfR) overexpressing CHO cells were generated from a 2-day transient transfection of ExpiCHO cells with an expression plasmid containing a full-length huTfR construct. Antibody concentrations used in this assay ranged from 0.017 μg/mL to 333.3 μg/mL.

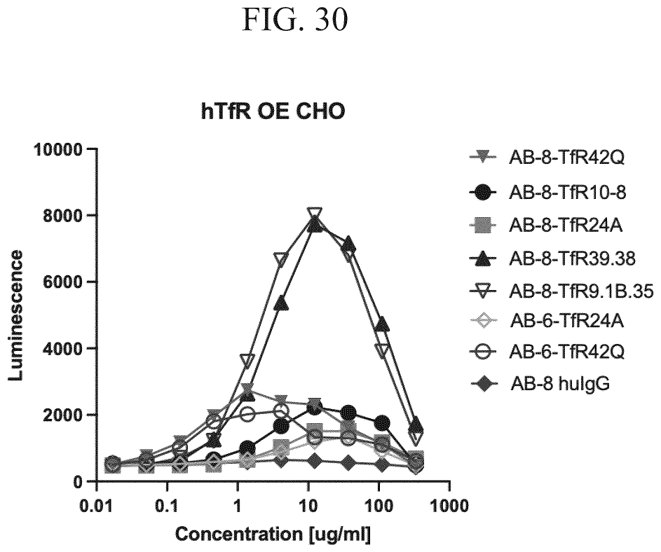

FIG. 30 shows that anti-pyroGlu3 Abeta (Aβ)-TfR antibodies AB-8-TfR39.38 and AB-8-TfR9.1B.35 induced a strong level of ADCC activity in this assay. In comparison, AB-8-TfR10-8, AB-8-TfR24A, AB-8-TfR42Q, AB-6-TfR24A, and AB-6-TfR42Q antibodies induced significantly less ADCC activity, despite having comparable or higher affinity to human TfR than AB-8-TfR39.38 and AB-8-TfR9.1B.35 (See Table 25 above). As expected, AB-8-huIgG did not display ADCC activation, as this IgG antibody does not have an anti-TfR moiety attached. The TfR10-8, TfR24A, and TfR42Q antibodies were engineered from the same lineage, and bind huTfR at the same epitope, while TfR39.38 and TfR9.1B.35 antibodies are from a different lineage and bind huTfR at a different epitope. Taken together, these results suggested that the lineage/epitope of TfR binding may influence the engagement of Fc to FcγRs and ADCC activation. The binding affinity of the antibody to huTfR does play a role but less so than the impact from the lineage/epitope, as the affinity of TfR39.38 and TfR9.1B.35 are in the μM range, while the affinity of TfR10-8, TfR 24A, and TfR 42Q ranges from double digit nM-single digit μM range. These results showed that certain anti-pyroGlu3 Abeta (Aβ)-TfR antibodies of the present disclosure (AB-8-TfR10-8, AB-8-TfR24A, AB-8-TfR42Q, AB-6-TfR24A, AB-6-TfR42Q) demonstrated reduced level of ADCC activation in comparison to other anti-pyroGlu3 Abeta (Aβ)-TfR antibodies (AB-8-TfR39.38 and AB-8-TfR9.1B.35).

### Example 36: Fc Gamma Receptor Binding of Anti-pyroGlu3 Abeta (Aβ)-huTfR Antibodies

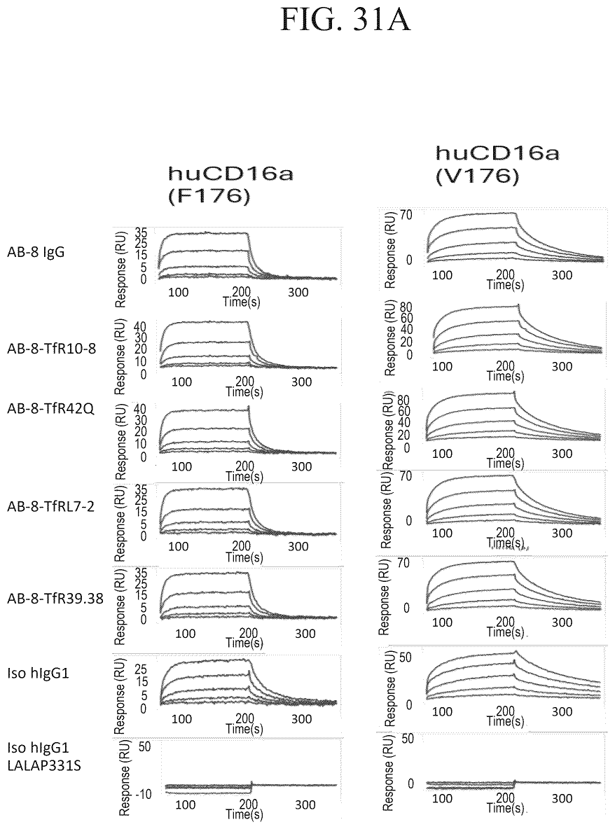

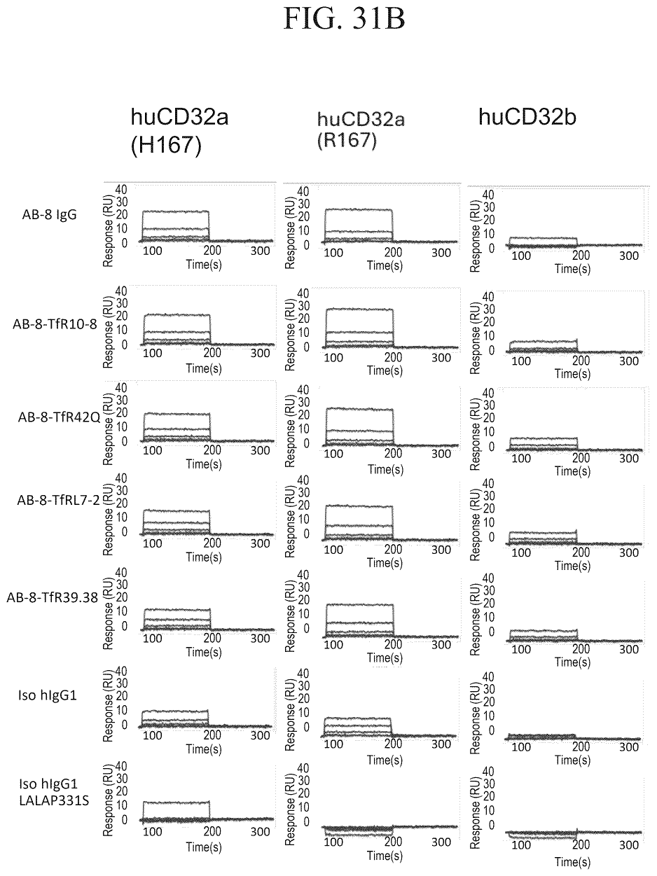

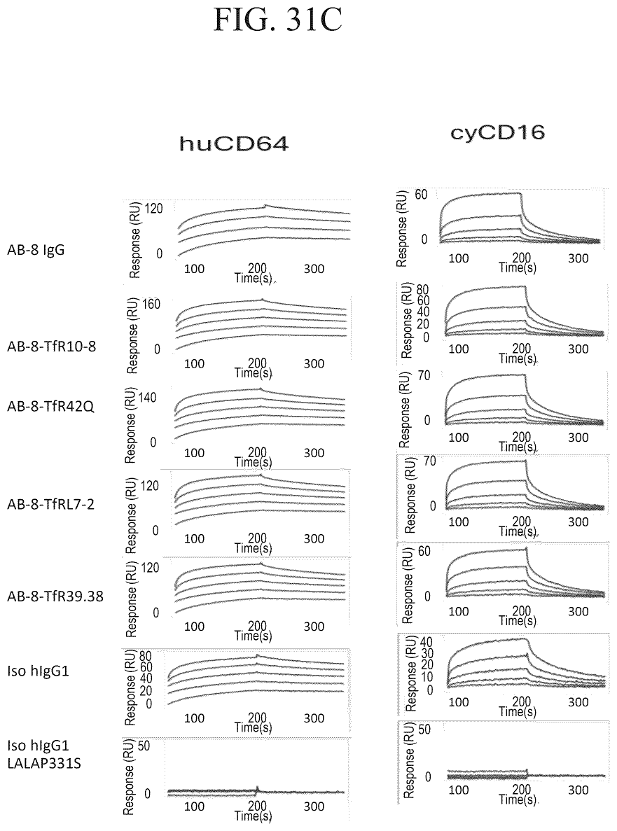

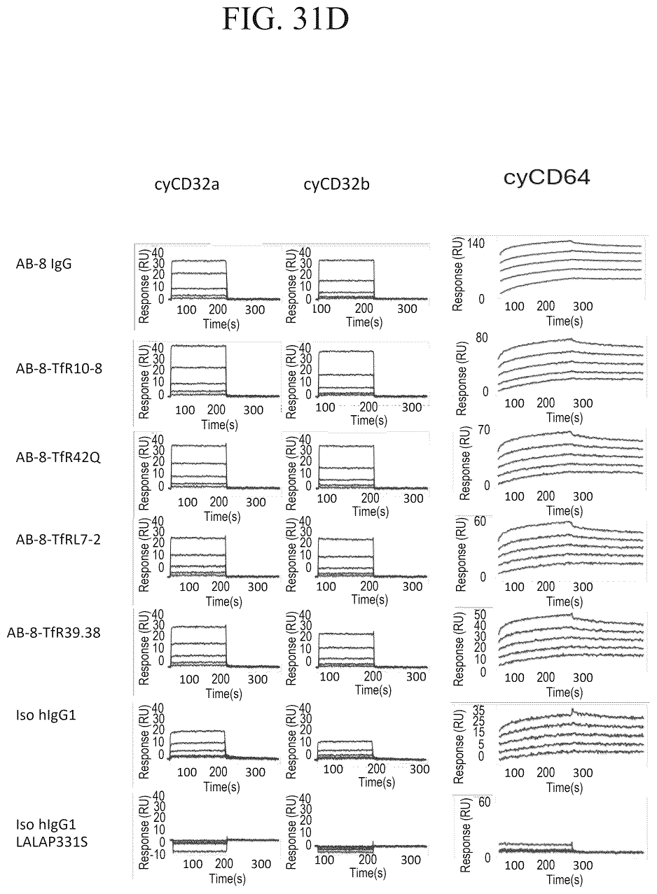

Because the engineered anti-pyroGlu3 Abeta (Aβ)-huTfR antibodies of the present disclosure carry an scFv at the C-terminus of the Fc domain, any potential effects on binding to Fc gamma receptors was evaluated as follows. Analysis of binding kinetics was carried out by Surface Plasmon Resonance (SPR) using the Carterra LSA-XT with recombinant human and cynomolgus monkey Fc gamma receptor extracellular domains and antibodies with or without various TfR variants. The comparability of the Fc gamma receptor binding kinetic profiles are shown in FIG. 31A-FIG. 31D. The results showed that the presence of the scFv did not measurably affect this interaction in this format. Furthermore, the antibodies bound to cynomolgus Fc gamma receptors with similar profiles as that observed with human Fc gamma receptors.

For the assessment, recombinant His-tagged Fc gamma receptor extracellular domains were immobilized onto an HC30M sensor chip by amine coupling at 30, 15, and 7.5 μg/mL concentrations in duplicate and 3.75 μg/mL in quadruplicate. Antibodies at five 3-fold serial dilutions from 3 μM to 37 nM were injected from low to high concentrations without regeneration between injections. The chip was regenerated with 10 mM glycine pH 2.5 between series. Data were analyzed using NextGenKIT software (Carterra).

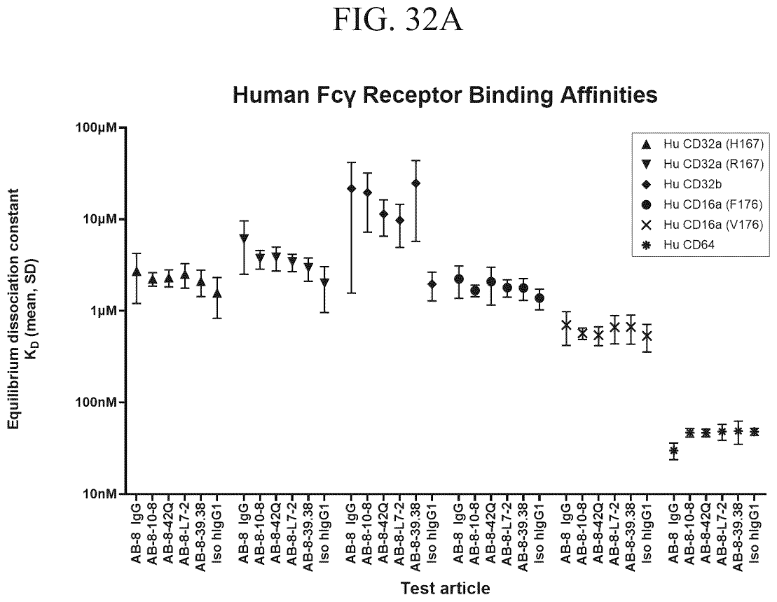

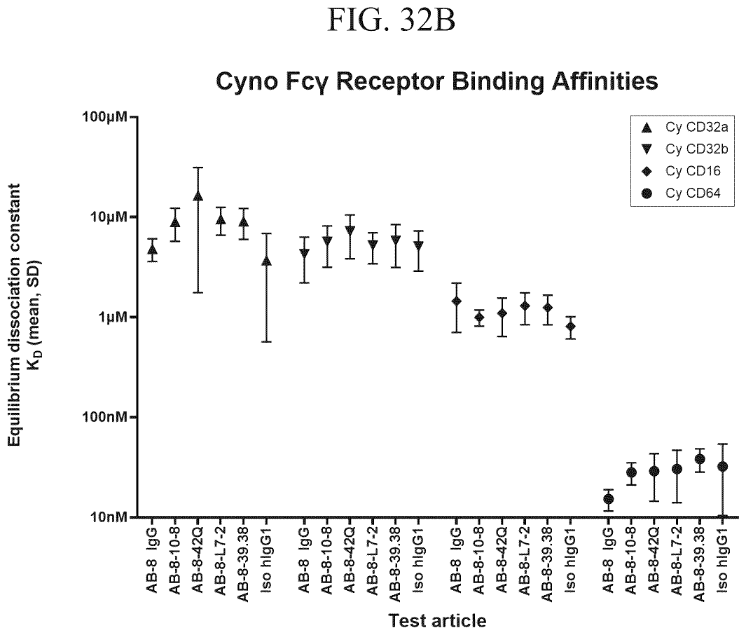

The similarity of the binding kinetic profiles was also evident in the overall affinities of the various receptors, as shown in FIGS. 32A and 32B and Table 30. The CD64 (Fc gamma RI) receptors bound with apparent affinities in the nanomolar range, while the other receptors bound in the micromolar range.

TABLE 30

| | huCD16a | huCD16a | huCD32a | huCD32a | | | | | | |
|---|---|---|---|---|---|---|---|---|---|---|
| | (F176) | (V176) | (H167) | (R167) | huCD32b | huCD64 | cyCD16 | cyCD32a | cyCD32b | cyCD64 |
| | (μM) | (μM) | (μM) | (μM) | (μM) | (nM) | (μM) | (μM) | (μM) | (nM) |
| AB-8 | 2.2 ± | 0.7 ± | 2.2 ± | 6.1 ± | 19 ± | 27 ± | 1.4 ± | 4.8 ± | 4.3 ± | 13 ± |
| IgG | 0.9 | 0.3 | 1.5 | 3.6 | 14 | 11 | 0.7 | 1.2 | 2.0 | 6 |
| WT | (6) | (10) | (10) | (8) | (5) | (9) | (7) | (5) | (7) | (8) |
| AB-8- | 1.7 ± | 0.6 ± | 2.2 ± | 3.7 ± | 20 ± | 47 ± | 1.0 ± | 9.0 ± | 5.7 ± | 28 ± |
| 10-8 | 0.2 | 0.1 | 0.4 | 0.9 | 12 | 5 | 0.2 | 3.3 | 2.5 | 7 |
| WT | (7) | (10) | (10) | (10) | (5) | (10) | (8) | (8) | (10) | (7) |
| AB-8- | 2.1 ± | 0.5 ± | 2.3 ± | 3.9 ± | 11 ± | 47 ± | 1.2 ± | 17 ± | 6.7 ± | 29 ± |
| 42Q | 0.9 | 0.1 | 0.5 | 1.1 | 5 | 4 | 0.5 | 15 | 3.6 | 14 |
| WT | (7) | (10) | (10) | (10) | (5) | (10) | (8) | (9) | (9) | (7) |
| AB-8- | 1.8 ± | 0.7 ± | 2.5 ± | 3.4 ± | 10 ± | 48 ± | 1.2 ± | 9.6 ± | 5.4 ± | 31 ± |
| L7-2 | 0.4 | 0.2 | 0.8 | 0.7 | 5 | 10 | 0.3 | 3.0 | 1.7 | 16 |
| WT | (6) | (10) | (10) | (10) | (6) | (10) | (7) | (8) | (8) | (7) |
| AB-8- | 1.8 ± | 0.7 ± | 2.1 ± | 3.0 ± | 21 ± | 49 ± | 1.1 ± | 9.1 ± | 8.7 ± | 38 ± |
| 39.38 | 0.5 | 0.2 | 0.7 | 0.8 | 22 | 14 | 0.4 | 3.1 | 6.9 | 10 |
| WT | (5) | (10) | (10) | (10) | (5) | (10) | (7) | (6) | (10) | (7) |
| Iso | 1.4 ± | 0.7 ± | 1.6 ± | 2.0 ± | 7 ± | 48 ± | 0.9 ± | 3.7 ± | 8.1 ± | 32 ± |
| hIgG1 | 0.4 | 0.1 | 0.7 | 1.0 | 6 | 4 | 0.4 | 3.2 | 8.2 | 22 |
| WT | (4) | (10) | (10) | (8) | (2) | (8) | (7) | (5) | (8) | (6) |
| Iso | No | No | No | No | No | No | No | No | No | No |
| hIgG1 | binding | binding | binding | binding | binding | binding | binding | binding | binding | binding |
| LALA | (10) | (10) | (10) | (10) | (10) | (10) | (10) | (10) | (10) | (10) |
| P331S | | | | | | | | | | |

Values are Mean, error is Standard Deviation, number of determinations, n, is in parentheses

This study was not designed to evaluate Fc_gamma receptor binding when the anti-pyroGlu3 Aβ-huTfR antibody is bound to TfR. Certain anti-pyroGlu3 Aβ-huTfR antibodies of the present disclosure that contain an anti-TfR moiety from a certain family (e.g., 42Q, L10-8, 24A) showed reduced levels of ADCC compared to other anti-pyroGlu3 Aβ-huTfR antibodies that contain anti-TfR moiety from a different family (e.g., 39.38, 9.1B.35) having a different binding epitope (as shown above), suggesting that an ADCC assay is a more relevant measure for this function of the antibodies.

### Example 37. In Vivo Serum PK and Brain Uptake of Anti-pyroGlu3 Aβ-huTfR Antibodies

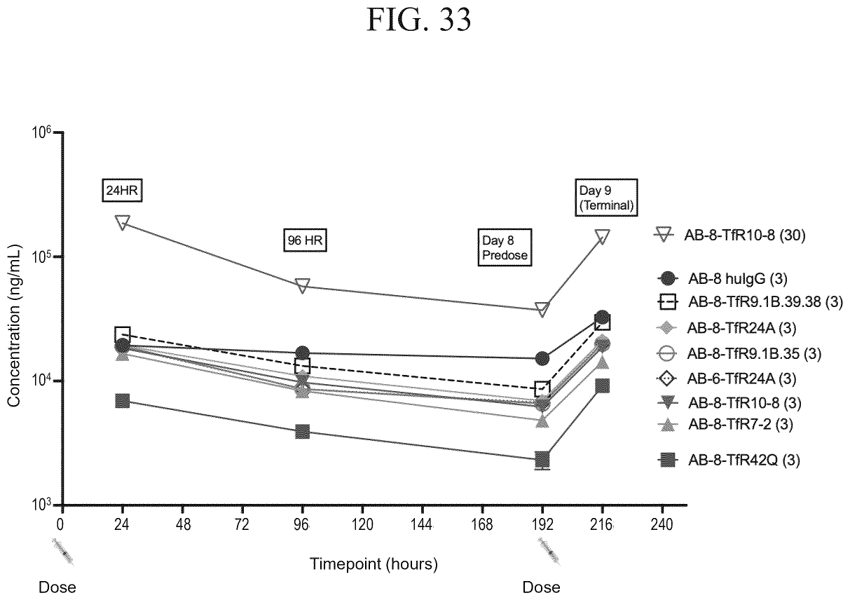

Serum PK and brain uptake of anti-pyroGlu3 Aβ-huTfR antibodies of the present disclosure with various anti-huTfR affinities were evaluated in human TfR knock in (KI) mice. The human TfR-KI mice express the human/mouse chimeric TfR (human TfR extracellular domain, all other domains remain mouse TfR) at levels similar to wild-type mouse expression of TfR. The huTfR-KI mice were dosed intravenously at 3 mg/kg for the range of TfR binding variants, and at 30 mg/kg for a single TfR variant 10-8. FIG. 33 shows serum pharmacokinetics of antibodies in huTfR KI that mice received two weekly doses of 3 mg/kg or 30 mg/kg of antibody AB-8 huIgG1 and various AB-8-TfR variants. Mice were dosed on D1 (day 1) and D8 (day 8). Serum PK was measured at D2 (day 2), D8 (day 8) before dosing, and D9 (day 9). Levels of antibodies in the serum followed expected target mediated drug disposition (TMDD) for peripheral TfR binding and ranked according to TfR affinities, with the highest affinity clone 42Q having the lowest and fastest serum PK. The 30 mg/kg dose of AB-8-TfR10-8 showed the highest serum concentrations.

Brain levels of antibodies were assessed 24 hours after the second dose in brain lysates depleted of brain vasculature. Peripherally dosed antibodies were detected at levels>10-fold higher than non-TfR binding control (AB-8 huIgG1) (data not shown), suggesting the observed enhanced brain uptake was mediated by interaction with TfR at the blood-brain barrier.

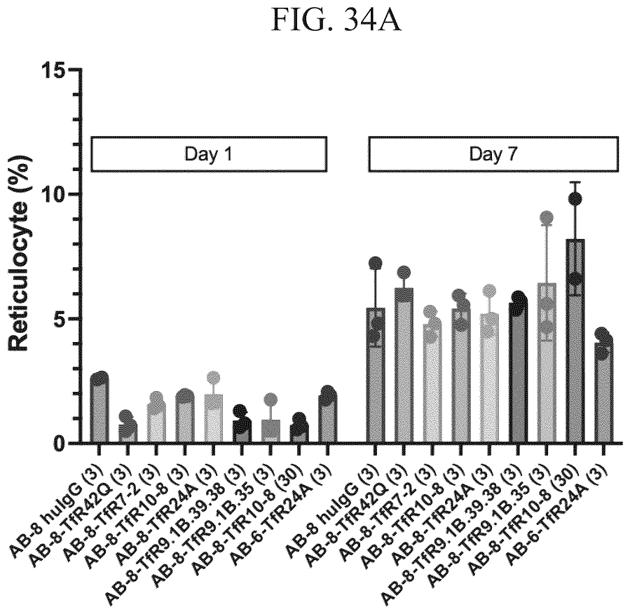

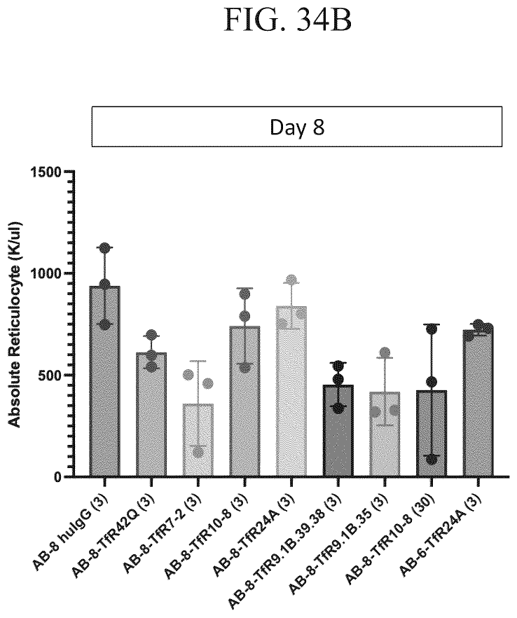

FIG. 34A and FIG. 34B show reticulocyte percentages in peripheral whole blood assessed at D1 (day 1) and D7 (day 7) post first dose and on D8 (day 8) at study termination in huTfR KI mice, respectively.

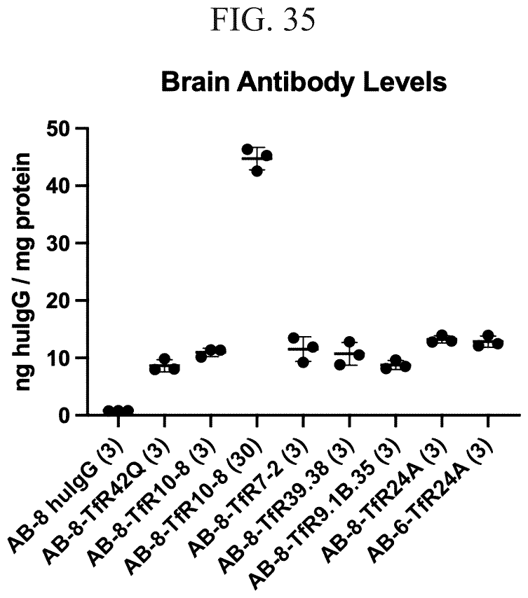

FIG. 35 shows brain uptake of various anti-pyroGlu3 Abeta (Aβ)-huTfR antibodies in huTfR KI mice that received two weekly doses of 3 mg/kg or 30 mg/kg of antibody AB-8 huIgG1 and various AB-8-TfR variants. Antibody levels (ng/mg protein) in vessel-depleted brain at 24 hours after the second dose were measured and are shown as ng huIgG/mg protein.

### Example 38: Anti-pyroGlu3 Aβ-huTfR Antibodies in NHPs

A repeat dose study in non-human primates following i.v. administration of anti-pyroGlu3Abeta (Aβ) antibodies of the present disclosure twice over 10 days was conducted to assess antibody levels in serum and safety of the dosed antibodies. Serum, CSF, peripheral, and brain tissues were collected. The study included 2 dose levels (3 mg/kg and 30 mg/kg) of AB-8 huIgG, AB-8-TfR10-8, and AB-8-TfR42Q. AB-8-TfR7-2 and AB-8-TfR39.38 antibodies were dosed at 3 mg/kg. Standard safety assessments (e.g., mortality, clinical signs, body weight, clinical pathology, as well as macro- and microscopic evaluation) were performed.

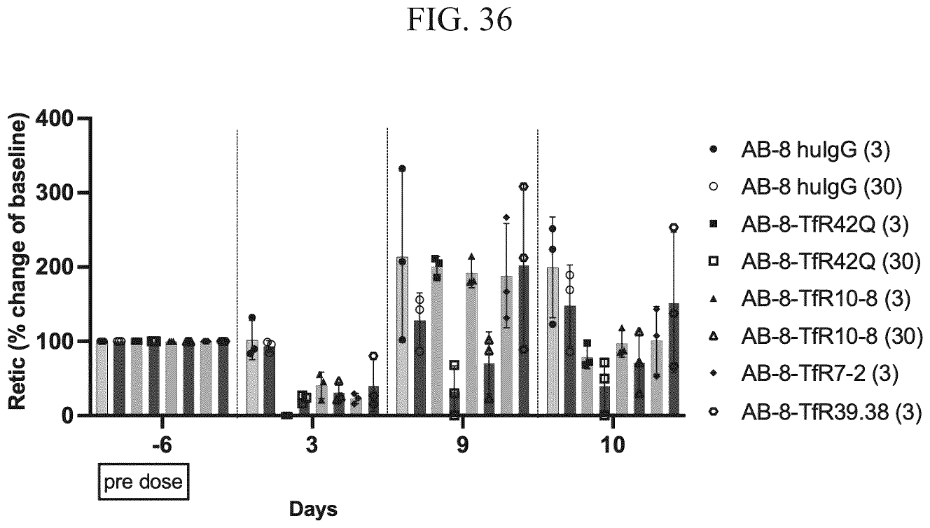

Reticulocytes, RBCs, and hemoglobin were assessed and compared to percent changes from baseline measures. Reticulocyte reduction was observed for AB-8-TfR42Q at day 3 (D3) post dosing and decreased ~70% for AB-8-TfR10-8 at 3 mg/kg and decreased for both to a similar extent at 30 mg/kg (FIG. 36). FIG. 36 also showed that antibody AB-8-TfR7-2 and AB-8-TfR39.38 showed similar reductions in reticulocytes. At Day 9, before the second dose, reticulocyte levels were similar to the control AB-8 huIgG for all groups dosed at 3 mg/kg while levels remained lower for 30 mg/kg dose level when compared to control. 24 hours after the second dose, reticulocyte responses were similar to the 48 hour levels for all groups compared to the control AB-8 huIgG.

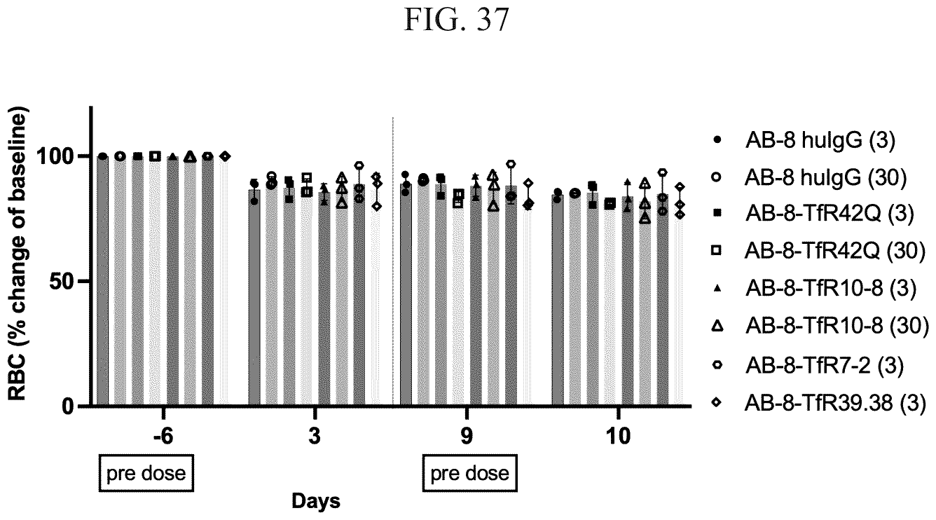

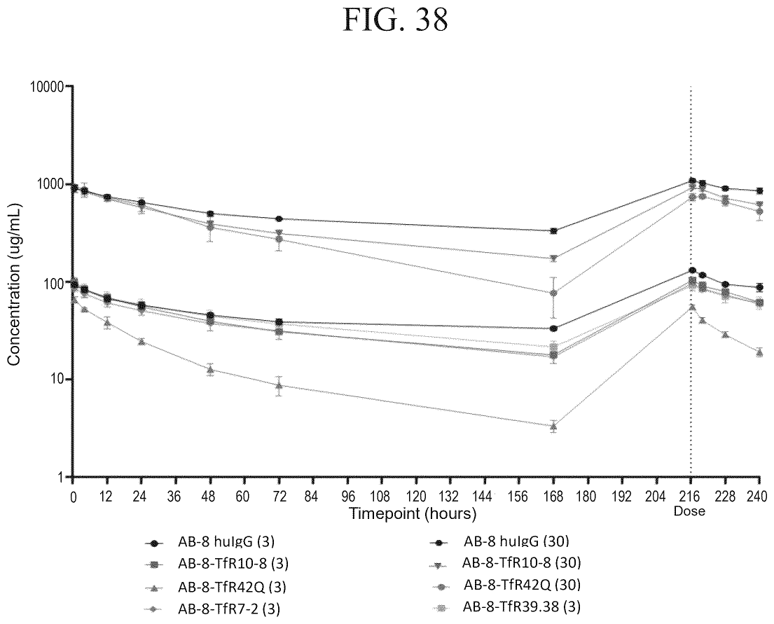

FIG. 37 shows levels of red blood cells (RBCs) as a percent change from baseline in whole blood at day 10 from NHPs receiving 2 doses over 8 days of antibody AB-8 huIgG1 and various AB-8-TfR variants dosed at 3 mg/kg and 30 mg/kg. FIG. 38 shows serum pharmacokinetic profiles from this NHP study with antibody AB-8 huIgG1 and AB-8-TfR variants.

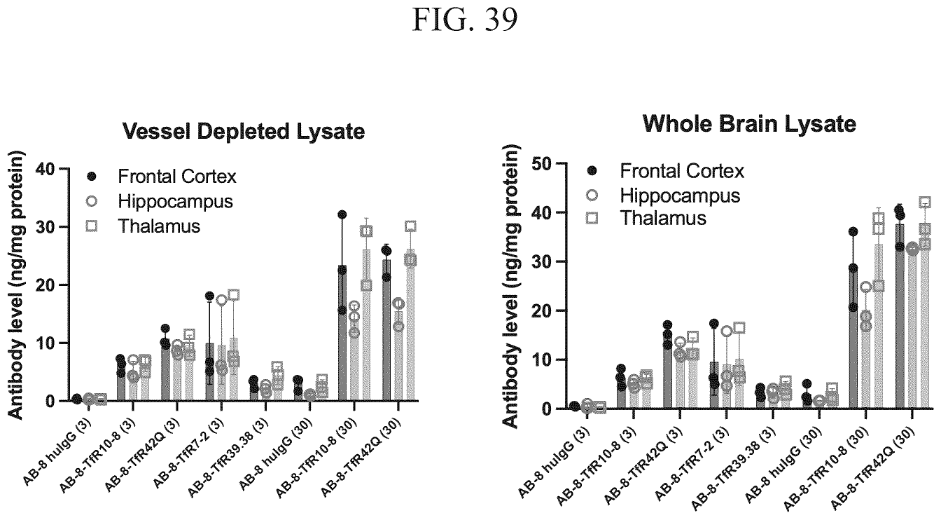

Enhanced brain uptake was consistently observed in 3 different brain regions, including frontal cortex, hippocampus, and thalamus (deep brain region) in vessel depleted lysates and whole brain lysates (FIG. 39). Enhanced brain uptake was observed at both 3 mg/kg and 30 mg/kg of all TfR antibodies compared to that observed with AB-8 hu IgG naked control antibody. In addition, for AB-8-TfR10-8 and AB-8-TfR42Q, brain uptake was dose-dependent albeit not dose proportional (FIG. 39). The highest brain uptake was noted for AB-8-TfR42Q, followed by AB-8-TfR10-8 at both dose levels.

Taken together, these results showed that administration of anti-Abeta antibody AB-8 conjugated to different huTfR antigen-binding domains resulted in higher brain uptake compared to that observed with a naked (unconjugated) antibody, thus demonstrating the ability huTfRs antigen-binding domains to facilitate uptake of antibody cargos into various brain regions, including deep regions of the brain.

### Example 39: AB-8-TfR42Q and AB-8-TfRL10-8 Colocalizes with Abeta (Aβ)Plaques on Human Alzheimer's Disease (AD) Brain Sections

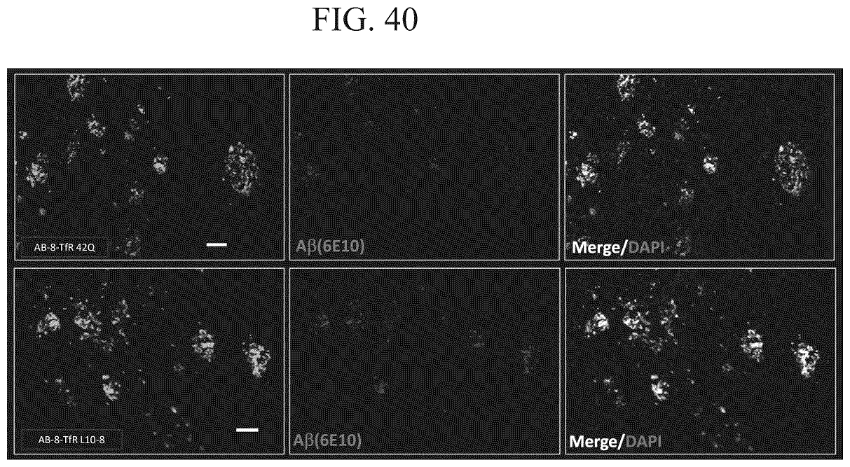

To demonstrate that the addition of the TfR 2+1 scFv variants to AB-8 does not alter binding to pyroglu3 Abeta (Aβ), unfixed, frozen human AD brain sections (medial temporal gyrus) were obtained from the Banner Institute, AZ for assessment of binding to Abeta (Aβ) plaques by immunofluorescent staining. FIG. 40 shows the binding of an anti-Abeta (Aβ) antibody (Clone 6E10, middle images), AB-8-TfR42Q and AB-8-TfRL10-8 (far left images) and the colocalization of the two primary antibodies on the far right panel. AlexaFluor labeled secondary antibodies directed against mouse for 6E10 and huIgG for AB-8 antibodies were used for detection of binding. Nuclei were counterstained with DAPI and images were acquired with a 10× objective on a Keyence microscope. Scale bar represents 50 μm. AB-8 alone (data not shown) showed similar colocalization with Abeta (Aβ) plaques as AB-8-TfR42Q and AB-8-TfRL10-8. Isotype hIgG1 control did not show binding to AD samples. AB-8 alone and AB-8-TfR42Q did not show binding to a normal control, non-AD sample (data not shown).

### Example 40: AB-8-msTfR Colocalizes with Abeta (Aβ)Plaques in the 5XFAD Mice

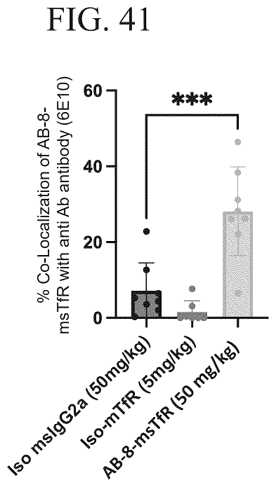

A chronic multidose efficacy study was performed to assess the extent of Abeta (Aβ) reductions over 4 weekly doses at 2 dose levels, 5 and 50 mg/kg. 8 male 5XFAD mice were dosed 4 times weekly with isotype (Iso) msIgG2a control (50 mg/kg), Iso-msTfR (5 mg/kg) or AB-8-msTfR (50 mg/kg) and assessed for plaques by immunofluorescence with anti-Abeta (Aβ) antibody (6E10). Brain tissue was harvested and fixed for plaque staining. The dosed antibodies were detected with anti-msIgG2a. Images were acquired with a 20×objective on an Axioscan platform (Zeiss). FIG. 41 shows quantification of plaque and dosed antibody colocalized area in the hippocampus. The area of colocalized staining of AB-8-msTfR (50 mg/kg) and 6E10 was ~28% (p<0.0001 vs Iso msIgG2a (50 mg/kg), one-way ANOVA with Dunnett's multiple comparisons test).

### Example 41: TfR-Enhanced CSF Uptake of AB-8-TfR42Q in NHP

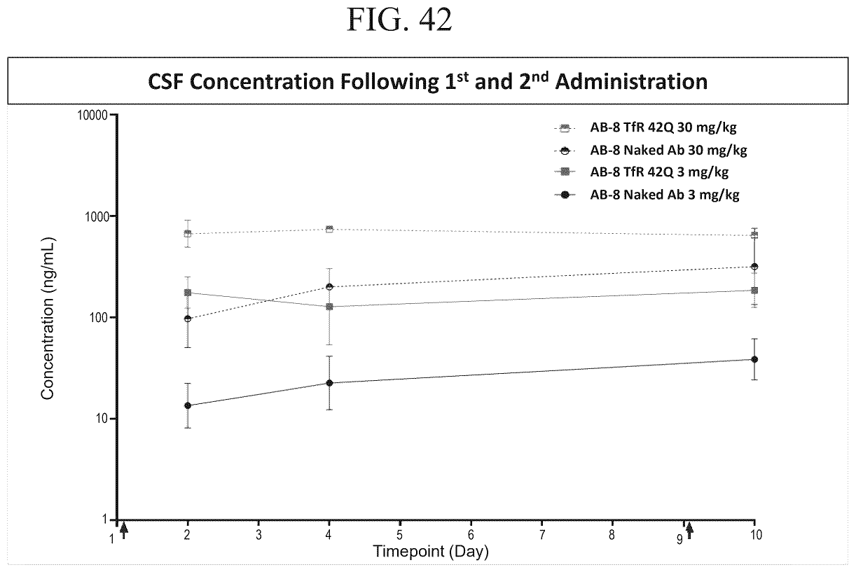

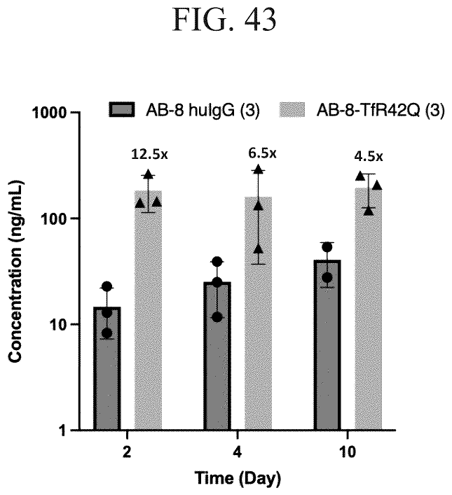

A repeat dose PK study in non-human primates (NHPs) following i.v. administration of anti-pyroGlu3Abeta (Aβ) antibodies twice over 10 days was conducted to assess antibody levels in serum and brain, and the safety of the dosed antibodies. Serum, CSF, peripheral and brain tissues were collected. The study included 2 dose levels (3 mg/kg and 30 mg/kg) of AB-8 huIgG and AB-8-TfR42Q. Standard safety assessments (e.g., mortality, clinical signs, body weight, clinical pathology) were performed. CSF samples were analyzed for the levels of AB-8 naked antibody (without TfR) and AB-8-TfR-42Q on day 2 (24 hr post 1^{st} dose), 4 (72 hr post 1^{st} dose) and 10 (24 hr post 2^{nd} dose). FIG. 42 shows the time-dependence profile of antibody levels in the CSF. Greater concentrations of both antibodies are observed with the 30 mg/kg dose and is further increased with TfR-42Q over AB-8 alone. Arrows indicate day of dosing (shown on the x-axis). FIG. 43 shows a bar graph of the time points for the CSF concentrations after the 3 mg/kg dose of AB-8 huIgG (circle data points) and AB-8-TfR42Q (triangle data points). Increases ranged from 4.5-fold to 12.5-fold for AB-8-TfR42Q over AB-8 at the 3 timepoints.

### Example 42: In Vivo Chronic Dosing Efficacy Studies with Anti-pyroGlu3 Abeta (Aβ)Murine TfR IgG2a Surrogates in a 2+1 Format

To investigate brain uptake and efficacy of anti-pyroGlu3 Abeta (Aβ) murine TfR surrogate (msTfR) antibodies in a 2+1 format, a chronic dosing efficacy study is conducted in aged 5XFAD mice (expressing murine TfR). Mice aged to 7 months are dosed weekly for 12 weeks at 10 mg/kg and at 24 h post dosing, the blood, brain, spleen, and liver are harvested. Blood is measured for complete blood count analysis. Vessel-depleted brain lysates and serum samples are assessed for antibody levels. TfR levels in brain, spleen, and liver are measured by western blot. Abeta (Aβ)₁₋₄₂ levels are assessed in 5M Guanidine HCl-lysed brain samples for reductions in Abeta (Aβ). Target engagement and plaque reduction at amyloid plaques is also assessed in brain sections of the animals by detection of dosed antibodies and Thioflavin S staining for amyloid.

### Example 43: In Vivo Low Dosing Studies with Anti-pyroGlu3 Abeta (Aβ)-huTfR IgG2a Antibodies in a 2+1 Format in 5XFAD x Hu TfR KI Mice

An additional study in aged 5XFAD mice crossed to human TfR knock-in mice is used to assess whether anti-pyroGlu3 Abeta (Aβ) huTfR antibodies in a 2+1 format show similar brain uptake as observed in single human TfR KI mice and can immunodecorate plaques similar to 5XFAD mice dosed with anti-pyroGlu3 Abeta (Aβ) surrogate mouse TfR antibodies in a 2+1 format. Mice are dosed twice weekly at 3 mg/kg subcutanously over 7 days. Blood, brain, spleen and liver are harvested 24 hours after the last dose. Blood is measured for complete blood count analysis. Vessel-depleted brain lysates and serum samples are assessed for antibody levels. Target engagement at amyloid plaques is assessed in brain sections of the animals by detecting bound human IgG using immunofluorescent staining.

### Example 44: Brain TfR levels in NHP after a low dose administration of AB-8-TfR42Q or AB-8-TfR10-8

A repeat dose PK study in non-human primates following intravenous (IV) administration of anti-pyroGlu3Abeta (Aβ) antibodies twice over 9 days was conducted to assess antibody levels in serum and safety of the dosed antibodies. Serum, CSF, peripheral and brain tissues were collected. The study included 2 dose levels (3 mg/kg and 30 mg/kg) of AB-8 huIgG and AB-8-TfR42Q, and AB-8-TfR10-8, in addition to 3 mg/kg of AB-8-TfR7-2 and AB-8-TfR39.38. Standard safety assessments (e.g., mortality, clinical signs, body weight, clinical pathology) were performed. TfR levels in brain lysates from frontal cortex, hippocampus, and thalamus were assessed by a TfR MSD assay.

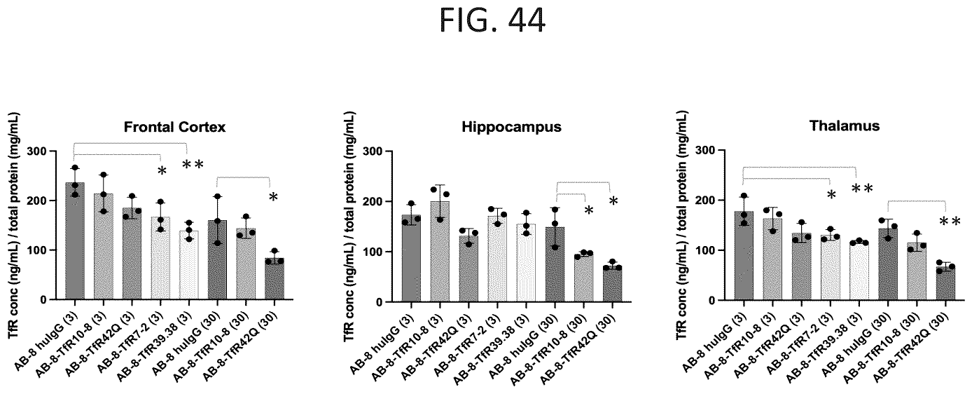

FIG. 44 shows the level of TfR detected in the brain regions normalized to total protein concentration as assessed by BCA. The higher 30 mg/kg dose of AB-8-TfR42Q was associated with a significant decrease in TfR in all brain regions assessed (*p<0.05, **p<0.01 by one-way ANOVA with Dunnet's multiple comparisons test). No significant decreases in TfR levels were observed with AB-8-TfR42Q or AB-8-TfR10-8 at the 3 mg/kg dose level.

### Example 45: Serum and Brain Pharmacokinetics of Intravenous and Subcutaneous Doses of pyroGlu3 Abeta (Aβ)-huTfR in Human TfR Knock-In Mice

To determine whether subcutaneous (SC) dosing of pyroGlu3Abeta (Aβ)-huTfR antibodies enable brain exposure similar to intravenous (IV) dosing, a single dose time-course study was conducted in human TfR KI mice. Mice were dosed at 3 mg/kg IV via the tail-vein or SC, and serum samples were collected at various timepoints (30 min, 1 h, 6 h, 24 h, 32 h, 48 h, 72 h, 96 h, and 168 h; n=3 per time point). Timepoints for brain harvest were at 24 h, 48 h, 96 h, and 168 h post dosing. Serum and brain antibody levels (from vessel depleted lysates) were assessed by an MSD detecting human IgG.

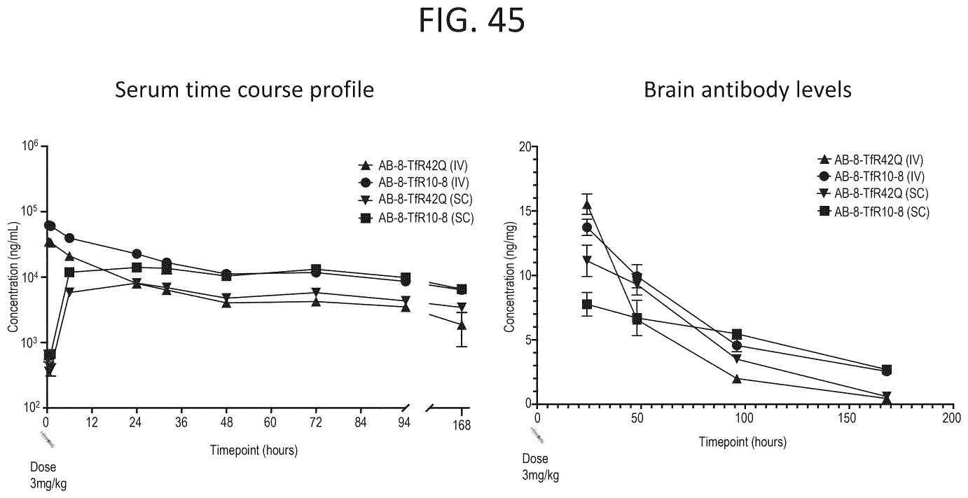

FIG. 45 (left) shows the serum time course profile of the administered antibodies and that SC dosing reached a Cmax concentration at 24 h. AB-8-TfR10-8 (having a lower anti-TfR affinity) showed greater levels in serum due to less target mediated clearance in the periphery mediated by TfR. FIG. 45 (right) shows brain antibody levels over the four timepoints. The calculated AUC for AB-8-TfR42Q via SC administration was slightly higher than for IV (700 vs 559 ng/mL/h), whereas the AUC for AB-8-TfR10-8 was lower for SC than IV (760 vs 888 ng/mL/h). Overall, the calculated AUC values were comparable between IV and SC dosing in a rodent model.

### Example 46: Comparison of Absolute Brain Concentration of pyroGlu3 Abeta (Aβ)-huTfR Antibodies and Other Anti-pyroGlu3Abeta (Aβ)-huTfR Antibodies

Brain uptake values for AB-8-TfR42Q and AB-8-TfR10-8 obtained from a repeat-dose pharmacokinetic study in non-human primates (NHP) following intravenous (IV) administration (as described in Example 38 and FIG. 39) were used to compare against published data for other anti-pyroGlu3Abeta (Aβ)-huTfR antibodies.

These brain uptake values were converted from ng antibody per mg total protein (ng Ab/mg protein) to nanomolar antibody concentration per brain volume (nM) to facilitate comparison with published brain uptake values of other brain delivery carriers. For unit conversion, the antibody concentration measured in the assay buffer (ng Ab/mL buffer) was multiplied by the total buffer volume used to process each brain sample to obtain the total mass of antibody recovered per sample (ng Ab). The total mass was normalized to tissue mass by dividing to the wet weight of the corresponding brain region (g), yielding ng Ab/g tissue. Assuming a brain tissue density of approximately 1 g/mL (see Barber, T. W., Brockway, J. A., & Higgins, L. S. (1970). The density of tissues in and about the head. Acta Neurologica Scandinavica, 46(S43), 85-92), the ng Ab/g brain tissue was treated as ng Ab/mL brain volume. The resulting concentration (ng Ab/mL brain) was converted to molar concentration (nM) using a molecular weight of 175 kDa.

Using this approach, the absolute brain concentrations (whole brain homogenate prepared from frontal cortex) at 24 hours after the second 3 mg/kg IV dose were calculated to be 8.4 nM for AB-8-TfR42Q and 3.8 nM for AB-8-TfR10-8.

Grimm H P et al. Delivery of the Brainshuttle™ amyloid-beta antibody fusion trontinemab to non-human primate brain and projected efficacious dose regimens in humans. MAbs. 2023 January-December; 15(1):2261509 also reported approximately 400 ng antibody per g brain in NHP cortex for an anti-Abeta (Aβ)-TfR antibody (trontinemab), corresponding to approximately 2.1 nM at 24 hours after a 10 mg/kg i.v. dose.

In comparison, AB-8-TfR42Q and AB-8-TfR10-8 achieved 8.4 nM and 3.8 nM, respectively, at 24 hours after a 3 mg/kg IV dose (3.3-fold lower administered dose). Accordingly, under the conditions evaluated, AB-8-TfR42Q and AB-8-TfR10-8 exhibited absolute frontal cortex concentrations that are greater than published values for related TfR-based brain delivery antibodies dosed at higher concentrations in NHP.

Differences in study design are noted. The values calculated using the antibodies of the present invention were determined at 24 hours, after a second dose administered 9 days after the first dose, whereas the Grimm et al. value was reported at 24 hours after a single dose. In Grimm et al., cortex concentrations peaked at approximately 24 hours and declined toward the baseline level observed for a conventional IgG lacking a brain-shuttle by approximately 240 hours, suggesting that residual brain exposure at day 10 would likely be low. Accordingly, accumulation is not expected to be a major contributor to the 24-hour post-second dose brain concentrations. In addition, these comparisons are made across the applicant's internal datasets and external datasets generated and reported in the literature.

1. An isolated antibody or antigen-binding fragment thereof that specifically binds to human N-terminal truncated pyroglutamate amyloid beta peptide (pyroGlu3 Abeta) wherein the antibody or antigen-binding fragment comprises a heavy chain variable region (VH) complementarity determining region (CDR) 1, VH CDR2, VH CDR3 and a light chain variable region (VL) CDR1, CDR2, and CDR3 comprising the sequences of:
(a) SEQ ID NOs: 13, 14, 15, 24, 30, and 31, respectively;
(b) SEQ ID NOs: 4, 5, 6, 21, 22, and 23, respectively;
(c) SEQ ID NOs: 7, 8, 9, 24, 25, and 26, respectively;
(d) SEQ ID NOs: 10, 11, 12, 27, 28, and 29, respectively;
(e) SEQ ID NOs: 16, 17, 18, 32, 33, and 34, respectively;
(f) SEQ ID NOs: 4, 5, 6, 21, 22, and 23, respectively;
(g) SEQ ID NOs: 7, 8, 9, 24, 25, and 26, respectively;
(h) SEQ ID NOs: 10, 19, 12, 27, 35, and 29, respectively;
(i) SEQ ID NOs: 13, 14, 15, 24, 30, and 31, respectively; or
(j) SEQ ID NOs: 16, 20, 18, 36, 33, and 34, respectively.

2.-3. (canceled)

4. The antibody or antigen-binding fragment of claim 1, wherein the antibody or antigen-binding fragment comprises a VH and a VL, wherein the VH and VL comprise amino acid sequences at least 80%, at least 85%, at least 90%, at least 95%, or at least 99% identical to the amino acid sequences of:
(a) SEQ ID NOs: 43 and 53, respectively;
(b) SEQ ID NOs: 47 and 48, respectively;
(c) SEQ ID NOs: 41 and 42, respectively;
(d) SEQ ID NOs: 43 and 44, respectively;
(e) SEQ ID NOs: 45 and 46, respectively;
(f) SEQ ID NOs: 39 and 40, respectively;
(g) SEQ ID NOs: 49 and 50, respectively;
(h) SEQ ID NOs: 51 and 52, respectively;
(i) SEQ ID NOs: 37 and 38, respectively; or
(j) SEQ ID NOs: 54 and 55, respectively.

5. An antibody or antigen-binding fragment thereof that specifically binds to human N-terminal truncated pyroglutamate amyloid beta peptide (pyroGlu3 Abeta), wherein the antibody or antigen-binding fragment comprises a VH and VL comprising the amino acid sequences of:
(a) SEQ ID NOs: 43 and 53, respectively;
(b) SEQ ID NOs: 47 and 48, respectively;
(c) SEQ ID NOs: 41 and 42, respectively;
(d) SEQ ID NOs: 43 and 44, respectively;
(e) SEQ ID NOs: 45 and 46, respectively;
(f) SEQ ID NOs: 39 and 40, respectively;
(g) SEQ ID NOs: 49 and 50, respectively;
(h) SEQ ID NOs: 51 and 52, respectively;
(i) SEQ ID NOs: 37 and 38, respectively; or
(j) SEQ ID NOs: 54 and 55, respectively.

6.-7. (canceled)

8. The antibody or antigen-binding fragment of claim 1, wherein the antibody or antigen-binding fragment comprises a heavy chain constant region and a light chain constant region, wherein the heavy chain constant region is an isotype selected from the group consisting of human IgG₁, IgG₂, IgG₃, and IgG₄ isotypes, and wherein the light chain constant region is a human IgGκ light chain constant region.

9. (canceled)

10. The antibody or antigen-binding fragment of claim 8, wherein the antibody or antigen-binding fragment comprises a heavy chain constant region and a light chain constant region, wherein the heavy chain constant region is a human IgG₁ heavy chain constant region, and wherein the light chain constant region is a human IgGκ light chain constant region.

11.-18. (canceled)

19. The antibody or antigen-binding fragment of claim 1, wherein the antibody or antigen-binding fragment promotes phagocytosis of Abeta plaques, optionally wherein 0.1 μg/ml increases phagocytosis of Abeta plaques by about 50% as compared to a control.

20.-21. (canceled)

22. The antibody or antigen-binding fragment of claim 1, wherein the antibody or antigen-binding fragment thereof is bispecific.

23. The antibody or antigen-binding fragment of claim 1, further comprising an antigen-binding domain that binds to blood brain barrier (BBB) target, wherein the BBB target is human transferrin receptor (TfR) or human CD98 heavy chain (CD98hc).

24.-26. (canceled)

27. The antibody or antigen-binding fragment of claim 23, wherein
(a) the BBB target is human TfR and wherein the antibody or antigen-binding fragment comprises a human TfR antigen-binding domain comprising heavy chain variable region (VH) complementarity determining region (CDR) 1, VH CDR2, VH CDR3 and light chain variable region (VL) CDR1, CDR2, and CDR3 sequences comprising the amino acid sequences of:
(i) SEQ ID NOs: 56, 57, 59, 69, 67, and 68, respectively;
(ii) SEQ ID NOs: 56, 57, 58, 66, 67, and 68, respectively;
(iii) SEQ ID NOs: 56, 57, 58, 69, 67, and 68, respectively;
(iv) SEQ ID NOs: 56, 57, 60, 69, 67, and 68, respectively;
(v) SEQ ID NOs: 56, 57, 59, 70, 67, and 68, respectively;
(vi) SEQ ID NOs: 56, 57, 59, 71, 67, and 68, respectively;
(vii) SEQ ID NOs: 56, 57, 59, 72, 67, and 68, respectively;
(viii) SEQ ID NOs: 56, 57, 59, 73, 67, and 68, respectively;
(ix) SEQ ID NOs: 56, 57, 61, 69, 67, and 68, respectively;
(x) SEQ ID NOs: 62, 63, 64, 74, 75, and 76, respectively;
(xi) SEQ ID NOs: 62, 65, 64, 77, 75, and 76, respectively;
(xii) SEQ ID NOs: 62, 63, 64, 78, 75, and 76, respectively;
(xiii) SEQ ID NOs: 62, 63, 64, 79, 75, and 76, respectively;
(xiv) SEQ ID NOs: 62, 65, 64, 80, 75, and 76, respectively;
(xv) SEQ ID NOs: 56, 57, 59, 66, 67, and 68, respectively;
(xvi) SEQ ID NOs: 62, 275, 64, 276, 75, and 277, respectively; or
(xvii) SEQ ID NOs: 62, 275, 64, 77, 75, and 76, respectively; or
(b) the BBB target is human CD98hc and wherein the antibody or antigen-binding fragment comprises a human CD98hc antigen-binding domain comprising heavy chain variable region (VH) complementarity determining region (CDR) 1, VH CDR2, VH CDR3 and light chain variable region (VL) CDR1, CDR2, and CDR3 sequences comprising the amino acid sequences of:
(i) SEQ ID NOs: 98, 99, 100, 122, 123, and 124, respectively;
(ii) SEQ ID NOs: 98, 101, 100, 122, 123, and 124, respectively;
(iii) SEQ ID NOs: 98, 102, 100, 122, 123, and 124, respectively;
(iv) SEQ ID NOs: 98, 103, 100, 122, 123, and 124, respectively;
(v) SEQ ID NOs:98, 104, 100, 122, 123, and 124, respectively;
(vi) SEQ ID NOs: 98, 105, 100, 122, 123, and 124, respectively;
(vii) SEQ ID NOs: 98, 106, 100, 122, 123, and 124, respectively;
(viii) SEQ ID NOs: 98, 107, 100, 122, 123, and 124, respectively;
(ix) SEQ ID NOs: 98, 108, 100, 122, 123, and 124, respectively;
(x) SEQ ID NOs: 98, 109, 100, 122, 123, and 124, respectively;
(xi) SEQ ID NOs: 98, 110, 100, 122, 123, and 124, respectively;
(xii) SEQ ID NOs: 98, 99, 100, 125, 123, and 124, respectively;
(xiii) SEQ ID NOs: 98, 99, 100, 126, 123, and 124, respectively;
(xiv) SEQ ID NOs: 98, 99, 100, 127, 123, and 124, respectively;
(xv) SEQ ID NOs: 98, 99, 100, 128, 123, and 124, respectively;
(xvi) SEQ ID NOs: 98, 99, 100, 129, 123, and 124, respectively;
(xvii) SEQ ID NOs: 98, 99, 100, 130, 123, and 124, respectively;
(xviii) SEQ ID NOs: 98, 99, 100, 131, 123, and 124, respectively;
(xix) SEQ ID NOs: 98, 106, 100, 127, 123, and 124, respectively;
(xx) SEQ ID NOs: 98, 111, 100, 122, 123, and 124, respectively;
(xxi) SEQ ID NOs: 98, 99, 112, 122, 123, and 124, respectively;
(xxii) SEQ ID NOs: 98, 99, 113, 122, 123, and 124, respectively;
(xxiii) SEQ ID NOs: 98, 99, 114, 122, 123, and 124, respectively;
(xxiv) SEQ ID NOs: 98, 99, 115, 122, 123, and 124, respectively;
(xxv) SEQ ID NOs: 98, 99, 116, 122, 123, and 124, respectively;
(xxvi) SEQ ID NOs: 98, 99, 117, 122, 123, and 124, respectively;
(xxvii) SEQ ID NOs: 98, 99, 118, 122, 123, and 124, respectively;
(xxviii)SEQ ID NOs: 98, 99, 119, 122, 123, and 124, respectively;
(xxix) SEQ ID NOs: 98, 99, 120, 122, 123, and 124, respectively;
(xxx) SEQ ID NOs: 98, 99, 100, 132, 123, and 124, respectively;
(xxxi) SEQ ID NOs: 98, 99, 100, 133, 123, and 124, respectively;
(xxxii) SEQ ID NOs: 98, 99, 100, 134, 123, and 124, respectively;
(xxxiii)SEQ ID NOs: 98, 99, 100, 135, 123, and 124, respectively;
(xxxiv)SEQ ID NOs: 98, 99, 100, 136, 123, and 124, respectively;
(xxxv) SEQ ID NOs: 98, 99, 100, 137, 123, and 124, respectively;
(xxxvi)SEQ ID NOs: 98, 99, 100, 122, 123, and 138, respectively;
(xxxvii) SEQ ID NOs: 98, 99, 100, 122, 123, and 139, respectively;
(xxxviii) SEQ ID NOs: 98, 99, 100, 122, 123, and 140, respectively;
(xxxix)SEQ ID NOs: 98, 99, 100, 122, 123, and 141, respectively;
(xl) SEQ ID NOs: 98, 99, 100, 122, 123, and 142, respectively;
(xli) SEQ ID NOs: 98, 99, 100, 122, 123, and 143, respectively;
(xlii) SEQ ID NOs: 98, 99, 100, 122, 123, and 144, respectively;
(xliii) SEQ ID NOs: 98, 99, 100, 122, 123, and 145, respectively;
(xliv) SEQ ID NOs: 98, 99, 100, 122, 123, and 146, respectively;
(xlv) SEQ ID NOs: 98, 99, 100, 122, 123, and 147, respectively;
(xlvi) SEQ ID NOs: 121, 99, 100, 122, 123, and 124, respectively; or
(xlvii) SEQ ID NOs: 121, 99, 100, 122, 123, and 147, respectively.

28. The antibody or antigen-binding fragment of claim 27, wherein
(a) the BBB target is human TfR and wherein the human TfR antigen-binding domain comprises a VH and a VL, wherein the VH and VL comprise the amino acid sequences of:
(i) SEQ ID NOs: 84 and 83, respectively;
(ii) SEQ ID NOs: 81 and 82, respectively;
(iii) SEQ ID NOs: 81 and 83, respectively;
(iv) SEQ ID NOs: 85 and 83, respectively;
(v) SEQ ID NOs: 84 and 86, respectively;
(vi) SEQ ID NOs: 84 and 87, respectively;
(vii) SEQ ID NOs: 84 and 88, respectively;
(viii) SEQ ID NOs: 84 and 89, respectively;
(ix) SEQ ID NOs: 90 and 83, respectively;
(x) SEQ ID NOs: 91 and 92, respectively;
(xi) SEQ ID NOs: 93 and 94, respectively;
(xii) SEQ ID NOs: 91 and 95, respectively;
(xiii) SEQ ID NOs: 91 and 96, respectively;
(xiv) SEQ ID NOs: 93 and 97, respectively;
(xv) SEQ ID NOs: 269 and 270, respectively;
(xvi) SEQ ID NOs: 271 and 272, respectively; or
(xvii) SEQ ID NOs: 273 and 274, respectively; or
(b) the BBB target is human CD98hc and wherein the human CD98hc antigen-binding domain comprises a VH and a VL, wherein the VH and VL comprise the amino acid sequences of:
(i) SEQ ID NOs: 148 and 149, respectively;
(ii) SEQ ID NOs: 150 and 149, respectively;
(iii) SEQ ID NOs: 151 and 149, respectively;
(iv) SEQ ID NOs: 152 and 149, respectively;
(v) SEQ ID NOs: 153 and 149, respectively;
(vi) SEQ ID NOs: 154 and 149, respectively;
(vii) SEQ ID NOs: 155 and 149, respectively;
(viii) SEQ ID NOs: 156 and 149, respectively;
(ix) SEQ ID NOs: 157 and 149, respectively;
(x) SEQ ID NOs: 158 and 149, respectively;
(xi) SEQ ID NOs: 159 and 149, respectively;
(xii) SEQ ID NOs: 148 and 160, respectively;
(xiii) SEQ ID NOs: 148 and 161, respectively;
(xiv) SEQ ID NOs: 148 and 162, respectively;
(xv) SEQ ID NOs: 148 and 163, respectively;
(xvi) SEQ ID NOs: 148 and 164, respectively;
(xvii) SEQ ID NOs: 148 and 165, respectively;
(xviii) SEQ ID NOs: 148 and 166, respectively;
(xix) SEQ ID NOs: 155 and 162, respectively;
(xx) SEQ ID NOs: 167 and 149, respectively;
(xxi) SEQ ID NOs: 168 and 149, respectively;
(xxii) SEQ ID NOs: 169 and 149, respectively;
(xxiii) SEQ ID NOs: 170 and 149, respectively;
(xxiv) SEQ ID NOs: 171 and 149, respectively;
(xxv) SEQ ID NOs: 172 and 149, respectively;
(xxvi) SEQ ID NOs: 173 and 149, respectively;
(xxvii) SEQ ID NOs: 174 and 149, respectively;
(xxviii)SEQ ID NOs: 175 and 149, respectively;
(xxix) SEQ ID NOs: 176 and 149, respectively;
(xxx) SEQ ID NOs: 148 and 177, respectively;
(xxxi) SEQ ID NOs: 148 and 178, respectively;
(xxxii) SEQ ID NOs: 148 and 179, respectively;
(xxxiii)SEQ ID NOs: 148 and 180, respectively;
(xxxiv)SEQ ID NOs: 148 and 181, respectively;
(xxxv) SEQ ID NOs: 148 and 182, respectively;
(xxxvi)SEQ ID NOs: 148 and 183, respectively;
(xxxvii) SEQ ID NOs: 148 and 184, respectively;
(xxxviii) SEQ ID NOs: 148 and 185, respectively;
(xxxix)SEQ ID NOs: 148 and 186, respectively;
(xl) SEQ ID NOs: 148 and 187, respectively;
(xli) SEQ ID NOs: 148 and 188, respectively;
(xlii) SEQ ID NOs: 148 and 189, respectively;
(xliii) SEQ ID NOs: 148 and 190, respectively;
(xliv) SEQ ID NOs: 148 and 191, respectively;
(xlv) SEQ ID NOs: 148 and 192, respectively;
(xlvi) SEQ ID NOs: 193 and 194, respectively; or
(xlvii) SEQ ID NOs: 193 and 192, respectively.

29. The antibody or antigen-binding fragment of claim 23, wherein the antibody or antigen-binding fragment has a fully active Fc region.

30. (canceled)

31. The antibody or antigen-binding fragment of claim 27, wherein the antibody or antigen-binding fragment thereof that specifically binds to human N-terminal truncated pyroglutamate amyloid beta peptide (pyroGlu3 Abeta) comprises
the VH CDR 1, VH CDR2, VH CDR3 and VL CDR1, CDR2, and CDR3 sequences of: SEQ ID NOs: 13, 14, 15, 24, 30, and 31, respectively; and the human TfR antigen-binding domain comprises VH CDR1, VH CDR2, VH CDR3 and VL CDR1, CDR2, and CDR3 sequences comprising the amino acid sequences of SEQ ID NOs: 56, 57, 59, 69, 67, and 68, respectively.

32. The antibody or antigen-binding fragment of claim 27, wherein the antibody or antigen-binding fragment thereof that specifically binds to human N-terminal truncated pyroglutamate amyloid beta peptide (pyroGlu3 Abeta) comprises the VH CDR 1, VH CDR2, VH CDR3 and VL CDR1, CDR2, and CDR3 sequences of: SEQ ID NOs: 13, 14, 15, 24, 30, and 31, respectively; and the human TfR antigen-binding domain comprises VH CDR1, VH CDR2, VH CDR3 and VL CDR1, CDR2, and CDR3 sequences comprising the amino acid sequences of SEQ ID NOs: 56, 57, 59, 72, 67, and 68, respectively.

33. (canceled)

34. The antibody or antigen-binding fragment of claim 28, wherein the antibody or antigen-binding fragment thereof that specifically binds to human N-terminal truncated pyroglutamate amyloid beta peptide (pyroGlu3 Abeta) comprises a VH and a VL comprising the amino acid sequences of SEQ ID NOs: 43 and 53, respectively; and the human TfR antigen-binding domain comprises a VH and a VL comprising the amino acid sequences of SEQ ID NOs: 84 and 83, respectively.

35. The antibody or antigen-binding fragment of claim 28, wherein the antibody or antigen-binding fragment thereof that specifically binds to human N-terminal truncated pyroglutamate amyloid beta peptide (pyroGlu3 Abeta) comprises a VH and a VL comprising the amino acid sequences of SEQ ID NOs: 43 and 53, respectively; and the human TfR antigen-binding domain comprises a VH and a VL comprising the amino acid sequences of SEQ ID NOs: 84 and 88, respectively.

36. (canceled)

37. The antibody or antigen-binding fragment of claim 23, wherein the anti-BBB antigen-binding domain is a single-chain fragment variable (scFv).

38.-39. (canceled)

40. The antibody or antigen-binding fragment of claim 27, wherein the antibody or antigen-binding fragment that binds to pyroGlu3 Abeta and the anti-BBB antigen-binding domain are connected by a linker.

41. The antibody or antigen-binding fragment of claim 40, wherein the linker connects the C-terminus of the heavy chain Fc of the antibody or antigen-binding fragment that binds to pyroGlu3 Abeta and the N-terminus of the anti-BBB antigen-binding domain.

42. The antibody or antigen-binding fragment of claim 41, wherein the linker comprises GGSGG (SEQ ID NO: 214).

43. The antibody or antigen-binding fragment of claim 27, wherein the antibody or antigen-binding fragment that binds to pyroGlu3 Abeta and the anti-BBB antigen-binding domain are directly connected.

44.-45. (canceled)

46. The antibody or antigen-binding fragment of claim 23, wherein the antibody or antigen-binding fragment is trivalent, optionally wherein the antibody or antigen-binding fragment comprises one antigen-binding domain that binds to the BBB target and two antigen-binding domains that bind to pyroGlu3 Abeta.

47. (canceled)

48. The antibody or antigen-binding fragment of claim 28, comprising the amino acid sequences of (a) SEQ ID NOs: 252, 230, and 231% (b) SEQ ID NOs: 223, 224, and 225; (c) SEQ ID NOs: 229, 230, and 231; (d) SEQ ID NOs: 241, 224, and 225; (e) SEQ ID NOs: 242, 224, and 225; (f) SEQ ID NOs: 243, 224, and 225; (g) SEQ ID NOs: 244, 224, and 225; (h) SEQ ID NOs 245, 224, and 225; (i) SEQ ID NOs: 246, 224, and 225; (j) SEQ ID NOs: 247, 224, and 225; (k) SEQ ID NOs: 248, 224, and 225; (1) SEQ ID NOs: 249, 224, and 225; (m) SEQ ID NOs: 250, 224, and 225; (n) SEQ ID NOs: 251, 230, and 231; (o) SEQ ID NOs: 226, 227, and 228(p) SEQ ID NOs: 253, 230, and 231; (q) SEQ ID NOs: 254, 230, and 231; (r) SEQ ID NOs: 255, 230, and 231; (s) SEQ ID NOs: 256, 230, and 231; (t) SEQ ID NOs: 257, 230, and 231; (u) SEQ ID NOs: 258, 230, and 231; (v) SEQ ID NOs: 259, 230, and 231; (w) SEQ ID NOs: 260, 230, and 231; (x) SEQ ID NOs: 261, 230, and 231; (y) SEQ ID NOs: 262, 230, and 231; (z) SEQ ID NOs: 263, 230, and 231; (aa) SEQ ID NOs: 264, 230, and 231; (bb) SEQ ID NOs: 265, 230, and 231; SEQ ID NOs: 266, 230, and 231; (cc) SEQ ID NOs: 278, 224, and 225; or (dd) SEQ ID NOs: 279, 230, and 231.

49.-54. (canceled)

55. An isolated polynucleotide comprising a nucleic acid molecule encoding the heavy chain variable region or heavy chain of the antibody or antigen-binding fragment thereof of claim 1 and the light chain variable region or light chain of the antibody or antigen-binding fragment thereof of claim 1.

56. An isolated vector comprising the polynucleotide of claim 55.

57. A host cell comprising the polynucleotide of claim 55.

58. (canceled)

59. A method of producing an antibody or antigen-binding fragment thereof that binds to human pyroGlu3 Abeta comprising culturing the host cell of claim 57 so that the nucleic acid molecule is expressed and the antibody or antigen-binding fragment thereof is produced, optionally wherein the method further comprises isolating the antibody or antigen-binding fragment thereof from the culture.

60. An isolated antibody or antigen-binding fragment thereof that specifically binds to human pyroGlu3 Abeta and is produced by the method of claim 59.

61. A pharmaceutical composition comprising the antibody or antigen-binding fragment thereof of claim 23 and a pharmaceutically acceptable excipient.

62. A method of promoting phagocytosis of Abeta plaques comprising contacting a composition comprising Abeta plaques and macrophages and/or microglia with the antibody or antigen binding fragment thereof of claim 23.

63.-64. (canceled)

65. A method of slowing the progression of neurodegeneration in a subject in need thereof, the method comprising administering to the subject a therapeutically effective amount of the antibody or antigen binding fragment thereof of claim 23.

66. A method of decreasing cognitive decline in a subject in need thereof, the method comprising administering to the subject a therapeutically effective amount of the antibody or antigen binding fragment thereof of claim 23.

67. (canceled)

68. A method of treating and/or preventing a neurodegenerative disease in a subject, the method comprising administering to the subject a therapeutically effective amount of the antibody or antigen binding fragment thereof of claim 23.

69. (canceled)

70. The method of claim 68, wherein the neurodegenerative disease is Alzheimer's disease, early Alzheimer's disease, or mild cognitive impairment (MCI).

71.-83. (canceled)

84. The method of claim 65, wherein the administration is intravenous or subcutaneous.

85.-87. (canceled)

88. An isolated polynucleotide comprising a nucleic acid molecule encoding the heavy chain variable region or heavy chain of the antibody or antigen-binding fragment thereof of claim 23 or the light chain variable region or light chain of the antibody or antigen-binding fragment thereof of claim 23.


SEQUENCE LISTING (282 sequences)

SEQ ID NO: 1 - protein, 42 aa, synthetic construct [sequence omitted]

SEQ ID NO: 2 - protein, 760 aa, Homo sapiens
  source 1..760: note = human TfR amino acid sequence
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 3 - protein, 630 aa, Homo sapiens
  source 1..630: note = human CD98hc
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 4 - protein, 7 aa, synthetic construct
  source 1..7: note = CDR-H1 of AB-1 and AB-6
SGSYYWG

SEQ ID NO: 5 - protein, 16 aa, synthetic construct
  source 1..16: note = CDR-H2 of AB-1 and AB-6
SFYYSGTIYYNPSLKS

SEQ ID NO: 6 - protein, 17 aa, synthetic construct
  source 1..17: note = CDR-H3 of AB-1 and AB-6
QREYSSSSNYYYYYMDV

SEQ ID NO: 7 - protein, 7 aa, synthetic construct
  source 1..7: note = CDR-H1 of AB-2 and AB-7
SSSYYWG

SEQ ID NO: 8 - protein, 16 aa, synthetic construct
  source 1..16: note = CDR-H2 of AB-2 and AB-7
SIYFSGSTYYNPSLKS

SEQ ID NO: 9 - protein, 17 aa, synthetic construct
  source 1..17: note = CDR-H3 of AB-2 and AB-7
HREMATIRDYHYYYMDV

SEQ ID NO: 10 - protein, 5 aa, synthetic construct
  source 1..5: note = CDR-H1 of AB-5 and AB-10
DYAMH

SEQ ID NO: 11 - protein, 17 aa, synthetic construct
  source 1..17: note = CDR-H2 of AB-5
GISWNNNNIDYADSVKG

SEQ ID NO: 12 - protein, 14 aa, synthetic construct
  source 1..14: note = CDR-H3 of AB-5 and AB-10
DREYSGSYYDAFDI

SEQ ID NO: 13 - protein, 5 aa, synthetic construct
  source 1..5: note = CDR-H1 of AB-3 and AB-8
SNYMS

SEQ ID NO: 14 - protein, 16 aa, synthetic construct
  source 1..16: note = CDR-H2 of AB-3 and AB-8
VIYSGGTKHYADSVKG

SEQ ID NO: 15 - protein, 14 aa, synthetic construct
  source 1..14: note = CDR-H3 of AB-3 and AB-8
SYYYGSGSSDAFDI

SEQ ID NO: 16 - protein, 5 aa, synthetic construct
  source 1..5: note = CDR-H1 of AB-4 and AB-9
NFGMS

SEQ ID NO: 17 - protein, 17 aa, synthetic construct
  source 1..17: note = CDR-H2 of AB-4
GIYWNGVSTSYPDSVKG

SEQ ID NO: 18 - protein, 13 aa, synthetic construct
  source 1..13: note = CDR-H3 of AB-4 and AB-9
GGSPYGDYGAFDI

SEQ ID NO: 19 - protein, 17 aa, synthetic construct
  source 1..17: note = CDR-H2 of AB-10
GISWNQGNIDYADSVKG

SEQ ID NO: 20 - protein, 17 aa, synthetic construct
  source 1..17: note = CDR-H2 of AB-9
GIYWNAVSTSYPDSVKG

SEQ ID NO: 21 - protein, 11 aa, synthetic construct
  source 1..11: note = CDR-L1 of AB-1 and AB-6
RASQSIRFYLN

SEQ ID NO: 22 - protein, 7 aa, synthetic construct
  source 1..7: note = CDR-L2 of AB-1 and AB-6
GASSLQS

SEQ ID NO: 23 - protein, 9 aa, synthetic construct
  source 1..9: note = CDR-L3 of AB-1 and AB-6
QQTYSTPIT

SEQ ID NO: 24 - protein, 11 aa, synthetic construct
  source 1..11: note = CDR-L1 of AB-2, AB-3, AB-7, and AB-8
RASQSISNYLN

SEQ ID NO: 25 - protein, 7 aa, synthetic construct
  source 1..7: note = CDR-L2 of AB-2 and AB-7
AASSLQS

SEQ ID NO: 26 - protein, 9 aa, synthetic construct
  source 1..9: note = CDR-L3 of AB-2 and AB-7
QQSYSTPLT

SEQ ID NO: 27 - protein, 11 aa, synthetic construct
  source 1..11: note = CDR-L1 of AB-5 and AB-10
RASQSISSWLA

SEQ ID NO: 28 - protein, 7 aa, synthetic construct
  source 1..7: note = CDR-L2 of AB-5
KASSLEN

SEQ ID NO: 29 - protein, 8 aa, synthetic construct
  source 1..8: note = CDR-L3 of AB-5 and AB-10
QEYNSYNT

SEQ ID NO: 30 - protein, 7 aa, synthetic construct
  source 1..7: note = CDR-L2 of AB-3 and AB-8
DAYSLQI

SEQ ID NO: 31 - protein, 9 aa, synthetic construct
  source 1..9: note = CDR-L3 of AB-3 and AB-8
QQSYSIPPT

SEQ ID NO: 32 - protein, 16 aa, synthetic construct
  source 1..16: note = CDR-L1 of AB-4
RSSQSLVHSDGNTYLS

SEQ ID NO: 33 - protein, 7 aa, synthetic construct
  source 1..7: note = CDR-L2 of AB-4 and AB-9
KISNRFS

SEQ ID NO: 34 - protein, 9 aa, synthetic construct
  source 1..9: note = CDR-L3 of AB-4 and AB-9
MQTTQFPYT

SEQ ID NO: 35 - protein, 7 aa, synthetic construct
  source 1..7: note = CDR-L2 of AB-10
KASSLES

SEQ ID NO: 36 - protein, 16 aa, synthetic construct
  source 1..16: note = CDR-L1 of AB-9
RSSQSLVHSEGNTYLS

SEQ ID NO: 37 - protein, 127 aa, synthetic construct
  source 1..127: note = AB-1 VH sequence
QLQLQESGPGLVKPSETLSLTCTVSGGSISSGSYYWGWIRQPPGKGLEWIGSFYYSGTIYYNPSLKSRVTISVDTSKNQFSLKLRSVTAADTAVYYCARQREYSSSSNYYYYYMDVWGKGTTVTVSS

SEQ ID NO: 38 - protein, 107 aa, synthetic construct
  source 1..107: note = AB-1 VL sequence
DIQMTQSPSSLSASVGDRVTITCRASQSIRFYLNWYQQKPGKAPKVLIYGASSLQSGVPSRFSGSGSGTDFTLTISSLQPEDFATYHCQQTYSTPITFGQGTRLEIK

SEQ ID NO: 39 - protein, 127 aa, synthetic construct
  source 1..127: note = AB-2 VH sequence
QLHLQESGPGLVKPSETLSLTCTVSGGSTSSSSYYWGWIRQPPGKGLEWIGSIYFSGSTYYNPSLKSRVTISVDTSKNQFSLKLKTVTAADTAVYYCVRHREMATIRDYHYYYMDVWGKGTTVTVSS

SEQ ID NO: 40 - protein, 107 aa, synthetic construct
  source 1..107: note = AB-2 VL sequence
DIQMTQSPSSLSASVGDRVTITCRASQSISNYLNWYQQKPGKAPKLLIYAASSLQSGVPSRFSGSGSGTDFTLTISSLQPEDFATYYCQQSYSTPLTFGGGTRVEIK

SEQ ID NO: 41 - protein, 123 aa, synthetic construct
  source 1..123: note = AB-5 VH sequence
EVQLVESGGGLVQPGRSLRLSCAASGFTFDDYAMHWVRQAPGKGLEWVSGISWNNNNIDYADSVKGRFTISRDNAKNSLYLQMNSLRAEDTALYYCVKDREYSGSYYDAFDIWGQGTMVTVSS

SEQ ID NO: 42 - protein, 106 aa, synthetic construct
  source 1..106: note = AB-5 VL sequence
DIQMTQSPSTLSASVGDRVTITCRASQSISSWLAWYQQKPGKAPKVLIYKASSLENGVPSRFSGSGSGTEFTLTISSLQPDDFATYYCQEYNSYNTFGQGTKLEIK

SEQ ID NO: 43 - protein, 122 aa, synthetic construct
  source 1..122: note = AB-3 and AB-8 VH sequence
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSS

SEQ ID NO: 44 - protein, 107 aa, synthetic construct
  source 1..107: note = AB-3 VL sequence
DIQMTQSPSSLSASVGDRVTITCRASQSISNYLNWYQQKPGKAPNLLIYDAYSLQIGVPSRFSGSGSGTDFTLTISSLQPEDFATYSCQQSYSIPPTFGGGTKVEIK

SEQ ID NO: 45 - protein, 122 aa, synthetic construct
  source 1..122: note = AB-4 VH sequence
EVQLVESGGGVVRPGGSLRLSCAASGFTFDNFGMSWVRQAPGKGLEWVSGIYWNGVSTSYPDSVKGRFTISRDNAKNSLYLQMNSLRGEDTALYYCARGGSPYGDYGAFDIWGQGTVVTVSS

SEQ ID NO: 46 - protein, 112 aa, synthetic construct
  source 1..112: note = AB-4 VL sequence
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQTTQFPYTFGQGTKLEIK

SEQ ID NO: 47 - protein, 127 aa, synthetic construct
  source 1..127: note = AB-6 VH sequence
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSS

SEQ ID NO: 48 - protein, 107 aa, synthetic construct
  source 1..107: note = AB-6 VL sequence
DIQMTQSPSSLSASVGDRVTITCRASQSIRFYLNWYQQKPGKAPKVVIYGASSLQSGVPSRFSGSGSGTDFTLTISSLQPEDFATYYCQQTYSTPITFGQGTRLEIK

SEQ ID NO: 49 - protein, 127 aa, synthetic construct
  source 1..127: note = AB-7 VH sequence
QLQLQESGPGLVKPSETLSLTCTVSGGSTSSSSYYWGWIRQPPGKGLEWIGSIYFSGSTYYNPSLKSRVTISVDTSKNQFSLKLSSVTAADTAVYYCVRHREMATIRDYHYYYMDVWGKGTTVTVSS

SEQ ID NO: 50 - protein, 107 aa, synthetic construct
  source 1..107: note = AB-7 VL sequence
DIQMTQSPSSLSASVGDRVTITCRASQSISNYLNWYQQKPGKAPKLLIYAASSLQSGVPSRFSGSGSGTDFTLTISSLQPEDFATYYCQQSYSTPLTFGGGTKVEIK

SEQ ID NO: 51 - protein, 123 aa, synthetic construct
  source 1..123: note = AB-10 VH sequence
EVQLVESGGGLVQPGRSLRLSCAASGFTFDDYAMHWVRQAPGKGLEWVSGISWNQGNIDYADSVKGRFTISRDNAKNSLYLQMNSLRAEDTALYYCVKDREYSGSYYDAFDIWGQGTMVTVSS

SEQ ID NO: 52 - protein, 106 aa, synthetic construct
  source 1..106: note = AB-10 VL sequence
DIQMTQSPSTLSASVGDRVTITCRASQSISSWLAWYQQKPGKAPKVVIYKASSLESGVPSRFSGSGSGTEFTLTISSLQPDDFATYYCQEYNSYNTFGQGTKLEIK

SEQ ID NO: 53 - protein, 107 aa, synthetic construct
  source 1..107: note = AB-8 VL sequence
DIQMTQSPSSLSASVGDRVTITCRASQSISNYLNWYQQKPGKAPKLLIYDAYSLQIGVPSRFSGSGSGTDFTLTISSLQPEDFATYYCQQSYSIPPTFGGGTKVEIK

SEQ ID NO: 54 - protein, 122 aa, synthetic construct
  source 1..122: note = AB-9 VH sequence
EVQLVESGGGVVRPGGSLRLSCAASGFTFDNFGMSWVRQAPGKGLEWVSGIYWNAVSTSYPDSVKGRFTISRDNAKNSLYLQMNSLRAEDTALYYCARGGSPYGDYGAFDIWGQGTMVTVSS

SEQ ID NO: 55 - protein, 112 aa, synthetic construct
  source 1..112: note = AB-9 VL sequence
DIVMTQTPLSSPVTLGQPASISCRSSQSLVHSEGNTYLSWLQQRPGQPPRLLIYKISNRFSGVPDRFSGSGAGTDFTLKISRVEAEDVGVYYCMQTTQFPYTFGQGTKLEIK

SEQ ID NO: 56 - protein, 5 aa, synthetic construct
  source 1..5: note = CDR-H1 of TfR.15.WH8.1.24A, TfR.15.WH8.1.24A.42Q, TfR.15.WH8.1.42Q, TfR.15.WH8.1.42Q.H6-4, TfR.15.WH8.1.42Q.L7-2, TfR.15.WH8.1.42Q.L10-1, TfR.15.WH8.1.42Q.L10-8, TfR.15.WH8.1.42Q.L10-16, TfR.15.WH8.1.42Q.H3-7, TfR.15.WH8.1, TfR.15.WH8.1.42Q.L10-16 HL scFv, and TfR.15.WH8.1.42Q.L10-16 LH scFv
SYYMH

SEQ ID NO: 57 - protein, 17 aa, synthetic construct
  source 1..17: note = CDR-H2 of TfR.15.WH8.1.24A, TfR.15.WH8.1.24A.42Q, TfR.15.WH8.1.42Q, TfR.15.WH8.1.42Q.H6-4, TfR.15.WH8.1.42Q.L7-2, TfR.15.WH8.1.42Q.L10-1, TfR.15.WH8.1.42Q.L10-8, TfR.15.WH8.1.42Q.L10-16, TfR.15.WH8.1.42Q.H3-7, TfR.15.WH8.1, TfR.15.WH8.1.42Q.L10-16 HL scFv, and TfR.15.WH8.1.42Q.L10-16 LH scFv
NIYPGSGSSNYAQKFQG

SEQ ID NO: 58 - protein, 12 aa, synthetic construct
  source 1..12: note = CDR-H3 of TfR.15.WH8.1.24A and TfR.15.WH8.1.24A.42Q
SAYYGNFDTMDN

SEQ ID NO: 59 - protein, 12 aa, synthetic construct
  source 1..12: note = CDR-H3 of TfR.15.WH8.1.42Q, TfR.15.WH8.1.42Q.L7-2, TfR.15.WH8.1.42Q.L10-1, TfR.15.WH8.1.42Q.L10-8, TfR.15.WH8.1.42Q.L10-16, TfR.15.WH8.1, TfR.15.WH8.1.42Q.L10-16 HL scFv, and TfR.15.WH8.1.42Q.L10-16 LH scFv
SSYYGNFDTMDN

SEQ ID NO: 60 - protein, 12 aa, synthetic construct
  source 1..12: note = CDR-H3 of TfR.15.WH8.1.42Q.H6-4
SSYYGRFDTMDN

SEQ ID NO: 61 - protein, 12 aa, synthetic construct
  source 1..12: note = CDR-H3 of TfR.15.WH8.1.42Q.H3-7
SSNYGNFDTMDN

SEQ ID NO: 62 - protein, 5 aa, synthetic construct
  source 1..5: note = CDR-H1 of TfR.9.1B.39.38, TfR.9.1B.39.27.L-35, TfR.9.1B.39.38.L-21, TfR.9.1B.39.38.L-6, TfR.9.1B.39.27.L-19, TfR.9.1B.35, and TfR.9.1B.39
EYAMH

SEQ ID NO: 63 - protein, 17 aa, synthetic construct
  source 1..17: note = CDR-H2 of TfR.9.1B.39.38, TfR.9.1B.39.38.L-21, and TfR.9.1B.39.38.L-6; note = CDR-H2 of TfR.9.1B.39.38, TfR.9.1B.39.38.L-21, and TfR.9.1B.39.38.L-6
GIAPNTGGTSYNQKFKG

SEQ ID NO: 64 - protein, 10 aa, synthetic construct
  source 1..10: note = CDR-H3 of TfR.9.1B.39.38, TfR.9.1B.39.27.L-35, TfR.9.1B.39.38.L-21, TfR.9.1B.39.38.L-6, TfR.9.1B.39.27.L-19, TfR.9.1B.35, and TfR.9.1B.39
QGWLLRGGDY

SEQ ID NO: 65 - protein, 17 aa, synthetic construct
  source 1..17: note = CDR-H2 of TfR.9.1B.39.27.L-35 and TfR.9.1B.39.27.L-19
GIAPGTGGTSYNQKFKG

SEQ ID NO: 66 - protein, 10 aa, synthetic construct
  source 1..10: note = CDR-L1 of TfR.15.WH8.1.24A and TfR.15.WH8.1
RASSSIRYMN

SEQ ID NO: 67 - protein, 7 aa, synthetic construct
  source 1..7: note = CDR-L2 of TfR.15.WH8.1.24A, TfR.15.WH8.1.24A.42Q, TfR.15.WH8.1.42Q, TfR.15.WH8.1.42Q.H6-4,TfR.15.WH8.1.42Q.L7-2, TfR.15.WH8.1.42Q.L10-1, TfR.15.WH8.1.42Q.L10-8, TfR.15.WH8.1.42Q.L10-16, TfR.15.WH8.1.42Q.H3-7, TfR.15.WH8.1, TfR.15.WH8.1.42Q.L10-16 HL scFv, and TfR.15.WH8.1.42Q.L10-16 LH scFv
DTSNRAT

SEQ ID NO: 68 - protein, 9 aa, synthetic construct
  source 1..9: note = CDR-L3 of TfR.15.WH8.1.24A, TfR.15.WH8.1.24A.42Q, TfR.15.WH8.1.42Q, TfR.15.WH8.1.42Q.H6-4, TfR.15.WH8.1.42Q.L7-2, TfR.15.WH8.1.42Q.L10-1, TfR.15.WH8.1.42Q.L10-8, TfR.15.WH8.1.42Q.L10-16, TfR.15.WH8.1.42Q.H3-7, TfR.15.WH8.1, TfR.15.WH8.1.42Q.L10-16 HL scFv, and TfR.15.WH8.1.42Q.L10-16 LH scFv
HQRSSYPWT

SEQ ID NO: 69 - protein, 10 aa, synthetic construct
  source 1..10: note = CDR-L1 of TfR.15.WH8.1.24A.42Q, TfR.15.WH8.1.42Q, TfR.15.WH8.1.42Q.H6-4, and TfR.15.WH8.1.42Q.H3-7
RASSSIRQMN

SEQ ID NO: 70 - protein, 10 aa, synthetic construct
  source 1..10: note = CDR-L1 of TfR.15.WH8.1.42Q.L7-2
RASSSIDQMN

SEQ ID NO: 71 - protein, 10 aa, synthetic construct
  source 1..10: note = CDR-L1 of TfR.15.WH8.1.42Q.L10-1
RASSSIRQMA

SEQ ID NO: 72 - protein, 10 aa, synthetic construct
  source 1..10: note = CDR-L1 of TfR.15.WH8.1.42Q.L10-8
RASSSIRQMS

SEQ ID NO: 73 - protein, 10 aa, synthetic construct
  source 1..10: note = CDR-L1 of TfR.15.WH8.1.42Q.L10-16, TfR.15.WH8.1.42Q.L10-16 HL scFv, and TfR.15.WH8.1.42Q.L10-16 LH scFv
RASSSIRQMH

SEQ ID NO: 74 - protein, 16 aa, synthetic construct
  source 1..16: note = CDR-L1 of TfR.9.1B.39.38; note = CDR-L1 of TfR.9.1B.39.38; note = CDR-L1 of TfR.9.1B.39.38
KASQSLLDEGGKTYLN

SEQ ID NO: 75 - protein, 7 aa, synthetic construct
  source 1..7: note = CDR-L2 of TfR.9.1B.39.38, TfR.9.1B.39.27.L-35, TfR.9.1B.39.38.L-21, TfR.9.1B.39.38.L-6, TfR.9.1B.39.27.L-19, TfR.9.1B.35, and TfR.9.1B.39
LVSKLDS

SEQ ID NO: 76 - protein, 9 aa, synthetic construct
  source 1..9: note = CDR-L3 of TfR.9.1B.39.38, TfR.9.1B.39.27.L-35, TfR.9.1B.39.38.L-21, TfR.9.1B.39.38.L-6, TfR.9.1B.39.27.L-19, TfR.9.1B.39
WQGTHFPQT

SEQ ID NO: 77 - protein, 16 aa, synthetic construct
  source 1..16: note = CDR-L1 of TfR.9.1B.39.27.L-35 and TfR.9.1B.39
KASQSLLDEDGKTYLN

SEQ ID NO: 78 - protein, 16 aa, synthetic construct
  source 1..16: note = CDR-L1 of TfR.9.1B.39.38.L-21
KASQSLLDEDNKTYLN

SEQ ID NO: 79 - protein, 16 aa, synthetic construct
  source 1..16: note = CDR-L1 of TfR.9.1B.39.38.L-6
KASQSLLDEDKKTYLN

SEQ ID NO: 80 - protein, 16 aa, synthetic construct
  source 1..16: note = CDR-L1 of TfR.9.1B.39.27.L-19
KASQSLLDEDAKTYLN

SEQ ID NO: 81 - protein, 121 aa, synthetic construct
  source 1..121: note = TfR.15.WH8.1.24A and TfR.15.WH8.1.24A.42Q VH sequence
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSAYYGNFDTMDNWGQGTTVTVSS

SEQ ID NO: 82 - protein, 106 aa, synthetic construct
  source 1..106: note = TfR.15.WH8.1.24A VL sequence
EIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 83 - protein, 106 aa, synthetic construct
  source 1..106: note = TfR.15.WH8.1.24A.42Q, TfR.15.WH8.1.42Q, TfR.15.WH8.1.42Q.H6-4, and TfR.15.WH8.1.42Q.H3-7 VL sequence
EIVLTQSPATLSLSPGERATLSCRASSSIRQMNWYQQKPGQAPRRLIYDTSNRATGIPARFSGSGSGTDFTLTISSLEPEDFAVYYCHQRSSYPWTFGGGTKVEIK

SEQ ID NO: 84 - protein, 121 aa, synthetic construct
  source 1..121: note = TfR.15.WH8.1.42Q, TfR.15.WH8.1.42Q.L7-2, TfR.15.WH8.1.42Q.L10-1, TfR.15.WH8.1.42Q.L10-8, and TfR.15.WH8.1.42Q.L10-16 VH sequence
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSS

SEQ ID NO: 85 - protein, 121 aa, synthetic construct
  source 1..121: note = TfR.15.WH8.1.42Q.H6-4 VH sequence
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGRFDTMDNWGQGTTVTVSS

SEQ ID NO: 86 - protein, 106 aa, synthetic construct
  source 1..106: note = TfR.15.WH8.1.42Q.L7-2 VL sequence
EIVLTQSPATLSLSPGERATLSCRASSSIDQMNWYQQKPGQAPRRLIYDTSNRATGIPARFSGSGSGTDFTLTISSLEPEDFAVYYCHQRSSYPWTFGGGTKVEIK

SEQ ID NO: 87 - protein, 106 aa, synthetic construct
  source 1..106: note = TfR.15.WH8.1.42Q.L10-1 VL sequence
EIVLTQSPATLSLSPGERATLSCRASSSIRQMAWYQQKPGQAPRRLIYDTSNRATGIPARFSGSGSGTDFTLTISSLEPEDFAVYYCHQRSSYPWTFGGGTKVEIK

SEQ ID NO: 88 - protein, 106 aa, synthetic construct
  source 1..106: note = TfR.15.WH8.1.42Q.L10-8 VL sequence
EIVLTQSPATLSLSPGERATLSCRASSSIRQMSWYQQKPGQAPRRLIYDTSNRATGIPARFSGSGSGTDFTLTISSLEPEDFAVYYCHQRSSYPWTFGGGTKVEIK

SEQ ID NO: 89 - protein, 106 aa, synthetic construct
  source 1..106: note = TfR.15.WH8.1.42Q.L10-16 VL sequence
EIVLTQSPATLSLSPGERATLSCRASSSIRQMHWYQQKPGQAPRRLIYDTSNRATGIPARFSGSGSGTDFTLTISSLEPEDFAVYYCHQRSSYPWTFGGGTKVEIK

SEQ ID NO: 90 - protein, 121 aa, synthetic construct
  source 1..121: note = TfR.15.WH8.1.42Q.H3-7 VH sequence
QVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSNYGNFDTMDNWGQGTTVTVSS

SEQ ID NO: 91 - protein, 119 aa, synthetic construct
  source 1..119: note = TfR.9.1B.39.38, TfR.9.1B.39.38.L-21, and TfR.9.1B.39.38.L-6 VH sequence
QVQLVQSGAEVKKPGASVKVSCKASGYTFTEYAMHWVRQAPGQGLEWMGGIAPNTGGTSYNQKFKGRVTMTVDTSTSTAYMELSSLRSEDTAVYYCASQGWLLRGGDYWGQGTLVTVSS

SEQ ID NO: 92 - protein, 112 aa, synthetic construct
  source 1..112: note = TfR.9.1B.39.38 VL sequence
DVVMTQSPLSLPVTLGQPASISCKASQSLLDEGGKTYLNWLQQRPGQSPRRLIYLVSKLDSGVPDRFSGSGSGTDFTLKISRVEAEDVGVYYCWQGTHFPQTFGGGTKVEIK

SEQ ID NO: 93 - protein, 119 aa, synthetic construct
  source 1..119: note = TfR.9.1B.39.27.L-35 and TfR.9.1B.39.27.L-19 VH sequence
QVQLVQSGAEVKKPGASVKVSCKASGYTFTEYAMHWVRQAPGQGLEWMGGIAPGTGGTSYNQKFKGRVTMTVDTSTSTAYMELSSLRSEDTAVYYCASQGWLLRGGDYWGQGTLVTVSS

SEQ ID NO: 94 - protein, 112 aa, synthetic construct
  source 1..112: note = TfR.9.1B.39.27.L-35 VL sequence
DVVMTQSPLSLPVTLGQPASISCKASQSLLDEDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 95 - protein, 112 aa, synthetic construct
  source 1..112: note = TfR.9.1B.39.38.L-21 VL sequence
DVVMTQSPLSLPVTLGQPASISCKASQSLLDEDNKTYLNWLQQRPGQSPRRLIYLVSKLDSGVPDRFSGSGSGTDFTLKISRVEAEDVGVYYCWQGTHFPQTFGGGTKVEIK

SEQ ID NO: 96 - protein, 112 aa, synthetic construct
  source 1..112: note = TfR.9.1B.39.38.L-6 VL sequence
DVVMTQSPLSLPVTLGQPASISCKASQSLLDEDKKTYLNWLQQRPGQSPRRLIYLVSKLDSGVPDRFSGSGSGTDFTLKISRVEAEDVGVYYCWQGTHFPQTFGGGTKVEIK

SEQ ID NO: 97 - protein, 112 aa, synthetic construct
  source 1..112: note = TfR.9.1B.39.27.L-19 VL sequence
DVVMTQSPLSLPVTLGQPASISCKASQSLLDEDAKTYLNWLQQRPGQSPRRLIYLVSKLDSGVPDRFSGSGSGTDFTLKISRVEAEDVGVYYCWQGTHFPQTFGGGTKVEIK

SEQ ID NO: 98 - protein, 5 aa, synthetic construct
  source 1..5: note = CDR-H1 of Anti-CD98hc Antibodies
AYDMS

SEQ ID NO: 99 - protein, 17 aa, synthetic construct
  source 1..17: note = CDR-H2 of Anti-CD98hc Antibodies
YISSGGGSTYYADSVKG

SEQ ID NO: 100 - protein, 10 aa, synthetic construct
  source 1..10: note = CDR-H3 of Anti-CD98hc Antibodies
QGGLPAWFVY

SEQ ID NO: 101 - protein, 17 aa, synthetic construct
  source 1..17: note = CDR-H2 of CD98hc.04.048.WH1.15
YISSGGGSTYYADEVKG

SEQ ID NO: 102 - protein, 17 aa, synthetic construct
  source 1..17: note = CDR-H2 of CD98hc.04.048.WH1.20
YISSGGGSTYYADFVKG

SEQ ID NO: 103 - protein, 17 aa, synthetic construct
  source 1..17: note = CDR-H2 of CD98hc.04.048.WH1.23
YISSGGGSTYYADYVKG

SEQ ID NO: 104 - protein, 17 aa, synthetic construct
  source 1..17: note = CDR-H2 of CD98hc.04.048.WH1.078
YKSSGGGSTYYADSVKG

SEQ ID NO: 105 - protein, 17 aa, synthetic construct
  source 1..17: note = CDR-H2 of CD98hc.04.048.WH1.083
YRSSGGGSTYYADSVKG

SEQ ID NO: 106 - protein, 17 aa, synthetic construct
  source 1..17: note = CDR-H2 of CD98hc.04.048.WH1.105 and CD98hc.04.048.WH1.296
YISSQGGSTYYADSVKG

SEQ ID NO: 107 - protein, 17 aa, synthetic construct
  source 1..17: note = CDR-H2 of CD98hc.04.048.WH1.118
YISSGKGSTYYADSVKG

SEQ ID NO: 108 - protein, 17 aa, synthetic construct
  source 1..17: note = CDR-H2 of CD98hc.04.048.WH1.119
YISSGHGSTYYADSVKG

SEQ ID NO: 109 - protein, 17 aa, synthetic construct
  source 1..17: note = CDR-H2 of CD98hc.04.048.WH1.121
YISSGRGSTYYADSVKG

SEQ ID NO: 110 - protein, 17 aa, synthetic construct
  source 1..17: note = CDR-H2 of CD98hc.04.048.WH1.133
YISSGGGHTYYADSVKG

SEQ ID NO: 111 - protein, 17 aa, synthetic construct
  source 1..17: note = CDR-H2 of CD98hc.04.048.WH1.151
YISSGGGSDYYADSVKG

SEQ ID NO: 112 - protein, 10 aa, synthetic construct
  source 1..10: note = CDR-H3 of CD98hc.04.048.WH1.158
QGSLPAWFVY

SEQ ID NO: 113 - protein, 10 aa, synthetic construct
  source 1..10: note = CDR-H3 of CD98hc.04.048.WH1.162
QGGHPAWFVY

SEQ ID NO: 114 - protein, 10 aa, synthetic construct
  source 1..10: note = CDR-H3 of CD98hc.04.048.WH1.165
QGGAPAWFVY

SEQ ID NO: 115 - protein, 10 aa, synthetic construct
  source 1..10: note = CDR-H3 of CD98hc.04.048.WH1.166
QGGTPAWFVY

SEQ ID NO: 116 - protein, 10 aa, synthetic construct
  source 1..10: note = CDR-H3 of CD98hc.04.048.WH1.167
QGGQPAWFVY

SEQ ID NO: 117 - protein, 10 aa, synthetic construct
  source 1..10: note = CDR-H3 of CD98hc.04.048.WH1.168
QGGLPALFVY

SEQ ID NO: 118 - protein, 10 aa, synthetic construct
  source 1..10: note = CDR-H3 of CD98hc.04.048.WH1.170
QGGLPAKFVY

SEQ ID NO: 119 - protein, 10 aa, synthetic construct
  source 1..10: note = CDR-H3 of CD98hc.04.048.WH1.171
QGGLPATFVY

SEQ ID NO: 120 - protein, 10 aa, synthetic construct
  source 1..10: note = CDR-H3 of CD98hc.04.048.WH1.174
QGGLPARFVY

SEQ ID NO: 121 - protein, 5 aa, synthetic construct
  source 1..5: note = CDR-H1 of CD98hc.04.048.WH1.55 and CD98hc.04.048.WH1.76
AYAMS

SEQ ID NO: 122 - protein, 17 aa, synthetic construct
  source 1..17: note = CDR-L1 of Anti-CD98hc Antibodies
KSSQSLLYSNTQKNYLA

SEQ ID NO: 123 - protein, 7 aa, synthetic construct
  source 1..7: note = CDR-L2 of Anti-CD98hc Antibodies
WASTRES

SEQ ID NO: 124 - protein, 9 aa, synthetic construct
  source 1..9: note = CDR-L3 of Anti-CD98hc Antibodies
QQYSYYPPT

SEQ ID NO: 125 - protein, 17 aa, synthetic construct
  source 1..17: note = CDR-L1 of CD98hc.04.048.WH1.193
KSSQSLKYSNTQKNYLA

SEQ ID NO: 126 - protein, 17 aa, synthetic construct
  source 1..17: note = CDR-L1 of CD98hc.04.048.WH1.217
KSSQSLLYQNTQKNYLA

SEQ ID NO: 127 - protein, 17 aa, synthetic construct
  source 1..17: note = CDR-L1 of CD98hc.04.048.WH1.218 and CD98hc.04.048.WH1.296
KSSQSLLYENTQKNYLA

SEQ ID NO: 128 - protein, 17 aa, synthetic construct
  source 1..17: note = CDR-L1 of CD98hc.04.048.WH1.222
KSSQSLLYSHTQKNYLA

SEQ ID NO: 129 - protein, 17 aa, synthetic construct
  source 1..17: note = CDR-L1 of CD98hc.04.048.WH1.224
KSSQSLLYSTTQKNYLA

SEQ ID NO: 130 - protein, 17 aa, synthetic construct
  source 1..17: note = CDR-L1 of CD98hc.04.048.WH1.232
KSSQSLLYSNSQKNYLA

SEQ ID NO: 131 - protein, 17 aa, synthetic construct
  source 1..17: note = CDR-L1 of CD98hc.04.048.WH1.236
KSSQSLLYSNKQKNYLA

SEQ ID NO: 132 - protein, 17 aa, synthetic construct
  source 1..17: note = CDR-L1 of CD98hc.04.048.WH1.185
KSSQSSLYSNTQKNYLA

SEQ ID NO: 133 - protein, 17 aa, synthetic construct
  source 1..17: note = CDR-L1 of CD98hc.04.048.WH1.208
KSSQSLLGSNTQKNYLA

SEQ ID NO: 134 - protein, 17 aa, synthetic construct
  source 1..17: note = CDR-L1 of CD98hc.04.048.WH1.231
KSSQSLLYSNIQKNYLA

SEQ ID NO: 135 - protein, 17 aa, synthetic construct
  source 1..17: note = CDR-L1 of CD98hc.04.048.WH1.235
KSSQSLLYSNHQKNYLA

SEQ ID NO: 136 - protein, 17 aa, synthetic construct
  source 1..17: note = CDR-L1 of CD98hc.04.048.WH1.250
KSSQSLLYSNTQDNYLA

SEQ ID NO: 137 - protein, 17 aa, synthetic construct
  source 1..17: note = CDR-L1 of CD98hc.04.048.WH1.253
KSSQSLLYSNTQGNYLA

SEQ ID NO: 138 - protein, 9 aa, synthetic construct
  source 1..9: note = CDR-L3 of CD98hc.04.048.WH1.262
QQKSYYPPT

SEQ ID NO: 139 - protein, 9 aa, synthetic construct
  source 1..9: note = CDR-L3 of CD98hc.04.048.WH1.269
QQYKYYPPT

SEQ ID NO: 140 - protein, 9 aa, synthetic construct
  source 1..9: note = CDR-L3 of CD98hc.04.048.WH1.271
QQYTYYPPT

SEQ ID NO: 141 - protein, 9 aa, synthetic construct
  source 1..9: note = CDR-L3 of CD98hc.04.048.WH1.274
QQYEYYPPT

SEQ ID NO: 142 - protein, 9 aa, synthetic construct
  source 1..9: note = CDR-L3 of CD98hc.04.048.WH1.276
QQYIYYPPT

SEQ ID NO: 143 - protein, 9 aa, synthetic construct
  source 1..9: note = CDR-L3 of CD98hc.04.048.WH1.279
QQYSVYPPT

SEQ ID NO: 144 - protein, 9 aa, synthetic construct
  source 1..9: note = CDR-L3 of CD98hc.04.048.WH1.281
QQYSEYPPT

SEQ ID NO: 145 - protein, 9 aa, synthetic construct
  source 1..9: note = CDR-L3 of CD98hc.04.048.WH1.282
QQYSLYPPT

SEQ ID NO: 146 - protein, 9 aa, synthetic construct
  source 1..9: note = CDR-L3 of CD98hc.04.048.WH1.283
QQYSQYPPT

SEQ ID NO: 147 - protein, 9 aa, synthetic construct
  source 1..9: note = CDR-L3 of CD98hc.04.048.WH1.39 and CD98hc.04.048.WH1.76
AQYSYYPPT

SEQ ID NO: 148 - protein, 119 aa, synthetic construct
  source 1..119: note = VH sequence of Anti-CD98hc Antibodies
EVQLLESGGGLVQPGGSLRLSCAASGFGISAYDMSWVRQAPGKGLEWVSYISSGGGSTYYADSVKGRFTISRDNSKNTLYLQMNSLRAEDTAVYYCARQGGLPAWFVYWGQGTLVTVSS

SEQ ID NO: 149 - protein, 113 aa, synthetic construct
  source 1..113: note = VL sequence of Anti-CD98hc Antibodies
DIVMTQSPDSLAVSLGERATINCKSSQSLLYSNTQKNYLAWYQQKPGQPPKLLIYWASTRESGVPDRFSGSGSGTDFTLTISSLQAEDVAVYYCQQYSYYPPTFGGGTKVEIK

SEQ ID NO: 150 - protein, 119 aa, synthetic construct
  source 1..119: note = CD98hc.04.048.WH1.15 VH sequence
EVQLLESGGGLVQPGGSLRLSCAASGFGISAYDMSWVRQAPGKGLEWVSYISSGGGSTYYADEVKGRFTISRDNSKNTLYLQMNSLRAEDTAVYYCARQGGLPAWFVYWGQGTLVTVSS

SEQ ID NO: 151 - protein, 119 aa, synthetic construct
  source 1..119: note = CD98hc.04.048.WH1.20 VH sequence
EVQLLESGGGLVQPGGSLRLSCAASGFGISAYDMSWVRQAPGKGLEWVSYISSGGGSTYYADFVKGRFTISRDNSKNTLYLQMNSLRAEDTAVYYCARQGGLPAWFVYWGQGTLVTVSS

SEQ ID NO: 152 - protein, 119 aa, synthetic construct
  source 1..119: note = CD98hc.04.048.WH1.23 VH sequence
EVQLLESGGGLVQPGGSLRLSCAASGFGISAYDMSWVRQAPGKGLEWVSYISSGGGSTYYADYVKGRFTISRDNSKNTLYLQMNSLRAEDTAVYYCARQGGLPAWFVYWGQGTLVTVSS

SEQ ID NO: 153 - protein, 119 aa, synthetic construct
  source 1..119: note = CD98hc.04.048.WH1.078 VH sequence
EVQLLESGGGLVQPGGSLRLSCAASGFGISAYDMSWVRQAPGKGLEWVSYKSSGGGSTYYADSVKGRFTISRDNSKNTLYLQMNSLRAEDTAVYYCARQGGLPAWFVYWGQGTLVTVSS

SEQ ID NO: 154 - protein, 119 aa, synthetic construct
  source 1..119: note = CD98hc.04.048.WH1.083 VH sequence
EVQLLESGGGLVQPGGSLRLSCAASGFGISAYDMSWVRQAPGKGLEWVSYRSSGGGSTYYADSVKGRFTISRDNSKNTLYLQMNSLRAEDTAVYYCARQGGLPAWFVYWGQGTLVTVSS

SEQ ID NO: 155 - protein, 119 aa, synthetic construct
  source 1..119: note = CD98hc.04.048.WH1.105 and CD98hc.04.048.WH1.296 VH sequence
EVQLLESGGGLVQPGGSLRLSCAASGFGISAYDMSWVRQAPGKGLEWVSYISSQGGSTYYADSVKGRFTISRDNSKNTLYLQMNSLRAEDTAVYYCARQGGLPAWFVYWGQGTLVTVSS

SEQ ID NO: 156 - protein, 119 aa, synthetic construct
  source 1..119: note = CD98hc.04.048.WH1.118 VH sequence
EVQLLESGGGLVQPGGSLRLSCAASGFGISAYDMSWVRQAPGKGLEWVSYISSGKGSTYYADSVKGRFTISRDNSKNTLYLQMNSLRAEDTAVYYCARQGGLPAWFVYWGQGTLVTVSS

SEQ ID NO: 157 - protein, 119 aa, synthetic construct
  source 1..119: note = CD98hc.04.048.WH1.119 VH sequence
EVQLLESGGGLVQPGGSLRLSCAASGFGISAYDMSWVRQAPGKGLEWVSYISSGHGSTYYADSVKGRFTISRDNSKNTLYLQMNSLRAEDTAVYYCARQGGLPAWFVYWGQGTLVTVSS

SEQ ID NO: 158 - protein, 119 aa, synthetic construct
  source 1..119: note = CD98hc.04.048.WH1.121 VH sequence
EVQLLESGGGLVQPGGSLRLSCAASGFGISAYDMSWVRQAPGKGLEWVSYISSGRGSTYYADSVKGRFTISRDNSKNTLYLQMNSLRAEDTAVYYCARQGGLPAWFVYWGQGTLVTVSS

SEQ ID NO: 159 - protein, 119 aa, synthetic construct
  source 1..119: note = CD98hc.04.048.WH1.133 VH sequence
EVQLLESGGGLVQPGGSLRLSCAASGFGISAYDMSWVRQAPGKGLEWVSYISSGGGHTYYADSVKGRFTISRDNSKNTLYLQMNSLRAEDTAVYYCARQGGLPAWFVYWGQGTLVTVSS

SEQ ID NO: 160 - protein, 113 aa, synthetic construct
  source 1..113: note = CD98hc.04.048.WH1.193 VL sequence
DIVMTQSPDSLAVSLGERATINCKSSQSLKYSNTQKNYLAWYQQKPGQPPKLLIYWASTRESGVPDRFSGSGSGTDFTLTISSLQAEDVAVYYCQQYSYYPPTFGGGTKVEIK

SEQ ID NO: 161 - protein, 113 aa, synthetic construct
  source 1..113: note = CD98hc.04.048.WH1.217 VL sequence
DIVMTQSPDSLAVSLGERATINCKSSQSLLYQNTQKNYLAWYQQKPGQPPKLLIYWASTRESGVPDRFSGSGSGTDFTLTISSLQAEDVAVYYCQQYSYYPPTFGGGTKVEIK

SEQ ID NO: 162 - protein, 113 aa, synthetic construct
  source 1..113: note = CD98hc.04.048.WH1.218 and CD98hc.04.048.WH1.296 VL sequence
DIVMTQSPDSLAVSLGERATINCKSSQSLLYENTQKNYLAWYQQKPGQPPKLLIYWASTRESGVPDRFSGSGSGTDFTLTISSLQAEDVAVYYCQQYSYYPPTFGGGTKVEIK

SEQ ID NO: 163 - protein, 113 aa, synthetic construct
  source 1..113: note = CD98hc.04.048.WH1.222 VL sequence
DIVMTQSPDSLAVSLGERATINCKSSQSLLYSHTQKNYLAWYQQKPGQPPKLLIYWASTRESGVPDRFSGSGSGTDFTLTISSLQAEDVAVYYCQQYSYYPPTFGGGTKVEIK

SEQ ID NO: 164 - protein, 113 aa, synthetic construct
  source 1..113: note = CD98hc.04.048.WH1.224 VL sequence
DIVMTQSPDSLAVSLGERATINCKSSQSLLYSTTQKNYLAWYQQKPGQPPKLLIYWASTRESGVPDRFSGSGSGTDFTLTISSLQAEDVAVYYCQQYSYYPPTFGGGTKVEIK

SEQ ID NO: 165 - protein, 113 aa, synthetic construct
  source 1..113: note = CD98hc.04.048.WH1.232 VL sequence
DIVMTQSPDSLAVSLGERATINCKSSQSLLYSNSQKNYLAWYQQKPGQPPKLLIYWASTRESGVPDRFSGSGSGTDFTLTISSLQAEDVAVYYCQQYSYYPPTFGGGTKVEIK

SEQ ID NO: 166 - protein, 113 aa, synthetic construct
  source 1..113: note = CD98hc.04.048.WH1.236 VL sequence
DIVMTQSPDSLAVSLGERATINCKSSQSLLYSNKQKNYLAWYQQKPGQPPKLLIYWASTRESGVPDRFSGSGSGTDFTLTISSLQAEDVAVYYCQQYSYYPPTFGGGTKVEIK

SEQ ID NO: 167 - protein, 119 aa, synthetic construct
  source 1..119: note = CD98hc.04.048.WH1.151 VH sequence
EVQLLESGGGLVQPGGSLRLSCAASGFGISAYDMSWVRQAPGKGLEWVSYISSGGGSDYYADSVKGRFTISRDNSKNTLYLQMNSLRAEDTAVYYCARQGGLPAWFVYWGQGTLVTVSS

SEQ ID NO: 168 - protein, 119 aa, synthetic construct
  source 1..119: note = CD98hc.04.048.WH1.158 VH sequence
EVQLLESGGGLVQPGGSLRLSCAASGFGISAYDMSWVRQAPGKGLEWVSYISSGGGSTYYADSVKGRFTISRDNSKNTLYLQMNSLRAEDTAVYYCARQGSLPAWFVYWGQGTLVTVSS

SEQ ID NO: 169 - protein, 119 aa, synthetic construct
  source 1..119: note = CD98hc.04.048.WH1.162 VH sequence
EVQLLESGGGLVQPGGSLRLSCAASGFGISAYDMSWVRQAPGKGLEWVSYISSGGGSTYYADSVKGRFTISRDNSKNTLYLQMNSLRAEDTAVYYCARQGGHPAWFVYWGQGTLVTVSS

SEQ ID NO: 170 - protein, 119 aa, synthetic construct
  source 1..119: note = CD98hc.04.048.WH1.165 VH sequence
EVQLLESGGGLVQPGGSLRLSCAASGFGISAYDMSWVRQAPGKGLEWVSYISSGGGSTYYADSVKGRFTISRDNSKNTLYLQMNSLRAEDTAVYYCARQGGAPAWFVYWGQGTLVTVSS

SEQ ID NO: 171 - protein, 119 aa, synthetic construct
  source 1..119: note = CD98hc.04.048.WH1.166 VH sequence
EVQLLESGGGLVQPGGSLRLSCAASGFGISAYDMSWVRQAPGKGLEWVSYISSGGGSTYYADSVKGRFTISRDNSKNTLYLQMNSLRAEDTAVYYCARQGGTPAWFVYWGQGTLVTVSS

SEQ ID NO: 172 - protein, 119 aa, synthetic construct
  source 1..119: note = CD98hc.04.048.WH1.167 VH sequence
EVQLLESGGGLVQPGGSLRLSCAASGFGISAYDMSWVRQAPGKGLEWVSYISSGGGSTYYADSVKGRFTISRDNSKNTLYLQMNSLRAEDTAVYYCARQGGQPAWFVYWGQGTLVTVSS

SEQ ID NO: 173 - protein, 119 aa, synthetic construct
  source 1..119: note = CD98hc.04.048.WH1.168 VH sequence
EVQLLESGGGLVQPGGSLRLSCAASGFGISAYDMSWVRQAPGKGLEWVSYISSGGGSTYYADSVKGRFTISRDNSKNTLYLQMNSLRAEDTAVYYCARQGGLPALFVYWGQGTLVTVSS

SEQ ID NO: 174 - protein, 119 aa, synthetic construct
  source 1..119: note = CD98hc.04.048.WH1.170 VH sequence
EVQLLESGGGLVQPGGSLRLSCAASGFGISAYDMSWVRQAPGKGLEWVSYISSGGGSTYYADSVKGRFTISRDNSKNTLYLQMNSLRAEDTAVYYCARQGGLPAKFVYWGQGTLVTVSS

SEQ ID NO: 175 - protein, 119 aa, synthetic construct
  source 1..119: note = CD98hc.04.048.WH1.171 VH sequence
EVQLLESGGGLVQPGGSLRLSCAASGFGISAYDMSWVRQAPGKGLEWVSYISSGGGSTYYADSVKGRFTISRDNSKNTLYLQMNSLRAEDTAVYYCARQGGLPATFVYWGQGTLVTVSS

SEQ ID NO: 176 - protein, 119 aa, synthetic construct
  source 1..119: note = CD98hc.04.048.WH1.174 VH sequence
EVQLLESGGGLVQPGGSLRLSCAASGFGISAYDMSWVRQAPGKGLEWVSYISSGGGSTYYADSVKGRFTISRDNSKNTLYLQMNSLRAEDTAVYYCARQGGLPARFVYWGQGTLVTVSS

SEQ ID NO: 177 - protein, 113 aa, synthetic construct
  source 1..113: note = CD98hc.04.048.WH1.185 VL sequence
DIVMTQSPDSLAVSLGERATINCKSSQSSLYSNTQKNYLAWYQQKPGQPPKLLIYWASTRESGVPDRFSGSGSGTDFTLTISSLQAEDVAVYYCQQYSYYPPTFGGGTKVEIK

SEQ ID NO: 178 - protein, 113 aa, synthetic construct
  source 1..113: note = CD98hc.04.048.WH1.208 VL sequence
DIVMTQSPDSLAVSLGERATINCKSSQSLLGSNTQKNYLAWYQQKPGQPPKLLIYWASTRESGVPDRFSGSGSGTDFTLTISSLQAEDVAVYYCQQYSYYPPTFGGGTKVEIK

SEQ ID NO: 179 - protein, 113 aa, synthetic construct
  source 1..113: note = CD98hc.04.048.WH1.231 VL sequence
DIVMTQSPDSLAVSLGERATINCKSSQSLLYSNIQKNYLAWYQQKPGQPPKLLIYWASTRESGVPDRFSGSGSGTDFTLTISSLQAEDVAVYYCQQYSYYPPTFGGGTKVEIK

SEQ ID NO: 180 - protein, 113 aa, synthetic construct
  source 1..113: note = CD98hc.04.048.WH1.235 VL sequence
DIVMTQSPDSLAVSLGERATINCKSSQSLLYSNHQKNYLAWYQQKPGQPPKLLIYWASTRESGVPDRFSGSGSGTDFTLTISSLQAEDVAVYYCQQYSYYPPTFGGGTKVEIK

SEQ ID NO: 181 - protein, 113 aa, synthetic construct
  source 1..113: note = CD98hc.04.048.WH1.250 VL sequence
DIVMTQSPDSLAVSLGERATINCKSSQSLLYSNTQDNYLAWYQQKPGQPPKLLIYWASTRESGVPDRFSGSGSGTDFTLTISSLQAEDVAVYYCQQYSYYPPTFGGGTKVEIK

SEQ ID NO: 182 - protein, 113 aa, synthetic construct
  source 1..113: note = CD98hc.04.048.WH1.253 VL sequence
DIVMTQSPDSLAVSLGERATINCKSSQSLLYSNTQGNYLAWYQQKPGQPPKLLIYWASTRESGVPDRFSGSGSGTDFTLTISSLQAEDVAVYYCQQYSYYPPTFGGGTKVEIK

SEQ ID NO: 183 - protein, 113 aa, synthetic construct
  source 1..113: note = CD98hc.04.048.WH1.262 VL sequence
DIVMTQSPDSLAVSLGERATINCKSSQSLLYSNTQKNYLAWYQQKPGQPPKLLIYWASTRESGVPDRFSGSGSGTDFTLTISSLQAEDVAVYYCQQKSYYPPTFGGGTKVEIK

SEQ ID NO: 184 - protein, 113 aa, synthetic construct
  source 1..113: note = CD98hc.04.048.WH1.269 VL sequence
DIVMTQSPDSLAVSLGERATINCKSSQSLLYSNTQKNYLAWYQQKPGQPPKLLIYWASTRESGVPDRFSGSGSGTDFTLTISSLQAEDVAVYYCQQYKYYPPTFGGGTKVEIK

SEQ ID NO: 185 - protein, 113 aa, synthetic construct
  source 1..113: note = CD98hc.04.048.WH1.271 VL sequence
DIVMTQSPDSLAVSLGERATINCKSSQSLLYSNTQKNYLAWYQQKPGQPPKLLIYWASTRESGVPDRFSGSGSGTDFTLTISSLQAEDVAVYYCQQYTYYPPTFGGGTKVEIK

SEQ ID NO: 186 - protein, 113 aa, synthetic construct
  source 1..113: note = CD98hc.04.048.WH1.274 VL sequence
DIVMTQSPDSLAVSLGERATINCKSSQSLLYSNTQKNYLAWYQQKPGQPPKLLIYWASTRESGVPDRFSGSGSGTDFTLTISSLQAEDVAVYYCQQYEYYPPTFGGGTKVEIK

SEQ ID NO: 187 - protein, 113 aa, synthetic construct
  source 1..113: note = CD98hc.04.048.WH1.276 VL sequence
DIVMTQSPDSLAVSLGERATINCKSSQSLLYSNTQKNYLAWYQQKPGQPPKLLIYWASTRESGVPDRFSGSGSGTDFTLTISSLQAEDVAVYYCQQYIYYPPTFGGGTKVEIK

SEQ ID NO: 188 - protein, 113 aa, synthetic construct
  source 1..113: note = CD98hc.04.048.WH1.279 VL sequence
DIVMTQSPDSLAVSLGERATINCKSSQSLLYSNTQKNYLAWYQQKPGQPPKLLIYWASTRESGVPDRFSGSGSGTDFTLTISSLQAEDVAVYYCQQYSVYPPTFGGGTKVEIK

SEQ ID NO: 189 - protein, 113 aa, synthetic construct
  source 1..113: note = CD98hc.04.048.WH1.281 VL sequence
DIVMTQSPDSLAVSLGERATINCKSSQSLLYSNTQKNYLAWYQQKPGQPPKLLIYWASTRESGVPDRFSGSGSGTDFTLTISSLQAEDVAVYYCQQYSEYPPTFGGGTKVEIK

SEQ ID NO: 190 - protein, 113 aa, synthetic construct
  source 1..113: note = CD98hc.04.048.WH1.282 VL sequence
DIVMTQSPDSLAVSLGERATINCKSSQSLLYSNTQKNYLAWYQQKPGQPPKLLIYWASTRESGVPDRFSGSGSGTDFTLTISSLQAEDVAVYYCQQYSLYPPTFGGGTKVEIK

SEQ ID NO: 191 - protein, 113 aa, synthetic construct
  source 1..113: note = CD98hc.04.048.WH1.283 VL sequence
DIVMTQSPDSLAVSLGERATINCKSSQSLLYSNTQKNYLAWYQQKPGQPPKLLIYWASTRESGVPDRFSGSGSGTDFTLTISSLQAEDVAVYYCQQYSQYPPTFGGGTKVEIK

SEQ ID NO: 192 - protein, 113 aa, synthetic construct
  source 1..113: note = CD98hc.04.048.WH1.39 and CD98hc.04.048.WH1.76 VL sequence
DIVMTQSPDSLAVSLGERATINCKSSQSLLYSNTQKNYLAWYQQKPGQPPKLLIYWASTRESGVPDRFSGSGSGTDFTLTISSLQAEDVAVYYCAQYSYYPPTFGGGTKVEIK

SEQ ID NO: 193 - protein, 119 aa, synthetic construct
  source 1..119: note = CD98hc.04.048.WH1.55 and CD98hc.04.048.WH1.76 VH sequence
EVQLLESGGGLVQPGGSLRLSCAASGFGISAYAMSWVRQAPGKGLEWVSYISSGGGSTYYADSVKGRFTISRDNSKNTLYLQMNSLRAEDTAVYYCARQGGLPAWFVYWGQGTLVTVSS

SEQ ID NO: 194 - protein, 113 aa, synthetic construct
  source 1..113: note = CD98hc.04.048.WH1.55 VL sequence
DIVMSQSPSSLAVSVGEKVTMSCKSSQSLLYSNTQKNYLAWYQQKPGQSPKLLIYWASTRESGVPDRFTGSGSGTDFTLTISSVKAEDLAIYYCQQYSYYPPTFGGGTKLEIK

SEQ ID NO: 195 - protein, 245 aa, synthetic construct
  source 1..245: note = antibody 8D3v12 scFv
EVQLVESGGGLVQPGNSLTLSCVASGFTFSNYGMHWIRQAPKKGLEWIAAIYYDSSKMNYADTVKGRFTISRDNSKNTLYLEMNSLRSEDTAMYYCAVPTSHYVVDVWGQGVSVTVSSGGSEGKSSGSGSESKSTGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELK

SEQ ID NO: 196 - protein, 5 aa, synthetic construct
  source 1..5: note = CDR-H1 of 8D3v12 scFv mouse surrogate TfR scFv (VH-linker-VL format)
NYGMH

SEQ ID NO: 197 - protein, 17 aa, synthetic construct
  source 1..17: note = CDR-H2 of 8D3v12 scFv mouse surrogate TfR scFv (VH-linker-VL format)
AIYYDSSKMNYADTVKG

SEQ ID NO: 198 - protein, 9 aa, synthetic construct
  source 1..9: note = CDR-H3 of 8D3v12 scFv mouse surrogate TfR scFv (VH-linker-VL format)
PTSHYVVDV

SEQ ID NO: 199 - protein, 11 aa, synthetic construct
  source 1..11: note = CDR-L1 of 8D3v12 scFv mouse surrogate TfR scFv (VH-linker-VL format)
QASQDIGNWLA

SEQ ID NO: 200 - protein, 7 aa, synthetic construct
  source 1..7: note = CDR-L2 of 8D3v12 scFv mouse surrogate TfR scFv (VH-linker-VL format)
GATSLAD

SEQ ID NO: 201 - protein, 9 aa, synthetic construct
  source 1..9: note = CDR-L3 of 8D3v12 scFv mouse surrogate TfR scFv (VH-linker-VL format)
LQAYNTPWT

SEQ ID NO: 202 - protein, 247 aa, synthetic construct
  source 1..247: note = Disulfide stapled TfR.15.WH8.1.42Q.L10-16 Mutate AA to cysteines at VH44 and VL100 (in VH-VL orientation)
QVQLVQSGAEVKKPGASVKVSCKASGYTFTSYYMHWVRQAPGQCLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMHWYQQKPGQAPRRLIYDTSNRATGIPARFSGSGSGTDFTLTISSLEPEDFAVYYCHQRSSYPWTFGCGTKVEIK

SEQ ID NO: 203 - protein, 247 aa, synthetic construct
  source 1..247: note = Disulfide stapled TfR.15.WH8.1.42Q.L10-16 Mutate AA to cysteines at VH44 and VL100 (in VL-VH orientation)
EIVLTQSPATLSLSPGERATLSCRASSSIRQMHWYQQKPGQAPRRLIYDTSNRATGIPARFSGSGSGTDFTLTISSLEPEDFAVYYCHQRSSYPWTFGCGTKVEIKGGSEGKSSGSGSESKSTGGSQVQLVQSGAEVKKPGASVKVSCKASGYTFTSYYMHWVRQAPGQCLEWMGNIYPGSGSSNYAQKFQGRVTMTRDTSTSTVYMELSSLRSEDTAVYYCTRSSYYGNFDTMDNWGQGTTVTVSS

SEQ ID NO: 204 - protein, 4 aa, synthetic construct
  source 1..4: note = glycine linker
GGGG

SEQ ID NO: 205 - protein, 6 aa, synthetic construct
  source 1..6: note = glycine-rich linker
  VARIANT 4: note = N is any amino acid
GGGNGG

SEQ ID NO: 206 - protein, 4 aa, synthetic construct
  source 1..4: note = glycine-serine linker
GGGS

SEQ ID NO: 207 - protein, 15 aa, synthetic construct
  source 1..15: note = (GGGGS)x3 linker
GGGGSGGGGSGGGGS

SEQ ID NO: 208 - protein, 15 aa, synthetic construct
  source 1..15: note = (GGSGG)x3 linker
GGSGGGGSGGGGSGG

SEQ ID NO: 209 - protein, 20 aa, synthetic construct
  source 1..20: note = linker
GGSEGKSSGSGSESKSTGGS

SEQ ID NO: 210 - protein, 20 aa, synthetic construct
  source 1..20: note = linker
GGGGSGGGGSGGGGSGGGGS

SEQ ID NO: 211 - protein, 330 aa, synthetic construct [sequence omitted]

SEQ ID NO: 212 - protein, 326 aa, synthetic construct
  source 1..326: note = Human IgG2 Fc
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 213 - protein, 327 aa, synthetic construct
  source 1..327: note = Human IgG4 Fc
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 214 - protein, 5 aa, synthetic construct
  source 1..5: note = Linker between c-terminus of anti-Abeta antibody heavy chain Fc and anti-TfR scFv
GGSGG

SEQ ID NO: 215 - protein, 14 aa, synthetic construct
  source 1..14: note = Human Pyroglutamated Glu3-16
  MOD_RES 1: note = Pyroglutamate
EFRHDSGYEVHHQK

SEQ ID NO: 216 - protein, 14 aa, synthetic construct
  source 1..14: note = Human Non-modified 3-16 Abeta
EFRHDSGYEVHHQK

SEQ ID NO: 217 - protein, 22 aa, synthetic construct
  source 1..22: note = Human Pyroglutamated Glu3-24 Abeta
  MOD_RES 1: note = Pyroglutamate
EFRHDSGYEVHHQKLVFFAEDV

SEQ ID NO: 218 - protein, 22 aa, synthetic construct
  source 1..22: note = Human Non-modified 3-24 Abeta
EFRHDSGYEVHHQKLVFFAEDV

SEQ ID NO: 219 - protein, 40 aa, synthetic construct
  source 1..40: note = Human Non-modified 1-40 Abeta
DAEFRHDSGYEVHHQKLVFFAEDVGSNKGAIIGLMVGGVV

SEQ ID NO: 220 - protein, 38 aa, synthetic construct
  source 1..38: note = Human Non-modified Abeta 3-40
EFRHDSGYEVHHQKLVFFAEDVGSNKGAIIGLMVGGVV

SEQ ID NO: 221 - protein, 14 aa, synthetic construct
  source 1..14: note = Human Pyroglutamated Glu11-24 Abeta
  MOD_RES 1: note = Pyroglutamate
EVHHQKLVFFAEDV

SEQ ID NO: 222 - protein, 12 aa, synthetic construct
  source 1..12: note = Human Nitrosylated Tyr10 Abeta 3-14
  MOD_RES 8: note = 3-nitrotyrosine (3NT)
EFRHDSGYEVHH

SEQ ID NO: 223 - protein, 709 aa, synthetic construct
  source 1..709: note = AB-6/TfRL10-1 - Anti-Abeta VH (WT huIgG1) + Knob Fc + Linker + scFv TfRL10-16 (2+1)
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMHWYQQKPGQAPRRLIYDTSNRATGIPARFSGSGSGTDFTLTISSLEPEDFAVYYCHQRSSYPWTFGGGTKVEIK

SEQ ID NO: 224 - protein, 457 aa, synthetic construct
  source 1..457: note = AB-6/TfRL10-16 - Anti-Abeta VH (WT huIgG1) + Hole Fc
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 225 - protein, 214 aa, synthetic construct
  source 1..214: note = AB-6/TfRL10-16 - Anti-Abeta VL + huKappa constant (LC)
DIQMTQSPSSLSASVGDRVTITCRASQSIRFYLNWYQQKPGKAPKVVIYGASSLQSGVPSRFSGSGSGTDFTLTISSLQPEDFATYYCQQTYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - protein, 709 aa, synthetic construct
  source 1..709: note = AB-7/TfRL10-16 - Anti-Abeta VH (WT huIgG1) + Knob Fc + Linker + scFv TfRL10-16 (2+1)
QLQLQESGPGLVKPSETLSLTCTVSGGSTSSSSYYWGWIRQPPGKGLEWIGSIYFSGSTYYNPSLKSRVTISVDTSKNQFSLKLSSVTAADTAVYYCVRHREMATIRDYHYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMHWYQQKPGQAPRRLIYDTSNRATGIPARFSGSGSGTDFTLTISSLEPEDFAVYYCHQRSSYPWTFGGGTKVEIK

SEQ ID NO: 227 - protein, 457 aa, synthetic construct
  source 1..457: note = AB-7/TfRL10-16 - Anti-Abeta VH (WT huIgG1) + Hole Fc
QLQLQESGPGLVKPSETLSLTCTVSGGSTSSSSYYWGWIRQPPGKGLEWIGSIYFSGSTYYNPSLKSRVTISVDTSKNQFSLKLSSVTAADTAVYYCVRHREMATIRDYHYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 228 - protein, 214 aa, synthetic construct
  source 1..214: note = AB-7/TfRL10-16 - Anti-Abeta VL + huKappa constant (LC)
DIQMTQSPSSLSASVGDRVTITCRASQSISNYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 704 aa, synthetic construct
  source 1..704: note = AB-8/TfRL10-16 - Anti-Abeta VH (WT huIgG1) + Knob Fc + Linker + scFv TfRL10-16 (2+1)
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMHWYQQKPGQAPRRLIYDTSNRATGIPARFSGSGSGTDFTLTISSLEPEDFAVYYCHQRSSYPWTFGGGTKVEIK

SEQ ID NO: 230 - protein, 452 aa, synthetic construct
  source 1..452: note = AB-8/TfRL10-16 - Anti-Abeta VH (WT huIgG1) + Hole Fc
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 231 - protein, 214 aa, synthetic construct
  source 1..214: note = AB-8/TfRL10-16 - Anti-Abeta VL + huKappa constant (LC)
DIQMTQSPSSLSASVGDRVTITCRASQSISNYLNWYQQKPGKAPKLLIYDAYSLQIGVPSRFSGSGSGTDFTLTISSLQPEDFATYYCQQSYSIPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 707 aa, synthetic construct
  source 1..707: note = AB-6/msTfR 8D3v12 - Anti-Abeta VH (WT huIgG1) + Knob Fc + Linker + scFv msTfR8D3v12 (2+1)
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGKGGSGGEVQLVESGGGLVQPGNSLTLSCVASGFTFSNYGMHWIRQAPKKGLEWIAAIYYDSSKMNYADTVKGRFTISRDNSKNTLYLEMNSLRSEDTAMYYCAVPTSHYVVDVWGQGVSVTVSSGGSEGKSSGSGSESKSTGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELK

SEQ ID NO: 233 - protein, 457 aa, synthetic construct
  source 1..457: note = AB-6/msTfR 8D3v12 - Anti-Abeta VH (WT huIgG1) + Hole Fc
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 234 - protein, 214 aa, synthetic construct
  source 1..214: note = AB-6/msTfR 8D3v12 - Anti-Abeta VL + huKappa constant (LC)
DIQMTQSPSSLSASVGDRVTITCRASQSIRFYLNWYQQKPGKAPKVVIYGASSLQSGVPSRFSGSGSGTDFTLTISSLQPEDFATYYCQQTYSTPITFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 235 - protein, 707 aa, synthetic construct
  source 1..707: note = AB-7/msTfR 8D3v12 - Anti-Abeta VH (WT huIgG1) + Knob Fc + Linker + scFv msTfR8D3v12 (2+1)
QLQLQESGPGLVKPSETLSLTCTVSGGSTSSSSYYWGWIRQPPGKGLEWIGSIYFSGSTYYNPSLKSRVTISVDTSKNQFSLKLSSVTAADTAVYYCVRHREMATIRDYHYYYMDVWGKGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGKGGSGGEVQLVESGGGLVQPGNSLTLSCVASGFTFSNYGMHWIRQAPKKGLEWIAAIYYDSSKMNYADTVKGRFTISRDNSKNTLYLEMNSLRSEDTAMYYCAVPTSHYVVDVWGQGVSVTVSSGGSEGKSSGSGSESKSTGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELK

SEQ ID NO: 236 - protein, 457 aa, synthetic construct
  source 1..457: note = AB-7/msTfR 8D3v12 - Anti-Abeta VH (WT huIgG1) + Hole Fc
QLQLQESGPGLVKPSETLSLTCTVSGGSTSSSSYYWGWIRQPPGKGLEWIGSIYFSGSTYYNPSLKSRVTISVDTSKNQFSLKLSSVTAADTAVYYCVRHREMATIRDYHYYYMDVWGKGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 237 - protein, 214 aa, synthetic construct
  source 1..214: note = AB-7/msTfR 8D3v12 - Anti-Abeta VL + huKappa constant (LC)
DIQMTQSPSSLSASVGDRVTITCRASQSISNYLNWYQQKPGKAPKLLIYAASSLQSGVPSRFSGSGSGTDFTLTISSLQPEDFATYYCQQSYSTPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 238 - protein, 702 aa, synthetic construct
  source 1..702: note = AB-8/msTfR 8D3v12 - Anti-Abeta VH (WT huIgG1) + Knob Fc + Linker + scFv msTfR8D3v12 (2+1)
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGKGGSGGEVQLVESGGGLVQPGNSLTLSCVASGFTFSNYGMHWIRQAPKKGLEWIAAIYYDSSKMNYADTVKGRFTISRDNSKNTLYLEMNSLRSEDTAMYYCAVPTSHYVVDVWGQGVSVTVSSGGSEGKSSGSGSESKSTGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELK

SEQ ID NO: 239 - protein, 452 aa, synthetic construct
  source 1..452: note = AB-8/msTfR 8D3v12 - Anti-Abeta VH (WT huIgG1) + Hole Fc
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPGK

SEQ ID NO: 240 - protein, 214 aa, synthetic construct
  source 1..214: note = AB-8/msTfR 8D3v12 - Anti-Abeta VL + huKappa constant (LC)
DIQMTQSPSSLSASVGDRVTITCRASQSISNYLNWYQQKPGKAPKLLIYDAYSLQIGVPSRFSGSGSGTDFTLTISSLQPEDFATYYCQQSYSIPPTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 241 - protein, 709 aa, synthetic construct
  source 1..709: note = AB-6 hIgG1 TfR.15.WH8.1 scFv 2+1 heavy chain knob
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 242 - protein, 709 aa, synthetic construct
  source 1..709: note = AB-6 hIgG1 TfR.15.WH8.1.42Q scFv 2+1 heavy chain knob
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMNWYQQKPGQAPRRLIYDTSNRATGIPARFSGSGSGTDFTLTISSLEPEDFAVYYCHQRSSYPWTFGGGTKVEIK

SEQ ID NO: 243 - protein, 709 aa, synthetic construct
  source 1..709: note = AB-6 hIgG1 TfR.15.WH8.1.42Q.H6-4 scFv 2+1 heavy chain knob
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGRFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 244 - protein, 709 aa, synthetic construct
  source 1..709: note = AB-6 hIgG1 TfR.15.WH8.1.42Q.L10-1 scFv 2+1 heavy chain knob
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMAWYQQKPGQAPRRLIYDTSNRATGIPARFSGSGSGTDFTLTISSLEPEDFAVYYCHQRSSYPWTFGGGTKVEIK

SEQ ID NO: 245 - protein, 709 aa, synthetic construct
  source 1..709: note = AB-6 hIgG1 TfR.15.WH8.1.42Q.L10-8 scFv 2+1 heavy chain knob
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMSWYQQKPGQAPRRLIYDTSNRATGIPARFSGSGSGTDFTLTISSLEPEDFAVYYCHQRSSYPWTFGGGTKVEIK

SEQ ID NO: 246 - protein, 709 aa, synthetic construct
  source 1..709: note = AB-6 hIgG1 TfR.15.WH8.1.24A scFv 2+1 heavy chain knob
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSAYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 247 - protein, 709 aa, synthetic construct
  source 1..709: note = AB-6 hIgG1 TfR.15.WH8.1.42Q.L7-2 scFv 2+1 heavy chain knob
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIDQMNWYQQKPGQAPRRLIYDTSNRATGIPARFSGSGSGTDFTLTISSLEPEDFAVYYCHQRSSYPWTFGGGTKVEIK

SEQ ID NO: 248 - protein, 713 aa, synthetic construct
  source 1..713: note = AB-6 hIgG1 TfR.9.1B.39.38 scFv 2+1 heavy chain knob
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTEYAMHWVRQAPGQGLEWMGGIAPNTGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEGGKTYLNWLQQRPGQSPRRLIYLVSKLDSGVPDRFSGSGSGTDFTLKISRVEAEDVGVYYCWQGTHFPQTFGGGTKVEIK

SEQ ID NO: 249 - protein, 713 aa, synthetic construct
  source 1..713: note = AB-6 hIgG1 TfR.9.1B.35 scFv2+1 heavy chain knob
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYHCWQGTHFPQSFGGGTKVEIK

SEQ ID NO: 250 - protein, 713 aa, synthetic construct
  source 1..713: note = AB-6 hIgG1 TfR.9.1B.39 scFv 2+1 heavy chain knob
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 251 - protein, 704 aa, synthetic construct
  source 1..704: note = AB-8 hIgG1 TfR.15.WH8.1 scFv 2+1 heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 252 - protein, 704 aa, synthetic construct
  source 1..704: note = AB-8 hIgG1 TfR.15.WH8.1.42Q scFv 2+1 heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMNWYQQKPGQAPRRLIYDTSNRATGIPARFSGSGSGTDFTLTISSLEPEDFAVYYCHQRSSYPWTFGGGTKVEIK

SEQ ID NO: 253 - protein, 704 aa, synthetic construct
  source 1..704: note = AB-8 hIgG1 TfR.15.WH8.1.42Q.H6-4 scFv 2+1 heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGRFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 254 - protein, 704 aa, synthetic construct
  source 1..704: note = AB-8 hIgG1 TfR.15.WH8.1.42Q.L10-1 scFv 2+1 heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMAWYQQKPGQAPRRLIYDTSNRATGIPARFSGSGSGTDFTLTISSLEPEDFAVYYCHQRSSYPWTFGGGTKVEIK

SEQ ID NO: 255 - protein, 704 aa, synthetic construct
  source 1..704: note = AB-8 hIgG1 TfR.15.WH8.1.42Q.L10-8 scFv 2+1 heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMSWYQQKPGQAPRRLIYDTSNRATGIPARFSGSGSGTDFTLTISSLEPEDFAVYYCHQRSSYPWTFGGGTKVEIK

SEQ ID NO: 256 - protein, 704 aa, synthetic construct
  source 1..704: note = AB-8 hIgG1 TfR.15.WH8.1.24A scFv 2+1 heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSAYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 257 - protein, 704 aa, synthetic construct
  source 1..704: note = AB-8 hIgG1 TfR.15.WH8.1.42Q.L7-2 scFv 2+1 heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIDQMNWYQQKPGQAPRRLIYDTSNRATGIPARFSGSGSGTDFTLTISSLEPEDFAVYYCHQRSSYPWTFGGGTKVEIK

SEQ ID NO: 258 - protein, 708 aa, synthetic construct
  source 1..708: note = AB-8 hIgG1 TfR9.1B.39.38 scFv 2+1 heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTEYAMHWVRQAPGQGLEWMGGIAPNTGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEGGKTYLNWLQQRPGQSPRRLIYLVSKLDSGVPDRFSGSGSGTDFTLKISRVEAEDVGVYYCWQGTHFPQTFGGGTKVEIK

SEQ ID NO: 259 - protein, 708 aa, synthetic construct
  source 1..708: note = AB-8 hIgG1 TfR.9.1B.35 scFv 2+1 heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKSSQSLLDSDGKTYLNWLQQRPGQSPRRLIYLVSKLDSGVPDRFSGSGSGTDFTLKISRVEAEDVGVYHCWQGTHFPQSFGGGTKVEIK

SEQ ID NO: 260 - protein, 708 aa, synthetic construct
  source 1..708: note = AB-8 hIgG1 TfR.9.1B.39 scFv 2+1 heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLLRGGDYWGQGTLVTVSSGGSEGKSSGSGSESKSTGGSDVVMTQSPLSLPVTLGQPASISCKASQSLLDEDGKTYLNWLQQRPGQSPRRLIYLVSKLDSGVPDRFSGSGSGTDFTLKISRVEAEDVGVYYCWQGTHFPQTFGGGTKVEIK

SEQ ID NO: 261 - protein, 699 aa, synthetic construct
  source 1..699: note = AB-8 hIgG1 TfR.15.WH8.1 scFv 2+1, no linker heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 262 - protein, 694 aa, synthetic construct
  source 1..694: note = AB-8 hIgG1 TfR.15.WH8.1 scFv 2+1, -5 linker heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGGGTKVEIK

SEQ ID NO: 263 - protein, 699 aa, synthetic construct
  source 1..699: note = AB-8 hIgG1 TfR.15.WH8.1 scFv 2+1, no linker, disulfide staple heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKQVQLVQSGAEVKKPGASVKVSCKASGYTFTSYYMHWVRQAPGQCLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRYMNWYQQKPGQAPRRLIYDTSNRATGIPARFSGSGSGTDFTLTISSLEPEDFAVYYCHQRSSYPWTFGCGTKVEIK

SEQ ID NO: 264 - protein, 699 aa, synthetic construct
  source 1..699: note = AB-8 hIgG1 TfR.15.WH8.1.42Q scFv 2+1 no linker heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKQVQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMNWYQQKPGQAPRRLIYDTSNRATGIPARFSGSGSGTDFTLTISSLEPEDFAVYYCHQRSSYPWTFGGGTKVEIK

SEQ ID NO: 265 - protein, 694 aa, synthetic construct
  source 1..694: note = AB-8 hIgG1 TfR.15.WH8.1.42Q scFV 2+1, -5 linker heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSQLVQSGAEVKKPGASVKVSCKASGYTFTSYYMHWVRQAPGQGLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMNWYQQKPGQAPRRLIYDTSNRATGIPARFSGSGSGTDFTLTISSLEPEDFAVYYCHQRSSYPWTFGGGTKVEIK

SEQ ID NO: 266 - protein, 699 aa, synthetic construct
  source 1..699: note = AB-8 hIgG1 TfR.15.WH8.1.42Q scFv 2+1 no linker, disulfide staple heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKQVQLVQSGAEVKKPGASVKVSCKASGYTFTSYYMHWVRQAPGQCLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMNWYQQKPGQAPRRLIYDTSNRATGIPARFSGSGSGTDFTLTISSLEPEDFAVYYCHQRSSYPWTFGCGTKVEIK

SEQ ID NO: 267 - protein, 330 aa, synthetic construct
  source 1..330: note = human IgG1 Fc polypeptide knob configuration
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 268 - protein, 330 aa, synthetic construct
  source 1..330: note = human IgG1 Fc polypeptide hole configuration
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 269 - protein, 121 aa, synthetic construct; identical to SEQ ID NO: 84
  source 1..121: note = TfR.15.WH8.1 VH sequence

SEQ ID NO: 270 - protein, 106 aa, synthetic construct; identical to SEQ ID NO: 82
  source 1..106: note = TfR.15.WH8.1 VL sequence

SEQ ID NO: 271 - protein, 119 aa, synthetic construct
  source 1..119: note = TfR.9.1B.35 VH sequence
QVQLVQSGAEVKKPGASVKVSCKASGYTFTEYAMHWVRQAPGQGLEWMGGINPNDGGTSYNQKFKGRVTMTVDTSTSTAYMELSSLRSEDTAVYYCASQGWLLRGGDYWGQGTLVTVSS

SEQ ID NO: 272 - protein, 112 aa, synthetic construct
  source 1..112: note = TfR.9.1B.35 VL sequence
DVVMTQSPLSLPVTLGQPASISCKSSQSLLDSDGKTYLNWLQQRPGQSPRRLIYLVSKLDSGVPDRFSGSGSGTDFTLKISRVEAEDVGVYHCWQGTHFPQSFGGGTKVEIK

SEQ ID NO: 273 - protein, 119 aa, synthetic construct; identical to SEQ ID NO: 271
  source 1..119: note = TfR.9.1B.39 VH sequence

SEQ ID NO: 274 - protein, 112 aa, synthetic construct; identical to SEQ ID NO: 94
  source 1..112: note = TfR.9.1B.39 VL sequence

SEQ ID NO: 275 - protein, 17 aa, synthetic construct
  source 1..17: note = CDR-H2 of TfR.9.1B.35 and TfR.9.1B.39
GINPNDGGTSYNQKFKG

SEQ ID NO: 276 - protein, 16 aa, synthetic construct
  source 1..16: note = CDR-L1 of TfR.9.1B.35
KSSQSLLDSDGKTYLN

SEQ ID NO: 277 - protein, 9 aa, synthetic construct
  source 1..9: note = CDR-L3 of TfR.9.1B.35
WQGTHFPQS

SEQ ID NO: 278 - protein, 709 aa, synthetic construct
  source 1..709: note = AB-6 hIgG1 TfR15.WH8.1.42Q..L10-16.dsFvHL heavy chain knob
QLQLQESGPGLVKPSETLSLTCTVSGGSISSGSYYWGWIRQPPGKGLEWIGSFYYSGTIYYNPSLKSRVTISVDTSKNQFSLKLSSVTAADTAVYYCARQREYSSSSNYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQCLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMHWYQQKPGQAPRRLIYDTSNRATGIPARFSGSGSGTDFTLTISSLEPEDFAVYYCHQRSSYPWTFGCGTKVEIK

SEQ ID NO: 279 - protein, 704 aa, synthetic construct
  source 1..704: note = AB-8 hIgG1 TfR.15.WH8.1.42Q.L10-16.dsFvHL heavy chain knob
EVQLVESGGGLIQPGGSLRLSCAASGFTVSSNYMSWVRQAPGKGLEWVSVIYSGGTKHYADSVKGRFTISRDNSKNTLYLQMNSLRAEDTAVYYCARSYYYGSGSSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGQVQLVQSGAEVKKPGASVKVSCKASGYTFTSYYMHWVRQAPGQCLEWMGNIYPGSGSSNYAQKFQGRVTMTRDTSTSTVYMELSSLRSEDTAVYYCTRSSYYGNFDTMDNWGQGTTVTVSSGGSEGKSSGSGSESKSTGGSEIVLTQSPATLSLSPGERATLSCRASSSIRQMHWYQQKPGQAPRRLIYDTSNRATGIPARFSGSGSGTDFTLTISSLEPEDFAVYYCHQRSSYPWTFGCGTKVEIK

SEQ ID NO: 280 - protein, 31 aa, synthetic construct
  source 1..31: note = aanti-pyroGlu3 Abeta antibody heavy chain Fc and anti-TfR scFv with 5 amino acid linker (GGSGG)
HEALHNHYTQKSLSLSPGKGGSGGQVQLVQS

SEQ ID NO: 281 - protein, 26 aa, synthetic construct
  source 1..26: note = anti-pyroGlu3 Abeta antibody heavy chain Fc and anti-TfR scFv with no linker
HEALHNHYTQKSLSLSPGKQVQLVQS

SEQ ID NO: 282 - protein, 21 aa, synthetic construct
  source 1..21: note = anti-pyroGlu3 Abeta antibody heavy chain Fc and anti-TfR scFv with -5 linker
HEALHNHYTQKSLSLSQLVQS